(19) United States
(12) Patent Application Publication
(10) Pub. No.: US 2026/0125391 A1
(43) Pub. Date: May 7, 2026

(54) PYRIMIDINE INDOLES AS CDK4 INHIBITORS

(71) Applicant: Incyte Corporation, Wilmington, DE (US)

(72) Inventors: Peter Niels Carlson, Claymont, DE (US); Liana Hie, Wilmington, DE (US); Joshua Hummel, Hockessin, DE (US); Jacob J. Lacharity, Chesterbrook, PA (US); Xiaolei Li, New Castle, DE (US); Sharada Manns, Wilmington, DE (US); Eli Ortiz, Glen Mills, PA (US); Robert Susick, Wilmington, DE (US); Xiaozhao Wang, Moorestown, NJ (US); Bo Wei, Claymont, DE (US)

(21) Appl. No.: 19/378,405

(22) Filed: Nov. 4, 2025

Related U.S. Application Data
(60) Provisional application No. 63/716,039, filed on Nov. 4, 2024.
(60) Provisional application No. 63/727,965, filed on Dec. 4, 2024.
(60) Provisional application No. 63/754,288, filed on Feb. 5, 2025.
(60) Provisional application No. 63/855,722, filed on Aug. 1, 2025.

(51) Int. Cl. C07D 491/107; A61K 31/506; A61K 45/06; C07B 59/00; C07D 401/14; C07D 405/14; C07D 487/10

(52) U.S. Cl. CPC C07D 491/107; A61K 31/506; A61K 45/06; C07B 59/002; C07D 401/14; C07D 405/14; C07D 487/10; C07B 2200/05

(57) ABSTRACT

The present application provides compounds of Formula (I):

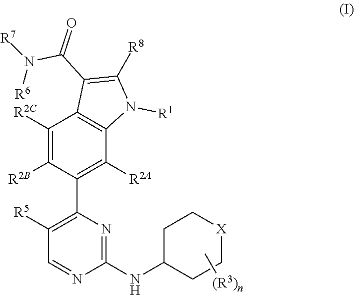

or a pharmaceutically acceptable salt thereof, that are inhibitors of cyclin-dependent kinase 4 (CDK4), as well as pharmaceutical compositions thereof, and methods of treating cancer using the same.

### TECHNICAL FIELD

This application is directed to pyrimidine indoles which inhibit cyclin-dependent kinase 4 (CDK4) and are useful for treating cancer.

### BACKGROUND

Cyclin-dependent kinases (CDKs) are a family of serine/threonine kinases. Heterodimerized with regulatory subunits known as cyclins, CDKs become fully activated and regulate key cellular processes including cell cycle progression and cell division (Morgan, D. O. Annu. Rev. Cell Dev. Biol. 1997, 13:261-291). Uncontrolled proliferation is a hallmark of cancer cells. The deregulation of the CDK activity is associated with abnormal regulation of cell-cycle and is detected in virtually all forms of human cancers (Sherr, C. J. and Roberts, J. M., Genes Dev. 1999, 13(12): 1501-1512).

CDK4, in complex with cyclin D, is crucial for the transition from the G1 phase to the S phase of the cell cycle. The availability and synthesis of cyclin D are influenced by extracellular signals such as growth factors and mitogens (Sherr, C. J. and Roberts, J. M., Genes Dev. 1999, 13(12): 1501-1512; and Malumbres, M. et al. Nat. Rev. Cancer 2009, 9(3): 153-166). This transition is essential for DNA replication and cell division. One of the primary targets of the CDK4/cyclin D complex is the retinoblastoma protein (Rb). In its hypophosphorylated form, Rb binds to and inhibits E2F transcription factors (Weinberg, R. A. Cell 1995, 81(3): 323-330; and Dyson, N., Genes Dev. 1998, 12(15): 2245-2262). CDK4/cyclin D phosphorylates Rb, leading to its inactivation. Phosphorylation of retinoblastoma protein (Rb) by the CDK4/cyclin D complex leads to the release of E2F transcription factors, which promote the expression of genes required for DNA synthesis and S phase entry, such as those coding for cyclin E and other proteins involved in DNA replication (Harbour, J. W. and Dean, D. C., Genes Dev., 2000, 14(19): 2393-2409; Nevins, J. R., Hum. Mol. Genet., 2001, 10(7): 699-703). The action of CDK4/cyclin D ensure that once the cell passes the G1/S checkpoint, it is committed to completing the cell cycle, thus ensuring controlled and regulated cell division.

Overexpression or amplification of CDK4 can lead to abnormal activity, resulting in uncontrolled cell proliferation, a hallmark of cancer. The CDK4-cyclin D pathway is often deregulated in many cancers, leading to excessive cell division and tumor growth. Dysregulation of CCND1 in cancer can occur through gene amplification, chromosomal translocations, mutations in regulatory pathways, miRNA alterations, and changes in protein stability and degradation (Swerdlow, S. H. et al., Hum. Pathol., 1995 26(9): 999-1004; Knudsen, E. S. and Wang, J. Y, Mol. Cell Biol., 1997, 17(10): 5771-5783; Asghar, U., A. K. et al., Nat. Rev. Drug Discov., 2015, 14(2): 130-146; Zheng, N., Z. Wang and Wei, W., Int. J. Biochem. Cell Biol., 2016, 73: 99-110; and Lines, K. E. et al., J. Endocrinol., 2018, 240(1): 41-50). Additionally, mutations or alterations in the Rb pathway can enhance CDK4 activity, further contributing to cancer development (Weinberg, R. A. Cell 1995, 81(3): 323-330; Sherr, C. J. and Roberts, J. M., Genes Dev. 1999, 13(12): 1501-1512; Harbour, J. W. and Dean, D. C., Genes Dev., 2000, 14(19): 2393-2409; Nevins, J. R., Hum. Mol. Genet., 2001, 10(7): 699-703).

Due to its central role in cell cycle regulation, CDK4 is a target for cancer therapy. CDK4/6 dual inhibitors, such as palbociclib, ribociclib, and abemaciclib, have been developed and approved for the treatment of certain cancers, including breast cancer. These inhibitors work by blocking the kinase activity of CDK4/6, blocking cell cycle progression, and inducing senescence thereby inhibiting tumor growth (Asghar, U., A. K. et al., Nat. Rev. Drug Discov., 2015, 14(2): 130-146).

Further, a compound which is selective for CDK4 over CDK6 can be useful to minimize dose limiting toxicity. In particular, CDK4 is the primary oncogenic driver in certain breast cancers, the co-inhibition of CDK6-cyclin D3 can result in dose limiting toxicities due to associated hematological adverse events (Maurer, B. et al., Haematologica., 2021, 106(10): 2624-2632). Neutropenia is reported as the most common adverse events for CDK4/6 inhibitors, particularly palbociclib and ribociclib, and to a lesser extent abemaciclib (Liu, Y et al., BMC Cancer, 2023, 23(1): 816). Despite the clinical success of CDK4/6 inhibitors, such dose limiting toxicities limit the dosing regimen thus limiting effective target coverage. A selective CDK4 inhibitor would facilitate higher target coverage, thus maximizing efficacy while also minimizing the CDK6-associated toxicity profile, thus improving overall safety and tolerability. A next-generation CDK4 inhibitor could have clinical benefit in CDK4/6 refractory patients and could address acquired resistance to standard of care. Additionally, functional inhibition of CDK4, through a CDK4 molecule, has clinical opportunity in other indications besides breast cancers. CCND1 dysregulated cancers such as sarcomas, melanoma, head & neck, etc. could potentially benefit from a CDK4 inhibitor, either as a single agent or in combination with their respective standard of care.

These data provide a rationale for considering CDK4 as a potential target for new drug development in cancer associated with CDK4 and cyclin D expression and activity. In the last decade there has been increasing interest in the development of CDK selective inhibitors and there remains a need to discover CDK inhibitors having novel activity profiles, in particular those targeting CDK4 that spare off-target CDKs with associated toxicities, including CDK6 and CDK1 associated hematopoietic and/or gastrointestinal toxicities. This application is directed to this need and others.

### SUMMARY

The present invention relates to, inter alia, compounds of Formula (I):

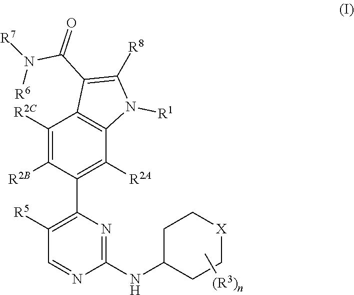

or pharmaceutically acceptable salts thereof, wherein the constituent members are defined herein.

The present invention further provides pharmaceutical compositions comprising a compound described herein, or a pharmaceutically acceptable salt thereof, and a pharmaceutically acceptable carrier.

The present invention further provides methods of inhibiting, comprising contacting the CDK4 with a compound described herein, or a pharmaceutically acceptable salt thereof.

The present invention further provides methods of inhibiting CDK4 in a patient, comprising administering to the patient a compound described herein, or a pharmaceutically acceptable salt thereof.

The present invention further provides methods of treating a disease or disorder associated with CDK4 in a patient, comprising administering to the patient a compound described herein, or a pharmaceutically acceptable salt thereof.

The present invention further provides compounds described herein, or a pharmaceutically acceptable salt thereof, for use in any of the methods described herein.

The present invention further provides uses of a compound described herein, or a pharmaceutically acceptable salt thereof, for the preparation of a medicament for use in any of the methods described herein.

### DETAILED DESCRIPTION

The present application provides, inter alia, a compound of Formula (I):

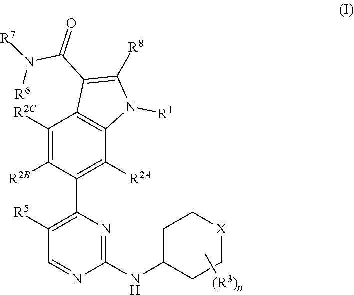

or a pharmaceutically acceptable salt thereof, wherein:
n is 0, 1, 2, 3, 4, 5, or 6;
X is O, S, S(O)₂, S(O)NR^{c}, or N(-L-R⁴);
L is S(O)₂, S(O)(═NR^{b}), or S(O)₂NR^{a};
R^{a}, R^{b}, and R^{c} are each independently selected from H, C₁₋₆ alkyl and C₁₋₆ haloalkyl; or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
R¹ is selected from C₁₋₆ alkyl, C₁₋₆ haloalkyl, C₃₋₁₀ cycloalkyl, 6-14 membered aryl, 4-15 membered heterocycloalkyl, 5-14 membered heteroaryl, C₃₋₁₀ cycloalkyl-C₁₋₄ alkyl, 6-14 membered aryl-C₁₋₄ alkyl, 4-15 membered heterocycloalkyl-C₁₋₄ alkyl, and 5-14 membered heteroaryl-C₁₋₄ alkyl, which are each optionally substituted with 1, 2, 3, or 4 independently selected R^{1A} substituents;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹) and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{C11} and R^{d11}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{e12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{e12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12} NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12}, is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{2A}, R^{2B}, and R^{2C} are each independently selected from H, D, halo, CN, C₁₋₆ alkyl, C₁₋₆ haloalkyl, C₃₋₇ cycloalkyl, phenyl, 4-8 membered heterocycloalkyl, 5-6 membered heteroaryl, C₃-7 cycloalkyl-C₁₋₄ alkyl, phenyl-C₁₋₄ alkyl, 4-8 membered heterocycloalkyl-C₁₋₄ alkyl, 5-6 membered heteroaryl-C₁₋₄ alkyl, OR^{a2}, SR^{a2}, NHOR^{a2}, C(O)R^{b2}, C(O)NR^{c2}R^{d2}, C(O)NR^{e2}(OR^{a2}), C(O)OR^{a2}, OC(O)R^{b2}, OC(O)NR^{c2}R^{d2}, NR^{c2}R^{d2} NR^{c2}NR^{c2}R^{d2} NR^{c2}C(O)R^{b2} NR^{c2}C(O)OR^{a2} NR^{c2}C(O)NR^{c2}R^{d2}, C(═NR^{e2})R^{b2}, C(═NR^{e2})R^{c2}R^{d2} NR^{c2}C(═NR^{e2})NR^{c2}R^{d2} NR^{c2}C(═NR^{e2})R^{b2} NR^{c2}S(O)NR^{c2}R^{d2} NR²S(O)R^{b2} NR²S(O)₂R^{b2} NR^{c2}S(O)(═NR^{e2})R^{b2} NR^{c2}S(O)₂NR^{c2}R^{d2}, S(O)R^{b2}, S(O)NR^{e2}R^{d2}, S(O)₂R^{b2}, and S(O)₂NR^{c2}R^{d2}, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a2}, R^{e2}, and R^{d2} is independently selected from H, C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c2} and R^{d2} attached to the same N atom, together with the N atom to which they are attached, form a 4-8 membered heterocycloalkyl group, which is optionally substituted with 1, 2, 3, or 4 independently selected R^{G} substituents;
each R^{b2} is independently selected from C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e2} is independently selected from H, OH, CN, C₁₋₆ alkyl, C₁₋₆ alkoxy, C₁₋₆ haloalkyl, and C₁₋₆ haloalkoxy;
each R³ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3} NR^{c3}C(O)NR^{c3}R^{d3}, C(═NR^{e3})R^{b3}, C(═NR^{e3})NR^{c3}R^{d3} NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; wherein said C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, NHOR^{a41}, C(O)R^{b41}, C(O)NR^{c41}R^{d41}, C(O)NR^{c41}(OR^{a41}) C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41} NR^{c41}C(═NR^{e41})R^{b41}, NR^{c41}S(O)NR^{c41}R^{d41}, NR^{c41}S(O)R^{b41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, and OS(O)₂R^{b41}, S(O)(═NR^{e41})R^{b41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{4B} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})R^{c42}R^{d42}, NR^{c42}C(═NR^{e42})R^{b42}, NR^{c42}S(O)NR^{c42}R^{d42}, NR^{c42}S(O)R^{b42}, NR^{c42}S(O)₂R^{b42}, NR^{e42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, and OS(O)₂R^{b42}, S(O)(═NR^{e42})R^{b42}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b42} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
R⁵ is selected from halo, CN, C₁₋₄ alkyl, C₁₋₄ haloalkyl, C₂₋₄ alkenyl, C₂₋₄ alkynyl, C₁₋₃ alkoxy, C₁₋₃ haloalkoxy, amino, C₁₋₃ alkylamino, di(C₁₋₃ alkyl)amino, cyano-C₁₋₄ alkyl, HO—C₁₋₄ alkyl, C₁₋₃ alkoxy-C₁₋₄ alkyl, and C₃₋₄ cycloalkyl;
R⁶ is selected from C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, 4, 5, or 6 independently selected R^{6A} substituents;
R⁷ is selected from H, C₁₋₆ alkyl, C₂₋₆ alkenyl, C₂₋₆ alkynyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or R⁶ and R⁷, together with the nitrogen atom to which they are attached, form a 4-15 membered heterocycloalkyl moiety or a 5-10 membered heteroaryl moiety, each of which is optionally substituted by 1, 2, 3, 4, 5, or 6 independently selected R^{6A} substituents;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61} NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R^{d61}, NR^{c61}R^{d61}, NR^{c61}NR^{c61}R^{c61} NR^{c61}C(O)R^{b61}, NR^{c61}C(O)OR^{a61}, NR^{c61}C(O)NR^{c61}R^{d61}, C(═NR^{e61})R^{b61}, C(═NR^{e61})NR^{c61}R^{d61}, NR^{c61}C(═NR^{e61})R^{e61}R^{d61}, NR^{e61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, OS(O)(═NR^{e61})R^{b61}, OS(O)₂R^{b61}, S(O)(═NR^{e61})R^{b61}, SF₅, P(O)R^{f61}R^{g61}, OP(O)(OR^{h61})(ORⁱ⁶¹), P(O)(OR^{h61})(ORⁱ⁶¹) and BR^{j61}R^{k61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, which is optionally substituted with 1, 2, 3, or 4 independently selected R⁶¹ substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{e61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j61} and R^{k61} is independently selected from OH, C₁₋₆ alkoxy, and C₁₋₆ haloalkoxy; or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a62}, SR^{a62}, NHOR^{a62}, C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62})NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62} OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6C} substituents;
or, any R^{e62} and R^{d62}, attached to the same N atom, together with the N atom to which they are attached, form a 4-7 membered heterocycloalkyl group, which is optionally substituted with 1, 2, 3, or 4 independently selected R^{6C} substituents;
each R^{b62}, is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6C} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{e62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j62} and R^{k62} is independently selected from OH, C₁₋₆ alkoxy, and C₁₋₆ haloalkoxy; or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a63}, SR^{a63}, NHOR^{a63}, C(O)R^{b63}, C(O)NR^{c63}R^{d63}, C(O)NR^{c63}(OR^{a63}), C(O)OR^{a63}, OC(O)R^{b63}, OC(O)NR^{c63}R^{d63}, NR^{c63}R^{d63}, NR^{c63}NR^{c63}R^{d63}, NR^{c63}C(O)R^{b63}, NR^{c63}C(O)OR^{a63}, NR^{c63}C(O)NR^{e63}R^{d63}, C(═NR^{e63})R^{b63}, C(═NR^{e63})NR^{c63}R^{d63}, NR^{c63}C(═NR^{e63})NR^{c63}R^{d63}, NR^{c63}C(═NR^{e63})R^{b63}, NR^{c63}S(O)NR^{c63}R^{d63}, NR^{c63}S(O)R^{b63}, NR^{c63}S(O)₂R^{b63}, NR^{c63}S(O)(═NR^{e63})R^{b63}, NR^{c63}S(O)₂NR^{c63}R^{d63}, S(O)R^{b63}, S(O)NR^{c63}R^{d63}, S(O)₂R^{b63}, S(O)₂NR^{c63}R^{d63}, OS(O)(═NR^{e63})R^{b63}, OS(O)₂R^{b63}, S(O)(═NR^{e63})R^{b63}, SF₅, P(O)R^{f63}R^{g63}, OP(O)(OR^{h63})(ORⁱ⁶³), P(O)(OR^{h63})(ORⁱ⁶³) and BR^{j63}R^{k63}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a63}, R^{c63}, and R^{d63}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c63} and R^{d63}, attached to the same N atom, together with the N atom to which they are attached, form a 4-7 membered heterocycloalkyl group, which is optionally substituted with 1, 2, 3, or 4 independently selected R^{G} substituents;
each R^{b63} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e63} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f63} and R^{g63} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h63} and Rⁱ⁶³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j63} and R^{k63} is independently selected from OH, C₁₋₆ alkoxy, and C₁₋₆ haloalkoxy;
or any R^{j63} and R^{k63} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁸ is selected from H, D, halo, CN, C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8} NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{c8} S(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a8}, R^{c8}, and R^{d8} is independently selected from H, C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c8} and R^{d8} attached to the same N atom, together with the N atom to which they are attached, form a 4-8 membered heterocycloalkyl group, which is optionally substituted with 1, 2, 3, or 4 independently selected R^{G} substituents;
each R^{b8} is independently selected from C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, and C₁₋₆ haloalkoxy; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, n is 0, 1, or 2.

In some embodiments, n is 1 or 2.

In some embodiments, n is 0.

In some embodiments, n is 1.

In some embodiments, n is 2.

In some embodiments, X is O, S(O)₂, or N(-L-R⁴).

In some embodiments, X is O.

In some embodiments, X is S.

In some embodiments, X is S(O)₂.

In some embodiments, X is S(O)NR^{c}.

In some embodiments, X is N(-L-R⁴).

In some embodiments, L is S(O)₂.

In some embodiments, L is S(O)₂NR^{a}, wherein R^{a} is H, C₁₋₆ alkyl, or C₁₋₆ haloalkyl, or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents.

In some embodiments, R¹ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents.

In some embodiments, R¹ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents.

In some embodiments, R¹ is selected from C₁₋₆ alkyl, C₁₋₆ haloalkyl, C₃₋₁₀ cycloalkyl, and 4-10 membered heterocycloalkyl, which are each optionally substituted with 1, 2, 3, or 4 independently selected R^{1A} substituents.

In some embodiments, R¹ is selected from C₁₋₆ alkyl, C₁₋₆ haloalkyl, and C₃₋₁₀ cycloalkyl, which are each optionally substituted with 1, 2, 3, or 4 independently selected R^{1A} substituents.

In some embodiments, R¹ is selected from C₁₋₆ alkyl, C₁₋₆ haloalkyl, C₃₋₇ cycloalkyl, and 4-7 membered heterocycloalkyl, which are each optionally substituted with 1, 2, 3, or 4 independently selected R^{1A} substituents.

In some embodiments, R¹ is selected from C₁₋₆ alkyl, C₁₋₆ haloalkyl, and C₃₋₇ cycloalkyl, which are each optionally substituted with 1, 2, 3, or 4 independently selected R^{1A} substituents.

In some embodiments, R¹ is selected from C₁₋₆ alkyl and C₁₋₆ haloalkyl, which are each optionally substituted with 1, 2, 3, or 4 independently selected R^{1A} substituents.

In some embodiments, R¹ is C₃₋₇ cycloalkyl, which is optionally substituted with 1, 2, 3, or 4 independently selected R^{1A} substituents.

In some embodiments, R¹ is selected from C₁₋₆ alkyl, C₁₋₆ haloalkyl, and C₃₋₇ cycloalkyl.

In some embodiments, R¹ is C₁₋₆ alkyl.

In some embodiments, R¹ is C₃₋₇ cycloalkyl.

In some embodiments:
each R^{1A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹¹ substituents;
each R^{a11}, R^{c11}, and R^{d11}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{e12}R^{d12}, C(O)OR^{a12}, OC(O)R^{b12} OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; and
each R^{b12}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents.

In some embodiments:
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{e12}R^{d12}, C(O)OR^{a12}, OC(O)R^{b12} OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl; and
each R^{b12}, is independently selected from C₁₋₆ alkyl and C₁₋₆ haloalkyl.

In some embodiments:
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{a11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{1B} substituents;
each R^{b11} is independently selected from C₁₋₆ alkyl and C₁₋₆ haloalkyl, which are each optionally substituted with 1 or 2 independently selected R^{1B} substituents;
each R^{1B} is independently selected from H, D, halo, CN, C₁₋₆ alkyl, C₁₋₆ haloalkyl, OR^{a12} SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12} NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl; and
each R^{b12}, is independently selected from C₁₋₆ alkyl and C₁₋₆ haloalkyl.

In some embodiments:
each R^{1A} is independently selected from H, D, halo, CN, C₁₋₆ alkyl, C₁₋₆ haloalkyl, C₃₋₅ cycloalkyl, 4-5 membered heterocycloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11}, is independently selected from H, C₁₋₆ alkyl, and C₁₋₆ haloalkyl; and
each R^{b11} is independently selected from C₁₋₆ alkyl and C₁₋₆ haloalkyl.

In some embodiments:
each R^{1A} is independently selected from H, D, halo, CN, C₁₋₆ alkyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{b11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}; and
each R^{a11}, R^{c11}, and R^{d11}, is independently selected from H, C₁₋₆ alkyl, and C₁₋₆ haloalkyl; each R^{b11} is independently selected from C₁₋₆ alkyl and C₁₋₆ haloalkyl.

In some embodiments:
each R^{1A} is independently selected from H, D, halo, CN, C₁₋₆ alkyl, C₁₋₆ haloalkyl, OR^{a11}, and NR^{c11}R^{d11}; and
each R^{a11}, R^{c11}, and R^{d11}, is independently selected from H, C₁₋₆ alkyl, and C₁₋₆ haloalkyl.

In some embodiments, R^{2A}, R^{2B}, and R^{2C} are each independently selected from H, D, halo, CN, C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, 5-6 membered heteroaryl-C₁₋₄ alkyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2} NR^{c2}C(O)NR^{c2}R^{d2} NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents.

In some embodiments, R^{2A}, R^{2B}, and R^{2C} are each independently selected from H, D, halo, CN, C₁₋₆ alkyl, C₁₋₆ haloalkyl, C(O)R^{b2}, C(O)NR^{c2}R^{d2}, C(O)OR^{a2}, OC(O)R^{b2}, OC(O)NR^{c2}R^{d2}, NR^{c2}R^{d2} NR^{c2}C(O)R^{b2} NR^{c2}C(O)OR^{a2}, NR^{c2}C(O)NR^{c2}R^{d2} NR^{c2}S(O)₂R^{b2} NR^{e2}S(O)₂NR^{e2}R^{d2}, S(O)₂R^{b2}, and S(O)₂NR^{c2}R^{d2}, wherein said C₁₋₆ alkyl and C₁₋₆ haloalkyl are each optionally substituted with 1 or 2 independently selected R^{G} substituents.

In some embodiments, R^{2A}, R^{2B}, and R^{2C} are each independently selected from H, D, halo, CN, C₁₋₆ alkyl, and C₁₋₆ haloalkyl.

In some embodiments, R^{2A}, R^{2B}, and R^{2C} are each independently selected from H, halo, CN, C₁₋₃ alkyl, and C₁₋₃ haloalkyl.

In some embodiments, R^{2A}, R^{2B}, and R^{2C} are each independently selected from H, halo, and CN.

In some embodiments, R^{2A}, R^{2B}, and R^{2C} are each independently selected from H, halo, and OR^{a2}.

In some embodiments, R^{2A}, R^{2B}, and R^{2C} are each independently selected from H and halo.

In some embodiments, R^{2A}, R^{2B}, and R^{2C} are each independently selected from H and F.

In some embodiments, R^{2A} is H or halo; R^{2B} is H; and R^{2C} is halo.

In some embodiments, R^{2A} is H or F; R^{2B} is H; and R^{2C} is F.

In some embodiments:
each R^{a2}, R^{c2}, and R^{d2} is independently selected from H, C₁₋₆ alkyl, and C₁₋₆ haloalkyl; and
each R^{b2} is independently selected from C₁₋₆ alkyl and C₁₋₆ haloalkyl.

In some embodiments, each R³ is independently selected from D, halo, CN, C₁₋₆ alkyl, C₁₋₆ haloalkyl, C₃₋₄ cycloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3} NR^{c3}C(O)OR^{a3}, NR^{c3}C(O)NR^{c3}R^{d3}, NR^{c3}S(O)₂R^{b3}, NR^{c3}S(O)₂NR^{c3}R^{d3} S(O)₂R^{b3}, and S(O)₂NR^{c3}R^{d3}, wherein said C₁₋₆ alkyl, C₁₋₆ haloalkyl, and C₃₋₄ cycloalkyl are each optionally substituted with 1 or 2 independently selected R^{G} substituents.

In some embodiments, each R³ is independently selected from D, halo, CN, C₁₋₆ alkyl, C₁₋₆ haloalkyl, OR^{a3}, and NR^{c3}R^{d3}, wherein said C₁₋₆ alkyl and C₁₋₆ haloalkyl are each optionally substituted with 1 or 2 independently selected R^{G} substituents.

In some embodiments, each R³ is independently selected from halo, CN, C₁₋₃ alkyl, C₁₋₃ haloalkyl, OR^{a3}, and NR^{c3}R^{d3}, wherein said C₁₋₃ alkyl and C₁₋₃ haloalkyl are each optionally substituted with 1 or 2 independently selected R^{G} substituents.

In some embodiments, each R^{a3}, R^{c3}, and R^{d3} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{G} substituents; and each R^{b3} is independently selected from C₁₋₆ alkyl and C₁₋₆ haloalkyl, which are each optionally substituted with 1, 2, 3, or 4 independently selected R^{G} substituents.

In some embodiments, each R^{a3}, R^{c3}, and R^{d3} is independently selected from H, C₁₋₆ alkyl, and C₁₋₆ haloalkyl; and each R^{b3} is independently selected from C₁₋₆ alkyl and C₁₋₆ haloalkyl.

In some embodiments, each R³ is independently selected from —C₁₋₄ alkylene-OR^{a3}, —C₁₋₄ alkylene-NR^{c3}R^{d3}, OR^{a3}, and NR^{c3}R^{d3}.

In some embodiments, each R³ is OH.

In some embodiments, R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents.

In some embodiments, R⁴ is selected from C₁₋₆ alkyl and C₁₋₆ haloalkyl; wherein said C₁₋₆ alkyl and C₁₋₆ haloalkyl are each optionally substituted by 1, 2, 3, or 4 independently selected R^{4A} substituents.

In some embodiments, R⁴ is selected from C₁₋₆ alkyl and C₁₋₆ haloalkyl.

In some embodiments:
each R^{4A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41} OC(O)NR^{c41}R^{d41}, NR^{c41}R^{d41} NR^{c41}C(O)R^{b41}, NR^{c41}C(O)OR^{a41} NR^{c41}C(O)NR^{c41}R^{d41} NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{e42}R^{d42}, NR^{c42}S(O)₂R^{b42}, NR^{c42}S(O)₂NR^{c42}R^{d42}, S(O)₂R^{b42}, and S(O)₂NR^{c42}R^{d42}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; and
each R^{b42} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents.

In some embodiments:
each R^{4A} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41} OC(O)NR^{c41}R^{d41}, NR^{c41}R^{d41} NR^{c41}C(O)R^{b41}, NR^{c41}C(O)OR^{a41} NR^{c41}C(O)NR^{c41}R^{d41} NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁴¹ substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from D, halo, CN, C₁₋₆ alkyl, C₁₋₆ haloalkyl, OR^{a42} SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR⁴²S(O)₂NR^{c42}R^{d42}, S(O)₂R^{b42}, and S(O)₂NR^{c42}R^{d42};
each R^{a42}, R^{c42}, and R^{d42}, is independently selected from H, C₁₋₆ alkyl, and C₁₋₆ haloalkyl; and each R^{b42} is independently selected from C₁₋₆ alkyl and C₁₋₆ haloalkyl.

In some embodiments:
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R⁴¹, and S(O)₂NR^{c41}R^{d4}1, wherein said C₁₋₆ alkyl and C₁₋₆ haloalkyl are each optionally substituted with 1, 2, 3, or 4 independently selected R^{4B} substituents;
each R^{a41}, R^{c41}, and R^{d41} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{4B} substituents;
each R^{b41} is independently selected from C₁₋₆ alkyl and C₁₋₆ haloalkyl, which are each optionally substituted with 1, 2, 3, or 4 independently selected R^{4B} substituents;
each R^{4B} is independently selected from D, halo, CN, C₁₋₆ alkyl, C₁₋₆ haloalkyl, OR^{a42} SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR⁴²S(O)₂NR^{c42}R^{d42}, S(O)₂R^{b42}, and S(O)₂NR^{c42}R^{d42};
each R^{a42}, R^{c42}, and R^{d42}, is independently selected from H, C₁₋₆ alkyl, and C₁₋₆ haloalkyl; and
each R^{b42} is independently selected from C₁₋₆ alkyl and C₁₋₆ haloalkyl.

In some embodiments:
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41};
each R^{a41}, R^{c41}, and R^{d41} is independently selected from H, C₁₋₆ alkyl, and C₁₋₆ haloalkyl; and
each R^{b41} is independently selected from C₁₋₆ alkyl and C₁₋₆ haloalkyl.

In some embodiments, R⁵ is selected from halo, CN, C₁₋₄ alkyl, C₁₋₄ haloalkyl, cyano-C₁₋₄ alkyl, HO—C₁₋₄ alkyl, C₁₋₃ alkoxy-C₁₋₄ alkyl, and C₃₋₄ cycloalkyl.

In some embodiments, R⁵ is selected from halo, CN, C₁₋₄ alkyl, and C₁₋₄ haloalkyl.

In some embodiments, R⁵ is selected from halo and CN.

In some embodiments, R⁵ is selected from F, Cl, and CN.

In some embodiments, R⁵ is halo.

In some embodiments, R⁵ is F.

In some embodiments, R⁵ is Cl.

In some embodiments, R⁵ is CN.

In some embodiments:

R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl, each of which are optionally substituted by 1, 2, 3, or 4 independently selected R^{G} substituents;
or R⁶ and R⁷, together with the nitrogen atom to which they are attached, form a 4-15 membered heterocycloalkyl moiety or a 5-10 membered heteroaryl moiety, each of which is optionally substituted by 1, 2, 3, 4, 5, or 6 independently selected R^{6A} substituents.

In some embodiments:
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl;
or R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents.

In some embodiments:
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents; and
R⁷ is selected from H, C₁₋₆ alkyl, and C₁₋₆ haloalkyl, each of which are optionally substituted by 1, 2, 3, or 4 independently selected R^{G} substituents.

In some embodiments:
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents; and
R⁷ is selected from H, C₁₋₆ alkyl, and C₁₋₆ haloalkyl.

In some embodiments, each R^{6A} is independently selected from halo, CN, C₁₋₆ alkyl, C₁₋₆ haloalkyl, C₃₋₁₀ cycloalkyl, 6-10 membered aryl, 4-10 membered heterocycloalkyl, 5-10 membered heteroaryl, OR^{a61}, NR^{c61}R^{d61}, and S(O)₂R^{b61}, wherein said C₁₋₆ alkyl, C₁₋₆ haloalkyl, C₃₋₁₀ cycloalkyl, 6-10 membered aryl, 4-10 membered heterocycloalkyl, and 5-10 membered heteroaryl are each optionally substituted with 1, 2, 3, or 4 independently selected R^{6B} substituents.

In some embodiments, each R^{6A} is independently selected from halo, CN, C₁₋₃ alkyl, C₁₋₃ haloalkyl, C₃₋₇ cycloalkyl, 4-6 membered heterocycloalkyl, 5-6 membered heteroaryl, OR^{a61} NR^{c61}R⁶¹, and S(O)₂R^{b61}, wherein said C₁₋₃ alkyl, C₃₋₇ cycloalkyl, and 5-6 membered heteroaryl are each optionally substituted with 1, 2, or 3 independently selected R^{6B} substituents.

In some embodiments:
each R^{6A} is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61} NR^{c61}C(O)R^{b61}, NR^{c61}C(O)OR^{a61}, NR^{c61}C(O)NR^{c61}R^{d61}, NR^{e61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, which is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{e62}R^{d62}, NR^{c62}R^{d62}, NR^{c62}C(O)R^{b62}, NR^{c62}C(O)OR^{a62} NR^{c62}C(O)NR^{c62}R^{d62}, NR^{e62}S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a63}, SR^{a63}, C(O)R^{b63}, C(O)NR^{c63}R^{d63}, C(O)OR^{a63}, OC(O)R^{b63}, OC(O)NR⁶³R^{d63}, NR^{c63}R^{d63}, NR^{c63}C(O)R^{b63}, NR^{c63}C(O)OR^{a63} NR^{c63}C(O)NR^{c63}R^{d63}, NR^{c63}S(O)₂R^{b63}, NR^{c63}S(O)₂NR^{c63}R^{d63}, S(O)₂R^{b63}, and S(O)₂NR^{c63}R^{d63}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a63}, R^{c63}, and R^{d63}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; and
each R^{b63} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents.

In some embodiments:
each R^{6A} is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61}, C(O)R^{b61}, C(O)NR^{c61}R^{a61}, C(O)OR^{a61}, OC(O)R^{b61}, OC(O)NR^{c61}R^{d61}, NR^{e61}R^{d61}, NR^{c61}C(O)R^{b61}, NR^{c61}C(O)OR^{a61}, NR^{c61}C(O)NR^{c61}R^{d61}, NR^{e61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR⁶²S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, OR^{a63} SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63}, NR^{c63}C(O)NR^{c63}R^{d63}, NR^{c63}S(O)₂R^{b63}, NR^{c63}S(O)₂NR^{c63}R^{d63}, S(O)₂R^{b63}, and S(O)₂NR^{c63}R^{d63};
each R^{a63}, R^{c63}, and R^{d63}, is independently selected from H, C₁₋₆ alkyl, and C₁₋₆ haloalkyl; and each R^{b63} is independently selected from C₁₋₆ alkyl and C₁₋₆ haloalkyl.

In some embodiments:
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a6}1 NR^{c61}C(O)NR^{e61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{e62}R^{d62}, NR^{c62}R^{d62}, NR^{c62}C(O)R^{b62}, NR^{c62}C(O)OR^{a62} NR^{c62}C(O)NR^{e62}R^{d62}, NR^{e62}S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, OR^{a63}, SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63}, NR^{c63}C(O)NR^{c63}R^{d63}, NR^{c63}S(O)₂R^{b63}, NR^{c63}S(O)₂NR^{c63}R^{d63}, S(O)₂R^{b63}, and S(O)₂NR^{c63}R^{d63};
each R^{a63}, R^{c63}, and R^{d63}, is independently selected from H, C₁₋₆ alkyl, and C₁₋₆ haloalkyl; and
each R^{b63} is independently selected from C₁₋₆ alkyl and C₁₋₆ haloalkyl.

In some embodiments:
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a6}1 NR^{c61}C(O)NR^{e61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{e62}R^{d62}, NR^{c62}S(O)₂R^{b62}, NR^{c62}S(O)₂NR^{e62}R^{d62}, S(O)₂R^{b62}, and S(O)₂NR^{c62}R^{d62};
each R^{a62}, R^{c62}, and R^{d62}, is independently selected from H, C₁₋₆ alkyl, and C₁₋₆ haloalkyl; and
each R^{b62}, is independently selected from C₁₋₆ alkyl and C₁₋₆ haloalkyl.

In some embodiments:
each R^{6A} is independently selected from D, halo, CN, C₁₋₆ alkyl, C₁₋₆ haloalkyl, C₃₋₅ cycloalkyl, 4-5 membered heterocycloalkyl, OR^{a61}, SR^{a61}, C(O)R^{b61}, C(O)NR^{e61}R^{a61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₅ cycloalkyl, and 4-5 membered heterocycloalkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₃₋₅ cycloalkyl, and 4-5 membered heterocycloalkyl, wherein said C₁₋₆ alkyl, C₁₋₆ haloalkyl, C₁₋₆ alkyl, C₁₋₆ haloalkyl, C₃₋₅ cycloalkyl, and 4-5 membered heterocycloalkyl are each optionally substituted with 1 or 2 independently selected R^{6B} substituents;
each R^{b61} is independently selected from C₁₋₆ alkyl, C₁₋₆ haloalkyl, C₁₋₆ alkyl, C₁₋₆ haloalkyl, C₃₋₅ cycloalkyl, and 4-5 membered heterocycloalkyl, which are each optionally substituted with 1 or 2 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{e62}R^{d62}, S(O)₂R^{b62}, and S(O)₂NR^{e62}R^{d62};
each R^{a62}, R^{c62}, and R^{d62}, is independently selected from H, C₁₋₆ alkyl, and C₁₋₆ haloalkyl; and
each R^{b62}, is independently selected from C₁₋₆ alkyl and C₁₋₆ haloalkyl.

In some embodiments:
each R^{6A} is independently selected from halo, CN, C₁₋₆ alkyl, C₁₋₆ haloalkyl, C₃₋₅ cycloalkyl, 4-5 membered heterocycloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₅ cycloalkyl, and 4-5 membered heterocycloalkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₃₋₅ cycloalkyl, and 4-5 membered heterocycloalkyl, wherein said C₁₋₆ alkyl, C₁₋₆ haloalkyl, C₁₋₆ alkyl, C₁₋₆ haloalkyl, C₃₋₅ cycloalkyl, and 4-5 membered heterocycloalkyl are each optionally substituted with 1 or 2 independently selected R^{6B} substituents;
each R^{b61} is independently selected from C₁₋₆ alkyl, C₁₋₆ haloalkyl, C₁₋₆ alkyl, C₁₋₆ haloalkyl, C₃₋₅ cycloalkyl, and 4-5 membered heterocycloalkyl, which are each optionally substituted with 1 or 2 independently selected R^{6B} substituents; and
each R^{6B} is independently selected from OH, CN, halo, C₁₋₃ alkyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, C₁₋₃ alkylsulfonyl, carbamyl, C₁₋₃ alkylcarbamyl, di(C₁₋₃ alkyl)carbamyl, C₁₋₃ alkylcarbonyl, C₁₋₃ alkoxycarbonyl, C₁₋₃ alkylcarbonyloxy, and C₁₋₃ alkylcarbonylamino.

In some embodiments:
each R^{6A} is independently selected from halo, CN, C₁₋₆ alkyl, and C₁₋₆ haloalkyl, wherein said C₁₋₆ alkyl and C₁₋₆ haloalkyl, are each optionally substituted with 1 or 2 independently selected R^{6B} substituents; and
each R^{6B} is independently selected from OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino.

In some embodiments:
each R^{6A} is independently selected from D, halo, CN, C₁₋₆ alkyl, C₁₋₆ haloalkyl, C₃₋₁₀ cycloalkyl, 6-10 membered aryl, 4-10 membered heterocycloalkyl, 5-10 membered heteroaryl, OR^{a61}, NR^{c61}R^{d61} and S(O)₂R^{b61}, wherein said C₁₋₆ alkyl, C₁₋₆ haloalkyl, C₃₋₁₀ cycloalkyl, 6-10 membered aryl, 4-10 membered heterocycloalkyl, and 5-10 membered heteroaryl are each optionally substituted with 1, 2, 3, or 4 independently selected R^{6B} substituents;
each R^{a61}, R^{c61}, and R^{d61} is independently selected from H, C₁₋₆ alkyl, and C₁₋₆ haloalkyl;
each R^{b61} is independently selected from C₁₋₆ alkyl, C₁₋₆ haloalkyl, and C₃₋₁₀ cycloalkyl;
each R^{6B} is independently selected from D, halo, CN, C₁₋₆ alkyl, C₁₋₆ haloalkyl, C₃₋₁₀ cycloalkyl, 6-10 membered aryl, 4-10 membered heterocycloalkyl, 5-10 membered heteroaryl, OR^{a62}, NR^{e62}R^{d62}, and S(O)₂R^{b62};
each R^{a62}, R^{c62}, and R^{d62}, is independently selected from H, C₁₋₆ alkyl, C₁₋₆ haloalkyl, C₃₋₁₀ cycloalkyl, 6-10 membered aryl, 4-10 membered heterocycloalkyl, and 5-10 membered heteroaryl; and
each R^{b62}, is independently selected from C₁₋₆ alkyl and C₁₋₆ haloalkyl.

In some embodiments:
each R^{6A} is independently selected from halo, CN, C₁₋₆ alkyl, C₁₋₆ haloalkyl, C₃₋₁₀ cycloalkyl, 4-10 membered heterocycloalkyl, 5-10 membered heteroaryl, OR^{a61}, NR^{c61}R^{d61}, and S(O)₂R^{b61}, wherein said C₁₋₆ alkyl, C₃₋₁₀ cycloalkyl, and 5-10 membered heteroaryl are each optionally substituted with 1, 2, or 3 independently selected R⁶¹ substituents;
each R^{a61}, R^{c61}, and R^{d61} is independently selected from H, C₁₋₆ alkyl, and C₁₋₆ haloalkyl;
each R^{b61} is independently selected from C₁₋₆ alkyl and C₃₋₁₀ cycloalkyl;
each R^{6B} is independently selected from D, halo, CN, C₁₋₆ alkyl, OR^{a62}, and NR^{c62}R^{d62}; and
each R^{a62}, R^{c62}, and R^{d62}, is independently selected from H, C₁₋₆ alkyl, and C₁₋₆ haloalkyl.

In some embodiments, R⁸ is selected from H, D, halo, CN, C₁₋₆ alkyl, C₁₋₆ haloalkyl, C₃₋₅ cycloalkyl, 4-6 membered heterocycloalkyl, OR^{a8}, SR^{a8}, C(O)R^{b8}, C(O)NR^{c8}R^{d8}, C(O)OR^{a8}, OC(O)R^{b8}, OC(O)NR^{c8}R^{d8}, NR^{c8}R^{d8} NR^{c8}C(O)R^{b8} NR^{c8}C(O)OR^{a8}, NR^{c8}C(O)NR^{c8}R^{d8}, NR^{c8}S(O)₂R^{b8}, NR^{c8}S(O)₂NR^{c8}R^{d8}, S(O)₂R^{b8}, and S(O)₂NR^{c8}R^{d8}, wherein said C₁₋₆ alkyl, C₁₋₆ haloalkyl, C₃₋₅ cycloalkyl, and 4-6 membered heterocycloalkyl are each optionally substituted with 1, 2, 3, or 4 independently selected R^{G} substituents.

In some embodiments, R⁸ is selected from H, D, halo, CN, C₁₋₆ alkyl, and C₁₋₆ haloalkyl, wherein said C₁₋₆ alkyl and C₁₋₆ haloalkyl are each optionally substituted with 1 or 2 independently selected R^{G} substituents.

In some embodiments, R⁸ is selected from H, D, halo, CN, C₁₋₆ alkyl, and C₁₋₆ haloalkyl.

In some embodiments, R⁸ is selected from H, D, halo, CN, C₁₋₃ alkyl, and C₁₋₃ haloalkyl.

In some embodiments, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents.

In some embodiments, R⁶ and R⁷, together with the nitrogen atom to which they are attached, form a 4-6 membered heterocycloalkyl moiety or a 5-6 membered heteroaryl moiety, each of which is optionally substituted by 1, 2, 3, or 4 independently selected R^{6A} substituents.

In some embodiments, R⁶ and R⁷, together with the nitrogen atom to which they are attached, form a 4-membered heterocycloalkyl, which is optionally substituted by 1, 2, 3, or 4 independently selected R^{6A} substituents.

In some embodiments, R⁶ and R⁷, together with the nitrogen atom to which they are attached, form a 5-membered heterocycloalkyl moiety, which is optionally substituted by 1, 2, 3, or 4 independently selected R^{6A} substituents.

In some embodiments, R⁶ and R⁷, together with the nitrogen atom to which they are attached, form a 6-membered heterocycloalkyl moiety, which is optionally substituted by 1, 2, 3, or 4 independently selected R^{6A} substituents.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents.

In some embodiments, each R^{6A} is independently selected from halogen, C₁₋₆ alkyl, and C₁₋₆ haloalkyl, wherein said C₁₋₆ alkyl and C₁₋₆ haloalkyl are each optionally substituted with 1, 2, 3, or 4 substituents selected from D, halo, CN, NO₂, and OH.

In some embodiments, each R^{6A} is independently selected from from F, methyl, —CF₃, —CH₂OH, and —CD₂OH.

In some embodiments, each R^{6A} is independently selected from halogen and —CH₂OH.

In some embodiments, each R^{6A} is independently selected from F and —CH₂OH.

In some embodiments, each R^{6A} is independently selected from Cl and —CH₂OH.

In some embodiments, each R^{6A} is independently selected from halogen and —CH₂OH, wherein the-CH₂OH is attached in the (S)-configuration.

In some embodiments, each R^{6A} is independently selected from F and —CH₂OH, wherein the-CH₂OH is attached in the (S)-configuration.

In some embodiments, each R^{6A} is independently selected from Cl and —CH₂OH, wherein the-CH₂OH is attached in the (S)-configuration.

In some embodiments, each R^{6A} is independently selected from F, methyl, —CF₃, —CH₂OH, and —CD₂OH, wherein the —CH₂OH and —CD₂OH are attached in the (S)-configuration.

In some embodiments, at least one R^{6A} is —CH₂OH.

In some embodiments, at least one R^{6A} is —CD₂OH.

In some embodiments, at least one R^{6A} is —CH₂OH attached in the (S)-configuration.

In some embodiments, at least one R^{6A} is —CD₂OH attached in the (S)-configuration.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from halogen and —CH₂OH.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from halogen, C₁₋₆ alkyl, and C₁₋₆ haloalkyl, wherein said C₁₋₆ alkyl and C₁₋₆ haloalkyl are each optionally substituted with 1, 2, 3, or 4 substituents selected from D, halo, CN, NO₂, and OH.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from F and —CH₂OH.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents selected from from F, methyl, —CF₃, —CH₂OH, and —CD₂OH.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from C₁ and —CH₂OH.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from halogen and —CH₂OH, wherein the-CH₂OH is attached in the (S)-configuration.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from halogen, C₁₋₆ alkyl, and C₁₋₆ haloalkyl, wherein said C₁₋₆ alkyl and C₁₋₆ haloalkyl are each optionally substituted with 1, 2, 3, or 4 substituents selected from D, halo, CN, NO₂, and OH, wherein the —CH₂OH and —CD₂OH are attached in the (S)-configuration.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from F and —CH₂OH, wherein the-CH₂OH is attached in the (S)-configuration.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from F, methyl, —CF₃, —CH₂OH, and —CD₂OH, wherein the —CH₂OH and —CD₂OH are attached in the (S)-configuration.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and each R^{6A} is independently selected from C₁ and —CH₂OH, wherein the-CH₂OH is attached in the (S)-configuration.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and at least one R^{6A} is selected from —CH₂OH, wherein the-CH₂OH is attached in the (S)-configuration.

In some embodiments, R⁶ and R⁷, together with the nitrogen atom to which they are attached, form a 4-6 membered heterocycloalkyl moiety, which is optionally substituted by 1, 2, 3, or 4 independently selected R^{6A} substituents; and at least one R^{6A} is —CD₂OH, wherein the —CD₂OH is attached in the (S)-configuration.

In some embodiments:
each R^{a8}, R^{c8}, and R^{d8} is independently selected from H, C₁₋₆ alkyl, C₁₋₆ haloalkyl, C₃₋₅ cycloalkyl, and 4-5 membered heterocycloalkyl, wherein said C₁₋₆ alkyl, C₁₋₆ haloalkyl, C₃₋₅ cycloalkyl, and 4-5 membered heterocycloalkyl are each optionally substituted with 1, 2, 3, or 4 independently selected R^{G} substituents; and
each R^{b8} is independently selected from C₁₋₆ alkyl, C₁₋₆ haloalkyl, C₃₋₅ cycloalkyl, and 4-5 membered heterocycloalkyl, which are each optionally substituted with 1, 2, 3, or 4 independently selected R^{G} substituents.

In some embodiments:
each R^{a8}, R^{c8}, and R^{d8} is independently selected from H, C₁₋₆ alkyl, and C₁₋₆ haloalkyl; and
each R^{b8} is independently selected from C₁₋₆ alkyl and C₁₋₆ haloalkyl.

In some embodiments:
n is 0, 1, or 2;
X is O, S(O)₂, or N(-L-R⁴);
L is S(O)₂ or S(O)₂NR^{a};
R^{a} is selected from H, C₁₋₆ alkyl and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
R¹ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{e12}R^{d12}, C(O)OR^{a12}, OC(O)R^{b12} OC(O)NR^{c12}R^{d12}, NR^{c12}R^{d12}, NR^{c12}C(O)R^{b12}, NR^{c12}C(O)OR^{a12} NR^{c12}C(O)NR^{c12}R^{d12}, NR^{c12}S(O)₂R^{b12}, NR¹²S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl;
each R^{b12}, is independently selected from C₁₋₆ alkyl and C₁₋₆ haloalkyl;
R^{2A}, R^{2B}, and R^{2C} are each independently selected from H, D, halo, CN, C₁₋₆ alkyl, C₁₋₆ haloalkyl, OR^{a2}, C(O)R^{b2}, C(O)NR^{c2}R¹², C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl and C₁₋₆ haloalkyl are each optionally substituted with 1 or 2 independently selected R^{G} substituents;
each R^{a2}, R^{c2}, and R^{d2} is independently selected from H, C₁₋₆ alkyl, and C₁₋₆ haloalkyl;
each R^{b2} is independently selected from C₁₋₆ alkyl and C₁₋₆ haloalkyl;
each R³ is independently selected from D, halo, CN, C₁₋₆ alkyl, C₁₋₆ haloalkyl, OR^{a3}, and NR^{c3}R^{d3} wherein said C₁₋₆ alkyl and C₁₋₆ haloalkyl are each optionally substituted with 1 or 2 independently selected R^{G} substituents;
each R^{a3}, R^{c3}, and R^{d3} is independently selected from H, C₁₋₆ alkyl, and C₁₋₆ haloalkyl;
each R^{b3} is independently selected from C₁₋₆ alkyl and C₁₋₆ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41} OC(O)NR^{c41}R^{d41}, NR^{c41}R^{d41} NR^{c41}C(O)R^{b41}, NR^{c41}C(O)OR^{a41} NR^{c41}C(O)NR^{c41}R^{d41} NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from D, halo, CN, C₁₋₆ alkyl, C₁₋₆ haloalkyl, OR^{a42} SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR^{c42}S(O)₂NR^{c42}R^{d42}, S(O)₂R^{b42}, and S(O)₂NR^{c42}R^{d42};
each R^{a42}, R^{c42}, and R^{d42}, is independently selected from H, C₁₋₆ alkyl, and C₁₋₆ haloalkyl;
each R^{b42} is independently selected from C₁₋₆ alkyl and C₁₋₆ haloalkyl;
R⁵ is selected from halo, CN, C₁₋₄ alkyl, and C₁₋₄ haloalkyl;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61} NR^{c61}C(O)R^{b61}, NR^{c61}C(O)OR^{a61}, NR^{c61}C(O)NR^{c61}R^{d61}, NR^{e61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R⁶¹ substituents;
each R^{a61}, R^{c61} and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, which is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{e62}R^{d62}, NR^{c62}R^{d62}, NR^{c62}C(O)R^{b62}, NR^{c62}C(O)OR^{a62} NR^{c62}C(O)NR^{e62}R^{d62}, NR^{c62}S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a63}, SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63} NR^{c63}C(O)NR^{c63}R^{d63}, NR^{c63}S(O)₂R^{b63} NR^{c63}S(O)₂NR^{c63}R^{d63}, S(O)₂R^{b63}, and S(O)₂NR^{c63}R^{d63}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a63}, R^{c63}, and R^{d63}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; and
each R^{b63} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R⁸ is selected from H, D, halo, CN, C₁₋₆ alkyl, and C₁₋₆ haloalkyl, wherein said C₁₋₆ alkyl and C₁₋₆ haloalkyl are each optionally substituted with 1 or 2 independently selected R^{G} substituents; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0, 1, or 2;
X is O, S(O)₂, or N(-L-R⁴);
L is S(O)₂ or S(O)₂NR^{a};
R^{a} is selected from H, C₁₋₆ alkyl and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
R¹ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{e12}R^{d12}, C(O)OR^{a12}, OC(O)R^{b12} OC(O)NR^{c12}R^{d12}, NR^{c12}R^{d12}, NR^{c12}C(O)R^{b12}, NR^{e12}C(O)OR^{a12}, NR^{e12}C(O)NR^{e12}R^{d12}, NR^{c12}S(O)₂R^{b12}, NR¹²S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl;
each R^{b12}, is independently selected from C₁₋₆ alkyl and C₁₋₆ haloalkyl;
R^{2A}, R^{2B}, and R^{2C} are each independently selected from H, D, halo, CN, C₁₋₆ alkyl, C₁₋₆ haloalkyl, C(O)R^{b2}, C(O)NR^{c2}R¹², C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl and C₁₋₆ haloalkyl are each optionally substituted with 1 or 2 independently selected R^{G} substituents;
each R³ is independently selected from D, halo, CN, C₁₋₆ alkyl, C₁₋₆ haloalkyl, OR^{a3}, and NR^{c3}R^{d3} wherein said C₁₋₆ alkyl and C₁₋₆ haloalkyl are each optionally substituted with 1 or 2 independently selected R^{G} substituents;
each R^{a3}, R^{c3}, and R^{d3} is independently selected from H, C₁₋₆ alkyl, and C₁₋₆ haloalkyl;
each R^{b3} is independently selected from C₁₋₆ alkyl and C₁₋₆ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41} OC(O)NR^{c41}R^{d41}, NR^{c41}R^{d41} NR^{c41}C(O)R^{b41}, NR^{c41}C(O)OR^{a41} NR^{c41}C(O)NR^{c41}R^{d41} NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from D, halo, CN, C₁₋₆ alkyl, C₁₋₆ haloalkyl, OR^{a42} SR^{a42}, C(O)R^{b42}, C(O)NR^{c42}R⁴², C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{e42}S(O)₂R^{b42}, NR⁴²S(O)₂NR^{c42}R^{d42}, S(O)₂R^{b42}, and S(O)₂NR^{c42}R^{d42};
each R^{a42}, R^{c42}, and R^{d42}, is independently selected from H, C₁₋₆ alkyl, and C₁₋₆ haloalkyl;
each R^{b42} is independently selected from C₁₋₆ alkyl and C₁₋₆ haloalkyl;
R⁵ is selected from halo, CN, C₁₋₄ alkyl, and C₁₋₄ haloalkyl;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl;
or R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61} NR^{c61}C(O)R^{b61}, NR^{c61}C(O)OR^{a61}, NR^{c61}C(O)NR^{e61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, which is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{e62}R^{d62}, NR^{c62}R^{d62}, NR^{c62}C(O)R^{b62}, NR^{c62}C(O)OR^{a62} NR^{c62}C(O)NR^{e62}R^{d62}, NR^{c62}S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a63}, SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63} NR^{c63}C(O)NR^{c63}R^{d63}, NR^{c63}S(O)₂R^{b63} NR^{c63}S(O)₂NR^{c63}R^{d63}, S(O)₂R^{b63}, and S(O)₂NR^{c63}R^{d63}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a63}, R^{c63}, and R^{d63}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; and
each R^{b63} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R⁸ is selected from H, D, halo, CN, C₁₋₆ alkyl, and C₁₋₆ haloalkyl, wherein said C₁₋₆ alkyl and C₁₋₆ haloalkyl are each optionally substituted with 1 or 2 independently selected R^{G} substituents; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0, 1, or 2;
X is O, S(O)₂, or N(-L-R⁴);
L is S(O)₂ or S(O)₂NR^{a};
R^{a} is selected from H, C₁₋₆ alkyl and C₁₋₆ haloalkyl;
R¹ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{1B} substituents;
each R^{b11} is independently selected from C₁₋₆ alkyl and C₁₋₆ haloalkyl, which are each optionally substituted with 1 or 2 independently selected R^{1B} substituents;
each R^{1B} is independently selected from H, D, halo, CN, C₁₋₆ alkyl, C₁₋₆ haloalkyl, OR^{a12} SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12} NR¹²C(O)NR^{c12}R^{d12}, NR^{e12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl;
each R^{b12}, is independently selected from C₁₋₆ alkyl and C₁₋₆ haloalkyl;
R^{2A}, R^{2B}, and R^{2C} are each independently selected from H, D, halo, CN, C₁₋₆ alkyl, C₁₋₆ haloalkyl, and OR^{a2};
each R^{a2} is independently selected from H and C₁₋₆ alkyl;
each R³ is independently selected from halo, CN, C₁₋₃ alkyl, C₁₋₃ haloalkyl, OR^{a3}, and NR^{c3}R^{d3} wherein said C₁₋₃ alkyl and C₁₋₃ haloalkyl are each optionally substituted with 1 or 2 independently selected R^{G} substituents.
each R^{a3}, R^{c3}, and R^{d3} is independently selected from H, C₁₋₆ alkyl, and C₁₋₆ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41};
each R^{a41}, R^{c41} and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from halo and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{e61}R^{d61}, C(O)OR^{a61}, OC(O)R^{b61}, OC(O)NR^{e61}R^{d61}, NR^{c61}R^{d61} NR^{c61}C(O)R^{b61}, NR^{c61}C(O)OR^{a6}1 NR^{c61}C(O)NR^{e61}R^{d61}, NR^{c61}S(O)₂R^{b61}, NR^{c61}S(O)₂NR^{c61}R^{c61}, S(O)₂R^{b61}, and S(O)₂NR^{c61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{a61}, R^{c61}, and R^{d61}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{e62}R^{d62}, NR^{c62}S(O)₂R^{b62}, NR^{c62}S(O)₂NR^{c62}R^{c62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, OR^{a63}, SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63} NR^{c63}C(O)NR^{c63}R^{d63}, NR^{c63}S(O)₂R^{b63}, NR^{c63}S(O)₂NR^{c63}R^{d63}, S(O)₂R^{b63}, and S(O)₂NR^{c63}R^{d63};
each R^{a63}, R^{c63}, and R^{d63}, is independently selected from H, C₁₋₆ alkyl, and C₁₋₆ haloalkyl;
each R^{b63} is independently selected from C₁₋₆ alkyl and C₁₋₆ haloalkyl;
R⁸ is selected from H, D, halo, CN, C₁₋₆ alkyl, and C₁₋₆ haloalkyl; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0, 1, or 2;
X is O, S(O)₂, or N(-L-R⁴);
L is S(O)₂ or S(O)₂NR^{a};
R^{a} is selected from H, C₁₋₆ alkyl and C₁₋₆ haloalkyl;
R¹ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{1B} substituents;
each R^{b11} is independently selected from C₁₋₆ alkyl and C₁₋₆ haloalkyl, which are each optionally substituted with 1 or 2 independently selected R¹¹ substituents;
each R^{1B} is independently selected from H, D, halo, CN, C₁₋₆ alkyl, C₁₋₆ haloalkyl, OR^{a12} SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl;
each R^{b12}, is independently selected from C₁₋₆ alkyl and C₁₋₆ haloalkyl;
R^{2A}, R^{2B}, and R^{2C} are each independently selected from H, D, halo, CN, C₁₋₆ alkyl, and C₁₋₆ haloalkyl;
each R³ is independently selected from halo, CN, C₁₋₃ alkyl, C₁₋₃ haloalkyl, OR^{a3}, and NR^{c3}R^{d3} wherein said C₁₋₃ alkyl and C₁₋₃ haloalkyl are each optionally substituted with 1 or 2 independently selected R^{G} substituents.
each R^{a3}, R^{c3}, and R^{d3} is independently selected from H, C₁₋₆ alkyl, and C₁₋₆ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41};
each R^{a41}, R^{c41} and R^{d41} is independently selected from H, C₁₋₆ alkyl, and C₁₋₆ haloalkyl;
each R^{b41} is independently selected from C₁₋₆ alkyl and C₁₋₆ haloalkyl
R⁵ is selected from halo and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl;
or R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a6}1 NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{e61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{c62}R^{c62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, OR^{a63}, SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63}, NR^{c63}C(O)NR^{e63}R^{d63}, NR^{c63}S(O)₂R^{b63}, NR^{c63}S(O)₂NR^{c63}R^{d63}, S(O)₂R^{b63}, and S(O)₂NR^{c63}R^{d63};
each R^{a63}, R^{c63}, and R^{d63}, is independently selected from H, C₁₋₆ alkyl, and C₁₋₆ haloalkyl;
each R^{b63} is independently selected from C₁₋₆ alkyl and C₁₋₆ haloalkyl;
R⁸ is selected from H, D, halo, CN, C₁₋₆ alkyl, and C₁₋₆ haloalkyl; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0 or 1;
X is O, S(O)₂, or N(-L-R⁴);
L is S(O)₂;
R¹ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₅ cycloalkyl, 4-5 membered heterocycloalkyl, OR^{a11}, SR^{a11}, C(O)R^{b11}, C(O)NR^{c11}R^{b11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, each R^{a11}, R^{c11}, and R^{d11}, is independently selected from H, C₁₋₆ alkyl, and C₁₋₆ haloalkyl;
each R^{b11} is independently selected from C₁₋₆ alkyl and C₁₋₆ haloalkyl;
R^{2A}, R^{2B}, and R^{2C} are each independently selected from H, halo, CN, and OR^{a2};
each R^{a2} is independently selected from H and C₁₋₆ alkyl;
each R³ is independently selected from halo, CN, C₁₋₃ alkyl, C₁₋₃ haloalkyl, OR^{a3}, and NR^{c3}R^{d3} wherein said C₁₋₃ alkyl and C₁₋₃ haloalkyl are each optionally substituted a substituent selected from OH, C₁₋₃ alkoxy, amino, C₁₋₃ alkylamino, and di(C₁₋₃ alkyl)amino;
each R^{a3}, R^{c3}, and R^{d3} is independently selected from H, C₁₋₃ alkyl, and C₁₋₃ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41},
each R^{a41}, R^{c41}, and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from F, Cl, and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d6}1 NR^{c61}C(O)R^{b61}, NR^{c61}C(O)OR^{a6}1 NR^{c61}C(O)NR^{e61}R^{d61}, NR^{e61}S(O)₂R^{b61}, NR^{e61}S(O)₂NR^{e61}R^{d61}, S(O)₂R^{b61}, and S(O)₂NR^{e61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61} and R^{d61} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62};
each R^{a62}, R^{c62}, and R^{d62}, is independently selected from H, C₁₋₆ alkyl, and C₁₋₆ haloalkyl;
each R^{b62}, is independently selected from C₁₋₆ alkyl and C₁₋₆ haloalkyl; and
R⁸ is selected from H, D, halo, CN, C₁₋₃ alkyl, and C₁₋₃ haloalkyl.

In some embodiments:
n is 0 or 1;
X is O, S(O)₂, or N(-L-R⁴);
L is S(O)₂;
R¹ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₅ cycloalkyl, 4-5 membered heterocycloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11},
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R^{2A}, R^{2B}, and R^{2C} are each independently selected from H, halo, and CN;
each R³ is independently selected from halo, CN, C₁₋₃ alkyl, C₁₋₃ haloalkyl, OR^{a3}, and NR^{c3}R^{d3} wherein said C₁₋₃ alkyl and C₁₋₃ haloalkyl are each optionally substituted a substituent selected from OH, C₁₋₃ alkoxy, amino, C₁₋₃ alkylamino, and di(C₁₋₃ alkyl)amino;
each R^{a3}, R^{c3}, and R^{d3} is independently selected from H, C₁₋₃ alkyl, and C₁₋₃ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41},
each R^{a41}, R^{c41}, and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from F, Cl, and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl;
or R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a6}1 NR^{c61}C(O)NR^{e61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62};
each R^{a62}, R^{c62}, and R^{d62}, is independently selected from H, C₁₋₆ alkyl, and C₁₋₆ haloalkyl;
each R^{b62}, is independently selected from C₁₋₆ alkyl and C₁₋₆ haloalkyl; and
R⁸ is selected from H, D, halo, CN, C₁₋₃ alkyl, and C₁₋₃ haloalkyl.

In some embodiments:
n is 0 or 1;
X is O, S(O)₂, or N(-L-R⁴);
L is S(O)₂;
R¹ is selected from C₁₋₆ alkyl, C₁₋₆ haloalkyl, C₃₋₁₀ cycloalkyl, and 4-10 membered heterocycloalkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11}, is independently selected from H, C₁₋₆ alkyl, and C₁₋₆ haloalkyl;
each R^{b11} is independently selected from C₁₋₆ alkyl and C₁₋₆ haloalkyl;
R^{2A}, R^{2B}, and R^{2C} are each independently selected from H, halo, and OR^{a2};
each R^{a2} is C₁₋₆ alkyl;
each R³ is independently selected from —C₁₋₄ alkylene-OR^{a3}, —C₁₋₄ alkylene-NR^{c3}R^{d3} OR^{a3}, and NR^{c3}R^{d3};
each R^{a3} is H;
each R^{c3} and R^{d3} is independently selected from H and C₁₋₃ alkyl;
R⁴ is selected from C₁₋₆ alkyl and C₁₋₆ haloalkyl, wherein said C₁₋₆ alkyl and C₁₋₆ haloalkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41},
each R^{a41}, R^{c41}, and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from F, Cl, and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₅ cycloalkyl, 4-5 membered heterocycloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₅ cycloalkyl, and 4-5 membered heterocycloalkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₃₋₅ cycloalkyl, and 4-5 membered heterocycloalkyl, wherein said C₁₋₆ alkyl, C₁₋₆ haloalkyl, C₁₋₆ alkyl, C₁₋₆ haloalkyl, C₃₋₅ cycloalkyl, and 4-5 membered heterocycloalkyl are each optionally substituted with 1 or 2 independently selected R^{6B} substituents;
each R^{b61} is independently selected from C₁₋₆ alkyl, C₁₋₆ haloalkyl, C₁₋₆ alkyl, C₁₋₆ haloalkyl, C₃₋₅ cycloalkyl, and 4-5 membered heterocycloalkyl, which are each optionally substituted with 1 or 2 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62};
each R^{a62}, R^{c62}, and R^{d62}, is independently selected from H, C₁₋₆ alkyl, and C₁₋₆ haloalkyl;
each R^{b62}, is independently selected from C₁₋₆ alkyl and C₁₋₆ haloalkyl; and
R⁸ is selected from H, C₁₋₃ alkyl, and C₁₋₃ haloalkyl.

In some embodiments:
n is 0 or 1;
X is O, S(O)₂, or N(-L-R⁴);
L is S(O)₂;
R¹ is selected from C₁₋₆ alkyl, C₁₋₆ haloalkyl, C₃₋₁₀ cycloalkyl, and 4-10 membered heterocycloalkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11}, is independently selected from H, C₁₋₆ alkyl, and C₁₋₆ haloalkyl;
each R^{b11} is independently selected from C₁₋₆ alkyl and C₁₋₆ haloalkyl;
R^{2A}, R^{2B}, and R^{2C} are each independently selected from H and halo;
each R³ is independently selected from —C₁₋₄ alkylene-OR^{a3}, —C₁₋₄ alkylene-NR^{c3}R^{d3} OR^{a3}, and NR^{c3}R^{d3};
each R^{a3} is H;
each R^{c3} and R^{d3} is independently selected from H and C₁₋₃ alkyl;
R⁴ is selected from C₁₋₆ alkyl and C₁₋₆ haloalkyl; wherein said C₁₋₆ alkyl and C₁₋₆ haloalkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R⁴¹, and S(O)₂NR^{c41}R^{d41},
each R^{a41}, R^{c41}, and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from F, Cl, and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl;
or R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₅ cycloalkyl, 4-5 membered heterocycloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₅ cycloalkyl, and 4-5 membered heterocycloalkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₃₋₅ cycloalkyl, and 4-5 membered heterocycloalkyl, wherein said C₁₋₆ alkyl, C₁₋₆ haloalkyl, C₁₋₆ alkyl, C₁₋₆ haloalkyl, C₃₋₅ cycloalkyl, and 4-5 membered heterocycloalkyl are each optionally substituted with 1 or 2 independently selected R^{6B} substituents;
each R^{b61} is independently selected from C₁₋₆ alkyl, C₁₋₆ haloalkyl, C₁₋₆ alkyl, C₁₋₆ haloalkyl, C₃₋₅ cycloalkyl, and 4-5 membered heterocycloalkyl, which are each optionally substituted with 1 or 2 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62};
each R^{a62}, R^{c62}, and R^{d62}, is independently selected from H, C₁₋₆ alkyl, and C₁₋₆ haloalkyl;
each R^{b62}, is independently selected from C₁₋₆ alkyl and C₁₋₆ haloalkyl; and
R⁸ is selected from H, C₁₋₃ alkyl, and C₁₋₃ haloalkyl.

In some embodiments:
n is 0 or 1;
X is O or N(-L-R⁴);
L is S(O)₂;
R¹ is selected from C₁₋₆ alkyl, C₁₋₆ haloalkyl, and C₃₋₁₀ cycloalkyl;
R^{2A}, R^{2B}, and R^{2C} are each independently selected from H, halo, and OR^{a2};
each R^{a2} is C₁₋₆ alkyl;
each R³ is OR^{a3};
each R^{a3} is H;
R⁴ is C₁₋₆ alkyl;
R⁵ is selected from halo and CN;
R⁶ is selected from C₁₋₆ alkyl and C₃₋₁₀ cycloalkyl-C₁₋₄ alkyl; which are each optionally substituted by 1 or 2 independently selected R^{6A} substituents;
R⁷ is selected from H and C₁₋₆ alkyl;
or, R⁶ and R⁷, together with the nitrogen atom to which they are attached, form a 4-15 membered heterocycloalkyl moiety, which is optionally substituted by 1, 2, 3, 4, or 5 independently selected R^{6A} substituents;
each R^{6A} is independently selected from halo, CN, C₁₋₆ alkyl, C₁₋₆ haloalkyl, C₃₋₁₀ cycloalkyl, 4-10 membered heterocycloalkyl, 5-10 membered heteroaryl, OR^{a61}, NR^{c61}R^{d61}, and S(O)₂R^{b61}, wherein said C₁₋₆ alkyl, C₃₋₁₀ cycloalkyl, and 5-10 membered heteroaryl are each optionally substituted with 1, 2, 3, or 4 independently selected R^{6B} substituents;
each R^{a61}, R^{c61}, and R^{d61} is independently selected from H, C₁₋₆ alkyl, and C₁₋₆ haloalkyl;
each R^{b61} is independently selected from C₁₋₆ alkyl and C₃₋₁₀ cycloalkyl;
each R^{6B} is independently selected from D, halo, CN, C₁₋₆ alkyl, OR^{a62}, and NR^{c62}R^{d62};
each R^{a62}, R^{c62}, and R^{d62}, is independently selected from H, C₁₋₆ alkyl, and C₁₋₆ haloalkyl; and
R⁸ is selected from H and C₁₋₆ alkyl.

In some embodiments, the compound is a compound of Formula (Ia):

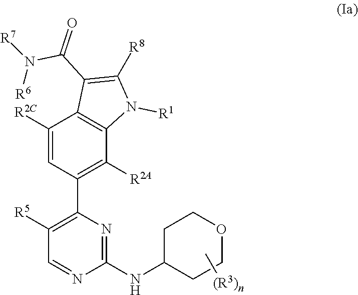

or a pharmaceutically acceptable salt thereof.

In some embodiments, the compound is a compound of Formula (Ib):

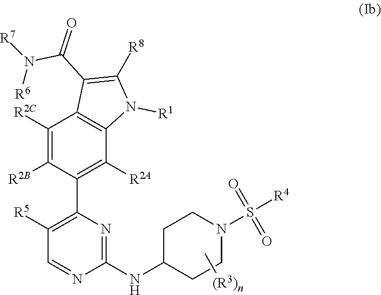

or a pharmaceutically acceptable salt thereof.

In some embodiments, the compound is perdeuterated.

In some embodiments, at least one alkyl group is perdeuterated.

In some embodiments, the compound comprises at least one deuterium atom.

In some embodiments, the compound comprises two or more deuterium atoms.

In some embodiments, the compound comprises from 1 to 6 deuterium atoms.

In some embodiments, the compound comprises from 1 to 3 deuterium atoms.

In some embodiments, the compound comprises 1 deuterium atom.

In some embodiments, the compound comprises 2 deuterium atoms.

In some embodiments, the compound comprises 3 deuterium atoms.

In some embodiments, 1 to 8 hydrogen atoms attached to carbon atoms of alkyl, alkenyl, alkynyl, haloalkyl, cycloalkyl, aryl, heterocycloalkyl, heteroaryl, cycloalkyl-alkyl, aryl-alkyl, heterocycloalkyl-alkyl, and heteroaryl-alkyl are replaced by deuterium atoms.

In some embodiments, 1 to 3 hydrogen atoms attached to carbon atoms of alkyl, alkenyl, alkynyl, haloalkyl, cycloalkyl, aryl, heterocycloalkyl, heteroaryl, cycloalkyl-alkyl, aryl-alkyl, heterocycloalkyl-alkyl, and heteroaryl-alkyl are replaced by deuterium atoms.

In some embodiments, the compound is selected from:
4-fluoro-6-(5-fluoro-2-(((3R,4R)-3-hydroxy-1-(methylsulfonyl)piperidin-4-yl)amino)pyrimidin-4-yl)-1-isopropyl-N-methyl-1H-indole-3-carboxamide;
4-fluoro-6-(5-fluoro-2-(((3S,4R)-3-hydroxytetrahydro-2H-pyran-4-yl)amino)pyrimidin-4-yl)-1-isopropyl-N-methyl-1H-indole-3-carboxamide;
4-fluoro-6-(5-fluoro-2-(((3R,4R)-3-hydroxy-1-(methylsulfonyl)piperidin-4-yl)amino)pyrimidin-4-yl)-1-isopropyl-N-(2-(pyrimidin-2-yl)ethyl)-1H-indole-3-carboxamide;
6-(5-chloro-2-(((3S,4R)-3-hydroxytetrahydro-2H-pyran-4-yl)amino)pyrimidin-4-yl)-4-fluoro-1-isopropyl-N-((1-methyl-1H-indazol-6-yl)methyl)-1H-indole-3-carboxamide;
6-(5-cyano-2-(((3S,4R)-3-hydroxytetrahydro-2H-pyran-4-yl)amino)pyrimidin-4-yl)-4-fluoro-1-isopropyl-N-((1-methyl-1H-indazol-6-yl)methyl)-1H-indole-3-carboxamide;
6-(5-chloro-2-(((3S,4R)-3-hydroxytetrahydro-2H-pyran-4-yl)amino)pyrimidin-4-yl)-4-fluoro-1-isopropyl-N,2-dimethyl-1H-indole-3-carboxamide;
1-cyclopentyl-4,7-difluoro-6-(5-fluoro-2-(((3S,4R)-3-hydroxytetrahydro-2H-pyran-4-yl)amino)pyrimidin-4-yl)-N-methyl-1H-indole-3-carboxamide;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(hydroxymethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-methylazetidin-1-yl)methanone;
((2S,4R)-4-fluoro-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone; and
((S)-4,4-difluoro-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
   or a pharmaceutically acceptable salt thereof.

In some embodiments, the compound is selected from:
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-4-(hydroxymethyl)-5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-4-(hydroxymethyl)-5-azaspiro[2.3]hexan-5-yl)methanone; and
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-2-(hydroxymethyl)azetidin-1-yl)methanone;
   or a pharmaceutically acceptable salt thereof.

In some embodiments, the compound is selected from:
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl-d₂)-3-methylazetidin-1-yl)methanone;
((2R,3S)-3-(difluoromethoxy)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
   or a pharmaceutically acceptable salt thereof.

In some embodiments, the compound is selected from:
(6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl-d₂)-3-methylazetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-(2-fluoropropan-2-yl)azetidin-1-yl)methanone;
(5,5-difluoro-2-azaspiro[3.3]heptan-2-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((R)-2-methylazetidin-1-yl)methanone;
(1,1-difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-chloro-2-(((3S,4R)-3-hydroxytetrahydro-2H-pyran-4-yl)amino)pyrimidin-4-yl)-4-fluoro-1-isopropyl-1H-indol-3-yl)((2S,3S)-2-(hydroxymethyl)-3-methylazetidin-1-yl)methanone;
(6-(5-chloro-2-(((3S,4R)-3-hydroxytetrahydro-2H-pyran-4-yl)amino)pyrimidin-4-yl)-4-fluoro-1-isopropyl-1H-indol-3-yl)((2S,3S)-2-(hydroxymethyl-d₂)-3-methylazetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-methylazetidin-1-yl)methanone;
((2R,3S)-3-(difluoromethoxy)-2-(hydroxymethyl)azetidin-1-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
N-((S)-1-cyclopropyl-2-hydroxyethyl)-1-ethyl-4-fluoro-6-(5-fluoro-2-(((3S,4R)-3-hydroxytetrahydro-2H-pyran-4-yl)amino)pyrimidin-4-yl)-1H-indole-3-carboxamide;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((S)-2-(trifluoromethyl)azetidin-1-yl)methanone;
(1,1-difluoro-5-azaspiro[2.3]hexan-5-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
(1,1-difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl)-3-(trifluoromethyl)azetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl-d₂)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-(trifluoromethyl)azetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl)-3-(trifluoromethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-methoxy-3-methylazetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2R,3R)-2-(hydroxymethyl)-3-methoxy-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2R,3R)-2-(hydroxymethyl)-3-methoxy-3-methylazetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2R,3S)-2-(hydroxymethyl)-3-(trifluoromethoxy)azetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2R,3S)-2-(hydroxymethyl)-3-(trifluoromethoxy)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3S)-2-(hydroxymethyl)-3-(trifluoromethoxy)azetidin-1-yl)methanone;
(6-(5-chloro-2-(((3S,4R)-3-hydroxytetrahydro-2H-pyran-4-yl)amino)pyrimidin-4-yl)-4-fluoro-1-isopropyl-1H-indol-3-yl)((2R,3S)-2-(hydroxymethyl)-3-(trifluoromethoxy)azetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2R,3S)-2-(hydroxymethyl)-3-(trifluoromethoxy)azetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((2R,3S)-2-(hydroxymethyl)-3-(trifluoromethoxy)azetidin-1-yl)methanone;
(1-(2,2-difluoroethyl)-4-fluoro-6-(5-fluoro-2-(((3S,4R)-3-hydroxytetra hydro-2H-pyran-4-yl)amino)pyrimidin-4-yl)-1H-indol-3-yl)(5-azaspiro[2.3]hexan-5-yl)methanone;
(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-methylazetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((S)-4-(hydroxymethyl)-5-azaspiro[2.3]hexan-5-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((R)-4-(hydroxymethyl)-5-azaspiro[2.3]hexan-5-yl)methanone;
(3-(tert-butyl)-3-hydroxyazetidin-1-yl)(6-(5-chloro-2-(((3S,4R)-3-hydroxytetrahydro-2H-pyran-4-yl)amino)pyrimidin-4-yl)-1-ethyl-4-fluoro-1H-indol-3-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)(3-((difluoromethoxy)methyl)azetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((2S,3S)-2-(hydroxymethyl-d₂)-3-methylazetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)(3-(difluoromethyl)azetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((2R,3S)-3-(difluoromethoxy)-2-(hydroxymethyl)azetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-4-(hydroxymethyl)-5-azaspiro[2.3]hexan-5-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((R)-4-(hydroxymethyl)-5-azaspiro[2.3]hexan-5-yl)methanone;
((4S)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone;
((4R)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-1-(hydroxymethyl)-2-azaspiro[3.3]heptan-2-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((R)-1-(hydroxymethyl)-2-azaspiro[3.3]heptan-2-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-methylazetidin-1-yl)methanone;
2-((S)-1-(6-(5-fluoro-2-(((3S,4R)-3-hydroxytetrahydro-2H-pyran-4-yl)amino)pyrimidin-4-yl)-1-methyl-1H-indole-3-carbonyl)azetidin-2-yl)acetonitrile;
((2R,3S)-3-(difluoromethoxy)-2-(hydroxymethyl)azetidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((2S,3S)-2-(hydroxymethyl-d₂)-3-methylazetidin-1-yl)methanone; and
(6-(5-chloro-2-(((3S,4R)-3-hydroxytetrahydro-2H-pyran-4-yl)amino)pyrimidin-4-yl)-1-ethyl-1H-indol-3-yl)((2R,3S)-3-(difluoromethoxy)-2-(hydroxymethyl)azetidin-1-yl)methanone;
   or a pharmaceutically acceptable salt thereof.

In some embodiments, the compound is selected from:
(6-(5-fluoro-2-(((3S,4R)-3-hydroxytetrahydro-2H-pyran-4-yl)amino)pyrimidin-4-yl)-1-methyl-1H-indol-3-yl)(5-azaspiro[2.3]hexan-5-yl)methanone;
(6-(5-fluoro-2-(((3S,4R)-3-hydroxytetrahydro-2H-pyran-4-yl)amino)pyrimidin-4-yl)-1-methyl-1H-indol-3-yl)(3-(trifluoromethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((R)-2-methylazetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-2-(hydroxymethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-2-((methylsulfonyl)methyl)azetidin-1-yl)methanone;
(5,5-difluoro-2-azaspiro[3.3]heptan-2-yl)(6-(5-fluoro-2-(((3S,4R)-3-hydroxytetrahydro-2H-pyran-4-yl)amino)pyrimidin-4-yl)-1-methyl-1H-indol-3-yl)methanone;
N-((S)-1-cyclopropyl-2-hydroxyethyl)-6-(5-fluoro-2-(((3S,4R)-3-hydroxytetrahydro-2H-pyran-4-yl)amino)pyrimidin-4-yl)-1-methyl-1H-indole-3-carboxamide;
(6-(5-fluoro-2-(((3S,4R)-3-hydroxytetrahydro-2H-pyran-4-yl)amino)pyrimidin-4-yl)-1-methyl-1H-indol-3-yl)(3-hydroxy-3-(trifluoromethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2R,3R)-3-hydroxy-2-methylazetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2R,3S)-3-hydroxy-2-methylazetidin-1-yl)methanone;
((2S,3S)-2-((cyclopropylsulfonyl)methyl)-3-methylazetidin-1-yl)(6-(5-fluoro-2-(((3S,4R)-3-hydroxytetrahydro-2H-pyran-4-yl)amino)pyrimidin-4-yl)-1-methyl-1H-indol-3-yl)methanone;
((2R,3 S)-3-(difluoromethoxy)-2-(hydroxymethyl)azetidin-1-yl)(6-(5-fluoro-2-(((3 S,4R)-3-hydroxytetrahydro-2H-pyran-4-yl)amino)pyrimidin-4-yl)-1-methyl-1H-indol-3-yl)methanone;
((2S,4R)-4-(difluoromethoxy)-2-(hydroxymethyl)pyrrolidin-1-yl)(6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethoxy)pyrrolidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethyl)pyrrolidin-1-yl)methanone;
((S)-4,4-difluoro-2-(hydroxymethyl)pyrrolidin-1-yl)(6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-2-(hydroxymethyl)-4,4-dimethylpyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(3-(trifluoromethyl)azetidin-1-yl)methanone;
((2S,4R)-4-(difluoromethoxy)-2-(hydroxymethyl)pyrrolidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,4R)-2-(hydroxymethyl)-4-(trifluoromethoxy)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,4R)-2-(hydroxymethyl)-4-(trifluoromethyl)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,4R)-4-fluoro-2-(hydroxymethyl)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((S)-2-(hydroxymethyl)-4,4-dimethylpyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((S)-6-(hydroxymethyl)-5-azaspiro[2.4]heptan-5-yl)methanone;
((S)-4,4-difluoro-2-(hydroxymethyl)pyrrolidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
((3S,6S)-1,1-difluoro-6-(hydroxymethyl)-5-azaspiro[2.4]heptan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
2-((S)-1-(1-ethyl-6-(5-fluoro-2-(((3S,4R)-3-hydroxytetrahydro-2H-pyran-4-yl)amino)pyrimidin-4-yl)-1H-indole-3-carbonyl)azetidin-2-yl)acetonitrile;
(1-ethyl-6-(5-fluoro-2-(((3S,4R)-3-hydroxytetrahydro-2H-pyran-4-yl)amino)pyrimidin-4-yl)-1H-indol-3-yl)((S)-2-((methylsulfonyl)methyl)azetidin-1-yl)methanone;
(1,1-difluoro-5-azaspiro[2.3]hexan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(5,5-difluoro-2-azaspiro[3.3]heptan-2-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(3-(2-fluoropropan-2-yl)azetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((R)-3-(trifluoromethoxy)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(5-azaspiro[2.3]hexan-5-yl)methanone;
N-((S)-1-cyclopropyl-2-hydroxyethyl)-1-ethyl-6-(5-fluoro-2-(((3S,4R)-3-hydroxytetrahydro-2H-pyran-4-yl)amino)pyrimidin-4-yl)-1H-indole-3-carboxamide;
(1-ethyl-6-(5-fluoro-2-(((3S,4R)-3-hydroxytetrahydro-2H-pyran-4-yl)amino)pyrimidin-4-yl)-1H-indol-3-yl)(5-azaspiro[2.4]heptan-5-yl)methanone;
(3-(difluoromethyl)-3-hydroxyazetidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(3-hydroxy-3-(trifluoromethyl)azetidin-1-yl)methanone;
((4S)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
((4R)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
((2S,3S)-2-((cyclopropylsulfonyl)methyl)-3-methylazetidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
2-((S)-1-(4-fluoro-6-(5-fluoro-2-(((3S,4R)-3-hydroxytetrahydro-2H-pyran-4-yl)amino)pyrimidin-4-yl)-1-methyl-1H-indole-3-carbonyl)azetidin-2-yl)acetonitrile;
((2S,4R)-4-(difluoromethoxy)-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethoxy)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethyl)pyrrolidin-1-yl)methanone;
((3S,6S)-1,1-difluoro-6-(hydroxymethyl)-5-azaspiro[2.4]heptan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4S)-2-(hydroxymethyl)-4-methoxy-4-(trifluoromethyl)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4S)-4-hydroxy-2-(hydroxymethyl)-4-(trifluoromethyl)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((R)-3-(trifluoromethoxy)pyrrolidin-1-yl)methanone;
((R)-3-(difluoromethoxy)pyrrolidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl-d2)-3-methylazetidin-1-yl)methanone;
((2R,3S)-3-(difluoromethoxy)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
((S)-2-(difluoromethyl)azetidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(S)-1-(4-fluoro-6-(5-fluoro-2-(((3S,4R)-3-hydroxytetrahydro-2H-pyran-4-yl)amino)pyrimidin-4-yl)-1-methyl-1H-indole-3-carbonyl)azetidine-2-carbonitrile;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-hydroxyazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-methoxyazetidin-1-yl)methanone;
(3-(1,1-difluoroethyl)-3-methylazetidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(1,1-difluoro-2,2-dimethyl-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-chloro-2-(((3S,4R)-3-hydroxytetrahydro-2H-pyran-4-yl)amino)pyrimidin-4-yl)-1-ethyl-1H-indol-3-yl)(3-(trifluoromethyl)azetidin-1-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((R)-2-methylazetidin-1-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((2S,4R)-4-(difluoromethoxy)-2-(hydroxymethyl)pyrrolidin-1-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((2S,4R)-4-fluoro-2-(hydroxymethyl)pyrrolidin-1-yl)methanone;
(5,5-difluoro-2-azaspiro[3.3]heptan-2-yl)(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d2)-3-methylazetidin-1-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)(3-(2-fluoropropan-2-yl)azetidin-1-yl)methanone;
(1,1-difluoro-5-azaspiro[2.3]hexan-5-yl)(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)methanone;
(6-(5-chloro-2-(((3S,4R)-3-hydroxytetrahydro-2H-pyran-4-yl)amino)pyrimidin-4-yl)-4-fluoro-1-methyl-1H-indol-3-yl)(3-(2-fluoropropan-2-yl)azetidin-1-yl)methanone;
(6-(5-chloro-2-(((3S,4R)-3-hydroxytetrahydro-2H-pyran-4-yl)amino)pyrimidin-4-yl)-4-fluoro-1-methyl-1H-indol-3-yl)(1,1-difluoro-2,2-dimethyl-5-azaspiro[2.3]hexan-5-yl)methanone;
(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone;
(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone;
((4S)-1,1-difluoro-4-(hydroxymethyl)-5-azaspiro[2.3]hexan-5-yl)(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)methanone;
((4R)-1,1-difluoro-4-(hydroxymethyl)-5-azaspiro[2.3]hexan-5-yl)(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((S)-1-(hydroxymethyl)-2-azaspiro[3.3]heptan-2-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((R)-1-(hydroxymethyl)-2-azaspiro[3.3]heptan-2-yl)methanone;
((4S)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((4R)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((2S,3S)-3-(difluoromethyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((2R,3R)-3-(difluoromethyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((1R,4S,5R)-5-(hydroxymethyl)-2-azabicyclo[2.1.0]pentan-2-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((1S,4R,5S)-5-(hydroxymethyl)-2-azabicyclo[2.1.0]pentan-2-yl)methanone;
(2S,3S)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)-2-(hydroxymethyl)azetidine-3-carbonitrile;
(2R,3R)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)-2-(hydroxymethyl)azetidine-3-carbonitrile;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-(methylsulfonyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-(methylsulfonyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3 S)-2-(hydroxymethyl)-3-(methylsulfonyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3R)-2-(hydroxymethyl)-3-(methylsulfonyl)azetidin-1-yl)methanone;
((4R)-1,1-difluoro-4-(hydroxymethyl)-5-azaspiro[2.3]hexan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
((4S)-1,1-difluoro-4-(hydroxymethyl)-5-azaspiro[2.3]hexan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
((4R)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-isopropyl-1H-indol-3-yl)methanone;
((4S)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-isopropyl-1H-indol-3-yl)methanone;
(6-(5-fluoro-2-(((3S,4R)-3-hydroxytetrahydro-2H-pyran-4-yl)amino)pyrimidin-4-yl)-1-isopropyl-1H-indol-3-yl)((S)-4-(hydroxymethyl)-5-azaspiro[2.3]hexan-5-yl)mehthanone;
(6-(5-fluoro-2-(((3S,4R)-3-hydroxytetrahydro-2H-pyran-4-yl)amino)pyrimidin-4-yl)-1-isopropyl-1H-indol-3-yl)((R)-4-(hydroxymethyl)-5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3 S)-2-(hydroxymethyl)-3-((trifluoromethoxy)methyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-((trifluoromethoxy)methyl)azetidin-1-yl)methanone;
((2S,3S)-3-((difluoromethoxy)methyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((2R,3R)-3-((difluoromethoxy)methyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((4S)-1,1-difluoro-4-(hydroxymethyl)-5-azaspiro[2.3]hexan-5-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
((4R)-1,1-difluoro-4-(hydroxymethyl)-5-azaspiro[2.3]hexan-5-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-1-(hydroxymethyl)-2-azaspiro[3.3]heptan-2-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-1-(hydroxymethyl)-2-azaspiro[3.3]heptan-2-yl)methanone;
((R)-3,3-difluoro-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((S)-3,3-difluoro-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
4-fluoro-6-(5-fluoro-2-(((3R,4R)-3-hydroxy-1-(methylsulfonyl)piperidin-4-yl)amino)pyrimidin-4-yl)-1-isopropyl-N,N-dimethyl-1H-indole-3-carboxamide;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(methoxymethyl)azetidin-1-yl)methanone;
2-((S)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)azetidin-2-yl)acetonitrile;
((S)-2-((dimethylamino)methyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((S)-2-(2,2-difluoroethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(1,1-difluoro-5-azaspiro[2.4]heptan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(5,5-difluoro-2-azaspiro[3.3]heptan-2-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(hydroxymethyl)-2-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3R)-2-(hydroxymethyl)-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(2-hydroxyethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(2-hydroxypropan-2-yl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3 S)-3-methyl-2-((methylsulfonyl)methyl)azetidin-1-yl)methanone;
((S)-2-((cyclopropylsulfonyl)methyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(1,1-difluoro-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(pyrrolidin-1-yl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(6-methyl-2,6-diazaspiro[3.3]heptan-2-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(6-oxa-2-azaspiro[3.4]octan-2-yl)methanone;
(3-(dimethylamino)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(fluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-hydroxyazetidin-1-yl)methanone;
(3-(difluoromethoxy)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3R,4R)-3-hydroxytetrahydro-2H-pyran-4-yl)amino)pyrimidin-4-yl)-1-isopropyl-1H-indol-3-yl)(3-(pyrrolidin-1-yl)azetidin-1-yl)methanone;
azetidin-1-yl(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(3-aminoazetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(3-(difluoromethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-fluoroazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-2-methylazetidin-1-yl)methanone;
((2R,4R)-2,4-dimethylazetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(5-azaspiro[2.3]hexan-5-yl)methanone;
1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)azetidine-3-carbonitrile;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-3-fluoropyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-3-hydroxypyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-3-hydroxypyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-hydroxy-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(hydroxymethyl)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-2-(hydroxymethyl)pyrrolidin-1-yl)methanone;
(3,3-difluoropyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((1R,2S,5S)-2-(hydroxymethyl)-3-azabicyclo[3.1.0]hexan-3-yl)methanone;
(S)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)pyrrolidine-3-carbonitrile;
(R)-1-(4-fluoro-6-(5-fluoro-2-(((3 S,4R)-3-hydroxytetrahydro-2H-pyran-4-yl)amino)pyrimidin-4-yl)-1-isopropyl-1H-indole-3-carbonyl)pyrrolidine-3-carbonitrile;
(3-fluoro-3-(trifluoromethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(5-azaspiro[2.4]heptan-5-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-methyl-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-isopropoxyazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(hydroxymethyl)azetidin-1-yl)methanone;
(3-((difluoromethoxy)methyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(2-(trifluoromethoxy)ethyl)azetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((S)-2-(hydroxymethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(trifluoromethoxy)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-((trifluoromethoxy)methyl)azetidin-1-yl)methanone;
(3-(difluoromethoxy)azetidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(1-hydroxycyclopropyl)azetidin-1-yl)methanone;
(4,7-difluoro-6-(5-fluoro-2-(((3S,4R)-3-hydroxytetrahydro-2H-pyran-4-yl)amino)pyrimidin-4-yl)-1-methyl-1H-indol-3-yl)(1,1-difluoro-5-azaspiro[2.3]hexan-5-yl)methanone;
(1,1-difluoro-5-azaspiro[2.3]hexan-5-yl)(7-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(7-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl-d2)-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1,7-dimethyl-1H-indol-3-yl)((2S,3S)-2-(hydroxymethyl-d2)-3-methylazetidin-1-yl)methanone;
(1,1-difluoro-5-azaspiro[2.3]hexan-5-yl)(6-(5-fluoro-2-(((3S,4R)-3-hydroxytetrahydro-2H-pyran-4-yl)amino)pyrimidin-4-yl)-1,7-dimethyl-1H-indol-3-yl)methanone;
(4,5-difluoro-6-(5-fluoro-2-(((3S,4R)-3-hydroxytetrahydro-2H-pyran-4-yl)amino)pyrimidin-4-yl)-1-methyl-1H-indol-3-yl)(1,1-difluoro-5-azaspiro[2.3]hexan-5-yl)methanone;
(1,1-difluoro-5-azaspiro[2.3]hexan-5-yl)(5-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(5-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl-d2)-3-methylazetidin-1-yl)methanone;
and
(1,1-difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-5-methoxy-1-methyl-1H-indol-3-yl)methanone;
   or a pharmaceutically acceptable salt thereof.

In some embodiments, 1, 2, 3, 4, 5, 6, 7, or 8 hydrogen atoms, attached to carbon atoms of “alkyl”, “alkenyl”, “alkynyl”, “aryl”, “phenyl”, “cycloalkyl”, “heterocycloalkyl”, or “heteroaryl” substituents or “—C₁₋₄ alkyl-” and “alkylene” linking groups, as described herein, are optionally replaced by deuterium atoms.

It is further appreciated that certain features of the invention, which are, for clarity, described in the context of separate embodiments, can also be provided in combination in a single embodiment as if the embodiments were claims written in multiple dependent form. Conversely, various features of the invention which are, for brevity, described in the context of a single embodiment, can also be provided separately or in any suitable subcombination.

At various places in the present specification, divalent linking substituents are described. Unless otherwise specified, it is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency, that the designated atom's normal valency is not exceeded, and that the substitution results in a stable compound.

As used herein, the term “independently selected from” means that each occurrence of a variable or substituent are independently selected at each occurrence from the applicable list.

As used herein, the phrase “each ‘variable’ is independently selected from” means substantially the same as wherein “at each occurrence ‘variable’ is selected from.”

When any variable (e.g., R^{G}) occurs more than one time in any constituent or formula for a compound, its definition at each occurrence is independent of its definition at every other occurrence. Thus, for example, if a group is shown to be substituted with 1, 2, 3, or 4 independently selected R^{G} substituents, then said group may optionally be substituted with up to four R^{G} groups and R^{G} at each occurrence is selected independently from the definition of R^{G}.

In some embodiments, substituents are indicated as floating substituents—e.g., (R³)ₙ in Formula (I):

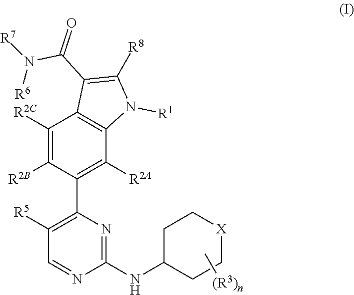

It is understood that substituent R³ can occur n number of times on the ring, and R³ can be a different moiety at each occurrence. It is to be understood that each R group may replace any hydrogen atom attached to a ring atom, except that R³ may not replace -L-R⁴ in the variable X. (R⁵)ₚ is another example of a floating substituent.

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. In some embodiments, the aryl group has 6 to 10 carbon atoms. In some embodiments, the aryl group is phenyl or naphthyl. In some embodiments, the aryl is phenyl.

As used herein, “halo” refers to F, Cl, Br, or I. In some embodiments, halo is F, Cl, or Br.

In some embodiments, halo is F or Cl. In some embodiments, halo is F. In some embodiments, halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl,” employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “thio” refers to a group of formula —SH.

As used herein, the term “Cₙ₋ₘ alkylamino” refers to a group of formula —NH(alkyl), wherein the alkyl group has n to m carbon atoms. In some embodiments, the alkyl group of the alkylamino has 1 to 6, 1 to 4, or 1 to 3 carbon atoms.

As used herein, the term “Cₙ₋ₘ alkoxycarbonyl” refers to a group of formula —C(O)O— alkyl, wherein the alkyl group has n to m carbon atoms. In some embodiments, the alkyl group of the alkoxycarbonyl has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group of the alkylcarbonyl has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group of the alkylcarbonylamino has 1 to 6, 1 to 4, or 1 to 3 carbon atoms.

As used herein, the term “Cₙ₋ₘ alkoxycarbonylamino” refers to a group of formula —NHC(O)O(Cₙ₋ₘ alkyl), wherein the alkyl group has n to m carbon atoms. In some embodiments, the alkyl group of the alkoxycarbonylamino has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group of the alkylsulfonylamino has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group of the alkylaminosulfonyl has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group of the dialkylaminosulfonyl has, independently, 1 to 6, 1 to 4, or 1 to 3 carbon atoms.

As used herein, the term “aminosulfonylamino” refers to a group of formula —NHS(O)₂ NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group of the alkylaminosulfonylamino has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group of the dialkylaminosulfonylamino has, independently, 1 to 6, 1 to 4, or 1 to 3 carbon atoms.

As used herein, the term “aminocarbonylamino,” employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group of the alkylaminocarbonylamino has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group of the dialkylaminocarbonylamino has, independently, 1 to 6, 1 to 4, or 1 to 3 carbon atoms.

As used herein, the term “Cₙ₋ₘ alkylcarbamyl” refers to a group of formula —C(O)— NH(alkyl), wherein the alkyl group has n to m carbon atoms. In some embodiments, the alkyl group of the alkylcarbamyl has 1 to 6, 1 to 4, or 1 to 3 carbon atoms.

As used herein, the term “Cₙ₋ₘ alkylthio” refers to a group of formula —S-alkyl, wherein the alkyl group has n to m carbon atoms. In some embodiments, the alkyl group of the alkylthio has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group of the alkylsulfinyl has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group of the alkylsulfonyl has 1 to 6, 1 to 4, or 1 to 3 carbon atoms.

As used herein, the term “cyano-Cₙ₋ₘ alkyl” refers to a group of formula —(Cₙ₋ₘ alkylene)-CN, wherein the alkylene group has n to m carbon atoms. As used herein, the term “cyano-C₁₋₆ alkyl” refers to a group of formula —(C₁₋₆ alkylene)-CN. As used herein, the term “cyano-C₁₋₃ alkyl” refers to a group of formula —(C₁₋₃ alkylene)-CN.

As used herein, the term “HO—Cₙ₋ₘ alkyl” refers to a group of formula —(Cₙ₋ₘ alkylene)-OH, wherein the alkylene group has n to m carbon atoms. As used herein, the term “HO—C₁₋₃ alkyl” refers to a group of formula —(C₁₋₃ alkylene)-OH.

As used herein, the term “Cₙ₋ₘ alkoxy-Cₒ₋ₚ alkyl” refers to a group of formula —(Cₙ₋ₘ alkylene)-O(Cₒ₋ₚ alkyl), wherein the alkylene group has n to m carbon atoms and the alkyl group has o to p carbon atoms. As used herein, the term “C₁₋₆ alkoxy-C₁₋₆ alkyl” refers to a group of formula —(C₁₋₆ alkylene)-O(C₁₋₆ alkyl). As used herein, the term “C₁₋₃ alkoxy-C₁₋₃ alkyl” refers to a group of formula —(C₁₋₃ alkylene)-O(C₁-3 alkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group of the dialkylamino independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group of the dialkylcarbamyl independently has 1 to 6, 1 to 4, or 1 to 3 carbon atoms.

As used herein, the term “Cₙ₋ₘ alkylcarbonyloxy” is a group of formula —OC(O)-alkyl, wherein the alkyl group has n to m carbon atoms. In some embodiments, the alkyl group of the alkylcarbonyloxy has 1 to 6, 1 to 4, or 1 to 3 carbon atoms.

As used herein, “aminocarbonyloxy” is a group of formula —OC(O)—NH₂.

As used herein, “Cₙ₋ₘ alkylaminocarbonyloxy” is a group of formula —OC(O)—NH-alkyl, wherein the alkyl group has n to m carbon atoms. In some embodiments, the alkyl group of the alkylaminocarbonyloxy has 1 to 6, 1 to 4, or 1 to 3 carbon atoms.

As used herein, “di(Cₙ₋ₘalkyl)aminocarbonyloxy” is a group of formula —OC(O)—N(alkyl)₂, wherein each alkyl group has, independently, n to m carbon atoms. In some embodiments, each alkyl group of the dialkylaminocarbonyloxy independently has 1 to 6, 1 to 4, or 1 to 3 carbon atoms.

As used herein “Cₙ₋ₘ alkoxycarbonylamino” refers to a group of formula —NHC(O)—O— alkyl, wherein the alkyl group has n to m carbon atoms.

As used herein, the term “carbamyl” to a group of formula —C(O)NH₂.

As used herein, the term “carbonyl,” employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group).

Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, or S. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridyl, pyrimidinyl, pyrazinyl, pyridazinyl, pyrrolyl, pyrazolyl, azolyl, oxazolyl, isoxazolyl, thiazolyl, isothiazolyl, imidazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Examples of heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, azabicyclo[2.2.1]heptan-7-yl, azabicyclo[2.2.1]heptan-2-yl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon ring members and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, the term “alkylene” refers a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “alkenylene” refers a divalent straight chain or branched alkenyl linking group. Examples of “alkenylene groups” include ethen-1,1-diyl, ethen-1,2-diyl, propen-1,3-diyl, 2-buten-1,4-diyl, 3-penten-1,5-diyl, 3-hexen-1,6-diyl, 3-hexen-1,5-diyl, and the like.

As used herein, the term “alkynylene” refers a divalent straight chain or branched alkynyl linking group. Examples of “alkynylene groups” include propyn-1,3-diyl, 2-butyn-1,4-diyl, 3-pentyn-1,5-diyl, 3-hexyn-1,6-diyl, 3-hexyn-1,5-diyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid.

Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H—, 2H—and 4H-1,2,4-triazole, 1H- and 2H—isoindole, 2-hydroxypyridine and 2-pyridone, and 1H- and 2H-pyrazole. Tautomeric forms can be in equilibrium or sterically locked into one form by appropriate substitution. It is intended that the claims to one tautomer cover both tautomers even if not specified.

All compounds, and pharmaceutically acceptable salts thereof, can be found together with other substances such as water and solvents (e.g., hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the Schemes below.

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “r.t.” as used herein, are understood in the art, and refer generally to a temperature, e.g., a reaction temperature, that is about the temperature of the room in which the reaction is carried out, for example, a temperature from about 20° C. to about 30° C.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

The Schemes below provide general guidance in connection with preparing the compounds of the invention. One skilled in the art would understand that the preparations shown in the Schemes can be modified or optimized using general knowledge of organic chemistry to prepare various compounds of the invention.

As will be appreciated by those skilled in the art, the compounds provided herein, including salts and stereoisomers thereof, can be prepared using known organic synthesis techniques and can be synthesized according to any of numerous possible synthetic routes.

Compounds of Formula 1-5 can be synthesized, for example, according to the process shown in Scheme 1. As depicted in Scheme 1, nucleophilic substitution reactions of compounds of Formula 1-1 (i.e., where Hal is a suitable halogen, such as Cl, Br, or I) and electrophiles of the general Formula 1-2 (i.e., where LG is a suitable leaving group, such as a halogen or mesylate group) under appropriate conditions (e.g., in the presence of a base, such as Cs₂CO₃, in a suitable solvent, such as dimethylformamide) generates compounds of Formula 1-3. Compounds of Formula 1-1 are commercially available or can be readily synthesized according to methods known by persons skilled in the art. Formylation of compounds of Formula 1-3 (e.g., via Vilsmeier-Haack reaction, for example, using POCl₃ and DMF) generates compounds of Formula 1-4. Oxidation of compounds of Formula 1-4 under appropriate conditions (e.g., via Pinnick oxidation) furnishes compounds of Formula 1-5.

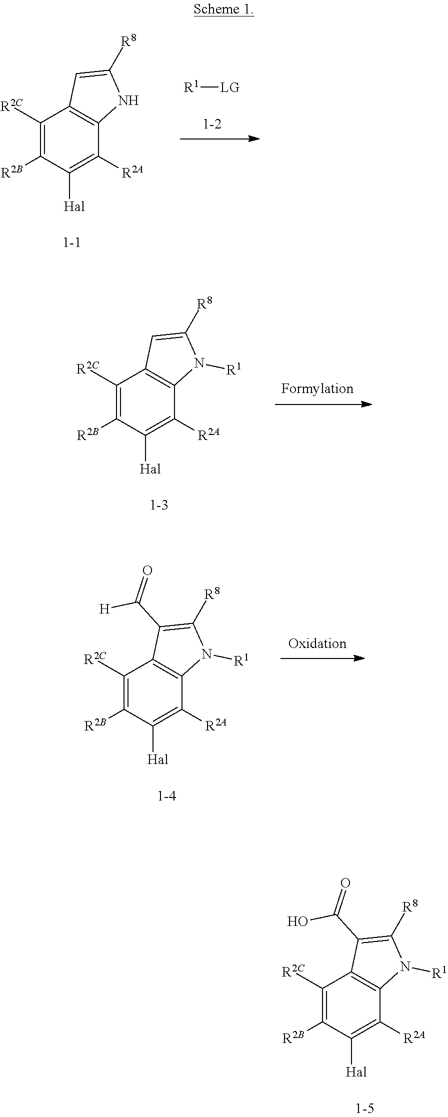

Compounds of Formula (I) can be synthesized, for example, using the process illustrated in Scheme 2. In the process depicted in Scheme 2, esterification of compounds of Formula 1-5 under appropriate conditions (e.g., in the presence of trimethylsilyldiazomethane) generates compounds of Formula 2-1. Borylation of compounds of Formula 2-1 under appropriate conditions (e.g., using a borylating reagent, such as bis(pinacolato)diboron, in the presence of a suitable catalyst, such as Pd(dppf)Cl₂, and a base, such as KOAc, in an appropriate solvent, such as 1,4-dioxane) affords compounds of Formula 2-2. Suzuki reactions of compounds of Formula 2-2 and pyrimidines of Formula 2-3 (i.e., where each Hal is a suitable halogen, such as Cl, Br, or I) under appropriate conditions (e.g., using a suitable palladium catalyst, such as Pd(dppf)Cl₂, and a base, such as K₂CO₃, in an appropriate solvent, such as 1,4-dioxane/H₂O) affords compounds of Formula 2-4. A number of methods (e.g., a suitable cross-coupling reaction or nucleophilic aromatic substitution) can be used to access compounds of Formula 2-6. For example, transition metal (e.g., Pd, Cu, Ni) catalyzed reactions (including, but not limited to, Buchwald couplings) of compounds 2-4 and appropriate amine nucleophiles of the general Formula 2-5 under appropriate conditions (e.g., in the presence of a suitable catalyst, such as GPhos Pd G6 TES, a base, such as NaOTMS, in a suitable solvent, such as THF) affords compounds of Formula 2-6. Alternatively, compounds of Formula 2-4 (i.e., where Hal is a suitable halogen, such as Cl) can be reacted with an appropriate amine nucleophile of Formula 2-5 under appropriate conditions (e.g., in the presence of a base, such as N,N-diisopropylethylamine, in an appropriate solvent, such as dimethylformamide) to generate compounds of Formula 2-6. Saponification of compounds of Formula 2-6 under appropriate conditions (e.g., using a suitable reagent, such as LiOH) furnishes compounds of Formula 2-7. Amidation of compounds of Formula 2-7 and amines of Formula 2-8 under appropriate conditions (e.g., in the presence of an amide coupling reagent, such as HATU, and a base, such as N,N-diisopropylethylamine) affords compounds of Formula (I).

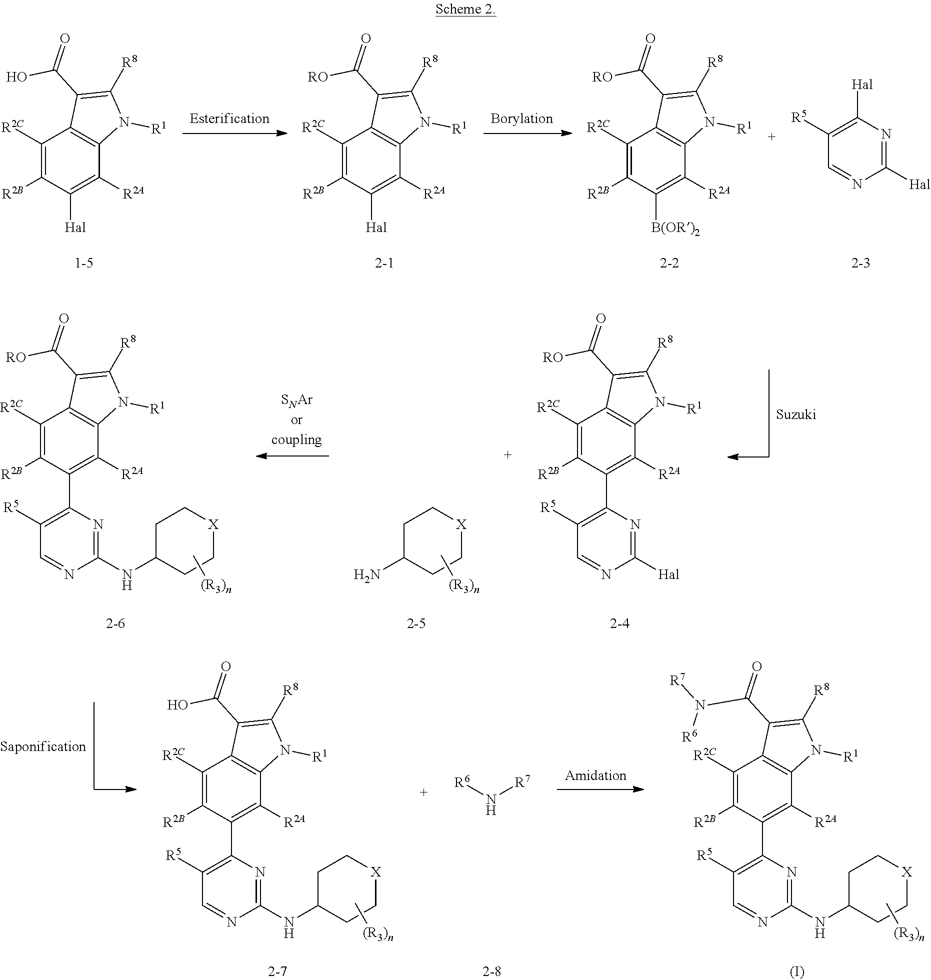

Compounds of Formula (I) can be synthesized, for example, according to the process shown in Scheme 3. As depicted in Scheme 3, amidation of compounds of Formula 1-5 and amines of Formula 2-8 under appropriate conditions (e.g., in the presence of an amide coupling reagent, such as HATU, and a base, such as N,N-diisopropylethylamine) affords compounds of Formula 3-1. Borylation of compounds of Formula 3-1 under appropriate conditions (e.g., using a borylating reagent, such as bis(pinacolato)diboron, in the presence of a suitable catalyst, such as Pd(dppf)Cl₂, and a base, such as KOAc, in an appropriate solvent, such as 1,4-dioxane) affords compounds of Formula 3-2. Suzuki reactions of compounds of Formula 3-2 and pyrimidines of Formula 2-3 (i.e., where each Hal is a suitable halogen, such as Cl, Br, or I) under appropriate conditions (e.g., using a suitable palladium catalyst, such as Pd(dppf)Cl₂, and a base, such as K₂CO₃, in an appropriate solvent, such as 1,4-dioxane/H₂O) affords compounds of Formula 3-3. A number of methods (e.g., a suitable cross-coupling reaction or nucleophilic aromatic substitution) can be used to access compounds of Formula 3-3. For example, transition metal (e.g., Pd, Cu, Ni) catalyzed reactions (including, but not limited to, Buchwald couplings) of compounds 3-3 and appropriate amine nucleophiles of the general Formula 2-5 under appropriate conditions (e.g., in the presence of a suitable catalyst, such as GPhos Pd G6 TES, a base, such as NaOTMS, in a suitable solvent, such as THF) affords compounds of Formula (I). Alternatively, compounds of Formula 3-3 (i.e., where Hal is a suitable halogen, such as Cl) can be reacted with an appropriate amine nucleophile of Formula 2-5 under appropriate conditions (e.g., in the presence of a base, such as N,N-diisopropylethylamine, in an appropriate solvent, such as dimethylformamide) to generate compounds of Formula (I).

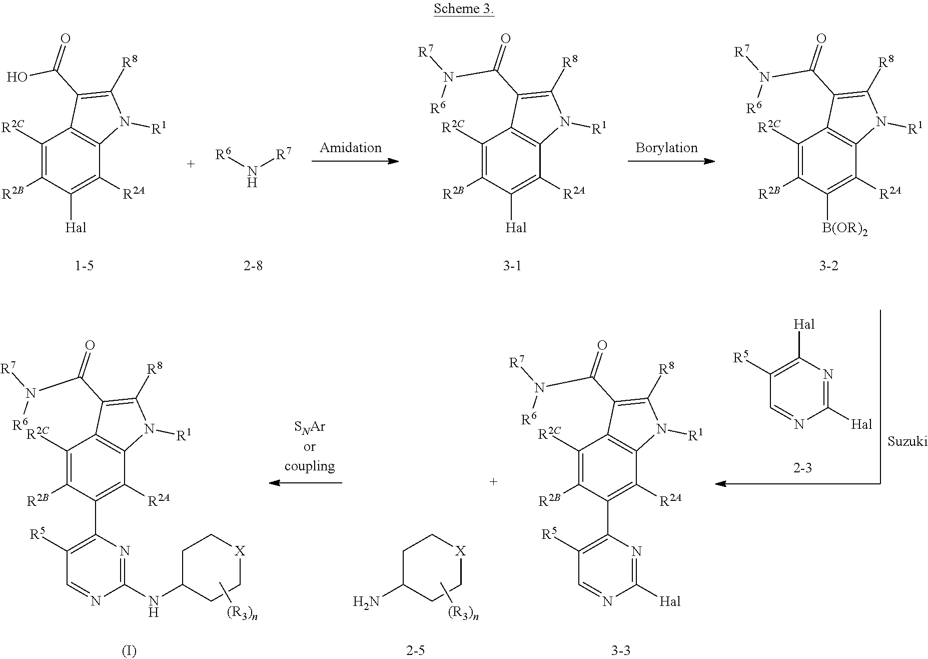

Compounds of Formula 4-5 can be synthesized, for example, using the process illustrated in Scheme 4. In the process depicted in Scheme 4, nucleophilic aromatic substitution reactions of compounds of Formula 4-1 (i.e., where Hal is a suitable halogen, such as Cl, Br, or I) and primary amines of the general Formula 4-2 under appropriate conditions (e.g., in the presence of a base, such as Cs₂CO₃, in a suitable solvent, such as dimethylformamide, followed by treatment with an acid, such as HCl) generates compounds of Formula 4-3. Compounds of Formula 4-1 are commercially available or can be readily synthesized according to methods known by persons skilled in the art. Formation of the indole ring occurs via reaction of compounds of Formula 4-3 with alpha-diazo esters of Formula 4-4 (e.g., ethyl diazoacetate) under appropriate conditions (e.g., in the presence of a Lewis acid, such as BF₃·Et₂O, in a suitable solvent, such as CH₂Cl₂) generates compounds of Formula 4-5. In certain cases, Formula 4-5 is a version of Formula 2-1 where R⁸ is H.

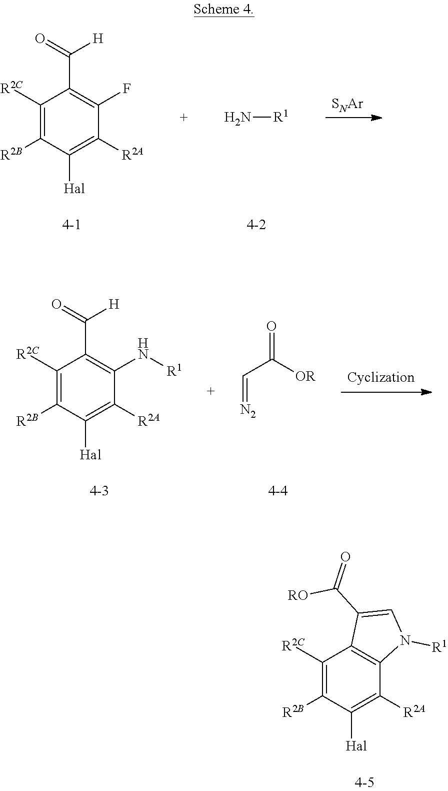

### Methods of Use

Compounds of the present disclosure (and pharmaceutically acceptable salts thereof) can inhibit CDK4 and therefore are useful for treating diseases wherein the underlying pathology is, wholly or partially, mediated by CDK4. In some embodiments, the compounds, and pharmaceutically acceptable salts thereof, inhibit CDK4. In some embodiments, the compounds, and pharmaceutically acceptable salts thereof, are selective for CDK4 over CDK1, CDK6, and/or CDK7. In some embodiments, the compounds, and pharmaceutically acceptable salts thereof, are selective for CDK4 over CDK1. In some embodiments, the compounds, and pharmaceutically acceptable salts thereof, are selective for CDK4 over CDK7. In some embodiments, the compounds, and pharmaceutically acceptable salts thereof, are selective for CDK4 over CDK6. In some embodiments, the compounds, and pharmaceutically acceptable salts thereof, inhibit CDK2 and CDK4. In some embodiments, the compounds, and pharmaceutically acceptable salts thereof, are selective for CDK4 over CDK6. In some embodiments, the compounds, and pharmaceutically acceptable salts thereof, are selective for CDK4 over CDK6. In some embodiments, the selectivity referenced in each of the embodiments of this paragraph is at least 2-fold, at least 3-fold, at least 5-fold, or at least 10-fold.

Such diseases include cancer and other diseases with proliferation disorder. In some embodiments, the present disclosure provides treatment of an individual or a patient in vivo using a compound of Formula (I) or a salt thereof such that growth of cancerous tumors is inhibited. A compound of Formula (I) or of any of the formulas as described herein, or a compound as recited in any of the claims and described herein, or a salt thereof, can be used to inhibit the growth of cancerous tumors with aberrations that activate the CDK4 kinase activity. These include, but are not limited to, disease (e.g., cancers) that are characterized by amplification or overexpression of CCNE1 such as ovarian cancer, uterine carcinosarcoma and breast cancer and p27 inactivation such as breast cancer and melanomas. Accordingly, in some embodiments of the methods, the patient has been previously determined to have an amplification of the cyclin E1 (CCNE1) gene and/or an expression level of CCNE1 in a biological sample obtained from the human subject that is higher than a control expression level of CCNE1. Alternatively, a compound of Formula (I) or of any of the formulas as described herein, or a compound as recited in any of the claims and described herein, or a salt thereof, can be used in conjunction with other agents or standard cancer treatments, as described below. In one embodiment, the present disclosure provides a method for inhibiting growth of tumor cells in vitro. The method includes contacting the tumor cells in vitro with a compound of Formula (I) or of any of the formulas as described herein, or of a compound as recited in any of the claims and described herein, or of a salt thereof. In another embodiment, the present disclosure provides a method for inhibiting growth of tumor cells with CCNE1 amplification and overexpression in an individual or a patient. The method includes administering to the individual or patient in need thereof a therapeutically effective amount of a compound of Formula (I) or of any of the formulas as described herein, or of a compound as recited in any of the claims and described herein, or a salt or a stereoisomer thereof.

In some embodiments, provided herein is a method of inhibiting CDK4, comprising contacting the CDK4 with a compound of Formula (I) or any of the formulas as described herein, a compound as recited in any of the claims and described herein, or a salt thereof. In some embodiments, provided herein is a method of inhibiting CDK4 in a patient, comprising administering to the patient a compound of Formula (I) or any of the formulas as described herein, a compound as recited in any of the claims and described herein, or a salt thereof.

In some embodiments, provided herein is a method for treating cancer. The method includes administering to a patient (in need thereof), a therapeutically effective amount of a compound of Formula (I) or any of the formulas as described herein, a compound as recited in any of the claims and described herein, or a salt thereof. In another embodiment, the cancer is characterized by amplification or overexpression of CCNE1. In some embodiments, the cancer is ovarian cancer or breast cancer, characterized by amplification or overexpression of CCNE1.

In some embodiments, provided herein is a method of treating a disease or disorder associated with CDK4 in a patient, comprising administering to the patient a therapeutically effective amount of a compound of Formula (I) or any of the formulas as described herein, a compound as recited in any of the claims and described herein, or a salt thereof. In some embodiments, the disease or disorder associated with CDK4 is associated with an amplification of the cyclin E1 (CCNE1) gene and/or overexpression of CCNE1.

In another embodiment, the cancer is characterized by amplification or overexpression of CCNE1. In some embodiments, the cancer is characterized by amplification or overexpression of CCND1. In some embodiments, the cancer is characterized by amplification or overexpression of CCND1 and CCNE1.

In some embodiments, the cancer is ovarian cancer or breast cancer, characterized by amplification or overexpression of CCNE1. In some embodiments, the cancer is ovarian cancer or breast cancer, characterized by amplification or overexpression of CCND1. In some embodiments, the cancer is ovarian cancer or breast cancer, characterized by amplification or overexpression of CCNE1 and CCND1.

In some embodiments, the cancer is hormone receptor positive (HR+) and human epidermal growth factor receptor 2 negative (HER2−). In some embodiments, the cancer is breast cancer which is hormone receptor positive (HR+) and human epidermal growth factor receptor 2 negative (HER2−). In some embodiments, the cancer is metastatic breast cancer which is hormone receptor positive (HR+) and human epidermal growth factor receptor 2 negative (HER2−). In some embodiments, the cancer is metastatic breast cancer.

In some embodiments, the cancer is a cancer which is platinum sensitive. In some embodiments, the cancer is gynecological cancer which is platinum sensitive. In some embodiments, the cancer is gynecological cancer which is platinum resistant. Such gynecological cancers include ovarian, breast, prostate, pancreatic or uterine cancer. In some embodiments, the cancer is ovarian cancer which is platinum sensitive. As used herein, platinum refers to platinum-based chemotherapy treatment.

In some embodiments, the cancer is a cancer which is platinum resistant. In some embodiments, the cancer is ovarian cancer which is platinum resistant. In some embodiments, the cancer is endometrioid cancer or a cancer with amplification or overexpression of CCNE1 or CCD1.

In some embodiments, the cancer is a cancer driven by CDK4 or CCND1 signaling (e.g, CDK+ solid tumors). In some embodiments, the CDK+ cancer is breast cancer, a gynecological cancer, a gastrointestinal cancer, prostate cancer, a sarcoma or melanoma. In some embodiments, the cancer is a cancer driven by CDK4 or CCND1 signaling (e.g, CDK+ solid tumors). In some embodiments, the CDK+ cancer is HR+/HER2− breast cancer, HR+/IER2+ breast cancer, metastatic castration-resistant prostate cancer, ovarian cancer, endometrial cancer, esophaghael cancer, gastric cancer, colorectal cancer, liposarcoma, Ewing sarcoma, or melanoma.

In some embodiments, the cancer is breast cancer. In some embodiments, the cancer is ovarian cancer. In some embodiments, the cancer is a gynecological cancer. In some embodiments, the cancer is a gastrointestinal cancer. In some embodiments, the cancer is prostate cancer. In some embodiments, the cancer is a sarcoma. In some embodiments, the cancer is melanoma. In some embodiments, the cancer is HR+/HER2− breast cancer. In some embodiments, the cancer is HR+/HER2+ breast cancer. In some embodiments, the cancer is metastatic castration-resistant prostate cancer. In some embodiments, the cancer is endometrial cancer. In some embodiments, the cancer is esophaghael cancer. In some embodiments, the cancer is gastric cancer. In some embodiments, the cancer is colorectal cancer. In some embodiments, the cancer is liposarcoma. In some embodiments, the cancer is Ewing sarcoma. In some embodiments, the cancer is melanoma.

In some embodiments, the disease or disorder associated with CDK4 is N-myc amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE1 overexpression (see Takada et al., Cancer Res, 2017. 77(18): 4881-4893).

In some embodiments, the disease or disorder associated with CDK4 is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with CDK4 is lung adenocarcinoma, breast invasive carcinoma, uterine carcinosarcoma, ovarian serous cystadenocarcinoma, or stomach adenocarcinoma.

In some embodiments, the disease or disorder associated with CDK4 is an adenocarcinoma, carcinoma, or cystadenocarcinoma.

In some embodiments, the disease or disorder associated with CDK4 is uterine cancer, ovarian cancer, stomach cancer, esophageal cancer, lung cancer, bladder cancer, pancreatic cancer, or breast cancer.

In some embodiments, the disease or disorder associated with CDK4 is a cancer.

In some embodiments, the cancer is characterized by amplification or overexpression of CCNE1. In some embodiments, the cancer is ovarian cancer or breast cancer, characterized by amplification or overexpression of CCNE1.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CMIL), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual”, “patient,” and “subject” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

In some embodiments, the one or more additional pharmaceutical agents is an aromatase inhibitor, luteinizing hormone-releasing hormone (LHRH) agonist, an anti-androgen, therapy targeting the PI3K/AKT/mTOR (PAM) pathway, a selective estrogen receptor degrader (SERD), a CDK2 inhibitor, a CDK4/6 inhibitor, a CDK4 inhibitor, or VEGF-A inhibitor, or a combination thereof.

In some embodiments, the VEGF-A inhibitor is bevacizumab.

In some embodiments, the PAM therapy is alpelisib, capivasertib, or everolimus, or a pharmaceutically acceptable salt thereof.

In some embodiments, the SERD is fluvestrant, camizestrant, amcenestrant, elacestrant, giredestrant, imlunestrant, rintodestrant, AZD9496, borestrant, taragarestrant, and ZN-c5, or a pharmaceutically acceptable salt thereof.

In some embodiments, the CDK4/6 inhibitor is albociclib, ribociclib, albociclib, abemaciclib, or dalpiciclib, or a pharmaceutically acceptable salt thereof. In some embodiments, the CDK4 inhibitor is atirmociclib or a pharmaceutically acceptable salt thereof.

In some embodiments, the CDK2 inhibitor is ebvaciclib, or a pharmaceutically acceptable salt thereof. In some embodiments, the CDK2 inhibitor is an Example compound (or a pharmaceutically acceptable salt thereof) disclosed in the specifications of U.S. Pat. Nos. 11,066,404, 11,384,083, 11,472,791, 11,919,904, 11,440,914, 11,447,494, 11,427,567, 11,851,426, and 11,981,671 and US Patent Publ. Nos. US 20220340579, US 20230192706, and US 20230183250, each of which is incorporated herein by reference in its entirety. In some embodiments, the CDK2 inhibitor is 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine; N-((3R,4S)-1-(cyclopropylsulfonyl)-3-methylpiperidin-4-yl)-8-ethoxy-7-(1H-pyrazol-4-yl)-[1,2,4]triazolo[1,5-a]pyridin-2-amine; 8-isopropoxy-N-((3R,4S)-3-methyl-1-(methylsulfonyl)piperidin-4-yl)-7-(1H-pyrazol-4-yl)-[1,2,4]triazolo[1,5-c]pyrimidin-2-amine; 8-(ethoxy-d5)—N-((3R,4S)-3-methyl-1-(methylsulfonyl)piperidin-4-yl)-7-(1H-pyrazol-4-yl)-[1,2,4]triazolo[1,5-a]pyridin-2-amine; or 8-isopropoxy-N-((3R,4S)-3-methyl-1-(methylsulfonyl)piperidin-4-yl)-7-(1H-pyrazol-4-yl)-[1,2,4]triazolo[1,5-a]pyridin-2-amine; or a

In some embodiments, the aromatase inhibitor is anastrozole, exemestane, letrozole, vorozole, formestane, or fadrozole (or a pharmaceutically acceptable salt thereof).

In some embodiments, the LHRH agonist is leuprolide, goserelin, triptorelin, histrelin, or buserelin (or a pharmaceutically acceptable salt thereof).

In some embodiments, the anti-androgen is spironolactone, finasteride, dutasteride, bicalutamide, flutamide, enzalutamide, apalutamide, cyproterone, abiraterone, chlormadinone, clascoterone, megestrol, nilutamide, or darolutamide (or a pharmaceutically acceptable salt thereof).

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of CDK4-associated diseases, disorders or conditions. Other agents such as therapeutic antibodies can be used in combination with the compounds of the present disclosure for treatment of CDK4-associated diseases, disorders or conditions. The one or more additional pharmaceutical agents can be administered to a patient simultaneously or sequentially.

In some embodiments, the compound or salt is administered or used in combination with a BCL2 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS—R, IDH2, IGF-1R, IR—R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SUP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3Kδ inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™ e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17-alpha-ethinylestradiol, diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a compound or salt of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self -antigens.

Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### II. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, CDK4, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F₉-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, RO7009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated.

Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), or more, such as about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating CDK4 in tissue samples, including human, and for identifying CDK4 activators by inhibition binding of a labeled compound. Substitution of one or more of the atoms of the compounds of the present disclosure can also be useful in generating differentiated ADME (Adsorption, Distribution, Metabolism and Excretion.) Accordingly, the present disclosure includes CDK4 assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). For example, a compound which is “isotopically-labeled” can have a deuterium atom in place of a hydrogen atom, a ¹³C atom in place of a ¹²C atom, or any other isotopic substitution of one or more atoms constituting the molecule. Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups of the disclosed Formulas (e.g., Formula (I)) can be perdeuterated. In some embodiments, compounds of the disclosure include deuterium.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

The compounds disclosed herein can be prepared so that they are isotopically enriched. When a compound is said to be “enriched” or “has an enrichment” in an isotope, such as deuterium, it is meant that the amount of that isotope in a preparation of a compound is above the natural abundance level. For example, a preparation enriched in deuterium would be understood to contain deuterium in an amount above the natural abundance of 0.0156 mol %. Additionally, if a sample is said to be about 10% enriched in a particular isotope (e.g., deuterium), then about 10% of the molecules in the sample include that isotope at a specified position in the molecule. The isotopic enrichment of preparations of the compounds provided herein can be determined using conventional analytical methods including mass spectrometry, nuclear magnetic resonance spectroscopy, or crystallography.

In some embodiments, the isotopic enrichment of a preparation of a compound disclosed herein is at least about 45%, at least about 50%, at least about 60%, at least about 70%, at least about 80%, at least about 90%, at least about 95%, at least about 97%, at least about 98%, at least about 99%, or at least about 99.5% at the specified position(s). In some embodiments, the deuterium enrichment of a preparation of a compound disclosed herein is at least about 45%, at least about 50%, at least about 60%, at least about 70%, at least about 80%, at least about 90%, at least about 95%, at least about 97%, at least about 98%, at least about 99%, or at least about 99.5% at the specified position(s).

In some embodiments, preparations of the compounds disclosed herein are not isotopically enriched. For example, preparations of the compounds disclosed herein do not contain deuterium or any other isotope in an amount more than the natural abundance of that isotope.

An isotopically enriched preparation can also include isotopologues (compounds of the same chemical structure but with different isotopic composition, for example, containing deuterium at positions other than the specified position(s)). In some embodiments, a deuterium enriched preparation of a compound described herein can contain less than 50%, less than 40%, less than 30%, less than 20%, less than 10%, less than 5%, less than 2%, less than 1%, or less than 0.5% of other isotopologues. In some embodiments, a deuterium enriched preparation of a compound described herein is substantially free of isotopologues.

When a compound herein is represented by name or structure to include one or more deuterium atoms (e.g., by using “D” instead of “H”), it is meant to encompass a single molecule where a hydrogen atom is replaced by a deuterium at the one or more specified positions, and also to encompass preparations of the compound having a deuterium enrichment level greater than the natural abundance level of deuterium at the one or more specified positions.

The level of isotopic enrichment of a preparation of a compound disclosed herein can also be characterized by the “isotopic enrichment factor” which is based on the ratio between the isotopic abundance and the natural abundance of a given isotope and is a measure of relative abundance. In some preparations, the isotopic enrichment factor can be at least about 2000, at least about 2500, at least about 3000, at least about 4000, at least about 5000 (75% deuterium enrichment), at least about 6000 (90% deuterium enrichment), at least about 6333.3 (95% deuterium enrichment), at least about 6466.7 (97% deuterium enrichment), at least about 6600 (99% deuterium enrichment), or at least about 6633.3 (99.5% deuterium enrichment) for deuterium at a specified position.

Synthetic methods for including isotopes into organic compounds are known in the art (Deuterium Labeling in Organic Chemistry by Alan F. Thomas, New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages. In some embodiments, the disclosed compounds can include compounds where one or more constituent Cₙ₋ₘ alkyl groups are substituted with deuterium. Example groups include trideuteromethyl (CD₃), pentadeuteroethyl (CD₂CD₃), and the like.

In some embodiments, the disclosed compounds can include compounds where one or more constituent Cₙ₋ₘ alkenyl groups are substituted with deuterium. Example groups include trideuteroethenyl (—CD═CD₂), tetradeuteropropenyl, (—CD═CD—CD₂), and the like.

In some embodiments, the disclosed compounds can include compounds where one or more constituent Cₙ₋ₘ alkynyl groups are substituted with deuterium. Example groups include deuteroethynyl (—C≡CD), trideuteropropyn-1-yl, (—C≡CCD₃), and the like.

In some embodiments, the disclosed compounds can include compounds where one or more constituent Cₙ₋ₘ alkoxy groups are substituted with deuterium. Example groups include trideuteromethoxy (—OCD₃), pentadeuteroethoxy (—OCD₂CD₃), and the like.

In some embodiments, the disclosed compounds can include compounds where one or more constituent aryl groups are substituted with deuterium. Example groups include pentadeuterophenyl (i.e., perdeuterophenyl, phenyl-d₅), perdeuteronaphthyl, and the like.

In some embodiments, the disclosed compounds can include compounds where one or more constituent Cₙ₋ₘ haloalkoxy groups are substituted with deuterium. Example groups include deuterodifluoromethoxy (—OCDF₂), dideuterofluoromethoxy (—OCD₂F), and the like.

In some embodiments, the disclosed compounds can include compounds where one or more constituent cycloalkyl groups are deuterated. Example groups include perdeuterocyclopropyl, perdeuterocyclobutyl, perdeuterocyclopentyl, perdeuterocyclohexyl, and the like.

In some embodiments, the disclosed compounds can include compounds where one or more constituent heteroaryl groups are deuterated. Example groups include perdeuteropyridinyl, perdeuteropyrazinyl, perdeuteropyrimidinyl, and the like.

In some embodiments, the disclosed compounds can include compounds where one or more constituent heterocycloalkyl groups are deuterated. Example groups include perdeuteroazetidinyl, perdeuteropyrrolidinyl, perdeuteropiperidinyl, and the like.

In some embodiments, the disclosed compounds can include compounds where linking groups such as an “alkyl linking group” and/or an “alkylene linking group” are deuterated.

In some embodiments, compounds which are isotopically-substituted can include a radionuclide.

The radionuclide that is incorporated in the instant radio-labeled compounds will depend on the specific application of that radio-labeled compound. For example, for in vitro CDK4 labeling and competition assays, compounds that incorporate ³H, ¹⁴C, ⁸²Br, ¹²⁵I, ¹³¹I, or ³⁵S can be useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present disclosure can further include synthetic methods for incorporating radio-isotopes into compounds of the disclosure. Synthetic methods for incorporating radio-isotopes into organic compounds are well known in the art, and one of ordinary skill in the art will readily recognize the methods applicable for the compounds of disclosure.

A radio-labeled compound of the disclosure can be used in a screening assay to identify/evaluate compounds. For example, a newly synthesized or identified compound (i.e., test compound) which is labeled can be evaluated for its ability to bind and activate CDK4 by monitoring its concentration variation when contacting with CDK4, through tracking of the labeling. For example, a test compound (labeled) can be evaluated for its ability to reduce binding of another compound which is known to inhibit CDK4 (i.e., standard compound). Accordingly, the ability of a test compound to compete with the standard compound for binding to CDK4 directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of CDK4-associated diseases or disorders (such as, e.g., cancer, an inflammatory disease, a cardiovascular disease, or a neurodegenerative disease) which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g., “Two-Pump at-Column Dilution Configuration for Preparative LC-MS,” K. Blom, J. Combi. Chem., 4, 295 (2002); “Optimizing Preparative LC-MS Configurations and Methods for Parallel Synthesis Purification,” K. Blom, R. Sparks, J. Doughty, G. Everlof, T. Haque, A. Combs, J. Combi. Chem., 5, 670 (2003); and “Preparative LC-MS Purification: Improved Compound Specific Method Optimization,” K. Blom, B. Glass, R. Sparks, A. Combs, J. Combi. Chem., 6, 874-883 (2004). The separated compounds were typically subjected to analytical liquid chromatography mass spectrometry (LCMS) for purity check under the following conditions: Instrument: Agilent 1100 series, LC/MSD; Column: Waters Sunfire™ C₁₈ 5 μm particle size, 2.1×5.0 mm; Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm particle size, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see “Preparative LCMS Purification: Improved Compound Specific Method Optimization,” K. Blom, B. Glass, R. Sparks, A. Combs, J. Comb. Chem., 6, 874-883 (2004)). Typically, the flow rate used with the 30×100 mm column was 60 mL/minute.

pH=10 purifications: Waters XBridge C₁₈ 5 μm particle size, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (See “Preparative LCMS Purification: Improved Compound Specific Method Optimization,” K. Blom, B. Glass, R. Sparks, A. Combs, J. Comb. Chem., 6, 874-883 (2004)). Typically, the flow rate used with 30×100 mm column was 60 mL/minute.

### Intermediate 1: 6-Bromo-4-fluoro-1-isopropyl-1H-indole-3-carboxylic Acid

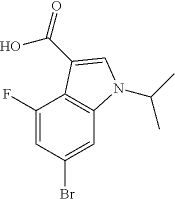 [SMILES: CC(C)n1cc(C(=O)O)c2c(F)cc(Br)cc21]

### Step 1. 6-Bromo-4-fluoro-1-isopropyl-1H-indole

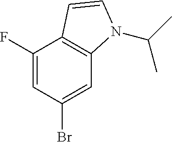

To a mixture of 6-bromo-4-fluoro-1H-indole (2.00 g, 9.34 mmol) and cesium carbonate (30.4 g, 93 mmol) in DMF (24.0 mL) was added 2-iodopropane (9.33 mL, 93 mmol). The reaction mixture was stirred at 90° C. for 1 h. After cooling to r.t., the reaction mixture was diluted with EtOAc, washed with water and brine, dried over MgSO₄, and concentrated in vacuo. The crude residue was purified by flash column chromatography (SiO₂, 0-50% EtOAc/hexanes) to afford the desired product (2.08 g, 87% yield). LC-MS calculated for C₁₁H₁₂BrFN (M+H)⁺: m/z=256.0; found 256.0.

### Step 2. 6-Bromo-4-fluoro-1-isopropyl-1H-indole-3-carbaldehyde

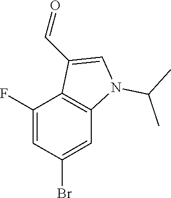 [SMILES: CC(C)n1cc(C=O)c2c(F)cc(Br)cc21]

To DMF (21 mL) at 0° C. was added POCl₃ (1.23 mL, 13.2 mmol) with vigorous stirring. The mixture was stirred at 0° C. for 10 min. A mixture of 6-bromo-4-fluoro-1-isopropyl-1H-indole (2.7 g, 10.54 mmol) in DMF (4 mL) was added dropwise, and the reaction mixture was allowed to warm to r.t. over 1 h and stirred at r.t. for 6 h. Aqueous sodium hydroxide (1 M, 42.2 mL) was added with vigorous stirring. The mixture was stirred at 80° C. for 30 min before cooling to r.t. The reaction mixture was poured into ice water (50 mL) and the mixture was slurried for 10 min to precipitate a solid. The solid was collected by filtration and dried under air to afford the desired product (2.7 g, 90% yield). The crude material obtained was used directly without further purification. LC-MS calculated for C₁₂H₁₂BrFNO (M+H)⁺: m/z=284.0; found 284.0.

### Step 3. 6-Bromo-4-fluoro-1-isopropyl-1H-indole-3-carboxylic Acid

To a mixture of 6-bromo-4-fluoro-1-isopropyl-1H-indole-3-carbaldehyde (3.36 g, 11.8 mmol) in t-BuOH/THF (1:1, 40 mL) was added 2-methyl-2-butene (12.5 mL, 118 mmol). A separate vial was charged with sodium chlorite (4.01 g, 35.5 mmol), NaH₂PO₄ (7.10 g, 59.1 mmol) and water (20 mL). After complete dissolution, the aqueous solution was added dropwise to the THF/t-BuOH solution at r.t. The resulting mixture was stirred overnight at r.t. The reaction mixture was diluted with water and extracted with EtOAc. After phase separation the organic phase was washed with brine, dried over MgSO₄, and concentrated to afford desired product (3.55 g, quant. yield). The crude material obtained was used directly without further purification. LC-MS calculated for C₁₂H₁₂BrFNO₂ (M+H)⁺: m/z=300.0; found 300.0.

### Intermediate 2: 6-(2-Chloro-5-fluoropyrimidin-4-yl)-4-fluoro-1-isopropyl-N-methyl-1H-indole-3-carboxamide

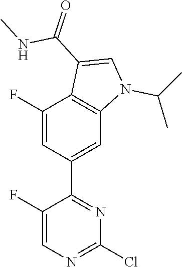 [SMILES: CNC(=O)c1cn(C(C)C)c2cc(-c3nc(Cl)ncc3F)cc(F)c12]

### Step 1. 6-Bromo-4-fluoro-1-isopropyl-N-methyl-1H-indole-3-carboxamide

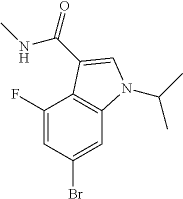 [SMILES: CNC(=O)c1cn(C(C)C)c2cc(Br)cc(F)c12]

To a mixture of 6-bromo-4-fluoro-1-isopropyl-1H-indole-3-carboxylic acid (Intermediate 1, 0.580 g, 1.93 mmol) in DMF (10 mL) was added methylamine hydrochloride (0.261 g, 3.87 mmol), N,N-diisopropylethylamine (1.35 mL, 7.73 mmol) and HATU (1.10 g, 2.90 mmol). The reaction mixture was stirred at r.t. for 1 h. The mixture was diluted with EtOAc, washed with water, brine, dried over MgSO₄, and concentrated in vacuo. The crude residue was purified by flash column chromatography (SiO₂, 0-100% EtOAc/hexanes) to afford the desired product (0.509 g, 84% yield). LC-MS calculated for C₁₃H₁₅BrFN₂O (M+H)⁺: m/z=313.0; found 313.1.

### Step 2. 4-Fluoro-1-isopropyl-N-methyl-6-(4,4,5,5-tetramethyl-1,3,2-dioxaborolan-2-yl)-1H-indole-3-carboxamide

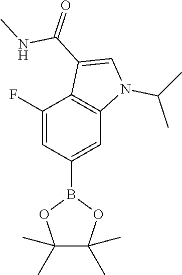 [SMILES: CNC(=O)c1cn(C(C)C)c2cc(B3OC(C)(C)C(C)(C)O3)cc(F)c12]

To a mixture of 6-bromo-4-fluoro-1-isopropyl-N-methyl-1H-indole-3-carboxamide (0.509 g, 1.63 mmol) in 1,4-dioxane (5 mL) was added bis(pinacolato)diboron (1.15 g, 4.55 mmol), potassium acetate (0.526 g, 5.36 mmol), and Pd(dppf)Cl₂·CH₂Cl₂ (0.265 g, 0.325 mmol). N₂ was sparged through the reaction mixture for 5 min, and the reaction mixture was stirred at 100° C. for 1 h. The reaction mixture was cooled to r.t., filtered, and concentrated. The crude residue was purified by flash column chromatography (SiO₂, 0-100% EtOAc/hexanes) to afford the desired product (0.513 g, 88% yield). LC-MS calculated for C₁₉H₂₇BFN₂O₃(M+H)⁺: m/z=361.2; found 361.3.

### Step 3. 6-(2-Chloro-5-fluoropyrimidin-4-yl)-4-fluoro-1-isopropyl-N-methyl-1H-indole-3-carboxamide

To a mixture of 4-fluoro-1-isopropyl-N-methyl-6-(4,4,5,5-tetramethyl-1,3,2-dioxaborolan-2-yl)-1H-indole-3-carboxamide (0.260 g, 0.722 mmol), 2,4-dichloro-5-fluoropyrimidine (0.241 g, 1.44 mmol), and potassium carbonate (0.400 g, 2.89 mmol) in 1,4-dioxane/water (5:1; 3.6 mL) was added Pd(dppf)Cl₂·CH₂Cl₂ (0.265 g, 0.325 mmol). The mixture was sparged with N₂ for 5 min and stirred at 80° C. for 2 h. The reaction mixture was cooled to r.t., diluted with water and extracted with EtOAc. The combined organic layers were washed with water and brine, dried over MgSO₄, and concentrated. The crude residue was purified by flash column chromatography (SiO₂, 0-100% EtOAc/hexanes) to afford the desired product (0.221 g, 84% yield). LC-MS calculated for C₁₇H₁₆ClF₂N₄O(M+H)⁺: m/z=365.2; found 365.1.

### Intermediate 3: Methyl 4-fluoro-1-isopropyl-6-(4,4,5,5-tetramethyl-1,3,2-dioxaborolan-2-yl)-1H-indole-3-carboxylate

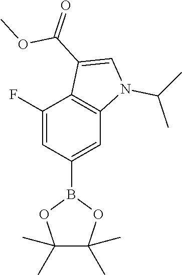 [SMILES: COC(=O)c1cn(C(C)C)c2cc(B3OC(C)(C)C(C)(C)O3)cc(F)c12]

### Step 1: Methyl 6-bromo-4-fluoro-1-isopropyl-1H-indole-3-carboxylate

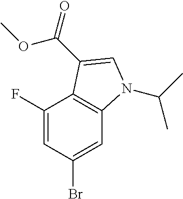 [SMILES: COC(=O)c1cn(C(C)C)c2cc(Br)cc(F)c12]

A mixture of 6-bromo-4-fluoro-1-isopropyl-1H-indole-3-carboxylic acid (Intermediate 1, 3.550 g, 11.83 mmol) in CH₂Cl₂ (22.75 mL) and MeOH (6.82 mL) was cooled to 0° C. To this mixture was added trimethylsilyldiazomethane (2 M in hexanes, 11.8 mL, 23.6 mmol) dropwise. The mixture was stirred at 0° C. for 10 min before warming to r.t. The mixture was stirred at r.t. overnight. The mixture was quenched with AcOH (2.03 mL, 35.5 mmol) and the resulting mixture was concentrated under reduced pressure. The crude residue was purified by flash column chromatography (SiO₂, 0-50% EtOAc/hexanes) to afford the desired product (3.11 g, 84% yield). LC-MS calculated for C₁₃H₁₄BrFNO₂ (M+H)⁺: m/z=314.0; found 314.1.

### Step 2: Methyl 4-fluoro-1-isopropyl-6-(4,4,5,5-tetramethyl-1,3,2-dioxaborolan-2-yl)-1H-indole-3-carboxylate

To a mixture of methyl 6-bromo-4-fluoro-1-isopropyl-1H-indole-3-carboxylate (3.11 g, 9.90 mmol) in 1,4-dioxane (50 mL) was added bis(pinacolato)diboron (7.04 g, 27.7 mmol), potassium acetate (3.21 g, 32.7 mmol), and Pd(dppf)Cl₂·CH₂Cl₂ (1.62 g, 1.98 mmol). The mixture was sparged with N₂ for 5 min and stirred at 100° C. for 1 h. The mixture was cooled to r.t., filtered, and concentrated. The crude residue was purified by flash column chromatography (SiO₂, 0-40% EtOAc/hexanes) to afford the desired product (3.39 g, 95% yield). LC-MS calculated for C₁₉H₂₆BFNO₄ (M+H)⁺: m/z=362.2; found 362.2.

### Intermediate 4: Methyl 6-(2-chloro-5-fluoropyrimidin-4-yl)-4-fluoro-1-isopropyl-1H-indole-3-carboxylate

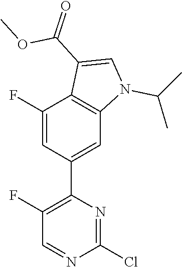 [SMILES: COC(=O)c1cn(C(C)C)c2cc(-c3nc(Cl)ncc3F)cc(F)c12]

Into a 40 mL vial was added methyl 4-fluoro-1-isopropyl-6-(4,4,5,5-tetramethyl-1,3,2-dioxaborolan-2-yl)-1H-indole-3-carboxylate (Intermediate 3, 0.500 g, 1.38 mmol), 1,4-dioxane/water (5:1; 6.92 mL), K₂CO₃ (0.765 g, 5.54 mmol), Pd(dppf)Cl₂ (0.081 g, 0.111 mmol) and 2,4-dichloro-5-fluoropyrimidine (0.462 g, 2.77 mmol). The mixture was sparged with N₂ for 5 min. The reaction was stirred at 80° C. for 2 h. The reaction mixture was diluted with water and extracted with EtOAc. The combined organic layers were dried over MgSO₄, concentrated, and purified by flash column chromatography (SiO₂, 0-50% EtOAc/CH₂Cl₂) to give the desired product (0.303 g, 60% yield). LC-MS calculated for C₁₇H₁₅ClF₂N₃O₂ (M+H)⁺: m/z=366.1; found 366.1.

### Intermediate 5: Methyl 6-(2,5-dichloropyrimidin-4-yl)-4-fluoro-1-isopropyl-1H-indole-3-carboxylate

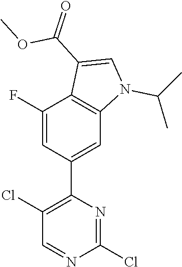 [SMILES: COC(=O)c1cn(C(C)C)c2cc(-c3nc(Cl)ncc3Cl)cc(F)c12]

This compound was prepared using the procedure described to prepare Intermediate 4, substituting 2,4,5-trichloropyrimidine for 2,4-dichloro-5-fluoropyrimidine. LC-MS calculated for C₁₇H₁₅C₂FN₃O₂(M+H)⁺: m/z=382.0; found 382.1.

### Intermediate 6. (3R,4R)-4-Amino-1-(methylsulfonyl)piperidin-3-ol hydrochloride

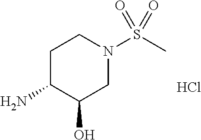

### Step 1: tert-Butyl ((3R,4R)-3-hydroxy-1-(methylsulfonyl)piperidin-4-yl)carbamate

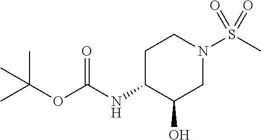 [SMILES: CC(C)(C)OC(=O)N[C@@H]1CCN(S(C)(=O)=O)C[C@H]1O]

To a mixture of tert-butyl ((3R,4R)-3-hydroxypiperidin-4-yl)carbamate (25.0 g, 116 mmol, PharmaBlock PBB1405081) and N,N-diisopropylethylamine (40.3 mL, 231 mmol) in THF (350 mL) and MeOH (17.5 mL, 433 mmol) was added methanesulfonyl chloride (9.91 mL, 127 mmol) and the reaction was stirred at r.t. for 30 min. The reaction mixture was quenched with sat. aq. NaHCO₃ (175 mL) and diluted with water (175 mL) and the resulting mixture was stirred at r.t. for 15 min. The solid precipitate that formed was collected by filtration, washed with water (350 mL), and dried to afford the desired product (27.0 g, 79% yield) as a white solid. LC-MS calculated for C₇H₁₅N₂O₅S (M+H−C₄H₈)⁺: m/z=239.1; found 239.1.

### Step 2: (3R,4R)-4-Amino-1-(methylsulfonyl)piperidin-3-ol hydrochloride

To a mixture of tert-butyl ((3R,4R)-3-hydroxy-1-(methylsulfonyl)piperidin-4-yl)carbamate (27.0 g, 92 mmol) in CH₂Cl₂/MeOH (20:1, 315 mL) was added HCl (4 M in 1,4-dioxane, 55.0 mL, 220 mmol) at r.t. The reaction mixture was stirred at 50° C. for 1 h. After cooling to r.t., the reaction mixture was diluted with Et₂O (300 mL) and slurried for 30 min. The solid precipitate was collected by filtration, washed with Et₂O (300 mL), and dried to afford the desired product (19.5 g, 92% yield) as its HCl salt in the form of a white solid. LC-MS calculated for C₆H₁₅N₂O₃S (M+H)⁺: m/z=195.1; found 195.1.

### Intermediate 7: 4-Fluoro-6-(5-fluoro-2-(((3R,4R)-3-hydroxy-1-(methylsulfonyl)piperidin-4-yl)amino)pyrimidin-4-yl)-1-isopropyl-1H-indole-3-carboxylic Acid

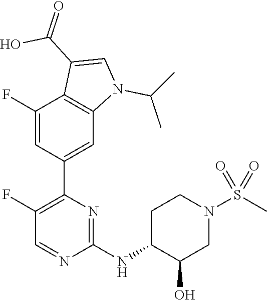 [SMILES: CC(C)n1cc(C(=O)O)c2c(F)cc(-c3nc(N[C@@H]4CCN(S(C)(=O)=O)C[C@H]4O)ncc3F)cc21]

### Step 1: Methyl 4-fluoro-6-(5-fluoro-2-(((3R,4R)-3-hydroxy-1-(methylsulfonyl)piperidin-4-yl)amino)pyrimidin-4-yl)-1-isopropyl-1H-indole-3-carboxylate

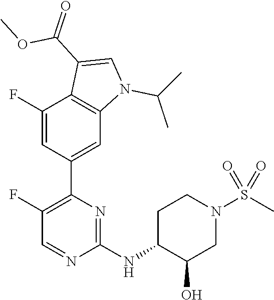

A vial containing methyl 6-(2-chloro-5-fluoropyrimidin-4-yl)-4-fluoro-1-isopropyl-1H-indole-3-carboxylate (Intermediate 4, 303 mg, 0.828 mmol), (3R,4R)-4-amino-1-(methylsulfonyl)piperidin-3-ol hydrochloride (Intermediate 6, 229 mg, 0.994 mmol), and GPhos Pd G6 TES (78 mg, 0.083 mmol, Aldrich 922900) was evacuated and backfilled with N₂ three times. Anhydrous THF (2 mL) was added, followed quickly by NaOTMS solution (1 M in THF, 2.5 mL, 2.5 mmol). The reaction mixture was stirred at 60° C. for 30 min. The reaction mixture was cooled to r.t., diluted with water and extracted with EtOAc. The combined organic layers were dried over MgSO₄ and concentrated. The crude residue was purified by flash column chromatography (SiO₂, 0-100% EtOAc/hexanes) to afford the desired product (275 mg, 63% yield). LC-MS calculated for C₂₃H₂₈F₂N₅O₅S (M+H)⁺: m/z=524.2 found 524.3.

### Step 2: 4-Fluoro-6-(5-fluoro-2-(((3R,4R)-3-hydroxy-1-(methylsulfonyl)piperidin-4-yl)amino)pyrimidin-4-yl)-1-isopropyl-1H-indole-3-carboxylic Acid

To a vial containing methyl 4-fluoro-6-(5-fluoro-2-(((3R,4R)-3-hydroxy-1-(methylsulfonyl)piperidin-4-yl)amino)pyrimidin-4-yl)-1-isopropyl-1H-indole-3-carboxylate (275 mg, 0.525 mmol) was added 1,4-dioxane (2 mL), MeOH (2 mL), water (2 mL), and lithium hydroxide (62.9 mg, 2.63 mmol). The mixture was stirred at 45° C. for 6 h. The reaction mixture was cooled to r.t. and acidified with aqueous HCl (1 M, 2.63 mL, 2.63 mmol). The mixture was diluted with water and extracted with EtOAc. The combined organic layers were dried over MgSO₄ and concentrated. The crude residue taken forward without further purification (268 mg, quant. yield). LC-MS calculated for C₂₂H₂₆F₂N₅O₅S (M+H)⁺: m/z=510.2 found 510.3.

### Intermediate 8: 6-(5-Chloro-2-(((3S,4R)-3-hydroxytetrahydro-2H-pyran-4-yl)amino)pyrimidin-4-yl)-4-fluoro-1-isopropyl-1H-indole-3-carboxylic Acid

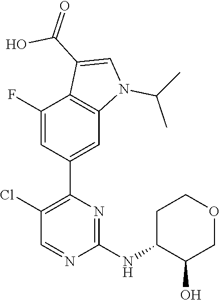 [SMILES: CC(C)n1cc(C(=O)O)c2c(F)cc(-c3nc(N[C@@H]4CCOC[C@H]4O)ncc3Cl)cc21]

### Step 1: Methyl 6-(5-chloro-2-(((3S,4R)-3-hydroxytetrahydro-2H-pyran-4-yl)amino)pyrimidin-4-yl)-4-fluoro-1-isopropyl-1H-indole-3-carboxylate

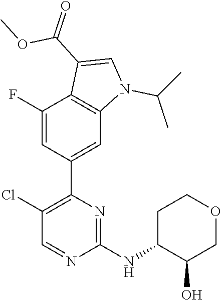 [SMILES: COC(=O)c1cn(C(C)C)c2cc(-c3nc(N[C@@H]4CCOC[C@H]4O)ncc3Cl)cc(F)c12]

To a vial containing methyl 6-(2,5-dichloropyrimidin-4-yl)-4-fluoro-1-isopropyl-1H-indole-3-carboxylate (Intermediate 5, 250 mg, 0.654 mmol) was added DMF (1.3 mL), (3S,4R)-4-aminotetrahydro-2H-pyran-3-ol hydrochloride (250 mg, 1.64 mmol, Pharmablock PBZJ8003-01), and N,N-diisopropylethylamine (0.8 mL, 4.58 mmol). The reaction mixture was stirred at 110° C. for 1 h. The reaction mixture was cooled to r.t., diluted with water, and extracted with EtOAc. The combined organic layers were dried over MgSO₄, concentrated, and purified by flash column chromatography (SiO₂, 0-100% EtOAc/hexanes) to afford the desired product (233 mg, 77% yield). LC-MS calculated for C₂₂H₂₅ClFN₄O₄(M+H)⁺: m/z=463.2 found 463.2.

### Step 2: 6-(5-Chloro-2-(((3S,4R)-3-hydroxytetrahydro-2H-pyran-4-yl)amino)pyrimidin-4-yl)-4-fluoro-1-isopropyl-1H-indole-3-carboxylic Acid

This material was prepared using the same procedure described in Intermediate 7, Step 2, substituting methyl 6-(5-chloro-2-(((3S,4R)-3-hydroxytetrahydro-2H-pyran-4-yl)amino)pyrimidin-4-yl)-4-fluoro-1-isopropyl-1H-indole-3-carboxylate for methyl 4-fluoro-6-(5-fluoro-2-(((3R,4R)-3-hydroxy-1-(methylsulfonyl)piperidin-4-yl)amino)pyrimidin-4-yl)-1-isopropyl-1H-indole-3-carboxylate. LC-MS calculated for C₂₁H₂₃ClFN₄O₄(M+H)⁺: m/z=449.1 found 449.2.

### Intermediate 9: Isopropyl 6-(2,5-dichloropyrimidin-4-yl)-4-fluoro-1-isopropyl-2-methyl-1H-indole-3-carboxylate

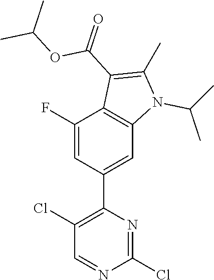 [SMILES: Cc1c(C(=O)OC(C)C)c2c(F)cc(-c3nc(Cl)ncc3Cl)cc2n1C(C)C]

### Step 1: Isopropyl 6-bromo-4-fluoro-1-isopropyl-1H-indole-3-carboxylate

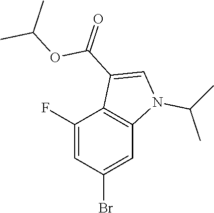 [SMILES: CC(C)OC(=O)c1cn(C(C)C)c2cc(Br)cc(F)c12]

To a mixture of 6-bromo-4-fluoro-1H-indole-3-carboxylic acid (258 mg, 1 mmol, AmBeed A979268) in MeCN (5 mL) was added 2-iodopropane (0.30 mL, 3.0 mmol) and cesium carbonate (977 mg, 3.0 mmol). The mixture was stirred at 90° C. overnight. After cooling to r.t., the mixture was filtered through a pad of Celite. The filter cake was washed with MeCN and the filtrate was concentrated under reduced pressure. The crude residue was purified by flash column chromatography (SiO₂, EtOAc/hexanes) to afford the desired product. LC-MS calculated for C₁₅H₁₈BrFNO₂ (M+H)⁺: m/z=342.1; found 342.3.

### Step 2: Isopropyl 6-bromo-4-fluoro-1-isopropyl-2-methyl-1H-indole-3-carboxylate

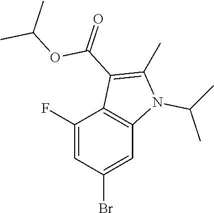 [SMILES: Cc1c(C(=O)OC(C)C)c2c(F)cc(Br)cc2n1C(C)C]

A mixture of isopropyl 6-bromo-4-fluoro-1-isopropyl-1H-indole-3-carboxylate (51 mg, 0.15 mmol) in THF (0.75 mL) was cooled to −78° C. before LDA (0.12 mL, 2M in THF, 0.225 mmol) was added dropwise. The reaction mixture was stirred at −78° C. for 30 min before the addition of methyl iodide (0.014 mL, 0.225 mmol). The mixture was stirred at −78° C. for an additional 30 min before warming to r.t. The mixture was stirred at r.t. overnight. The reaction mixture was diluted with water and extracted with EtOAc. The combined organic layers were dried over MgSO₄, concentrated, and purified by flash column chromatography (SiO₂, EtOAc/hexanes) to afford the desired product. LC-MS calculated for C₁₆H₂₀BrFNO₂ (M+H)⁺: m/z=356.1; found 356.2.

### Step 3: Isopropyl 4-fluoro-1-isopropyl-2-methyl-6-(4,4,5,5-tetramethyl-1,3,2-dioxaborolan-2-yl)-1H-indole-3-carboxylate

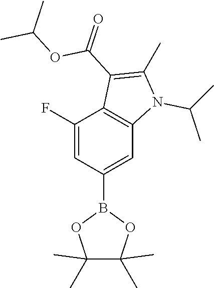 [SMILES: Cc1c(C(=O)OC(C)C)c2c(F)cc(B3OC(C)(C)C(C)(C)O3)cc2n1C(C)C]

To a mixture of isopropyl 6-bromo-4-fluoro-1-isopropyl-2-methyl-1H-indole-3-carboxylate (53 mg, 0.15 mmol) in 1,4-dioxane (0.75 mL) was added bis(pinacolato)diboron (57 mg, 0.225 mmol), potassium acetate (22 mg, 0.225 mmol), and Pd(dppf)Cl₂ (11 mg, 0.015 mmol). The mixture was sparged with N₂ for 5 min and stirred at 90° C. for 1 h. The mixture was cooled to r.t., filtered, and concentrated. The crude residue was purified by flash column chromatography (SiO₂, EtOAc/hexanes) to afford the desired product. LC-MS calculated for C₂₂H₃₂BFNO₄ (M+H)⁺: m/z=404.2; found 404.1.

### Step 4: Isopropyl 6-(2,5-dichloropyrimidin-4-yl)-4-fluoro-1-isopropyl-2-methyl-1H-indole-3-carboxylate

To a mixture of isopropyl 4-fluoro-1-isopropyl-2-methyl-6-(4,4,5,5-tetramethyl-1,3,2-dioxaborolan-2-yl)-1H-indole-3-carboxylate (Step 3) in 1,4-dioxane (0.75 mL) and water (0.15 mL) was added 2,4,5-trichloropyrimidine (41 mg, 0.225 mmol), sodium carbonate (24 mg, 0.225 mmol), and Pd(dppf)Cl₂ (11 mg, 0.015 mmol). The mixture was sparged with N₂ for 5 min and stirred at 90° C. for 3 h. The mixture was cooled to r.t., filtered, and concentrated. The crude residue was purified by flash column chromatography (SiO₂, EtOAc/hexanes) to afford the desired product. LC-MS calculated for C₂₀H₂₁Cl₂FN₃O₂ (M+H)⁺: m/z=424.1; found 424.2.

### Intermediate 10: Ethyl 6-(2-chloro-5-fluoropyrimidin-4-yl)-1-cyclopentyl-4,7-difluoro-1H-indole-3-carboxylate

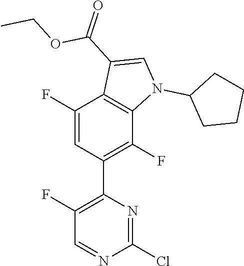 [SMILES: CCOC(=O)c1cn(C2CCCC2)c2c(F)c(-c3nc(Cl)ncc3F)cc(F)c12]

### Step 1: 4-Bromo-2-(cyclopentylamino)-3,6-difluorobenzaldehyde

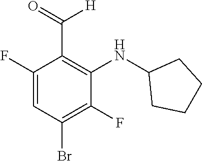 [SMILES: O=Cc1c(F)cc(Br)c(F)c1NC1CCCC1]

To a mixture of 4-bromo-2,3,6-trifluorobenzaldehyde (239 mg, 1 mmol) in DMF (5 mL) was added cyclopentylamine (0.25 mL, 2.5 mmol) and cesium carbonate (489 mg, 1.5 mmol). The mixture was stirred at 90° C. overnight. Aqueous 1 M HCl (5 mL) was added and the mixture was stir at 90° C. for an additional 2 h. After cooling to r.t., the reaction mixture was diluted with aqueous 5% LiCl and extracted with EtOAc. The combined organic layers were dried over MgSO₄, concentrated, and purified by flash column chromatography (SiO₂, EtOAc/hexanes) to afford the desired product. LC-MS calculated for C₁₂H₁₃BrF₂NO (M+H)⁺: m/z=304.0; found 304.1.

### Step 2: Ethyl 6-bromo-1-cyclopentyl-4,7-difluoro-1H-indole-3-carboxylate

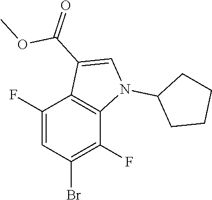 [SMILES: COC(=O)c1cn(C2CCCC2)c2c(F)c(Br)cc(F)c12]

A mixture of 4-bromo-2-(cyclopentylamino)-3,6-difluorobenzaldehyde (152 mg, 0.5 mmol) and ethyl diazoacetate (0.24 mL, 2.25 mmol) in CH₂Cl₂ (3 mL) was cooled to 0° C. To this mixture was added BF₃·Et₂O (0.091 mL, 0.75 mmol) dropwise. The mixture was stirred at 0° C. for 30 min before warming to r.t. The mixture was stirred at r.t. for 2 h. The reaction mixture was diluted with water and extracted with CH₂Cl₂. The combined organic layers were dried over MgSO₄, concentrated, and purified by flash column chromatography (SiO₂, EtOAc/hexanes) to afford the desired product. LC-MS calculated for C₁₆H₁₇BrF₂NO₂ (M+H)⁺: m/z=372.0; found 372.1.

### Step 3: Ethyl 1-cyclopentyl-4,7-difluoro-6-(4,4,5,5-tetramethyl-1,3,2-dioxaborolan-2-yl)-1H-indole-3-carboxylate

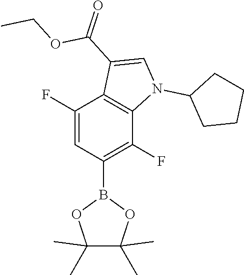 [SMILES: CCOC(=O)c1cn(C2CCCC2)c2c(F)c(B3OC(C)(C)C(C)(C)O3)cc(F)c12]

To a mixture of ethyl 6-bromo-1-cyclopentyl-4,7-difluoro-1H-indole-3-carboxylate (Step 2) in 1,4-dioxane (2 mL) was added bis(pinacolato)diboron (190 mg, 0.75 mmol), potassium acetate (74 mg, 0.75 mmol), and Pd(dppf)Cl₂ (37 mg, 0.051 mmol). The mixture was sparged with N₂ for 5 min and stirred at 90° C. for 1 h. The mixture was cooled to r.t., filtered, and concentrated. The crude residue was purified by flash column chromatography (SiO₂, EtOAc/hexanes) to afford the desired product. LC-MS calculated for C₂₂H₂₉BF₂NO₄ (M+H)⁺: m/z=420.2; found 420.3.

### Step 4: Ethyl 6-(2-chloro-5-fluoropyrimidin-4-yl)-1-cyclopentyl-4,7-difluoro-1H-indole-3-carboxylate

To a mixture of ethyl 1-cyclopentyl-4,7-difluoro-6-(4,4,5,5-tetramethyl-1,3,2-dioxaborolan-2-yl)-1H-indole-3-carboxylate (Step 3) in 1,4-dioxane (2.0 mL) and water (0.4 mL) was added 2,4-dichloro-5-fluoropyrimidine (125 mg, 0.75 mmol), sodium carbonate (79 mg, 0.75 mmol), and Pd(dppf)Cl₂ (37 mg, 0.051 mmol). The mixture was sparged with N₂ for 5 min and stirred at 90° C. for 3 h. The mixture was cooled to r.t., filtered, and concentrated. The crude residue was purified by flash column chromatography (SiO₂, EtOAc/hexanes) to afford the desired product. LC-MS calculated for C₂₀H₁₈ClF₃N₃O₂ (M+H)⁺: m/z=424.1; found 424.2.

### Intermediate 11: 4-Fluoro-6-(5-fluoro-2-(((3S,4R)-3-hydroxytetrahydro-2H-pyran-4-yl)amino)pyrimidin-4-yl)-1-isopropyl-1H-indole-3-carboxylic Acid

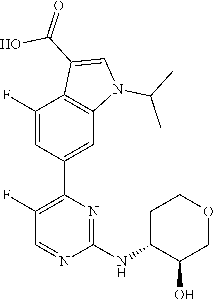 [SMILES: CC(C)n1cc(C(=O)O)c2c(F)cc(-c3nc(N[C@@H]4CCOC[C@H]4O)ncc3F)cc21]

### Step 1: Methyl 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylate

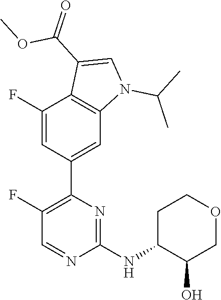 [SMILES: COC(=O)c1cn(C(C)C)c2cc(-c3nc(N[C@@H]4CCOC[C@H]4O)ncc3F)cc(F)c12]

A vial was charged with a stir bar, methyl 6-(2-chloro-5-fluoropyrimidin-4-yl)-4-fluoro-1-isopropyl-1H-indole-3-carboxylate (Intermediate 4, 1.00 g, 2.73 mmol), (3S,4R)-4-aminotetrahydro-2H-pyran-3-ol hydrochloride (504 mg, 3.28 mmol, Pharmablock PBZJ8003-01), and GPhos Pd G6 TES (258 mg, 0.273 mmol, Aldrich 922900). The vial was evacuated and backfilled with N₂ three times. To the vial was added THF (6.83 mL) and NaOTMS (1 M in THF, 8.20 mL, 8.20 mmol), and the reaction mixture was stirred at 60° C. for 30 min. The reaction mixture was cooled to r.t., diluted with water and extracted with EtOAc. The combined organic layers were dried over MgSO₄ and concentrated. The crude residue was purified by flash column chromatography (SiO₂, 0-20% CH₂Cl₂/MeOH) to afford the desired product (842 mg, 69% yield). LC-MS calculated for C₂₂H₂₅F₂N₄O₄(M+H)⁺: m/z=447.2; found 447.2.

### Step 2: 4-Fluoro-6-(5-fluoro-2-(((3S,4R)-3-hydroxytetrahydro-2H-pyran-4-yl)amino)pyrimidin-4-yl)-1-isopropyl-1H-indole-3-carboxylic Acid

To a mixture of methyl 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylate (842 mg, 1.89 mmol) in 1,4-dioxane/MeOH/H₂O/THF (1:1:1:1, 3.77 mL) was added lithium hydroxide (452 mg, 18.9 mmol) and the reaction mixture was stirred at 55° C. for 18 h. After cooling to r.t. the reaction mixture was acidified to pH 1 with aqueous concentrated HCl (12 M). The mixture was diluted with water and extracted with EtOAc. The combined organic layers were dried over MgSO₄ and concentrated to afford the title compound (720 mg, 88% yield). The crude material obtained was used directly without further purification. LC-MS calculated for C₂₁H₂₃F₂N₄O₄(M+H)⁺: m/z=433.2; found 433.2.

### Intermediate 12: Methyl (2S,3S)-3-methylazetidine-2-carboxylate 2,2,2-trifluoroacetate

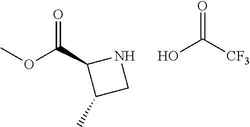

### Step 1: 1-(tert-Butyl) 2-methyl (2S,3S)-3-methylazetidine-1,2-dicarboxylate

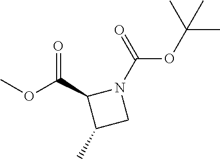 [SMILES: COC(=O)[C@@H]1[C@@H](C)CN1C(=O)OC(C)(C)C]

To a mixture of (2S,3S)-1-(tert-butoxycarbonyl)-3-methylazetidine-2-carboxylic acid (1.08 g, 5.02 mmol, PharmaBlock PBHT8060) in toluene/MeOH (3:2, 41.8 mL) was added (trimethylsilyl)diazomethane (2.0 M in THF, 3.8 mL, 7.53 mmol) dropwise. After stirring for 5 min, the reaction was quenched by slow addition of AcOH (15 mL, 262 mmol). The mixture was concentrated in vacuo. The residue taken up in CH₂Cl₂ (2.5 mL) and aq. NaOH (1 M, 2.5 mL) and the reaction mixture was stirred at r.t. for 10 min. The organic layer was dried over Na₂SO₄ and concentrated. The crude material obtained was used directly without further purification. LC-MS calculated for C₇H₁₂NO₄ (M+H−C₄H₈)⁺: m/z=174.1; found 174.2.

### Step 2: Methyl (2S,3S)-3-methylazetidine-2-carboxylate 2,2,2-trifluoroacetate

To a mixture of 1-(tert-butyl) 2-methyl (2S,3S)-3-methylazetidine-1,2-dicarboxylate (1.09 g, 4.75 mmol) in CH₂Cl₂ (12.7 mL) was added TFA (3.2 mL) and the reaction mixture was stirred at r.t. for 18 h. The mixture was concentrated in vacuo to afford the desired product as a dark yellow oil (1.2 g, 4.93 mmol, quant. yield) as its TFA salt. The crude material obtained was used without further purification. LC-MS calculated for C₆H₁₂NO₂ (M+H)⁺: m/z=130.1; found 130.2.

### Intermediate 13: (+)-(5-Azaspiro[2.3]hexan-4-yl)methanol hydrochloride

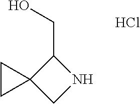

### Step 1. (±)-tert-Butyl 4-(hydroxymethyl)-5-azaspiro[2.3]hexane-5-carboxylate

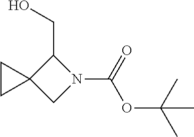

A mixture of (±)-5-(tert-butoxycarbonyl)-5-azaspiro[2.3]hexane-4-carboxylic acid (1.00 g, 4.4 mmol, Enamine EN300-1722891) in THF (8.8 mL) was cooled to 0° C. before BH₃—S(CH₃)₂ (1.6 mL, 17.6 mmol) was added dropwise. The reaction mixture was warmed to r.t. and stirred for 3 h. The reaction mixture was cooled to 0° C. and quenched by the dropwise addition of MeOH (3.5 mL). After warming to r.t., the mixture was concentrated in vacuo. The crude residue was taken up in EtOAc and H₂O. The layers were separated, and the aqueous layer extracted with EtOAc. The combined organic layers were dried over MgSO₄ and concentrated to afford the desired product (929 mg, 99% yield). The crude material obtained was used directly without further purification. LC-MS calculated for C₇H₁₁NO₃ (M+H−C₄H₉)⁺: m/z=158.1; found 158.1.

### Step 2. (+)-(5-Azaspiro[2.3]hexan-4-yl)methanol hydrochloride

To a mixture of (±)-tert-butyl 4-(hydroxymethyl)-5-azaspiro[2.3]hexane-5-carboxylate (929 mg, 4.36 mmol) in CH₂Cl₂ (20 mL) and MeOH (1 mL) was added HCl (4 M in 1,4-dioxane, 4.3 mL, 17.4 mmol). The reaction mixture was stirred at r.t. for 5 h. The mixture was concentrated to dryness to afford the desired product as an oil (652 mg, quantitative yield). The crude material obtained was used directly without further purification. LC-MS calculated for C₆H₁₂NO (M+H)⁺: m/z=114.1; found 114.2.

### Intermediate 14: ((2S,3S)-3-Methylazetidin-2-yl)methan-d2-ol hydrochloride

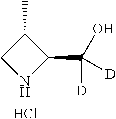

### Step 1: 1-(tert-Butyl) 2-methyl (2S,3S)-3-methylazetidine-1,2-dicarboxylate

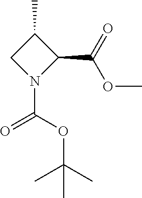 [SMILES: COC(=O)[C@@H]1[C@@H](C)CN1C(=O)OC(C)(C)C]

A mixture of (2S,3S)-1-(tert-butoxycarbonyl)-3-methylazetidine-2-carboxylic acid (1.80 g, 8.36 mmol) in CH₂Cl₂ (21.4 mL) and MeOH (6.43 mL) was cooled to 0° C. To this mixture was added trimethylsilyldiazomethane (2 M in hexanes, 8.36 mL, 16.7 mmol) dropwise and the mixture was stirred at 0° C. for 30 min. The reaction mixture was warmed to r.t. and stirred at r.t. for 3 h. The mixture was quenched with AcOH (2.25 mL, 39.3 mmol) and the resulting mixture was concentrated under reduced pressure. The crude residue was taken up in CH₂Cl₂ and washed with aq. NaOH (1 M). The organic layer was removed, and the aqueous layer was extracted twice more with CH₂Cl₂. The combined organic layers were dried over Na₂SO₄ and concentrated to afford the desired product (1.50 g, 78% yield). The crude material obtained was used directly without further purification. LC-MS calculated for C₇H₁₂NO₄ (M+H-C₄H₈)⁺: m/z=174.1; found 174.2.

### Step 2: tert-Butyl (2S,3S)-2-(hydroxymethyl-d2)-3-methylazetidine-1-carboxylate

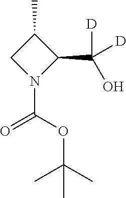 [SMILES: [2H]C([2H])(O)[C@@H]1[C@@H](C)CN1C(=O)OC(C)(C)C]

A mixture of 1-(tert-butyl) 2-methyl (2S,3S)-3-methylazetidine-1,2-dicarboxylate (Step 1, 1.50 g, 6.54 mmol) in THF (21.8 mL) was cooled to 0° C. before LiAlD₄ (550 mg, 13.1 mmol) was added portionwise. The reaction mixture was stirred at 0° C. for 30 min. The mixture was quenched with sat. aq. potassium sodium tartrate tetrahydrate (10 mL, Rochelle's salt) and the mixture was stirred at r.t. for 30 min. The mixture was transferred to a separatory funnel and extracted with EtOAc (3×20 mL). The combined organic layers were washed with brine (10 mL), dried over Na₂SO₄, and concentrated. The crude residue was purified by flash column chromatography (SiO₂, EtOAc/hexanes) to afford the desired product (895 mg, 67% yield) as a clear oil. LC-MS calculated for C₆H₁₀D₂NO₃ (M+H-C₄H₈)⁺: m/z=148.1; found 148.1.

### Step 3: ((2S,3S)-3-Methylazetidin-2-yl)methan-d2-ol hydrochloride

To a mixture of tert-butyl (2S,3S)-2-(hydroxymethyl-d₂)-3-methylazetidine-1-carboxylate (Step 2, 895 mg, 4.4 mmol) in CH₂Cl₂ (14 mL) and MeOH (0.7 mL) was added HCl (4 M in 1,4-dioxane, 5.5 mL, 22 mmol). The reaction mixture was stirred at r.t. for 90 min. The mixture was concentrated to dryness to afford the desired product (585 mg, 95% yield). The crude material obtained was used directly without further purification. LC-MS calculated for C₅H₁₀D₂NO (M+H)⁺: m/z=104.1; found 104.2.

### Intermediate 15: tert-Butyl (2R,3S)-2-((benzyloxy)methyl)-3-hydroxyazetidine-1-carboxylate

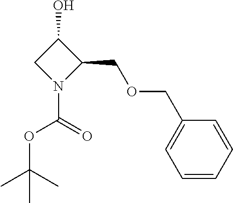 [SMILES: CC(C)(C)OC(=O)N1C[C@H](O)[C@H]1COCc1ccccc1]

### Step 1: Methyl O-(tert-butyldimethylsilyl)-L-allothreoninate

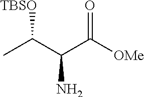 [SMILES: COC(=O)[C@@H](N)[C@H](C)O[Si](C)(C)C(C)(C)C]

To a mixture of methyl L-allothreoninate hydrochloride (5.00 g, 29.5 mmol) in CH₂Cl₂ (98 mL) was added tert-butyldimethylsilyl chloride (4.89 g, 32.4 mmol) and imidazole (6.02 g, 88 mmol) and the reaction mixture was stirred at r.t. overnight. The mixture was diluted with water, and 1 M aqueous NaOH was added until the aqueous layer reached a pH ˜12. The layers were separated, and the aqueous layer was extracted with CH₂Cl₂. The combined organic extracts were dried over MgSO₄ and concentrated. The residue was purified by flash column chromatography (120 g SiO₂, MeOH/CH₂Cl₂) to give the desired product (5.23 g, 72% yield) as a clear oil. LC-MS calculated for C₁₁H₂₆NO₃Si (M+H)⁺: m/z=248.2; found 248.2.

### Step 2: Methyl O-(tert-butyldimethylsilyl)-N-(3-(((tert-butyldimethylsilyl)oxy)methyl)picolinoyl)-L-allothreoninate

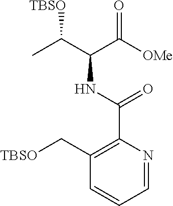 [SMILES: COC(=O)[C@@H](NC(=O)c1ncccc1CO[Si](C)(C)C(C)(C)C)[C@H](C)O[Si](C)(C)C(C)(C)C]

To a mixture of methyl O-(tert-butyldimethylsilyl)-L-allothreoninate (Step 1, 1.50 g, 6.06 mmol), 3-(((tert-butyldimethylsilyl)oxy)methyl)picolinic acid (1.62 g, 6.06 mmol, J&W Pharmlab 69R1633), and N,N-diisopropylethylamine (2.12 mL, 12.1 mmol) in MeCN (20.2 mL) was added HATU (2.42 g, 6.37 mmol) and the mixture was stirred at r.t. for 1 h. The mixture was diluted with CH₂Cl₂ and water and the layers were separated. The aqueous layer was extracted with CH₂Cl₂. The combined organic layers were dried over MgSO₄ and concentrated. The residue was purified by flash column chromatography (12 g SiO₂, EtOAc/hexanes) to give the desired product (2.64 g, 88% yield) as a clear oil. LC-MS calculated for C₂₄H₄₅N₂O₅Si₂ (M+H)⁺: m/z=497.3; found 497.3.

### Step 3: Methyl (2S,3S)-3-((tert-butyldimethylsilyl)oxy)-1-(3-(((tert-butyldimethylsilyl)oxy)methyl)picolinoyl)azetidine-2-carboxylate

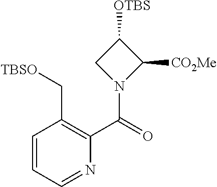 [SMILES: COC(=O)[C@@H]1[C@@H](O[Si](C)(C)C(C)(C)C)CN1C(=O)c1ncccc1CO[Si](C)(C)C(C)(C)C]

A mixture of methyl O-(tert-butyldimethylsilyl)-N-(3-(((tert-butyldimethylsilyl)oxy)methyl)picolinoyl)-L-allothreoninate (Step 2, 1.32 g, 2.66 mmol) and iodobenzene diacetate (2.140 g, 6.64 mmol) in PhMe (12.1 mL) was cooled in a dry ice/i-PrOH bath. Palladium(II) acetate (0.060 g, 0.266 mmol) was added and the mixture was degassed by three vacuum/purge cycles with N₂. The reaction mixture was warmed to r.t. and irradiated at 110° C. in a microwave reactor for 3 h. After cooling to r.t., the mixture was diluted with EtOAc and saturated aqueous NaHCO₃. The layers were separated, and the aqueous layer was extracted with EtOAc. The combined organic layers were dried over MgSO₄ and concentrated. The residue was purified by flash column chromatography (SiO₂, EtOAc/hexanes) to give the desired product (891 mg, 68% yield) as an orange oil. LC-MS calculated for C₂₄H₄₃N₂O₅Si₂ (M+H)⁺: m/z=495.3; found 495.3.

### Step 4: ((2R,3S)-3-((tert-Butyldimethylsilyl)oxy)-2-(hydroxymethyl)azetidin-1-yl)(3-(((tert-butyldimethylsilyl)oxy)methyl)pyridin-2-yl)methanone

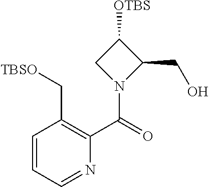 [SMILES: CC(C)(C)[Si](C)(C)OCc1cccnc1C(=O)N1C[C@H](O[Si](C)(C)C(C)(C)C)[C@H]1CO]

A mixture of methyl (2S,3S)-3-((tert-butyldimethylsilyl)oxy)-1-(3-(((tert-butyldimethylsilyl)oxy)methyl)picolinoyl)azetidine-2-carboxylate (Step 3, 685 mg, 1.38 mmol) in THF (3.46 mL) and MeOH (3.46 mL) was cooled to 0° C. before lithium borohydride (0.090 g, 4.15 mmol) was added. The reaction mixture was warmed to r.t. and stirred for 2 h. The mixture was cooled to 0° C., quenched with saturated aqueous NH₄Cl, and diluted with EtOAc. The layers were separated, and the aqueous layer was extracted with EtOAc. The combined organic layers were dried over MgSO₄ and concentrated. The residue was purified by flash column chromatography (40 g SiO₂, EtOAc/hexanes) to give the desired product (550 mg, 85% yield) as a light brown oil. LC-MS calculated for C₂₃H₄₃N₂O₄Si₂ (M+H)⁺: m/z=467.3; found 467.2.

### Step 5: ((2R,3S)-2-((Benzyloxy)methyl)-3-((tert-butyldimethylsilyl)oxy)azetidin-1-yl)(3-(((tert-butyldimethylsilyl)oxy)methyl)pyridin-2-yl)methanone

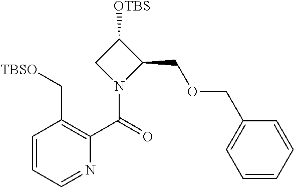 [SMILES: CC(C)(C)[Si](C)(C)OCc1cccnc1C(=O)N1C[C@H](O[Si](C)(C)C(C)(C)C)[C@H]1COCc1ccccc1]

To a mixture of ((2R,3S)-3-((tert-butyldimethylsilyl)oxy)-2-(hydroxymethyl)azetidin-1-yl)(3-(((tert-butyldimethylsilyl)oxy)methyl)pyridin-2-yl)methanone (Step 4, 550 mg, 1.18 mmol) and benzyl bromide (0.175 mL, 1.47 mmol) in DMF (2.36 mL) was added NaH (60% dispersion in mineral oil, 94 mg, 2.36 mmol) and the reaction mixture was stirred at r.t. for 2 h. Additional benzyl bromide (0.175 mL, 1.47 mmol) and NaH (60% dispersion in mineral oil, 94 mg, 2.36 mmol) were added and the reaction mixture was stirred at r.t. for 1 h. The mixture was quenched with saturated aqueous NH₄Cl and diluted with water and EtOAc. The layers were separated, and the aqueous layer was extracted with EtOAc. The combined organic layers were washed twice with brine, dried over MgSO₄, and concentrated. The residue was purified by flash column chromatography (40 g SiO₂, EtOAc/hexanes) to give the desired product (340 mg, 52% yield) as a clear oil. LC-MS calculated for C₃₀H₄₉N₂O₄Si₂ (M+H)⁺: m/z=557.3; found 557.4.

### Step 6: tert-Butyl (2R,3S)-2-((benzyloxy)methyl)-3-hydroxyazetidine-1-carboxylate

A mixture of ((2R,3S)-2-((benzyloxy)methyl)-3-((tert-butyldimethylsilyl)oxy)azetidin-1-yl)(3-(((tert-butyldimethylsilyl)oxy)methyl)pyridin-2-yl)methanone (Step 5, 367 mg, 0.659 mmol) in THF (1.65 mL) was treated with HCl (4 M in 1,4-dioxane, 1.65 mL, 6.6 mmol) and stirred at r.t. for 20 h. The mixture was concentrated in vacuo. The residue was taken up in 1,4-dioxane (3.3 mL) and Cs₂CO₃ (1.074 g, 3.30 mmol) was added, followed by di-tert-butyl dicarbonate (0.306 mL, 1.32 mmol). The mixture was stirred at r.t. for 30 minutes. Additional Cs₂CO₃ (1.074 g, 3.30 mmol) and di-tert-butyl dicarbonate (0.306 mL, 1.32 mmol) was added and the mixture was stirred at r.t. for 30 min. The mixture was diluted with CH₂Cl₂ and water, and the layers were separated. The aqueous layer was extracted with CH₂Cl₂. The combined organic layers were dried over MgSO₄ and concentrated. The residue was purified by flash column chromatography (12 g SiO₂, EtOAc/hexanes) to give the desired product (137 mg, 71% yield) as a clear oil. LC-MS calculated for C₁₂H₁₆NO₄ (M+H-C₄H₈)⁺: m/z=238.1; found 238.1.

### Intermediate 16: ((2R,3S)-3-(Difluoromethoxy)azetidin-2-yl)methanol hydrochloride

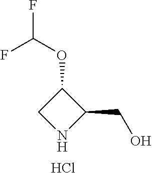

### Step 1: tert-Butyl (2R,3S)-2-((benzyloxy)methyl)-3-(difluoromethoxy)azetidine-1-carboxylate

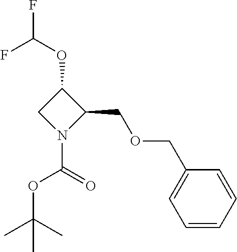 [SMILES: CC(C)(C)OC(=O)N1C[C@H](OC(F)F)[C@H]1COCc1ccccc1]

To a stirred mixture of tert-butyl (2R,3S)-2-((benzyloxy)methyl)-3-hydroxyazetidine-1-carboxylate (Intermediate 15, 137 mg, 0.467 mmol) and copper(I) iodide (17.8 mg, 0.093 mmol) in MeCN (1.20 mL) at 50° C. was added a solution of 2,2-difluoro-2-(fluorosulfonyl)acetic acid (72 μL, 0.70 mmol) in MeCN (0.6 mL) dropwise over 30 minutes. Following the addition, the reaction mixture was stirred at 50° C. for 30 minutes. After cooling to r.t., the mixture was concentrated in vacuo. The residue was taken up in EtOAc and the solids were removed through filtration. The filtrate was concentrated in vacuo and the residue was purified by flash column chromatography (4 g SiO₂, EtOAc/hexanes) to give the desired product (89 mg, 56% yield) as a clear oil. LC-MS calculated for C₁₃H₁₆F₂NO₄ (M+H−C₄H₈)⁺: m/z=288.1; found 288.1.

### Step 2: tert-Butyl (2R,3S)-3-(difluoromethoxy)-2-(hydroxymethyl)azetidine-1-carboxylate

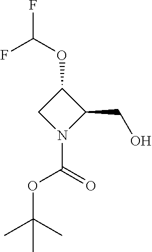 [SMILES: CC(C)(C)OC(=O)N1C[C@H](OC(F)F)[C@H]1CO]

To a mixture of tert-butyl (2R,3S)-2-((benzyloxy)methyl)-3-(difluoromethoxy)azetidine-1-carboxylate (Step 1, 89 mg, 0.26 mmol) in MeOH (2.59 mL) was added Pd—C(10 wt. %, 27.6 mg, 0.026 mmol) and the reaction mixture was equipped with a hydrogen balloon. The mixture was stirred at r.t. for 1 h, filtered through Celite, and concentrated in vacuo. The residue was purified by flash column chromatography (4 g SiO₂, EtOAc/hexanes) to give the desired product (67.9 mg, quantitative yield) as a clear oil. ¹H NMR (400 MHz, CDCl₃) δ 6.42-6.01 (m, 1H), 4.72-4.61 (m, 1H), 4.37-4.31 (m, 1H), 4.08-4.00 (m, 1H), 3.93-3.84 (m, 2H), 3.78-3.70 (m, 1H), 2.90 (br s, 1H), 1.45 (s, 9H). LC-MS calculated for C₆H₁₀F₂NO₄ (M+H−C₄H₈)⁺: m/z=198.1; found 198.1.

### Step 3: ((2R,3S)-3-(Difluoromethoxy)azetidin-2-yl)methanol hydrochloride

To a mixture of tert-butyl (2R,3S)-3-(difluoromethoxy)-2-(hydroxymethyl)azetidine-1-carboxylate (Step 2, 39.0 mg, 0.154 mmol) in THF (385 μL) was added HCl (4 M in 1,4-dioxane, 385 μL, 1.54 mmol). The reaction mixture was stirred at r.t. for 3 h. The mixture was concentrated to dryness to afford the desired product (29.2 mg, quantitative yield). The crude material obtained was used directly without further purification. LC-MS calculated for C₅H₁₀F₂NO₂ (M+H)⁺: m/z=154.1; found 154.1.

### Intermediate 17: 6-(5-Fluoro-2-(((3S,4R)-3-hydroxytetrahydro-2H-pyran-4-yl)amino)pyrimidin-4-yl)-1-methyl-1H-indole-3-carboxylic Acid

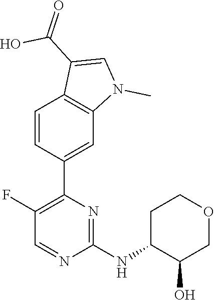 [SMILES: Cn1cc(C(=O)O)c2ccc(-c3nc(N[C@@H]4CCOC[C@H]4O)ncc3F)cc21]

### Step 1: Methyl 6-bromo-1-methyl-1H-indole-3-carboxylate

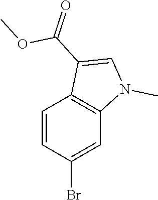

To a mixture of methyl 6-bromo-1H-indole-3-carboxylate (1.20 g, 4.72 mmol) and Cs₂CO₃ (3.85 g, 11.8 mmol) in MeCN (9.5 mL) was added iodomethane (0.735 mL, 11.8 mmol). The reaction mixture was sealed and stirred at 90° C. for 4 h. After cooling to r.t. the mixture was diluted with EtOAc and filtered through a plug of celite. The filtrate was concentrated, and the residue taken up in EtOAc, washed with brine, dried over Na₂SO₄, filtered and concentrated to afford the desired product (1.17 g, 92% yield). The crude material obtained was used directly without further purification. LC-MS calculated for C₁₁H₁₁BrNO₂ (M+H)⁺: m/z=268.0; found 268.0.

### Step 2: Methyl 1-methyl-6-(4,4,5,5-tetramethyl-1,3,2-dioxaborolan-2-yl)-1H-indole-3-carboxylate

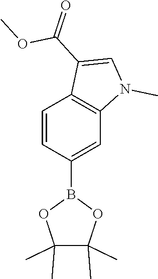

To a mixture of methyl 6-bromo-1-methyl-1H-indole-3-carboxylate (Step 1, 1.17 g, 4.36 mmol) in 1,4-dioxane (14.6 mL) was added bis(pinacolato)diboron (3.21 g, 12.7 mmol), KOAc (1.41 g, 14.4 mmol), and Pd(dppf)Cl₂·CH₂Cl₂ (0.713 g, 0.873 mmol). The reaction mixture was spared with N₂ for 5 minutes, sealed, and stirred at 100° C. for 1 h. The reaction mixture was cooled to r.t., diluted with EtOAc, filtered over celite, and concentrated. The crude residue was purified by flash column chromatography (SiO₂, 0-65% EtOAc/hexanes) to afford the desired product (1.06 g, 77% yield). LC-MS calculated for C₁₇H₂₃BNO₄ (M+H)⁺: m/z=316.2; found 316.2.

### Step 3: Methyl 6-(2-chloro-5-fluoropyrimidin-4-yl)-1-methyl-1H-indole-3-carboxylate

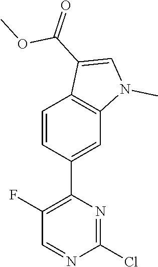

To a mixture of methyl 1-methyl-6-(4,4,5,5-tetramethyl-1,3,2-dioxaborolan-2-yl)-1H-indole-3-carboxylate (Step 2, 500 mg, 1.59 mmol), 2,4-dichloro-5-fluoropyrimidine (530 mg, 3.17 mmol), and K₂CO₃ (877 mg, 6.35 mmol) in 1,4-dioxane/water (5:1; 7.9 mL) was added Pd(dppf)Cl₂·CH₂Cl₂ (104 mg, 0.127 mmol). The mixture was sparged with N₂ for 5 min and stirred at 80° C. for 2 h. The reaction mixture was cooled to r.t., diluted with water and extracted with EtOAc. The combined organic layers were washed with sat. aq. NaHCO₃ and brine, dried over Na₂SO₄, and concentrated to afford the desired product (507 mg, quant. yield). The crude material obtained was used directly without further purification. LC-MS calculated for C₁₅H₁₂ClFN₃O₂(M+H)⁺: m/z=320.1; found 320.0.

### Step 4: Methyl 6-(5-fluoro-2-(((3S,4R)-3-hydroxytetrahydro-2H-pyran-4-yl)amino)pyrimidin-4-yl)-1-methyl-1H-indole-3-carboxylate

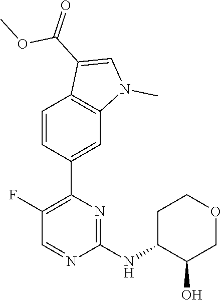 [SMILES: COC(=O)c1cn(C)c2cc(-c3nc(N[C@@H]4CCOC[C@H]4O)ncc3F)ccc12]

To a mixture of methyl 6-(2-chloro-5-fluoropyrimidin-4-yl)-1-methyl-1H-indole-3-carboxylate (Step 3, 0.600 g, 1.88 mmol), CsF (1.43 g, 9.38 mmol), and DIPEA (1.3 mL, 7.51 mmol) in DMSO (11 mL) was added (3S,4R)-4-aminotetrahydro-2H-pyran-3-ol hydrochloride (0.404 g, 2.63 mmol). The mixture stirred at 110° C. for 5 h. After cooling to r.t., the mixture was diluted with sat. aq. NaHCO₃ and extracted with EtOAc. The organic layer was separated and washed with 5% LiCl (aq) and brine, dried over Na₂SO₄, filtered, and concentrated. The crude residue was purified by flash column chromatography (SiO₂, 0-100% EtOAc/hexanes) to afford the desired product (191 mg, 25% yield). LC-MS calculated for C₂₀H₂₂FN₄O₄(M+H)⁺: m/z=401.2; found 401.2.

### Step 5: 6-(5-Fluoro-2-(((3S,4R)-3-hydroxytetrahydro-2H-pyran-4-yl)amino)pyrimidin-4-yl)-1-methyl-1H-indole-3-carboxylic Acid

To a mixture of methyl 6-(5-fluoro-2-(((3S,4R)-3-hydroxytetrahydro-2H-pyran-4-yl)amino)pyrimidin-4-yl)-1-methyl-1H-indole-3-carboxylate (Step 4, 234 mg, 0.584 mmol) in THF (3.9 mL), MeOH (3.9 mL), and H₂O (3.9 mL) was added LiOH H₂O (396 mg, 9.44 mmol). The reaction mixture was stirred at 65° C. for 1 h. After cooling to r.t, the reaction mixture was acidified with aqueous HCl (1 N, 16.5 mL, 16.5 mmol). The resulting mixture was cooled in an ice bath and stirred for 1 h. The yellow precipitate that formed was collected via filtration and dried under air to afford the desired product (142 mg, 63% yield). The crude material obtained was used directly without further purification. LC-MS calculated for C₁₉H₂OFN₄O₄(M+H)⁺: m/z=387.1; found 387.2.

### Intermediate 18: 1-Ethyl-6-(5-fluoro-2-(((3S,4R)-3-hydroxytetrahydro-2H-pyran-4-yl)amino)pyrimidin-4-yl)-1H-indole-3-carboxylic Acid

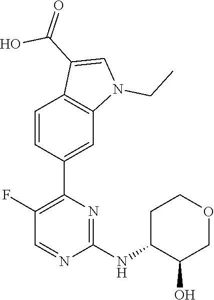 [SMILES: CCn1cc(C(=O)O)c2ccc(-c3nc(N[C@@H]4CCOC[C@H]4O)ncc3F)cc21]

### Step 1: Methyl 6-bromo-1-ethyl-1H-indole-3-carboxylate

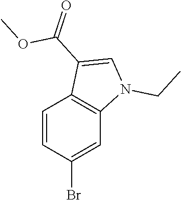 [SMILES: CCn1cc(C(=O)OC)c2ccc(Br)cc21]

To a mixture of methyl 6-bromo-1H-indole-3-carboxylate (10 g, 39 mmol) and Cs₂CO₃ (32.1 g, 98.0 mmol) in MeCN (80 mL) was added iodoethane (7.91 mL, 98.0 mmol). The reaction mixture was sealed and stirred at 90° C. for 4 h. After cooling to r.t. the mixture was diluted with EtOAc and filtered through a plug of celite. The filtrate was concentrated, and the residue taken up in EtOAc, washed with brine, dried over Na₂SO₄, filtered and concentrated. The crude residue was purified by flash column chromatography (SiO₂, 0-50% EtOAc/hexanes) to obtain the desired product (9.91 g, 89% yield). LC-MS calculated for Cl₂H₁₃BrNO₂(M+H)⁺: m/z=282.0; found 282.0.

### Step 2: Methyl 1-ethyl-6-(4,4,5,5-tetramethyl-1,3,2-dioxaborolan-2-yl)-1H-indole-3-carboxylate

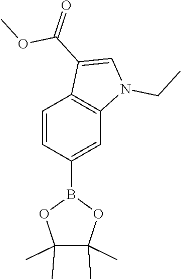 [SMILES: CCn1cc(C(=O)OC)c2ccc(B3OC(C)(C)C(C)(C)O3)cc21]

To a mixture of methyl 6-bromo-1-ethyl-1H-indole-3-carboxylate (Step 1, 9.91 g, 35.1 mmol) in 1,4-dioxane (100 mL) was added bis(pinacolato)diboron (25.9 g, 102 mmol), KOAc (11.38 g, 116 mmol), and Pd(dppf)Cl₂·CH₂Cl₂ (5.74 g, 7.02 mmol). N₂ was sparged through the reaction mixture for 5 minutes. The reaction mixture was stirred at 100° C. for 1 h. The reaction was cooled to r.t., filtered through a plug of Celite, and concentrated. The crude residue was purified by flash column chromatography (SiO₂, 0-50% EtOAc/hexanes) to afford the desired product (11.56 g, quant. yield). LC-MS calculated for C₁₈H₂₅BNO₄ (M+H)⁺: m/z=330.2; found 330.2.

### Step 3: Methyl 6-(2-chloro-5-fluoropyrimidin-4-yl)-1-ethyl-1H-indole-3-carboxylate

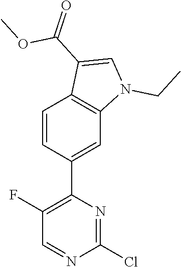 [SMILES: CCn1cc(C(=O)OC)c2ccc(-c3nc(Cl)ncc3F)cc21]

To a mixture of methyl 1-ethyl-6-(4,4,5,5-tetramethyl-1,3,2-dioxaborolan-2-yl)-1H-indole-3-carboxylate (Step 2, 7.09 g, 21.5 mmol), 2,4-dichloro-5-fluoropyrimidine (5.39 g, 32.3 mmol), and K₂CO₃ (11.91 g, 86 mmol) in 1,4-dioxane/water (5:1; 120 mL) was added Pd(dppf)Cl₂·CH₂Cl₂ (1.41 g, 1.72 mmol). The mixture was sparged with N₂ for 5 min and stirred at 80° C. for 2 h. The reaction mixture was cooled to r.t., diluted with water and extracted with EtOAc. The combined organic layers were washed with sat. aq. NaHCO₃ and brine, dried over Na₂SO₄, and concentrated. The crude residue was purified by flash column chromatography (SiO₂, 0-80% EtOAc/hexanes) to afford the desired product (4.51 g, 63% yield). LC-MS calculated for C₁₆H₁₄ClFN₃O₂ (M+H)⁺: m/z=334.1; found 334.1.

### Step 4: Methyl 1-ethyl-6-(5-fluoro-2-(((3S,4R)-3-hydroxytetrahydro-2H-pyran-4-yl)amino)pyrimidin-4-yl)-1H-indole-3-carboxylate

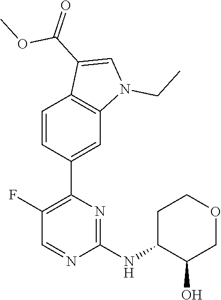 [SMILES: CCn1cc(C(=O)OC)c2ccc(-c3nc(N[C@@H]4CCOC[C@H]4O)ncc3F)cc21]

To a mixture of methyl 6-(2-chloro-5-fluoropyrimidin-4-yl)-1-ethyl-1H-indole-3-carboxylate (Step 3, 4.51 g, 13.51 mmol), CsF (8.21 g, 54.1 mmol), and DIPEA (11.8 mL, 67.6 mmol) in DMSO (100 mL) was added (3S,4R)-4-aminotetrahydro-2H-pyran-3-ol hydrochloride (3.11 g, 20.3 mmol). The mixture was stirred at 125° C. for 5 h. After cooling to r.t., the reaction mixture was cooled to room temperature, diluted with water, and stirred rapidly. The resulting suspension was filtered to afford the desired product as a solid paste (5.6 g, quant. yield). The crude material obtained was used directly without further purification. LC-MS calculated for C₂₁H₂₄FN₄O₄ (M+H)⁺: m/z=415.2; found 415.3.

### Step 5: 1-Ethyl-6-(5-fluoro-2-(((3S,4R)-3-hydroxytetrahydro-2H-pyran-4-yl)amino)pyrimidin-4-yl)-1H-indole-3-carboxylic Acid

To a mixture of methyl 1-ethyl-6-(5-fluoro-2-(((3S,4R)-3-hydroxytetrahydro-2H-pyran-4-yl)amino)pyrimidin-4-yl)-1H-indole-3-carboxylate (Step 4, 6.00 g, 14.5 mmol) in THF (20 mL), MeOH (20 mL), and H₂O (20 mL) was added LiOH·H₂O (3.04 g, 72.4 mmol). The reaction mixture was stirred at 70° C. for 5 h. After cooling to r.t., the reaction mixture was acidified to pH=4 with aqueous HCl (1 N) and the resulting mixture was stirred in an ice bath for 1 h. The yellow precipitate was collected via filtration to afford the desired product (5.80 g, quant. yield). The crude material obtained was used directly without further purification. LC-MS calculated for C₂₀H₂₂FN₄O₄ (M+H)⁺: m/z=401.2; found 401.2.

### Intermediate 19: 4-Fluoro-6-(5-fluoro-2-(((3S,4R)-3-hydroxytetrahydro-2H-pyran-4-yl)amino)pyrimidin-4-yl)-1-methyl-1H-indole-3-carboxylic Acid

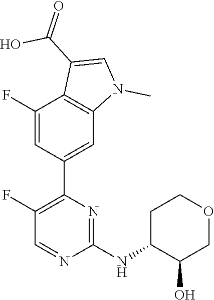 [SMILES: Cn1cc(C(=O)O)c2c(F)cc(-c3nc(N[C@@H]4CCOC[C@H]4O)ncc3F)cc21]

### Step 1. 6-Bromo-4-fluoro-1-methyl-1H-indole

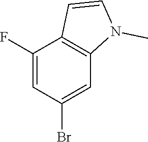

To a mixture of 6-bromo-4-fluoro-1H-indole (11.0 g, 51.4 mmol) and Cs₂CO₃ (41.9 g, 128 mmol) in DMF (100 mL) was added iodomethane (8.0 mL, 128 mmol). The reaction mixture was stirred at 90° C. for 1 h. After cooling to r.t., the reaction mixture was diluted with EtOAc, washed with water and brine, dried over MgSO₄, and concentrated in vacuo. The crude residue was purified by flash column chromatography (SiO₂, 0-50% EtOAc/hexanes) to afford the desired product (11.3 g, 97% yield). LC-MS calculated for C₉H₈BrFN (M+H)⁺: m/z=228.0; found 228.0.

### Step 2. 6-Bromo-4-fluoro-1-methyl-1H-indole-3-carbaldehyde

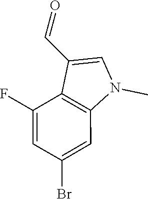

To DMF (50 mL) at 0° C. was added POCl₃ (5.79 mL, 62.2 mmol) with vigorous stirring. The mixture was stirred at 0° C. for 10 min. A mixture of 6-bromo-4-fluoro-1-methyl-1H-indole (Step 1, 11.3 g, 49.7 mmol) in DMF (25 mL) was added dropwise, and the reaction mixture was allowed to warm to r.t. over 1 h and stirred for 6 h. Aqueous NaOH (1 M, 200 mL) was added with vigorous stirring. The mixture was stirred at 80° C. for 30 min before cooling to r.t. The reaction mixture was poured into ice water (50 mL) and the mixture was slurried for 10 min to precipitate a solid. The solid was collected by filtration and dried under air to afford the desired product (11.5 g, 90% yield). The crude material obtained was used directly without further purification. LC-MS calculated for C₁₀H₈BrFNO (M+H)⁺: m/z=256.0; found 255.9.

### Step 3. 6-Bromo-4-fluoro-1-methyl-1H-indole-3-carboxylic Acid

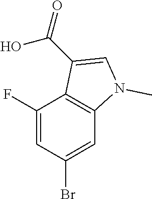

To a mixture of 6-bromo-4-fluoro-1-methyl-1H-indole-3-carbaldehyde (Step 2, 11.5 g, 44.8 mmol) in t-BuOH/THF (1:1, 140 mL) was added 2-methyl-2-butene (56.9 mL, 448 mmol). A solution of sodium chlorite (19.0 g, 168 mmol), NaH₂PO₄ (33.6 g, 280 mmol) and water (70 mL) was added dropwise, and the reaction mixture was stirred at r.t. overnight. The mixture was diluted with water and extracted with EtOAc. After phase separation the organic phase was washed with brine, dried over MgSO₄, and concentrated to afford desired product (8.68 g, 71% yield). The crude material obtained was used directly without further purification. LC-MS calculated for C₁₀H₈BrFNO₂ (M+H)⁺: m/z=272.0; found 272.0.

### Step 4: Methyl 6-bromo-4-fluoro-1-methyl-1H-indole-3-carboxylate

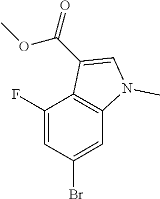

A mixture of 6-bromo-4-fluoro-1-methyl-1H-indole-3-carboxylic acid (Step 3, 8.68 g, 31.9 mmol) in CH₂Cl₂ (75 mL) and MeOH (10 mL) was cooled to 0° C. To this mixture was added trimethylsilyldiazomethane (1.8 M in hexanes, 63.8 mL, 35.4 mmol) dropwise. The mixture was stirred at 0° C. for 10 min before warming to r.t. The mixture was stirred at r.t. overnight. The mixture was quenched with AcOH (4.06 mL, 70.0 mmol) and the resulting mixture was concentrated under reduced pressure. The crude residue was purified by flash column chromatography (SiO₂, 0-50% EtOAc/hexanes) to afford the desired product (8.74 g, 96% yield). LC-MS calculated for C₁₁H₁₀BrFNO₂ (M+H)⁺: m/z=286.0; found 286.1.

### Step 5: Methyl 4-fluoro-1-methyl-6-(4,4,5,5-tetramethyl-1,3,2-dioxaborolan-2-yl)-1H-indole-3-carboxylate

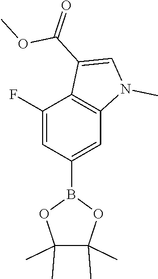

To a mixture of methyl 6-bromo-4-fluoro-1-methyl-1H-indole-3-carboxylate (Step 4, 8.74 g, 30.5 mmol) in 1,4-dioxane (50 mL) was added bis(pinacolato)diboron (14.0 g, 55.0 mmol), KOAc (9.90 g, 101 mmol), and Pd(dppf)Cl₂·CH₂Cl₂ (2.24 g, 3.05 mmol). The mixture was sparged with N₂ for 5 min and stirred at 100° C. for 1 h. The mixture was cooled to r.t., filtered, and concentrated. The crude residue was purified by flash column chromatography (SiO₂, 0-40% EtOAc/hexanes) to afford the desired product (10.2 g, 99% yield). LC-MS calculated for C₁₇H₂₂BFNO₄ (M+H)⁺: m/z=334.2; found 334.0.

### Step 6: Methyl 6-(2-chloro-5-fluoropyrimidin-4-yl)-4-fluoro-1-methyl-1H-indole-3-carboxylate

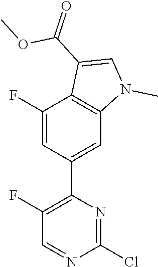

Into a 40 mL vial was added methyl 4-fluoro-1-methyl-6-(4,4,5,5-tetramethyl-1,3,2-dioxaborolan-2-yl)-1H-indole-3-carboxylate (Step 5, 10.2 g, 30.6 mmol), 1,4-dioxane/water (5:1; 150 mL), Na₂CO₃ (12.95 g, 122 mmol), Pd(dppf)Cl₂ (2.50 g, 3.06 mmol) and 2,4-dichloro-5-fluoropyrimidine (10.2 g, 61.1 mmol). The mixture was sparged with N₂ for 5 min. The reaction was stirred at 80° C. for 2 h. The reaction mixture was diluted with water and extracted with EtOAc. The combined organic layers were dried over MgSO₄, concentrated, and purified by flash column chromatography (SiO₂, 0-50% EtOAc/CH₂Cl₂) to give the desired product (6.48 g, 63% yield). LC-MS calculated for C₁₅H₁₁ClF₂N₃O₂ (M+H)⁺: m/z=338.1; found 338.1.

### Step 7: Methyl 4-fluoro-6-(5-fluoro-2-(((3S,4R)-3-hydroxytetrahydro-2H-pyran-4-yl)amino)pyrimidin-4-yl)-1-methyl-1H-indole-3-carboxylate

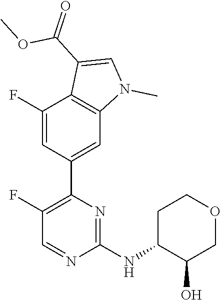 [SMILES: COC(=O)c1cn(C)c2cc(-c3nc(N[C@@H]4CCOC[C@H]4O)ncc3F)cc(F)c12]

To a mixture of methyl 6-(2-chloro-5-fluoropyrimidin-4-yl)-4-fluoro-1-methyl-1H-indole-3-carboxylate (Step 6, 5.23 g, 15.5 mmol), CsF (9.41 g, 61.9 mmol), and DIPEA (10.8 mL, 61.9 mmol) in DMSO (50 mL) was added (3S,4R)-4-aminotetrahydro-2H-pyran-3-ol hydrochloride (0.404 g, 2.63 mmol). The mixture stirred at 110° C. for 5 h. After cooling to r.t., the mixture was diluted with sat. aq. NaHCO₃ and extracted with EtOAc. The organic layer was separated and washed with 5% LiCl (aq) and brine, dried over Na₂SO₄, filtered, and concentrated. The crude residue was purified by flash column chromatography (SiO₂, 0-100% EtOAc/hexanes) to afford the desired product (6.48 g, 99% yield). LC-MS calculated for C₂₀H₂₁F₂N₄O₄ (M+H)⁺: m/z=419.2; found 419.2.

### Step 8: 4-Fluoro-6-(5-fluoro-2-(((3S,4R)-3-hydroxytetrahydro-2H-pyran-4-yl)amino)pyrimidin-4-yl)-1-methyl-1H-indole-3-carboxylic Acid

To a mixture of methyl 4-fluoro-6-(5-fluoro-2-(((3S,4R)-3-hydroxytetrahydro-2H-pyran-4-yl)amino)pyrimidin-4-yl)-1-methyl-1H-indole-3-carboxylate (Step 7, 1.0 g, 2.4 mmol) in THF (3.19 mL), MeOH (3.19 mL), and H₂O (3.19 mL) was added LiOH H₂O (600 mg, 23.9 mmol). The reaction mixture was stirred at 65° C. for 1 h. After cooling to r.t, the reaction mixture was acidified with aqueous HCl (12 N, 2 mL, 23.9 mmol). The resulting mixture was cooled in an ice bath and stirred for 1 h. The yellow precipitate that formed was collected via filtration and dried under air to afford the desired product (950 mg, 98% yield). The crude material obtained was used directly without further purification. LC-MS calculated for C₁₉H₁₉F₂N₄O₄(M+H)⁺: m/z=405.1; found 405.1.

### Intermediate 20. 1-Ethyl-4-fluoro-6-(5-fluoro-2-(((3S,4R)-3-hydroxytetrahydro-2H-pyran-4-yl)amino)pyrimidin-4-yl)-1H-indole-3-carboxylic Acid

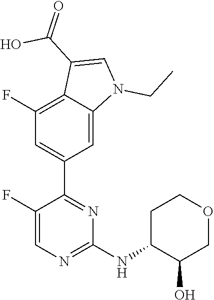 [SMILES: CCn1cc(C(=O)O)c2c(F)cc(-c3nc(N[C@@H]4CCOC[C@H]4O)ncc3F)cc21]

The title compound was prepared according to the procedures outlined for Intermediate 19, with iodoethane replacing iodomethane in step 1. LC-MS calculated for C₂₀H₂₁F₂N₄O₄ (M+H)⁺: m/z=419.2; found 419.2.

### Intermediate 21. ((2S,3S)-3-(Trifluoromethyl)azetidin-2-yl)methanol

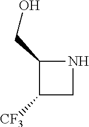

### Step 1: (S)-2-Methyl-N—((E)-3,3,3-trifluoro-2-methylpropylidene)propane-2-sulfinamide

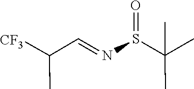

To a mixture of 3,3,3-trifluoro-2-methylpropanal (1.00 g, 7.93 mmol, Astatech F16383) in anhydrous THF (20 mL) was added (S)-2-methylpropane-2-sulfinamide (1.15 g, 9.52 mmol) and titanium(IV) ethanolate (2.45 mL, 11.90 mmol). The reaction mixture was stirred at 60° C. overnight. After cooling to rt, the mixture was poured into 75 mL of a saturated aqueous solution of NaHCO₃, and the precipitate removed by filtration over Celite. The filter cake was washed with EtOAc. The filtrate was then extracted with EtOAc (3×50 mL). The combined organic layers were washed with water, dried over Na₂SO₄, and concentrated. The crude residue was purified by flash column chromatography (SiO₂, EtOAc/hexanes) to afford the desired product as a yellow oil. LC-MS calculated for C₈H₁₅F₃NOS (M+H)⁺: m/z=230.1; found 230.1.

### Step 2: (S)—N-((1S)-1-Cyano-3,3,3-trifluoro-2-methylpropyl)-2-methylpropane-2-sulfinamide

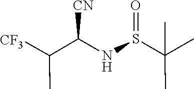 [SMILES: CC([C@@H](C#N)N[S@@](=O)C(C)(C)C)C(F)(F)F]

To a mixture of (S)-2-methyl-N—((E)-3,3,3-trifluoro-2-methylpropylidene)propane-2-sulfinamide (Step 1, 1.38 g, 6.00 mmol) in anhydrous DCM (50 mL) at 0° C. was added scandium(III) trifluoromethanesulfonate (443 mg, 0.90 mmol) and trimethylsilyl cyanide (1.77 mL, 13.20 mmol). The reaction mixture was allowed to warm to rt and stirred for 2 days. The reaction mixture was diluted with saturated aqueous NaHCO₃ and extracted with EtOAc. The combined organic layers were washed with water, dried over Na₂SO₄, and concentrated. The crude residue was purified by flash column chromatography (SiO₂, EtOAc/hexanes) to afford the desired product as a yellow oil (830 mg, 54%). LC-MS calculated for C₉H₁₆F₃N₂OS (M+H)⁺: m/z=257.1; found 257.1.

### Step 3: Methyl (2S)-2-amino-4,4,4-trifluoro-3-methylbutanoate hydrochloride

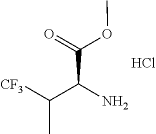

To a mixture of (S)—N-((1S)-1-cyano-3,3,3-trifluoro-2-methylpropyl)-2-methylpropane-2-sulfinamide (Step 2, 830 mg, 3.24 mmol) in MeOH (10 mL) was added HCl (4 N in dioxane, 10 mL, 40 mmol). The mixture was allowed to stir at 80° C. for 2 days. The mixture was evaporated to dryness under reduced pressure. The crude material obtained was used directly without further purification. LC-MS calculated for C₆H₁₁F₃NO₂ (M+H)⁺: m/z=186.1; found 186.0.

### Step 4: Methyl (2S)-4,4,4-trifluoro-3-methyl-2-(picolinamido)butanoate

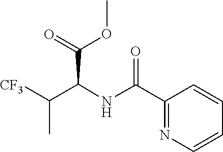 [SMILES: COC(=O)[C@@H](NC(=O)c1ccccn1)C(C)C(F)(F)F]

To a mixture of methyl (2S)-2-amino-4,4,4-trifluoro-3-methylbutanoate hydrochloride (Step 3) in Acetonitrile (20 mL) was added picolinic acid (554 mg, 4.50 mmol), HATU (2.28 g, 6.00 mmol) and N,N-diisopropylethylamine (1.31 mL, 7.50 mmol). The mixture was allowed to stir at rt for 2 h. The reaction mixture was diluted with water and extracted with EtOAc. The combined organic layers were washed with water, dried over Na₂SO₄, and concentrated. The crude residue was purified by flash column chromatography (SiO₂, EtOAc/hexanes) to afford the desired product as a yellow oil (382 mg, 44%). LC-MS calculated for C₁₂H₁₄F₃N₂O₃(M+H)⁺: m/z=291.1; found 291.0.

### Step 5: Methyl (2S,3S)-1-picolinoyl-3-(trifluoromethyl)azetidine-2-carboxylate

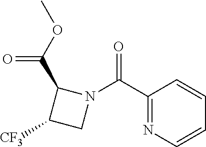 [SMILES: COC(=O)[C@@H]1[C@@H](C(F)(F)F)CN1C(=O)c1ccccn1]

To a mixture of methyl (2S)-4,4,4-trifluoro-3-methyl-2-(picolinamido)butanoate (Step 4, 382 mg, 1.3 mmol) in toluene (7 mL) was added (diacetoxyiodo)benzene (1.06 g, 3.29 mmol) and palladium(II) acetate (29.5 mg, 0.13 mmol). The mixture was sparged with N₂ for 5 min before it was allowed to stir at 110° C. overnight. The reaction mixture was diluted with water and extracted with EtOAc. The combined organic layers were washed with water, dried over Na₂SO₄, and concentrated. The crude residue was purified by flash column chromatography (SiO₂, EtOAc/hexanes) to afford the desired product as a yellow oil (100 mg, 26%). LC-MS calculated for C₁₂H₁₂F₃N₂O₃(M+H)⁺: m/z=289.1; found 289.2.

### Step 6: ((2S,3S)-3-(Trifluoromethyl)azetidin-2-yl)methanol

To a mixture of methyl (2S,3S)-1-picolinoyl-3-(trifluoromethyl)azetidine-2-carboxylate (Step 5, 100 mg, 0.35 mmol) in THF (2 mL) at −20° C. was added LAH (1 M in THF, 0.69 mL, 0.69 mmol). The mixture was allowed to stir at that temperature for 2 h. The reaction mixture was quenched with 0.1 mL water at 0° C. carefully, followed by addition of 0.1 mL aqueous NaOH (1 N) and 0.3 mL water. The mixture was further allowed to stir at rt for 30 min before it was filtered through a pad of Celite. The filtrate was concentrated under reduced pressure. The crude material obtained was used directly without further purification. LC-MS calculated for C₅H₉F₃NO (M+H)⁺: m/z=156.1; found 156.1.

### Intermediate 22. ((2S,3S)-3-(Trifluoromethyl)azetidin-2-yl)methan-d2-ol

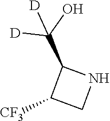 [SMILES: [2H]C([2H])(O)[C@H]1NC[C@@H]1C(F)(F)F]

The title compound was prepared according to the procedures outlined for Intermediate 21, with LiAlD₄ (Strem 03-0750) replacing LiAlH₄ in step 6. LC-MS calculated for C₅H₇D₂F₃NO (M+H)⁺: m/z=158.1; found 158.0.

### Intermediate 23. ((2R,3R)-3-Methoxy-3-methylazetidin-2-yl)methanol

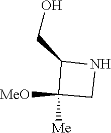

### Step 1: Tosyl-D-serine

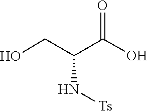 [SMILES: Cc1ccc(S(=O)(=O)N[C@H](CO)C(=O)O)cc1]

To a mixture of D-serine (8.00 g, 76 mmol) in aq. sodium hydroxide (2 M, 76 mL) at 0° C. was added p-toluenesulfonyl chloride (14.51 g, 76 mmol). The mixture was stirred at 0° C. for 30 minutes before an additional aq. NaOH (2 M, 10 mL) was added, and the mixture was warmed to room temperature and stirred for 24 h. Concentrated HCl was added until the pH was ˜1. The resulting precipitate was collected by filtration, washed with brine, water, and EtOH to give the product tosyl-D-serine as a white solid (11.7 g, 59%). LC-MS calculated for C₁₀H₁₄NO₅S (M+H)⁺: m/z=260.1; found 260.1.

### Step 2: O-(tert-Butyldiphenylsilyl)N-tosyl-D-serine

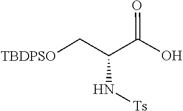

To a mixture of tosyl-D-serine (Step 1, 5.19 g, 20 mmol) and imidazole (2.04 g, 30 mmol) in DMF (50 mL) at 0° C. was added tert-butyl (chloro)diphenylsilane (7.71 mL, 30 mmol). The mixture was warmed to room temperature and stirred for 24 h. The reaction mixture was diluted with EtOAc, and the mixture was washed with LiCl (10% aq.), water, and brine. The combined organic layers were dried over Na₂SO₄, and concentrated. The crude residue was purified by flash column chromatography (SiO₂, EtOAc/hexanes) to afford the desired product as a white solid. LC-MS calculated for C₂₀H₂₆NO₅SSi (M−C₆H₅)⁺: m/z=420.1; found 420.4.

### Step 3: (R)—N-(1-((tert-Butyldiphenylsilyl)oxy)-4-diazo-3-oxobutan-2-yl)-4-methylbenzenesulfonamide

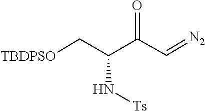

To a mixture of O-(tert-butyldiphenylsilyl)-N-tosyl-D-serine (Step 2, 4.98 g, 10 mmol) in DCM (50.0 mL) at 0° C. was added oxalyl chloride (1.05 mL, 12.0 mmol), followed by 2 drops of DMF. The mixture was warmed to room temperature and stirred for 2 h, then concentrated in vacuo. The resulting residue was taken up in THF (50.0 mL) and cooled to 0° C. (Trimethylsilyl)diazomethane (2 M in diethyl ether, 11.0 mL, 22.0 mmol) was added dropwise, and the mixture was stirred at 0° C. overnight. The mixture was evaporated to dryness under reduced pressure, and resulting residue was purified by flash column chromatography (SiO₂, EtOAc/hexanes) to afford the desired product as a yellow oil. LC-MS calculated for C₂₁H₂₆N₃O₄SSi (M−C₆H₅)⁺: m/z=444.1; found 444.1.

### Step 4: (R)-2-(((tert-Butyldiphenylsilyl)oxy)methyl)-1-tosylazetidin-3-one

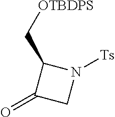

To a mixture of (R)—N-(1-((tert-butyldiphenylsilyl)oxy)-4-diazo-3-oxobutan-2-yl)-4-methylbenzenesulfonamide (Step 3) in PhMe (50 mL) at 90° C. was added copper(II) acetylacetonate (0.262 g, 1.0 mmol) in one portion. The mixture was stirred at that temperature for 5 minutes, then cooled to room temperature and concentrated in vacuo. The resulting residue was purified by flash column chromatography (SiO₂, EtOAc/hexanes) to afford the desired product as a yellow solid (3.5 g, 71% over two steps). LC-MS calculated for C₂₁H₂₆NO₄SSi (M−C₆H₅)⁺: m/z=416.1; found 416.2.

### Step 5: (2R,3R)-2-(((tert-Butyldiphenylsilyl)oxy)methyl)-3-methyl-1-tosylazetidin-3-ol

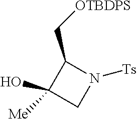

To a mixture of (R)-2-(((tert-butyldiphenylsilyl)oxy)methyl)-1-tosylazetidin-3-one (Step 4, 725 mg, 1.47 mmol) in THF (4.0 mL) at 0° C. was added methylmagnesium bromide (3.0 M in THF, 0.73 mL, 2.2 mmol). The mixture was stirred at that temperature for 2 h. The reaction mixture was diluted with water and extracted with EtOAc. The combined organic layers were washed with water, dried over Na₂SO₄, and concentrated. The crude residue was purified by flash column chromatography (SiO₂, EtOAc/hexanes) to afford the desired product as a colorless oil. LC-MS calculated for C₂₂H₃₀NO₄SSi (M-C₆H₅)⁺: m/z=432.2; found 432.2.

### Step 6: (2R,3R)-2-(((tert-Butyldiphenylsilyl)oxy)methyl)-3-methoxy-3-methyl-1-tosylazetidine

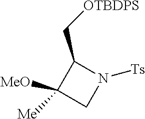

To a mixture of (2R,3R)-2-(((tert-butyldiphenylsilyl)oxy)methyl)-3-methyl-1-tosylazetidin-3-ol (Step 5, 60 mg, 0.12 mmol) in THF (0.5 mL) at 0° C. was added sodium hydride (60% in mineral oil, 7.1 mg, 0.18 mmol). The mixture was stirred at that temperature for 0.5 h before iodomethane (0.011 mL, 0.18 mmol) was added. The reaction mixture was stirred for 1 h and diluted with water and extracted with EtOAc. The combined organic layers were washed with water, dried over Na₂SO₄, and concentrated. The crude material obtained was used directly without further purification. LC-MS calculated for C₂₃H₃₂NO₄SSi (M−C₆H₅)⁺: m/z=446.2; found 446.2.

### Step 7: (2R,3R)-2-(((tert-Butyldiphenylsilyl)oxy)methyl)-3-methoxy-3-methylazetidine

To a mixture of (2R,3R)-2-(((tert-butyldiphenylsilyl)oxy)methyl)-3-methoxy-3-methyl-1-tosylazetidine (Step 6) in MeOH (0.5 mL) at 0° C. was added magnesium (100 mg, 4.11 mmol). The mixture is sonicated under room temperature for 1 h. The reaction mixture was diluted with EtOAc and filtered through a pad of Celite. The filtrate was concentrated under reduced pressure, and the crude residue was purified by flash column chromatography (SiO₂, EtOAc/hexanes) to afford the desired product as a colorless oil. LC-MS calculated for C₂₂H₃₂NO₂Si (M+H)⁺: m/z=370.2; found 370.3.

### Intermediate 24: ((2S,3S)-3-Methylazetidin-2-yl)methanol hydrochloride

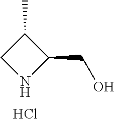

The title compound was prepared according to the procedures described in Intermediate 14, using LiAlH₄ instead of LiAlD₄ in Step 2. LC-MS calculated for C₅H₁₂NO (M+H)⁺: m/z=102.1; found 102.1.

### Intermediate 25. Methyl (2S,3S)-3-((tert-butyldimethylsilyl)oxy)-1-picolinoylazetidine-2-carboxylate

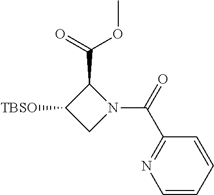 [SMILES: COC(=O)[C@@H]1[C@@H](O[Si](C)(C)C(C)(C)C)CN1C(=O)c1ccccn1]

### Step 1: Methyl picolinoyl-L-allothreoninate

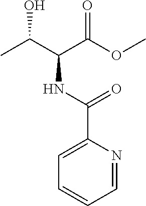 [SMILES: COC(=O)[C@@H](NC(=O)c1ccccn1)[C@H](C)O]

Methyl L-allothreoninate hydrochloride (AA Blocks, AA00364O, 34.4 g, 203 mmol) was added to a mixture of picolinic acid (25 g, 203 mmol), HATU (79 g, 207 mmol), and N,N-diisopropylethylamine (89 mL, 508 mmol) in THF (677 mL). The mixture was stirred at r.t. for 2 h, concentrated in vacuo, and the residue was taken up in EtOAc (˜700 mL). The organic layer was washed with water and brine, dried over magnesium sulfate, and concentrated in vacuo. The residue was taken up in CH₂Cl₂, filtered, and concentrated in vacuo. The residue was purified by flash column chromatography (330 g SiO₂, EtOAc/hexanes) to give the product as an impure mixture, which was used without further purification. LC-MS calculated for C₁₁H₁₅N₂O₄ (M+H)⁺: m/z=239.1; found 239.1.

### Step 2: Methyl O-(tert-butyldimethylsilyl)-N-picolinoyl-L-allothreoninate

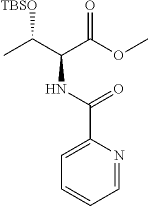 [SMILES: COC(=O)[C@@H](NC(=O)c1ccccn1)[C@H](C)O[Si](C)(C)C(C)(C)C]

TBSCl (27.1 g, 180 mmol) was added to a mixture of methyl picolinoyl-L-allothreoninate (Step 1) and imidazole (13.9 g, 204 mmol) in CH₂Cl₂ (327 mL) at 0° C. The reaction mixture was warmed to r.t. and stirred for 2 h. The mixture was diluted with water and the layers were separated. The organic layer was dried over magnesium sulfate, filtered, and concentrated in vacuo. The residue was purified by flash column chromatography (330 g SiO₂, EtOAc/hexanes) to give the title compound (33.0 g, 46% yield over 2 steps) as a clear oil. LC-MS calculated for C₁₇H₂₉N₂O₄Si (M+H)⁺: m/z=353.2; found 353.2.

### Step 3: Methyl (2S, 3S)-3-((tert-butyldimethylsilyl)oxy)-1-picolinoylazetidine-2-carboxylate

In two separate flasks, a suspension of methyl 0-(tert-butyldimethylsilyl)-N-picolinoyl-L-allothreoninate (Step 2, 16.5 g, 46.8 mmol) and iodobenzene diacetate (37.7 g, 117 mmol) in toluene (213 mL) was cooled in a to −78° C. Pd(OAc)₂ (0.525 g, 2.34 mmol) was added, a reflux condenser was attached, and the reaction set-up was degassed by three vacuum/purge cycles with nitrogen. The mixture was warmed to r.t. in a tepid water bath and the reaction mixture was heated to 110° C. for 3 h. After cooling to r.t., the mixture was diluted with EtOAc and saturated aqueous potassium phosphate, dibasic. The mixture was filtered through Celite, and the layers were separated. The organic layer was dried over magnesium sulfate and concentrated in vacuo. The two reactions were combined, and the residue was purified by flash column chromatography (330 g SiO₂, EtOAc/hexanes) to give the title compound (26.6 g, 81% yield) as a light yellow oil. LC-MS calculated for C₁₇H₂₇N₂O₄Si (M+H)⁺: m/z=351.2; found 351.1.

### Intermediate 26. tert-Butyl (2R,3S)-3-((tert-butyldimethylsilyl)oxy)-2-(hydroxymethyl)azetidine-1-carboxylate

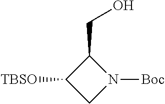 [SMILES: CC(C)(C)OC(=O)N1C[C@H](O[Si](C)(C)C(C)(C)C)[C@H]1CO]

### Step 1: ((2R,3S)-3-((tert-butyldimethylsilyl)oxy)azetidin-2-yl)methanol

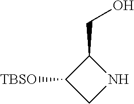 [SMILES: CC(C)(C)[Si](C)(C)O[C@H]1CN[C@@H]1CO]

A solution of methyl (2S,3S)-3-((tert-butyldimethylsilyl)oxy)-1-picolinoylazetidine-2-carboxylate (Intermediate 25, 26.6 g, 76 mmol) in THF (100 mL) was added dropwise to a suspension of lithium aluminum hydride (5.75 g, 152 mmol) in THF (280 mL) cooled to 0° C. via addition funnel over the course of 1 h. Following addition, the mixture was stirred for 10 minutes and carefully quenched via sequential addition of 5.75 mL of water, 5.75 mL of 15 wt % aq. NaOH, and 17.3 mL of water at 0° C. After warming to r.t., the mixture was stirred vigorously overnight, filtered through magnesium sulfate, and concentrated in vacuo to give the product as an orange oil. The crude material obtained was used without further purification. LC-MS calculated for C₁₀H₂₄NO₂Si (M+H)⁺: m/z=218.1; found 218.1.

### Step 2: tert-Butyl (2R,3S)-3-((tert-butyldimethylsilyl)oxy)-2-(hydroxymethyl)azetidine-1-carboxylate

((2R,3S)-3-((tert-Butyldimethylsilyl)oxy)azetidin-2-yl)methanol (Step 1) was taken up in EtOH (278 mL) and cooled to 0° C. Di-tert-butyl dicarbonate (15.2 g, 69.5 mmol) was added and the mixture was stirred at this temperature for 2 h. The mixture was concentrated in vacuo and the residue was purified by flash column chromatography (330 g SiO₂, EtOAc/hexanes) to give the title compound (9.70 g, 55% yield) as a clear oil. LC-MS calculated for C₁₁H₂₄NO₄Si (M−C₄H₈+H)⁺: m/z=262.2; found 262.2.

### Intermediate 27. tert-Butyl (2R,3S)-2-((benzyloxy)methyl)-3-((tert-butyldimethylsilyl)oxy)azetidine-1-carboxylate

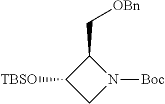 [SMILES: CC(C)(C)OC(=O)N1C[C@H](O[Si](C)(C)C(C)(C)C)[C@H]1COCc1ccccc1]

Sodium hydride (60% dispersion in mineral oil, 2.44 g, 61.1 mmol) was added to a solution of tert-butyl (2R,3S)-3-((tert-butyldimethylsilyl)oxy)-2-(hydroxymethyl)azetidine-1-carboxylate (Intermediate 26, 9.70 g, 30.6 mmol) and benzyl bromide (7.27 mL, 61.1 mmol) in THF (153 mL) at 0° C. The mixture was warmed to r.t. and stirred for 20 h. The mixture was quenched with saturated aqueous ammonium chloride, diluted with water and ethyl acetate, and the layers were separated. The aqueous layer was extracted with EtOAc, and the combined organic layers were washed twice with brine, dried over magnesium sulfate, and concentrated in vacuo. The residue was purified by flash column chromatography (330 g SiO₂, EtOAc/hexanes) to give the title compound (5.77 g, 46% yield) as a clear oil. LC-MS calculated for C₁₇H₃₀NO₂Si (M−C₅H₈O₂+H)⁺: m/z=308.2; found 308.1.

### Intermediate 28. tert-Butyl (2R,3S)-2-((benzyloxy)methyl)-3-hydroxyazetidine-1-carboxylate

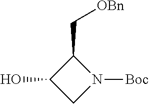 [SMILES: CC(C)(C)OC(=O)N1C[C@H](O)[C@H]1COCc1ccccc1]

Tetra-n-butylammonium fluoride (1.0 M in THF, 17.0 mmol, 17.0 mL) was added to a solution of tert-butyl (2R,3S)-2-((benzyloxy)methyl)-3-((tert-butyldimethylsilyl)oxy)azetidine-1-carboxylate (Intermediate 27, 5.77 g, 14.2 mmol) and acetic acid (0.81 mL, 14.2 mmol) in THF (71 mL) at 0° C. and the mixture was stirred at this temperature for 5 h. The reaction was quenched with saturated aqueous sodium bicarbonate and the layers were separated. The aqueous layer was extracted with EtOAc. The combined organic layers were washed with brine, dried over magnesium sulfate, and concentrated in vacuo. The residue was purified by flash column chromatography (12 g SiO₂, EtOAc/hexanes) to give the title compound (3.76 g, 90% yield) as a clear oil. LC-MS calculated for C₁₂H₁₆NO₄ (M-C₄H₈+H)⁺: m/z=238.1; found 238.1.

### Intermediate 29. tert-Butyl (2R,3S)-2-((benzyloxy)methyl)-3-(trifluoromethoxy)azetidine-1-carboxylate

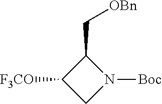 [SMILES: CC(C)(C)OC(=O)N1C[C@H](OC(F)(F)F)[C@H]1COCc1ccccc1]

In four separate 40 mL vials, a mixture of tert-butyl (2R,3S)-2-((benzyloxy)methyl)-3-hydroxyazetidine-1-carboxylate (Intermediate 28, 0.469 g, 1.60 mmol), silver trifluoromethanesulfonate (1.23 g, 4.80 mmol), Selectfluor™ (0.850 g, 2.40 mmol), and potassium fluoride (0.372 g, 6.39 mmol) were taken up in EtOAc (8.0 mL). 2-Fluoropyridine (0.41 mL, 4.8 mmol) and trimethyl(trifluoromethyl)silane (0.762 mL, 4.80 mmol) were added sequentially. The mixture was stirred vigorously at r.t. overnight. The four reactions were combined, filtered through Celite, and concentrated in vacuo. The residue was purified by flash column chromatography (40 g SiO₂, EtOAc/hexanes) to give the title compound (1.28 g, 56% yield) as a clear oil. LC-MS calculated for C₁₃H₁₅F₃NO₄ (M-C₄H₈+H)⁺: m/z=306.1; found 306.1.

### Intermediate 30. tert-Butyl (2R,3S)-2-(hydroxymethyl)-3-(trifluoromethoxy)azetidine-1-carboxylate

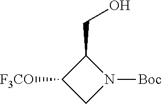 [SMILES: CC(C)(C)OC(=O)N1C[C@H](OC(F)(F)F)[C@H]1CO]

In a 100 mL round bottom flask, tert-butyl (2R,3S)-2-((benzyloxy)methyl)-3-(trifluoromethoxy)azetidine-1-carboxylate (Intermediate 29, 2.53 g, 7.00 mmol) was taken up in MeOH (35 mL). Palladium on carbon (10 wt %, 0.745 g, 0.700 mmol) was added and the flask was purged with hydrogen gas for 5 minutes. The mixture was stirred at r.t. for 2 h under a hydrogen balloon, then filtered and concentrated in vacuo. The residue was purified by flash column chromatography (40 g SiO₂, EtOAc/hexanes) to give the title compound (1.62 g, 86% yield) as a clear oil. LC-MS calculated for C₆H₉F₃NO₄ (M-C₄H₈+H)⁺: m/z=216.1; found 216.

### Intermediate 31. ((2R,3S)-3-(Trifluoromethoxy)azetidin-2-yl)methanol hydrochloride

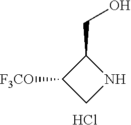 [SMILES: OC[C@H]1NC[C@@H]1OC(F)(F)F ; Cl]

HCl (4 M in 1,4-dioxane, 0.504 mL, 2.02 mmol) was added to a solution of tert-butyl (2R,3S)-2-(hydroxymethyl)-3-(trifluoromethoxy)azetidine-1-carboxylate (Intermediate 30, 68.0 mg, 0.252 mmol) in CH₂Cl₂ (1.20 mL) and MeOH (60 μL). The reaction was stirred at r.t. and progress was monitored by TLC (1:1 EtOAc/hexanes, staining with KMnO₄). After 1 h, complete consumption of starting material had occurred. The reaction was concentrated in vacuo to give the title compound as a white solid. The crude material obtained was used without further purification. LC-MS calculated for C₅H₉F₃NO₂ (M+H)⁺: m/z=172.1; found 172.1.

### Intermediate 32. 6-(5-Chloro-2-(((3S,4R)-3-hydroxytetrahydro-2H-pyran-4-yl)amino)pyrimidin-4-yl)-1-ethyl-4-fluoro-1H-indole-3-carboxylic Acid

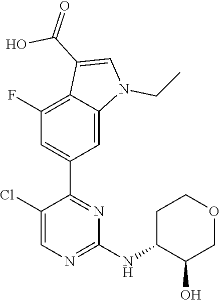 [SMILES: CCn1cc(C(=O)O)c2c(F)cc(-c3nc(N[C@@H]4CCOC[C@H]4O)ncc3Cl)cc21]

The title compound was prepared according to the procedures outlined for Intermediate 19, with iodoethane replacing iodomethane in step 1, and 2,4,5-trichloropyrimidine replacing 2,4-dichloro-5-fluoropyrimidine in step 6. LC-MS calculated for C₂₀H₂₁ClFN₄O₄(M+H)⁺: m/z=435.1; found 435.2.

### Intermediate 33: 1-(2,2-Difluoroethyl)-4-fluoro-6-(5-fluoro-2-(((3S,4R)-3-hydroxytetrahydro-2H-pyran-4-yl)amino)pyrimidin-4-yl)-1H-indole-3-carboxylic acid

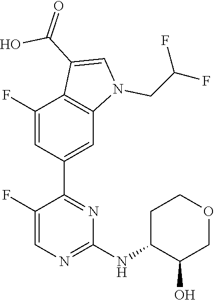 [SMILES: O=C(O)c1cn(CC(F)F)c2cc(-c3nc(N[C@@H]4CCOC[C@H]4O)ncc3F)cc(F)c12]

The title compound was prepared according to the procedures outlined for Intermediate 19, with 1,1-difluoro-2-iodoethane relacing iodomethane in step 1. LC-MS calculated for C₂₀H₁₉F₄N₄O₄(M+H)⁺: m/z=455.1; found 455.1.

### Intermediate 34: 1-(2,2-Difluoroethyl)-6-(5-fluoro-2-(((3S,4R)-3-hydroxytetrahydro-2H-pyran-4-yl)amino)pyrimidin-4-yl)-1H-indole-3-carboxylic Acid

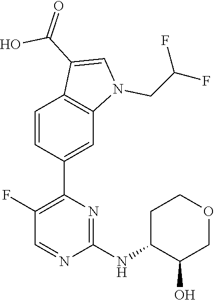 [SMILES: O=C(O)c1cn(CC(F)F)c2cc(-c3nc(N[C@@H]4CCOC[C@H]4O)ncc3F)ccc12]

The title compound was prepared according to the procedures outlined for Intermediate 17, with 1,1-difluoro-2-iodoethane relacing iodomethane in step 1. LC-MS calculated for C₂₀H₂₀F₃N₄O₄(M+H)⁺: m/z=437.1; found 437.1.

### Intermediate 35: 6-(5-Chloro-2-(((3S,4R)-3-hydroxytetrahydro-2H-pyran-4-yl)amino)pyrimidin-4-yl)-1-ethyl-1H-indole-3-carboxylic Acid

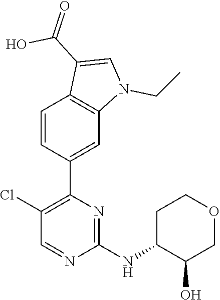 [SMILES: CCn1cc(C(=O)O)c2ccc(-c3nc(N[C@@H]4CCOC[C@H]4O)ncc3Cl)cc21]

### Step 1: methyl 6-(2,5-dichloropyrimidin-4-yl)-1-ethyl-1H-indole-3-carboxylate

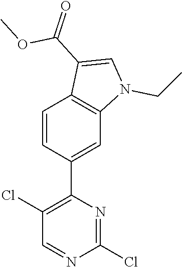 [SMILES: CCn1cc(C(=O)OC)c2ccc(-c3nc(Cl)ncc3Cl)cc21]

To a mixture of methyl 1-ethyl-6-(4,4,5,5-tetramethyl-1,3,2-dioxaborolan-2-yl)-1H-indole-3-carboxylate (Step 2, Intermediate 18, 0.334 g, 1.02 mmol), 2,4,5-trichloropyrimidine (0.372 g, 2.01 mmol), and K₂CO₃ (0.561 g, 4.06 mmol) in 1,4-dioxane/water (5:1; 5 mL) was added Pd(dppf)Cl₂·CH₂Cl₂ (0.066 g, 0.081 mmol). The mixture was sparged with N₂ for 3 min and stirred at 80° C. for 1 h. The reaction mixture was cooled to r.t., diluted with water and extracted with EtOAc. The combined organic layers were washed with sat. aq. NaHCO₃ and brine, dried over Na₂SO₄, and concentrated. The crude residue was purified by flash column chromatography (SiO₂, 0-80% EtOAc/hexanes) to afford the desired product (0.327 g, 92% yield). LC-MS calculated for C₁₆H₁₄Cl₂N₃O₂(M+H)⁺: m/z=350.1; found 350.2.

### Step 2: methyl 6-(5-chloro-2-(((3S,4R)-3-hydroxytetrahydro-2H-pyran-4-yl)amino)pyrimidin-4-yl)-1-ethyl-1H-indole-3-carboxylate

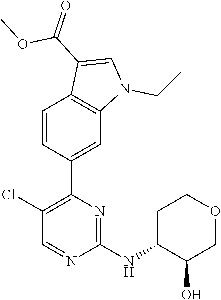 [SMILES: CCn1cc(C(=O)OC)c2ccc(-c3nc(N[C@@H]4CCOC[C@H]4O)ncc3Cl)cc21]

To a mixture of methyl 6-(2,5-dichloropyrimidin-4-yl)-1-ethyl-1H-indole-3-carboxylate (Step 1, 0.327 g, 0.934 mmol), CsF (0.709 g, 4.67 mmol), and DIPEA (0.652 mL, 3.74 mmol) in DMSO (5.5 mL) was added (3S,4R)-4-aminotetrahydro-2H-pyran-3-ol hydrochloride (0.359 g, 2.33 mmol). The mixture was stirred at 90° C. for 5 h. After cooling to r.t., the reaction mixture was diluted with saturated aqueous sodium bicarbonate and extracted with EtOAc. The combined organic layers were washed with 5% LiCl (aqueous) and brine, dried over sodium sulfate, filtered and concentrated. The crude residue was purified by chromatography (SiO₂, 0-100% EtOAc/hexanes) to afford the desired product (185 mg, 46% yield) as a yellow oil. LC-MS calculated for C₂₁H₂₄ClN₄O₄ (M+H)⁺: m/z=431.1; found 431.2.

### Step 3: 6-(5-chloro-2-(((3S,4R)-3-hydroxytetrahydro-2H-pyran-4-yl)amino)pyrimidin-4-yl)-1-ethyl-1H-indole-3-carboxylic Acid

To a mixture of methyl 6-(5-chloro-2-(((3S,4R)-3-hydroxytetrahydro-2H-pyran-4-yl)amino)pyrimidin-4-yl)-1-ethyl-1H-indole-3-carboxylate (Step 2, 185 mg, 0.429 mmol) in THF (2.8 mL), MeOH (2.8 mL), and H₂O (2.8 mL) was added LiOH H₂O (510 mg, 12.2 mmol). The reaction mixture was stirred at 65° C. for 1 h. After cooling to r.t, the reaction mixture was acidified with aqueous HCl (1 N, 16.5 mL, 16.5 mmol). The resulting mixture was cooled in an ice bath and stirred for 1 h. The yellow precipitate that formed was collected via filtration and dried under air to afford the desired product (105 mg, 59% yield). The crude material obtained was used directly without further purification. LC-MS calculated for C₂₀H₂₂ClN₄O₄(M+H)⁺: m/z=417.1; found 417.1.

### Intermediate 36. 6-(5-Chloro-2-(((3S,4R)-3-hydroxytetrahydro-2H-pyran-4-yl)amino)pyrimidin-4-yl)-1-ethyl-4-fluoro-1H-indole-3-carboxylic Acid

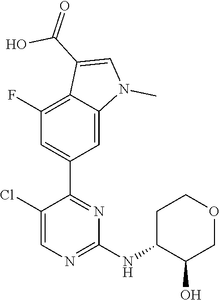 [SMILES: Cn1cc(C(=O)O)c2c(F)cc(-c3nc(N[C@@H]4CCOC[C@H]4O)ncc3Cl)cc21]

The title compound was prepared according to the procedures outlined for Intermediate 19, with 2,4,5-trichloropyrimidine replacing 2,4-dichloro-5-fluoropyrimidine in step 6. LC-MS calculated for C₁₉H₁₉ClFN₄O₄(M+H)⁺: m/z=421.1; found 421.2.

### Intermediate 37: (±)-(2S,3S)-2-(Hydroxymethyl)azetidine-3-carbonitrile 2,2,2-trifluoroacetate

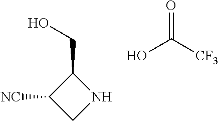 [SMILES: N#C[C@H]1CN[C@@H]1CO ; O=C(O)C(F)(F)F]

### Step 1. (±)-1-(tert-Butyl) 2-methyl (2S,3S)-3-phenylazetidine-1,2-dicarboxylate

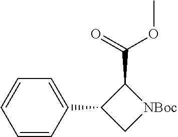 [SMILES: COC(=O)[C@@H]1[C@@H](c2ccccc2)CN1C(=O)OC(C)(C)C]

A mixture of (±)-(2R,3R)-1-[(tert-butoxy)carbonyl]-3-phenylazetidine-2-carboxylic Acid (5.13 g, 18.50 mmol, Enamine EN300-1692931) in DCM (36 mL) and MeOH (11 mL) was cooled to 0° C. before (trimethylsilyl)diazomethane (2.1 M in hexanes, 17.6 mL, 37 mmol) was added dropwise. The reaction mixture was warmed to r.t. and stirred for 2 h. The reaction mixture was cooled to 0° C. and quenched by the dropwise addition of HOAc (3.2 mL, 55.5 mmol). After warming to r.t., the mixture was concentrated in vacuo. The crude residue was purified by flash column chromatography (SiO₂, 0-50% EtOAc in hexanes) to afford the desired product (5.05 g, 94% yield). LC-MS calculated for C₁₂H₁₄NO₄ (M+H−C₄H₈)⁺: m/z=236.1; found 236.1.

### Step 2. (±)-(2S,3S)-1-(tert-Butoxycarbonyl)-2-(methoxycarbonyl)azetidine-3-carboxylic Acid

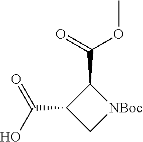 [SMILES: COC(=O)[C@@H]1[C@@H](C(=O)O)CN1C(=O)OC(C)(C)C]

To a suspension of sodium periodate (74.1 g, 347 mmol) in acetonitrile (180 mL), carbon tetrachloride (180 mL) and water (180 mL), was added ruthenium(III) chloride hydrate (0.72 g, 3.47 mmol). The suspension was stirred rapidly at room temperature for 30 minutes. (±)-1-(tert-butyl) 2-methyl (2S,3S)-3-phenylazetidine-1,2-dicarboxylate (5.05 g, 17.33 mmol) dissolved in acetonitrile (10 mL) was added dropwise with rapid stirring. The reaction mixture was stirred rapidly at room temperature for 24 h. The reaction mixture was diluted with water and ethyl acetate. The layers were separated, and the aqueous layer was extracted with ethyl acetate. The combined organic fractions were filtered over a plug of MgSO₄ and concentrated. The crude residue was purified by flash column chromatography (SiO₂, 0-100% EtOAc in hexanes) to afford the desired product (2.9 g, 65% yield). LC-MS calculated for C₆H₁₀NO₄ (M+H-C₅H₈O₂)⁺: m/z=160.1; found 160.1.

### Step 3. (±)-1-(tert-Butyl) 2-methyl (2S,3S)-3-carbamoylazetidine-1,2-dicarboxylate

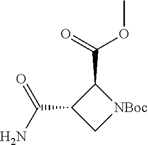 [SMILES: COC(=O)[C@@H]1[C@@H](C(N)=O)CN1C(=O)OC(C)(C)C]

A mixture of (±)-(2S,3S)-1-(tert-butoxycarbonyl)-2-(methoxycarbonyl)azetidine-3-carboxylic acid (0.43 g, 1.659 mmol), conc. aq. ammonia (12.44 mL, 4.98 mmol), and N,N-diisopropylethylamine (0.869 mL, 4.98 mmol) in ethyl acetate (8 mL) at 0° C. was treated with propylphosphonic anhydride (50% in ethyl acetate, 1.522 mL, 2.488 mmol). The reaction mixture was warmed to r.t. and stirred rapidly for 6 h. The reaction mixture was diluted with water and ethyl acetate. The layers were separated, and the aqueous layer was extracted with ethyl acetate. The combined organic fractions were filtered over a plug of MgSO₄ and concentrated. The crude residue was purified by flash column chromatography (SiO₂, 0-100% EtOAc in hexanes) to afford the desired product (0.253 g, 59% yield). LC-MS calculated for C₆H₁₁N₂O₃(M+H−C₅H₈O₂)⁺: m/z=159.1; found 159.0.

### Step 4. (±)-1-(tert-Butyl) 2-methyl (2S,3S)-3-cyanoazetidine-1,2-dicarboxylate

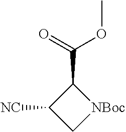 [SMILES: COC(=O)[C@@H]1[C@@H](C#N)CN1C(=O)OC(C)(C)C]

To a 0° C. mixture of (±)-1-(tert-butyl) 2-methyl (2S,3S)-3-carbamoylazetidine-1,2-dicarboxylate (253 mg, 0.980 mmol) and pyridine (0.32 mL, 3.92 mmol) in dry THF (5 mL) was added trifluoroacetic anhydride (0.33 mL, 2.35 mmol) dropwise. The mixture was stirred at 0° C. for 30 minutes then quenched with water and extracted with CH₂Cl₂. The combined organic fractions were dried over a plug of MgSO₄ and concentrated. The crude residue was purified by flash column chromatography (SiO₂, 0-100% EtOAc in hexanes) to afford the desired product (0.225 g, 96% yield). LC-MS calculated for C₇H₉N₂O₄(M+H−C₄H₈)⁺: m/z=185.1; found 185.1.

### Step 5. (±)-tert-Butyl (2S,3S)-3-cyano-2-(hydroxymethyl)azetidine-1-carboxylate

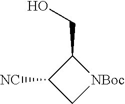 [SMILES: CC(C)(C)OC(=O)N1C[C@H](C#N)[C@H]1CO]

To a 0° C. mixture of (±)-1-(tert-butyl) 2-methyl (2S,3S)-3-cyanoazetidine-1,2-dicarboxylate (225 mg, 0.936 mmol) in MeOH (5 mL) was added sodium borohydride (89 mg, 2.341 mmol). The reaction mixture was stirred at 0° C. for 30 minutes then quenched with sat. aq. NaHCO₃ mixture and diluted with ethyl acetate and water. The layers were separated and the aqueous layer was extracted with ethyl acetate. The combined organic fractions were dried over a plug of MgSO₄ and concentrated. The crude residue was purified by flash column chromatography (SiO₂, 0-100% EtOAc in hexanes) to afford the desired product (0.185 g, 93% yield). LC-MS calculated for C₆H₉N₂O₃(M+H−C₄H₈)⁺: m/z=157.1; found 157.0.

### Step 6. (±)-(2S,3S)-2-(Hydroxymethyl)azetidine-3-carbonitrile 2,2,2-trifluoroacetate

To a mixture of (±)-tert-butyl (2S,3S)-3-cyano-2-(hydroxymethyl)azetidine-1-carboxylate (185 mg, 0.872 mmol) in DCM (9 mL) was added trifluoroacetic acid (0.67 mL, 8.72 mmol). The reaction mixture was stirred at r.t. for 1 hour then concentrated. The crude residue was further co-evaporated 3× with DCM. The crude material obtained was used directly without further purification. LC-MS calculated for C₅H₉N₂O (M+H)⁺: m/z=113.1; found 113.0.

### Intermediates 38 and 39: (±)-Methyl (2R,3S)-1-(3-(((tert-butyldimethylsilyl)oxy)methyl)picolinoyl)-3-(methylsulfonyl)azetidine-2-carboxylate and (±)-Methyl (2R,3R)-1-(3-(((tert-butyldimethylsilyl)oxy)methyl)picolinoyl)-3-(methylsulfonyl)azetidine-2-carboxylate

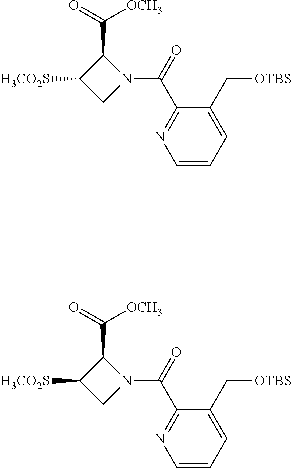

### Step 1. Methyl 2-((tert-butoxycarbonyl)amino)-3-(methylthio)butanoate

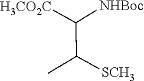 [SMILES: COC(=O)C(NC(=O)OC(C)(C)C)C(C)SC]

A mixture of 2-((tert-butoxycarbonyl)amino)-3-(methylthio)butanoic acid (0.95 g, 3.81 mmol, Enamine EN300-6729925) in DCM (7 mL) and MeOH (3 mL) was cooled to 0° C. before (trimethylsilyl)diazomethane (2.1 M in hexanes, 4.5 mL, 9.5 mmol) was added dropwise. The reaction mixture was warmed to r.t. and stirred for 2 h. The reaction mixture was cooled to 0° C. and quenched by the dropwise addition of HOAc (0.76 mL, 13.3 mmol). After warming to r.t., the mixture was concentrated in vacuo. The crude residue was purified by flash column chromatography (SiO₂, 0-50% EtOAc in hexanes) to afford the desired product (0.92 g, 92% yield). LC-MS calculated for C₆H₁₄NO₂S (M+H−C₅H₈O₂)⁺: m/z=164.1; found 164.1.

### Step 2. Methyl 2-amino-3-(methylthio)butanoate 2,2,2-trifluoroacetate

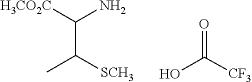

To a mixture of methyl 2-((tert-butoxycarbonyl)amino)-3-(methylthio)butanoate (924 mg, 3.51 mmol) in DCM (15 mL) was added trifluoroacetic acid (2.0 mL, 26.3 mmol). The reaction mixture was stirred at r.t. for 1 hour then concentrated. The crude residue was further co-evaporated 3× with DCM. The crude material obtained was used directly without further purification. LC-MS calculated for C₆H₁₄NO₂S (M+H)⁺: m/z=164.1; found 164.0.

### Step 3. Methyl 2-(3-(((tert-butyldimethylsilyl)oxy)methyl)picolinamido)-3-(methylthio)butanoate

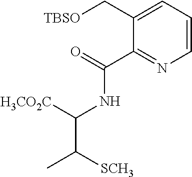 [SMILES: COC(=O)C(NC(=O)c1ncccc1CO[Si](C)(C)C(C)(C)C)C(C)SC]

To a mixture of methyl 2-amino-3-(methylthio)butanoate 2,2,2-trifluoroacetate (970 mg, 3.50 mmol), 3-(((tert-butyldimethylsilyl)oxy)methyl)picolinic acid (850 mg, 3.18 mmol, J&W Pharmlab 69R1633), and N,N-diisopropylethylamine (1.67 mL, 9.54 mmol) in DMF (10 mL) was added HATU (1.27 g, 3.34 mmol). The reaction mixture was stirred at r.t. for 1 hour then quenched with water and diluted with ethyl acetate. The organic layer was washed 3× with water, then brine, then filtered over a plug of MgSO₄ and concentrated. The crude residue was purified by flash column chromatography (SiO₂, 0-50% EtOAc in hexanes) to afford the desired product (1.05 g, 80% yield). LC-MS calculated for C₁₉H₃₃N₂O₄SSi (M+H)⁺: m/z=413.2; found 413.3.

### Step 4. Methyl 2-(3-(((tert-butyldimethylsilyl)oxy)methyl)picolinamido)-3-(methylsulfonyl)butanoate

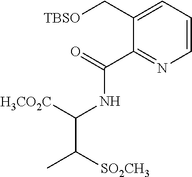 [SMILES: COC(=O)C(NC(=O)c1ncccc1CO[Si](C)(C)C(C)(C)C)C(C)S(C)(=O)=O]

To a mixture of methyl 2-(3-(((tert-butyldimethylsilyl)oxy)methyl)picolinamido)-3-(methylthio)butanoate (1.05 g, 2.53 mmol) in DCM (13 mL) cooled to 0° C. was added m-CPBA (77% w/w, 1.14 g, 5.07 mmol). The reaction mixture was stirred at 0° C. for 1 hour then diluted with DCM and quenched sat. aq. NaHCO₃ and sat. aq. sodium thiosulfate. The biphasic mixture was stirred rapidly for 5 minutes. The layers were separated, and the aqueous layer was extracted with DCM. The combined organic fractions were filtered over a plug of MgSO₄ and concentrated. The crude residue was purified by flash column chromatography (SiO₂, 0-100% EtOAc in hexanes) to afford the desired product (0.982 g, 87% yield). LC-MS calculated for C₁₉H₃₃N₂O₆SSi (M+H)⁺: m/z=445.2; found 445.3.

### Step 5. (±)-Methyl (2R,3S)-1-(3-(((tert-butyldimethylsilyl)oxy)methyl)picolinoyl)-3-(methylsulfonyl)azetidine-2-carboxylate and (±)-methyl (2R,3R)-1-(3-(((tert-butyldimethylsilyl)oxy)methyl)picolinoyl)-3-(methylsulfonyl)azetidine-2-carboxylate

To a vial containing methyl 2-(3-(((tert-butyldimethylsilyl)oxy)methyl)picolinamido)-3-(methylsulfonyl)butanoate (0.982 g, 2.209 mmol) and toluene (11.04 mL) were added iodobenzene diacetate (1.778 g, 5.52 mmol) and palladium(II) acetate (0.050 g, 0.221 mmol). The mixture was sparged with N₂ for 5 minutes and then stirred and heated at 120° C. for 2 h. The reaction mixture was concentrated, and the crude residue was purified by flash column chromatography (SiO₂, 0-100% EtOAc in hexanes) to afford the desired products:

First eluting peak, corresponding to (±)-methyl (2R,3S)-1-(3-(((tert-butyldimethylsilyl)oxy)methyl)picolinoyl)-3-(methylsulfonyl)azetidine-2-carboxylate, (Intermediate 38, 0.114 g, 0.258 mmol). LC-MS calculated for C₁₉H₃₁N₂O₆SSi (M+H)⁺: m/z=443.2; found 443.3. ¹H NMR (500 MHz, DMSO-d₆, major rotomer) δ 8.40 (d, J=4.6 Hz, 1H), 8.13 (d, J=7.9 Hz, 1H), 7.61 (dd, J=7.9, 4.6 Hz, 1H), 5.50 (d, J=4.8 Hz, 1H), 5.21-5.03 (m, 2H), 4.58-4.50 (m, 1H), 4.40-4.32 (m, 1H), 4.22 (dd, J=10.7, 4.8 Hz, 1H), 3.63 (s, 3H), 3.12 (s, 3H), 0.94 (s, 9H), 0.12 (s, 6H).

Second eluting peak, corresponding to (±)-methyl (2R,3R)-1-(3-(((tert-butyldimethylsilyl)oxy)methyl)picolinoyl)-3-(methylsulfonyl)azetidine-2-carboxylate, (Intermediate 39, 0.091 g, 0.206 mmol). LC-MS calculated for C₁₉H₃₁N₂O₆SSi (M+H)⁺: m/z=443.2; found 443.3. ¹H NMR (500 MHz, DMSO-d₆, major rotomer) δ 8.38 (d, J=4.6 Hz, 1H), 8.12 (d, J=8.0 Hz, 1H), 7.58 (dd, J=8.0, 4.6 Hz, 1H), 5.81 (d, J=9.1 Hz, 1H), 5.12-4.98 (m, 2H), 4.91-4.83 (m, 1H), 4.31-4.24 (m, 2H), 3.50 (s, 3H), 3.07 (s, 3H), 0.95 (s, 9H), 0.12 (s, 6H).

### Intermediate 40: (±)-((2R,3S)-3-(Methylsulfonyl)azetidin-2-yl)methanol hydrochloride

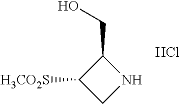

### Step 1. (±)-1-(tert-Butyl) 2-methyl (2R,3S)-3-(methylsulfonyl)azetidine-1,2-dicarboxylate

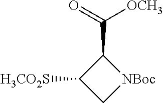

Aqueous HCl (1M, 2.6 mL, 2.6 mmol) was added to a mixture of (±)-methyl (2R,3S)-1-(3-(((tert-butyldimethylsilyl)oxy)methyl)picolinoyl)-3-(methylsulfonyl)azetidine-2-carboxylate (Intermediate 38, 234 mg, 0.529 mmol) in 1,4-dioxane (5 mL). The mixture was stirred at room temperature overnight. Sodium bicarbonate (222 mg, 2.6 mmol), N,N-diisopropylethylamine (0.185 mL, 1.06 mmol), and Boc₂O (231 mg, 1.06 mmol) were added to the mixture sequentially. The mixture was stirred at room temperature for 1 h. The mixture was diluted with water and DCM, and the layers were separated. The aqueous layer was extracted with DCM and the combined organic layers were dried over magnesium sulfate and concentrated in vacuo. The crude residue was purified by flash column chromatography (SiO₂, 0-100% EtOAc in hexanes) to afford the desired product (0.118 g, 76% yield). LC-MS calculated for C₆H₁₂NO₄S (M+H-C₅H₈O₂)⁺: m/z=194.0; found 194.0.

### Step 2. (±)-tert-Butyl (2R,3S)-2-(hydroxymethyl)-3-(methylsulfonyl)azetidine-1-carboxylate

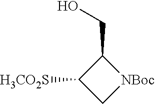

To a mixture of (±)-1-(tert-butyl) 2-methyl (2R,3S)-3-(methylsulfonyl)azetidine-1,2-dicarboxylate (119 mg, 0.406 mmol) in THF (2 mL) at 0° C. was added lithium borohydride (2M in THF, 609 μl, 1.2 mmol) dropwise. The reaction mixture was stirred at 0° C. for 15 min and warmed to room temperature for 1 hour. The reaction mixture was quenched with sat. aq. NaHCO₃ and extracted with ethyl acetate. The combined organic fractions were filtered over a plug of MgSO₄ and concentrated. The crude residue was purified by flash column chromatography (SiO₂, 0-100% EtOAc in hexanes) to afford the desired product (88 mg, 82% yield). LC-MS calculated for C₅H₁₂NO₃S (M+H−C₅H₈O₂)⁺: m/z=166.1; found 166.0.

### Step 3. (±)-((2R,3S)-3-(Methylsulfonyl)azetidin-2-yl)methanol hydrochloride

To a mixture of (±)-tert-butyl (2R,3S)-2-(hydroxymethyl)-3-(methylsulfonyl)azetidine-1-carboxylate (88 mg, 0.332 mmol) in DCM (1.6 mL) and MeOH (0.08 mL) at r.t. was added HCl (4M in dioxane, 663 μl, 2.65 mmol) dropwise. The reaction mixture was stirred at r.t. for 1 hour, then diluted with ether and filtered. The filter cake was washed with ether, then dried overnight to furnish the desired product as a white solid, which was used directly without further purification. LC-MS calculated for C₅H₁₂NO₃S (M+H)⁺: m/z=166.1; found 166.1.

### Intermediate 41: (±)-((2R,3R)-3-(Methylsulfonyl)azetidin-2-yl)methanol hydrochloride

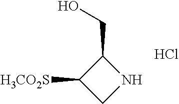

The title compound was prepared according to the procedures outlined in Intermediate 40, with (±)-methyl (2R,3R)-1-(3-(((tert-butyldimethylsilyl)oxy)methyl)picolinoyl)-3-(methylsulfonyl)azetidine-2-carboxylate (Intermediate 39) replacing (±)-methyl (2R,3S)-1-(3-(((tert-butyldimethylsilyl)oxy)methyl)picolinoyl)-3-(methylsulfonyl)azetidine-2-carboxylate (Intermediate 38). LC-MS calculated for C₅H₁₂NO₃S (M+H)⁺: m/z=166.1; found 166.1.

### Intermediate 42: (±)-((2S,3S)-3-((Trifluoromethoxy)methyl)azetidin-2-yl)methanol hydrochloride

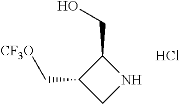

### Step 1. (±)-1-(tert-butyl) 2-methyl (2S,3S)-3-(hydroxymethyl)azetidine-1,2-dicarboxylate

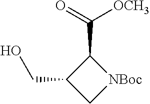 [SMILES: COC(=O)[C@@H]1[C@@H](CO)CN1C(=O)OC(C)(C)C]

A mixture of (±)-(2S,3S)-1-(tert-butoxycarbonyl)-2-(methoxycarbonyl)azetidine-3-carboxylic acid (from intermediate 37, step 2, 2.9 g, 11.19 mmol) in THF (56 mL) was cooled to 0° C. before BH₃-DMS (3.2 mL, 33.6 mmol) was added dropwise. The reaction mixture was warmed to r.t. and stirred for 2 h. The reaction mixture was cooled to 0° C. and quenched by the dropwise addition of MeOH (9 mL). After warming to r.t., the mixture was concentrated in vacuo.

The crude residue was purified by flash column chromatography (SiO₂, 0-100% EtOAc in hexanes) to afford the desired product (2.41 g, 88% yield). LC-MS calculated for C₆H₁₂NO₃ (M+H−C₅H₈O₂)⁺: m/z=146.1; found 146.3.

### Step 2. (±)-1-(tert-Butyl) 2-methyl (2S,3S)-3-((trifluoromethoxy)methyl)azetidine-1,2-dicarboxylate

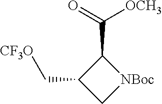

To a flame-dried vial was added (±)-1-(tert-butyl) 2-methyl (2S,3S)-3-(hydroxymethyl)azetidine-1,2-dicarboxylate (400 mg, 1.63 mmol), silver trifluoromethanesulfonate (838 mg, 3.26 mmol), 1-chloromethyl-4-fluoro-1,4-diazoniabicyclo[2.2.2]octane bis(tetrafluoroborate) (867 mg, 2.446 mmol), and potassium fluoride (284 mg, 4.89 mmol, dried at 170 degrees overnight). The vial was evacuated and backfilled 3× with N₂. Then dry ethyl acetate (4 mL) was added, followed by 2-fluoropyridine (280 μl, 3.26 mmol) and trimethyl(trifluoromethyl)silane (518 μl, 3.26 mmol). The suspension was stirred rapidly overnight at r.t. in the darkness. The suspension was filtered over a plug of diatomaceous earth, then concentrated. The crude residue was purified by flash column chromatography (SiO₂, 0-100% EtOAc in hexanes) to afford the desired product (272 mg, 53% yield). LC-MS calculated for C₇H₁₁F₃NO₃ (M+H−C₅H₈O₂)⁺: m/z=214.1; found 214.2.

### Step 3. (±)-tert-Butyl (2S,3S)-2-(hydroxymethyl)-3-((trifluoromethoxy)methyl)azetidine-1-carboxylate

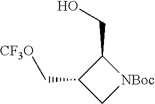

To a mixture of (±)-1-(tert-butyl) 2-methyl (2S,3S)-3-((trifluoromethoxy)methyl)azetidine-1,2-dicarboxylate (272 mg, 0.868 mmol) in THF (3.5 mL) at 0° C. was added lithium borohydride (2M in THF, 1.3 mL, 2.6 mmol) dropwise. The reaction mixture was stirred at 0° C. for 15 min and warmed to room temperature for 1 hour. The reaction mixture was quenched with sat. aq. NaHCO₃ and extracted with ethyl acetate. The combined organic fractions were filtered over a plug of MgSO₄ and concentrated. The crude residue was purified by flash column chromatography (SiO₂, 0-100% EtOAc in hexanes) to afford the desired product (215 mg, 87% yield). LC-MS calculated for C₇H₁₁F₃NO₄ (M+H−C₄H₈)⁺: m/z=230.1; found 230.2.

### Step 4. (±)-((2S,3S)-3-((Trifluoromethoxy)methyl)azetidin-2-yl)methanol hydrochloride

To a mixture of (±)-tert-butyl (2S,3S)-2-(hydroxymethyl)-3-((trifluoromethoxy)methyl)azetidine-1-carboxylate (215 mg, 0.754 mmol) in DCM (3.6 mL) and MeOH (0.18 mL) at r.t. was added HCl (4M in dioxane, 1.5 mL, 6.0 mmol) dropwise. The reaction mixture was stirred at r.t. for 1 hour, then diluted with ether. The solvent was removed under vacuum, furnishing the desired product as a sticky oil, which was used without further purification. LC-MS calculated for C₆H₁₁F₃NO₂ (M+H)⁺: m/z=186.1; found 186.2.

### Intermediate 43: (±)-((2S,3S)-3-((Difluoromethoxy)methyl)azetidin-2-yl)methanol hydrochloride

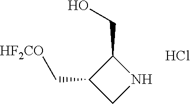

### Step 1. (±)-1-(tert-butyl) 2-methyl (2S,3S)-3-((difluoromethoxy)methyl)azetidine-1,2-dicarboxylate

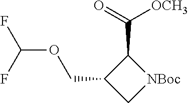 [SMILES: COC(=O)[C@@H]1[C@@H](COC(F)F)CN1C(=O)OC(C)(C)C]

1-(tert-butyl) 2-methyl (2S,3S)-3-(hydroxymethyl)azetidine-1,2-dicarboxylate (from Intermediate 42, Step 1, 0.4 g, 1.631 mmol) was dissolved in dry acetonitrile (5.44 mL). CuI (0.062 g, 0.326 mmol) was added. The mixture was sparged with N₂ for 5 minutes, then heated to 50° C. A mixture of 2-fluorosulfonyl-2,2-difluoroacetic acid (0.202 mL, 1.957 mmol) in 4 mL of acetonitrile was added dropwise over a period of 30 min. The reaction mixture was heated for an additional 30 min at 50° C. Then the reaction mixture was concentrated, and the crude residue was dissolved in EtOAc and filtered. The resulting mixture of EtOAc was concentrated and the crude residue was purified by flash column chromatography (SiO₂, 0-100% EtOAc in hexanes) to afford the desired product (237 mg, 49% yield). LC-MS calculated for C₈H₁₂F₂NO₅ (M+H−C₄H₈)⁺: m/z=240.1; found 240.1.

### Step 2. (±)-tert-Butyl (2S,3S)-3-((difluoromethoxy)methyl)-2-(hydroxymethyl)azetidine-1-carboxylate

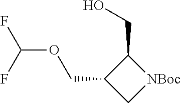 [SMILES: CC(C)(C)OC(=O)N1C[C@H](COC(F)F)[C@H]1CO]

The title compound was prepared according to the procedures outlined in Intermediate 42, step 3, with (±)-1-(tert-butyl) 2-methyl (2S,3S)-3-((difluoromethoxy)methyl)azetidine-1,2-dicarboxylate replacing (±)-1-(tert-butyl) 2-methyl (2S,3S)-3-((trifluoromethoxy)methyl)azetidine-1,2-dicarboxylate. LC-MS calculated for C₇H₁₂F₂NO₄ (M+H−C₄H₈)⁺: m/z=212.1; found 212.2.

### Step 3. (±)-((2S,3S)-3-((Difluoromethoxy)methyl)azetidin-2-yl)methanol hydrochloride

The title compound was prepared according to the procedures outlined in Intermediate 42, step 4, with (±)-tert-butyl (2S,3S)-3-((difluoromethoxy)methyl)-2-(hydroxymethyl)azetidine-1-carboxylate replacing (±)-tert-butyl (2S,3S)-2-(hydroxymethyl)-3-((trifluoromethoxy)methyl)azetidine-1-carboxylate. LC-MS calculated for C₆H₁₂F₂NO₂ (M+H)⁺: m/z=168.1; found 168.2.

### Example 1: 4-Fluoro-6-(5-fluoro-2-(((3R,4R)-3-hydroxy-1-(methylsulfonyl)piperidin-4-yl)amino)pyrimidin-4-yl)-1-isopropyl-N-methyl-1H-indole-3-carboxamide

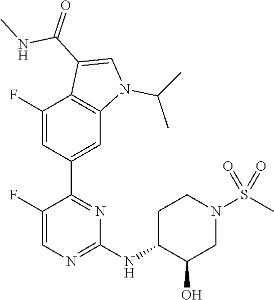

A vial was charged with a stir bar, 6-(2-chloro-5-fluoropyrimidin-4-yl)-4-fluoro-1-isopropyl-N-methyl-1H-indole-3-carboxamide (Intermediate 2, 70 mg, 0.192 mmol), (3R,4R)-4-amino-1-(methylsulfonyl)piperidin-3-ol hydrochloride (Intermediate 6, 53 mg, 0.227 mmol), and GPhos Pd G6 TES (18 mg, 0.019 mmol, Aldrich 922900). The vial was evacuated and backfilled with N₂ three times. To the vial was added THF (0.5 mL) and NaOTMS (1 M in THF, 0.57 mL, 0.57 mmol), and the reaction mixture was stirred at 60° C. for 30 min. After cooling to r.t., the mixture was diluted with MeCN, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₃H₂₉F₂N₆O₄S (M+H)⁺: m/z=523.2; found 523.3. ¹H NMR (500 MHz, DMSO-d₆) δ 8.45 (d, J=3.8 Hz, 1H), 8.15 (s, 1H), 8.08 (s, 1H), 7.81 (s, 1H), 7.58 (d, J=12.8 Hz, 1H), 7.22 (s, 1H), 4.93-4.80 (m, 1H), 3.79 (s, 1H), 3.71-3.59 (m, 2H), 3.55-3.48 (m, 1H), 2.97-2.85 (m, 4H), 2.80 (d, J=4.4 Hz, 3H), 2.75-2.67 (m, 1H), 2.15-2.06 (m, 1H), 1.61-1.46 (m, 7H).

### Example 2: 4-Fluoro-6-(5-fluoro-2-(((3S,4R)-3-hydroxytetrahydro-2H-pyran-4-yl)amino)pyrimidin-4-yl)-1-isopropyl-N-methyl-1H-indole-3-carboxamide

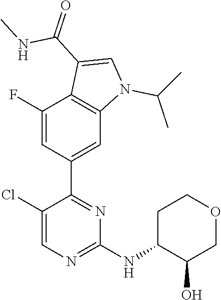 [SMILES: CNC(=O)c1cn(C(C)C)c2cc(-c3nc(N[C@@H]4CCOC[C@H]4O)ncc3Cl)cc(F)c12]

The title compound was prepared according to the procedures described in Example 1, using (3S,4R)-4-aminotetrahydro-2H-pyran-3-ol hydrochloride instead of (3R,4R)-4-amino-1-(methylsulfonyl)piperidin-3-ol hydrochloride. LC-MS calculated for C₂₃H₂₆F₂N₆O₃(M+H)⁺: m/z=446.2; found 446.4.

### Example 3: 4-Fluoro-6-(5-fluoro-2-(((3R,4R)-3-hydroxy-1-(methylsulfonyl)piperidin-4-yl)amino)pyrimidin-4-yl)-1-isopropyl-N-(2-(pyrimidin-2-yl)ethyl)-1H-indole-3-carboxamide

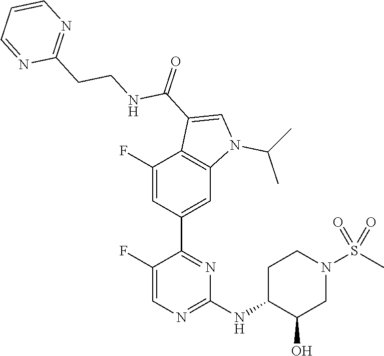 [SMILES: CC(C)n1cc(C(=O)NCCc2ncccn2)c2c(F)cc(-c3nc(N[C@@H]4CCN(S(C)(=O)=O)C[C@H]4O)ncc3F)cc21]

To a vial containing 4-fluoro-6-(5-fluoro-2-(((3R,4R)-3-hydroxy-1-(methylsulfonyl)piperidin-4-yl)amino)pyrimidin-4-yl)-1-isopropyl-1H-indole-3-carboxylic acid (Intermediate 7, 10 mg, 0.02 mmol), 2-(pyrimidin-2-yl)ethan-1-amine dihydrochloride (7.7 mg, 0.04 mmol), DMF (0.4 mL) and N,N-diisopropylethylamine (0.01 mL, 0.06 mmol) was added HATU (11.2 mg, 0.03 mmol). The reaction mixture was stirred at r.t. for 1 h. The mixture was diluted with MeCN, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₈H₃₃F₂N₈O₄S (M+H)⁺: m/z=615.2; found 615.4.

### Example 4: 6-(5-Chloro-2-(((3S,4R)-3-hydroxytetrahydro-2H-pyran-4-yl)amino)pyrimidin-4-yl)-4-fluoro-1-isopropyl-N-((1-methyl-1H-indazol-6-yl)methyl)-1H-indole-3-carboxamide

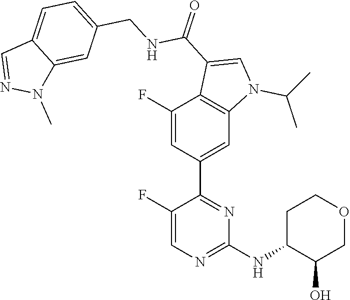 [SMILES: CC(C)n1cc(C(=O)NCc2ccc3cnn(C)c3c2)c2c(F)cc(-c3nc(N[C@@H]4CCOC[C@H]4O)ncc3F)cc21]

The title compound was prepared according to the procedures described in Example 3, using 6-(5-chloro-2-(((3S,4R)-3-hydroxytetrahydro-2H-pyran-4-yl)amino)pyrimidin-4-yl)-4-fluoro-1-isopropyl-1H-indole-3-carboxylic acid (Intermediate 8) instead of 4-fluoro-6-(5-fluoro-2-(((3R,4R)-3-hydroxy-1-(methylsulfonyl)piperidin-4-yl)amino)pyrimidin-4-yl)-1-isopropyl-1H-indole-3-carboxylic acid, and (1-methyl-1H-indazol-6-yl)methanamine instead of 2-(pyrimidin-2-yl)ethan-1-amine dihydrochloride. LC-MS calculated for C₃₀H₃₂ClFN₇O₃(M+H)⁺: m/z=592.2; found 592.3.

### Example 5: 6-(5-Cyano-2-(((3S,4R)-3-hydroxytetrahydro-2H-pyran-4-yl)amino)pyrimidin-4-yl)-4-fluoro-1-isopropyl-N-((1-methyl-1H-indazol-6-yl)methyl)-1H-indole-3-carboxamide

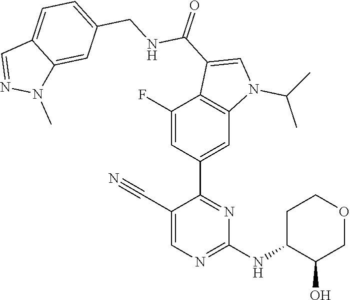 [SMILES: CC(C)n1cc(C(=O)NCc2ccc3cnn(C)c3c2)c2c(F)cc(-c3nc(N[C@@H]4CCOC[C@H]4O)ncc3C#N)cc21]

To a vial containing 6-(5-chloro-2-(((3S,4R)-3-hydroxytetrahydro-2H-pyran-4-yl)amino)pyrimidin-4-yl)-4-fluoro-1-isopropyl-N-((1-methyl-1H-indazol-6-yl)methyl)-1H-indole-3-carboxamide (32 mg, 0.054 mmol), potassium acetate (2.1 mg, 0.022 mmol), potassium hexacyanoferrate(II) trihydrate (11.4 mg, 0.027 mmol), and tBuXphos Pd G4 (8.7 mg, 0.01 mmol), was added 1,4-dioxane/water (1:1; 0.5 mL). The mixture was sparged with N₂ for 1 min and stirred at 110° C. for 1 h. The reaction mixture was cooled to r.t., diluted with MeCN, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₁H₃₂FN₈O₃(M+H)⁺: m/z=583.3; found 583.4.

### Example 6: 6-(5-Chloro-2-(((3S,4R)-3-hydroxytetrahydro-2H-pyran-4-yl)amino)pyrimidin-4-yl)-4-fluoro-1-isopropyl-N,2-dimethyl-1H-indole-3-carboxamide

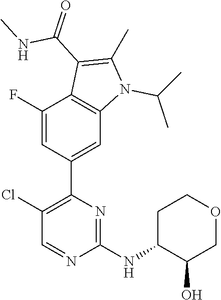 [SMILES: CNC(=O)c1c(C)n(C(C)C)c2cc(-c3nc(N[C@@H]4CCOC[C@H]4O)ncc3Cl)cc(F)c12]

### Step 1: Isopropyl 6-(5-chloro-2-(((3S,4R)-3-hydroxytetrahydro-2H-pyran-4-yl)amino)pyrimidin-4-yl)-4-fluoro-1-isopropyl-2-methyl-1H-indole-3-carboxylate

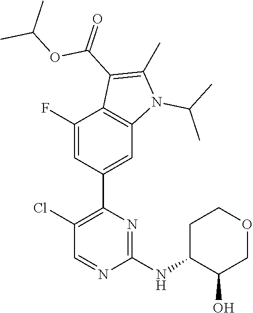 [SMILES: Cc1c(C(=O)OC(C)C)c2c(F)cc(-c3nc(N[C@@H]4CCOC[C@H]4O)ncc3Cl)cc2n1C(C)C]

To a mixture of isopropyl 6-(2,5-dichloropyrimidin-4-yl)-4-fluoro-1-isopropyl-2-methyl-1H-indole-3-carboxylate (Intermediate 9, 20 mg, 0.047 mmol) in MeCN (0.5 mL) was added (3S,4R)-4-aminotetrahydro-2H-pyran-3-ol hydrochloride (10.9 mg, 0.071 mmol, Pharmablock PBZJ8003-01) and N,N-diisopropylethylamine (0.025 mL, 0.14 mmol). The mixture was stirred at 90° C. overnight. After cooling to r.t., the mixture was concentrated under reduced pressure. The crude residue was purified by flash column chromatography (SiO₂, MeOH/CH₂Cl₂) to afford the desired product. LC-MS calculated for C₂₅H₃₁ClFN₄O₄(M+H)⁺: m/z=505.2; found 505.2.

### Step 2: 6-(5-Chloro-2-(((3S,4R)-3-hydroxytetrahydro-2H-pyran-4-yl)amino)pyrimidin-4-yl)-4-fluoro-1-isopropyl-2-methyl-1H-indole-3-carboxylic Acid

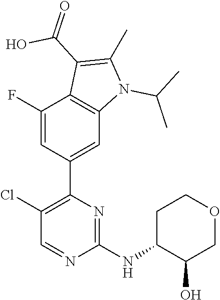 [SMILES: Cc1c(C(=O)O)c2c(F)cc(-c3nc(N[C@@H]4CCOC[C@H]4O)ncc3Cl)cc2n1C(C)C]

To a mixture of isopropyl 6-(5-chloro-2-(((3S,4R)-3-hydroxytetrahydro-2H-pyran-4-yl)amino)pyrimidin-4-yl)-4-fluoro-1-isopropyl-2-methyl-1H-indole-3-carboxylate (Step 1) in MeOH (1.0 mL) was added sodium hydroxide (6.0 mg, 0.15 mmol). The mixture was stirred at 60° C. for 1 h. The reaction mixture was diluted with aqueous 1 M HCl and extracted with EtOAc. The combined organic layers were dried over MgSO₄ and concentrated. The crude material obtained was used directly without further purification. LC-MS calculated for C₂₂H₂₅ClFN₄O₄(M+H)⁺: m/z=463.2; found 463.3.

### Step 3: 6-(5-Chloro-2-(((3S,4R)-3-hydroxytetrahydro-2H-pyran-4-yl)amino)pyrimidin-4-yl)-4-fluoro-1-isopropyl-N,2-dimethyl-1H-indole-3-carboxamide

To a mixture of 6-(5-chloro-2-(((3S,4R)-3-hydroxytetrahydro-2H-pyran-4-yl)amino)pyrimidin-4-yl)-4-fluoro-1-isopropyl-2-methyl-1H-indole-3-carboxylic acid (Step 2) in THF (1.0 mL) was added methylamine (2.0 M in THF, 0.125 mL, 0.25 mmol) and HATU (38 mg, 0.1 mmol). The reaction mixture was stirred at r.t. for 1 h. The mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₃H₂₈ClFN₅O₃(M+H)⁺: m/z=476.2; found 476.3.

### Example 7: 1-Cyclopentyl-4,7-difluoro-6-(5-fluoro-2-(((3S,4R)-3-hydroxytetrahydro-2H-pyran-4-yl)amino)pyrimidin-4-yl)-N-methyl-1H-indole-3-carboxamide

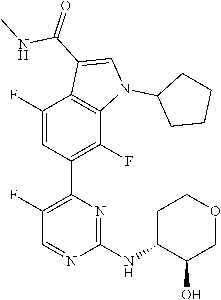 [SMILES: CNC(=O)c1cn(C2CCCC2)c2c(F)c(-c3nc(N[C@@H]4CCOC[C@H]4O)ncc3F)cc(F)c12]

### Step 1: I-Cyclopentyl-4,7-difluoro-6-(5-fluoro-2-(((3S,4R)-3-hydroxytetrahydro-2H-pyran-4-yl)amino)pyrimidin-4-yl)-1H-indole-3-carboxylic Acid

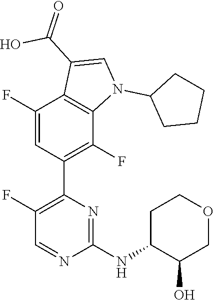 [SMILES: O=C(O)c1cn(C2CCCC2)c2c(F)c(-c3nc(N[C@@H]4CCOC[C@H]4O)ncc3F)cc(F)c12]

A vial was charged with a stir bar, ethyl 6-(2-chloro-5-fluoropyrimidin-4-yl)-1-cyclopentyl-4,7-difluoro-1H-indole-3-carboxylate (Intermediate 10, 40 mg, 0.097 mmol), (3S,4R)-4-aminotetrahydro-2H-pyran-3-ol hydrochloride (44.8 mg, 0.291 mmol, Pharmablock PBZJ8003-01), and GPhos Pd G6 TES (9.2 mg, 0.0097 mmol, Aldrich 922900). The vial was evacuated and backfilled with N₂ three times. To the vial was added THF (1.0 mL) and NaOTMS (1 M in THF, 0.49 mL, 0.49 mmol), and the reaction mixture was stirred at 60° C. for 30 min. After cooling to r.t., the reaction mixture was diluted with aqueous 1 M HCl and extracted with EtOAc. The combined organic layers were dried over MgSO₄ and concentrated. The crude residue was purified by flash column chromatography (SiO₂) to afford desired product. LC-MS calculated for C₂₃H₂₄F₃N₄O₄(M+H)⁺: m/z=477.2; found 477.1.

### Step 2: 1-Cyclopentyl-4, 7-difluoro-6-(5-fluoro-2-(((3S,4R)-3-hydroxytetrahydro-2H-pyran-4-yl)amino)pyrimidin-4-yl)-N-methyl-1H-indole-3-carboxamide

To a mixture of 1-cyclopentyl-4,7-difluoro-6-(5-fluoro-2-(((3S,4R)-3-hydroxytetrahydro-2H-pyran-4-yl)amino)pyrimidin-4-yl)-1H-indole-3-carboxylic acid (10 mg, 0.022 mmol) in THF (0.5 mL) was added methylamine (2.0 M in THF, 0.017 mL, 0.033 mmol) and HATU (12.6 mg, 0.033 mmol). The reaction mixture was stirred at r.t. for 1 h. The mixture was diluted with MeOH, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₄H₂₇F₃N₅O₃(M+H)⁺: m/z=490.2; found 490.3.

### Example 8: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(trifluoromethyl)azetidin-1-yl)methanone

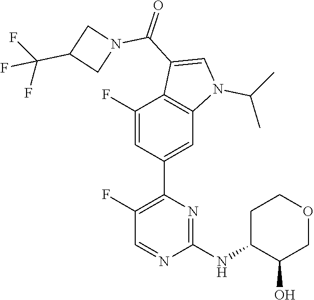 [SMILES: CC(C)n1cc(C(=O)N2CC(C(F)(F)F)C2)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 500 mg, 1.16 mmol), 3-(trifluoromethyl)azetidine hydrochloride (187 mg, 1.16 mmol), HATU (879 mg, 2.31 mmol), N,N-diisopropylethylamine (0.61 mL, 3.5 mmol), CH₃CN (9.6 mL), and N,N-dimethylformamide (1.92 mL) was stirred at 65° C. for 2 h. After cooling to r.t., the mixture was concentrated in vacuo. The crude residue was purified by flash column chromatography (SiO₂, 0-20% CH₂Cl₂/MeOH) to afford the desired product (509 mg, 82% yield) as a pale yellow solid. The material was further purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₅H₂₇F₅N₅O₃(M+H)⁺: m/z=540.2; found 540.2. ¹H NMR (500 MHz, DMSO-d₆) δ 8.43 (d, J=3.8 Hz, 1H), 8.11 (s, 1H), 8.10 (s, 1H), 7.57 (d, J=12.3 Hz, 1H), 7.26-7.11 (m, 1H), 4.86 (hept, J=6.9 Hz, 1H), 4.48-4.00 (m, 4H), 3.87-3.77 (m, 3H), 3.73-3.62 (m, 1H), 3.56-3.48 (m, 1H), 3.40-3.32 (m, 1H), 3.11-3.04 (m, 1H), 2.06-1.97 (m, 1H), 1.58-1.45 (m, 7H).

### Example 9: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(hydroxymethyl)azetidin-1-yl)methanone

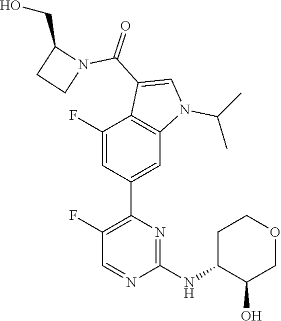 [SMILES: CC(C)n1cc(C(=O)N2CC[C@H]2CO)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic Acid (Intermediate 11, 190 mg, 0.439 mmol), (S)-azetidin-2-ylmethanol (38.3 mg, 0.439 mmol), HATU (334 mg, 0.879 mmol), N,N-diisopropylethylamine (0.23 mL, 1.3 mmol), CH₃CN (7.32 mL), and N,N-dimethylformamide (1.47 mL) was stirred at 65° C. for 1 h. After cooling to r.t., the mixture was diluted with MeCN, filtered, and purified by prep-HPLC (Sunfire C18 column, eluting with a gradient of acetonitrile/water containing 0.1% NH₄OH, at flow rate of 60 mL/min) to afford the desired product. LC-MS calculated for C₂₅H₃₀F₂N₅O₄ (M+H)⁺: m/z=502.2; found 502.2. ¹H NMR (500 MHz, DMSO-d₆) δ 8.43 (d, J=3.8 Hz, 1H), 8.08 (s, 1H), 8.02 (s, 1H), 7.54 (d, J=12.2 Hz, 1H), 7.17 (d, J=7.7 Hz, 1H), 4.92 (s, 2H), 4.84 (h, J=6.7 Hz, 1H), 4.54-4.47 (m, 1H), 4.24-3.90 (m, 2H), 3.87-3.76 (m, 3H), 3.75-3.57 (m, 2H), 3.57-3.48 (m, 1H), 3.40-3.32 (m, 1H), 3.07 (t, J=10.3 Hz, 1H), 2.34-2.24 (m, 1H), 2.14-2.05 (m, 1H), 2.05-1.97 (m, 1H), 1.58-1.45 (m, 7H).

### Example 10: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-methylazetidin-1-yl)methanone

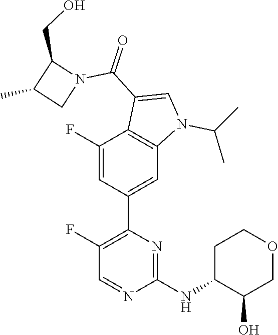 [SMILES: CC(C)n1cc(C(=O)N2C[C@H](C)[C@H]2CO)c2c(F)cc(-c3nc(N[C@@H]4CCOC[C@H]4O)ncc3F)cc21]

### Step 1: Methyl (2S,3S)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)-3-methylazetidine-2-carboxylate

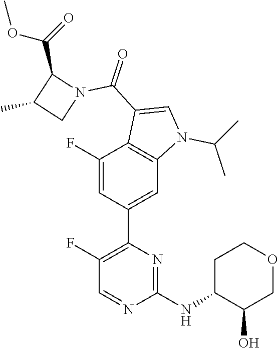 [SMILES: COC(=O)[C@@H]1[C@@H](C)CN1C(=O)c1cn(C(C)C)c2cc(-c3nc(N[C@@H]4CCOC[C@H]4O)ncc3F)cc(F)c12]

To a mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 257 mg, 0.594 mmol) and HATU (565 mg, 1.49 mmol) in DMF (3.0 mL) was added N,N-diisopropylethylamine (2.0 mL, 11.9 mmol) and methyl (2S,3S)-3-methylazetidine-2-carboxylate 2,2,2-trifluoroacetate (Intermediate 12, 519 mg, 2.13 mmol) and the mixture was stirred at 65° C. for 5 h. After cooling to r.t., the mixture was diluted with EtOAc. The organic phase was washed with 5% LiCl (aq.) and brine, dried over Na₂SO₄, filtered, and concentrated. The crude residue was purified by flash column chromatography (SiO₂, 0-90% EtOAc/CH₂Cl₂) to afford the desired product (150 mg, 47% yield) as a pale yellow solid. LC-MS calculated for C₂₇H₃₂F₂N₅O₅ (M+H)⁺: m/z=544.2; found 544.4.

### Step 2: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-methylazetidin-1-yl)methanone

A mixture of methyl (2S,3S)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)-3-methylazetidine-2-carboxylate (99 mg, 0.182 mmol) in THF (1.8 mL) was cooled to 0° C. in an ice bath before lithium aluminum hydride (2.0 M in THF, 100 μL, 0.200 mmol) was added dropwise. Rapid evolution of gas and formation of emulsion occurred. The reaction mixture was stirring at 0° C. for 5 min. The reaction was quenched by dropwise addition of sat. aq. Rochelle's salt and stirred at r.t. for 10 min. The mixture was diluted with EtOAc and the layers separated. The organic layer was washed with brine, dried over Na₂SO₄, filtered, and concentrated. The crude mixture was purified by preparative LC (Phenomenex Lux 5 μm cellulose-2, 21.2×250 mm, eluting with an isocratic mobile phase of 85% EtOH in hexanes, at flow rate of 20 mL/min) to afford the desired product. LC-MS calculated for C₂₆H₃₂F₂N₅O₄(M+H)⁺: m/z=516.2; found 516.3.

### Example 11: ((2S,4R)-4-Fluoro-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone

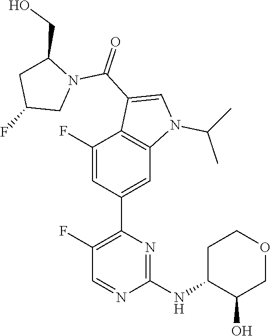 [SMILES: CC(C)n1cc(C(=O)N2C[C@H](F)C[C@H]2CO)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 200 mg, 0.463 mmol), ((2S,4R)-4-fluoropyrrolidin-2-yl)methanol hydrochloride (79.0 mg, 0.509 mmol, AA Blocks AA00IAXW), HATU (193 mg, 0.509 mmol), N,N-diisopropylethylamine (0.24 mL, 1.4 mmol), CH₃CN (2.76 mL), and N,N-dimethylformamide (1.85 mL) was stirred at 65° C. for 1 h. After cooling to r.t., the mixture was concentrated in vacuo. The crude residue was purified by flash column chromatography (SiO₂, 0-20% CH₂Cl₂/MeOH) to afford the desired product (100 mg, 40% yield) as a pale yellow solid. The material was further purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₆H₃₁F₃N₅O₄ (M+H)⁺: m/z=534.2; found 534.3.

### Example 12: ((S)-4,4-Difluoro-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone

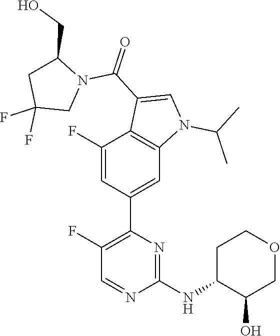 [SMILES: CC(C)n1cc(C(=O)N2CC(F)(F)C[C@H]2CO)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 200 mg, 0.463 mmol), (S)-(4,4-difluoropyrrolidin-2-yl)methanol hydrochloride (88 mg, 0.509 mmol, AmBeed A535765), HATU (193 mg, 0.509 mmol), N,N-diisopropylethylamine (0.24 mL, 1.4 mmol), CH₃CN (2.76 mL), and N,N-dimethylformamide (1.85 mL) was stirred at 65° C. for 1 h. After cooling to r.t., the mixture was concentrated in vacuo. The crude residue was purified by flash column chromatography (SiO₂, 0-20% CH₂Cl₂/MeOH) to afford the desired product (130 mg, 51% yield) as a pale yellow solid. The material was further purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₆H₃₀F₄N₅O₄ (M+H)⁺: m/z=552.2; found 552.2.

### Examples 13 and 14: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-4-(hydroxymethyl)-5-azaspiro[2.3]hexan-5-yl)methanone and (4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-4-(hydroxymethyl)-5-azaspiro[2.3]hexan-5-yl)methanone

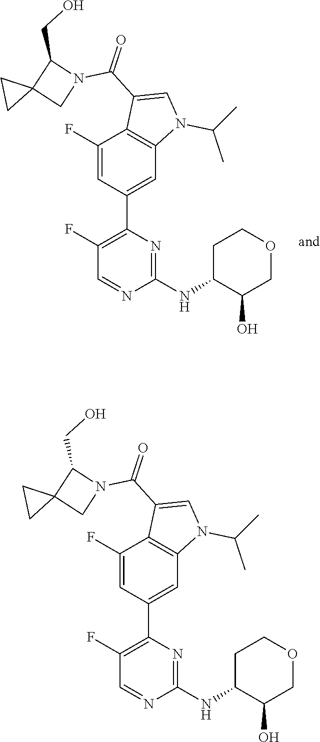 [SMILES: CC(C)n1cc(C(=O)N2CC3(CC3)[C@H]2CO)c2c(F)cc(-c3nc(N[C@@H]4CCOC[C@H]4O)ncc3F)cc21 ; CC(C)n1cc(C(=O)N2CC3(CC3)[C@@H]2CO)c2c(F)cc(-c3nc(N[C@@H]4CCOC[C@H]4O)ncc3F)cc21]

To a mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 400 mg, 0.925 mmol), (±)-(5-azaspiro[2.3]hexan-4-yl)methanol hydrochloride (Intermediate 13, 165 mg, 1.11 mmol) and N,N-diisopropylethylamine (485 μL, 2.8 mmol) in N,N-dimethylformamide (4.0 mL) was added HATU (387 mg, 1.02 mmol) and the mixture was stirred at r.t. for 1 h. The reaction mixture was diluted with EtOAc (40 mL) and washed with H₂O (3×8 mL) and sat. aq. NaCl (8 mL). The organic layer was dried over MgSO₄ and concentrated. The crude residue was purified by flash column chromatography (SiO₂, 0-20% CH₂Cl₂/MeOH) to afford the desired product (328 mg, 67% yield) as a mixture of diastereomers. The diastereomeric mixture was further purified by chiral SFC (CHIRALPAK 1H 5 μm, 21×250 mm, eluting with an isocratic mobile phase of 25% MeOH in CO₂, at flow rate of 65 mL/min) to afford each diastereomer.

### Example 13

Retention time on chiral SFC tᵣ=5.5 min, LC-MS calculated for C₂₇H₃₂F₂N₅O₄(M+H)⁺: m/z=528.2; found 528.2. ¹H NMR (600 MHz, DMSO-d₆) δ 8.43 (d, J=3.8 Hz, 1H), 8.12-8.05 (m, 2H), 7.55 (d, J=12.2 Hz, 1H), 7.18 (d, J=7.7 Hz, 1H), 4.93 (d, J=5.3 Hz, 1H), 4.89-4.70 (m, 2H), 4.45 (t, J=5.2 Hz, 1H), 4.33-4.16 (m, 1H), 4.14-3.99 (m, 1H), 3.86-3.48 (m, 6H), 3.39-3.33 (m, 1H), 3.11-3.03 (m, 1H), 2.08-1.96 (m, 1H), 1.56-1.45 (m, 7H), 0.95-0.86 (m, 1H), 0.68-0.56 (m, 2H), 0.53-0.46 (m, 1H).

### Example 14

Retention time on chiral SFC tᵣ=6.8 min, LC-MS calculated for C₂₇H₃₂F₂N₅O₄(M+H)⁺: m/z=528.2; found 528.2

Based on the difference in activity between Example 9 (having an (S)-hydroxymethyl group on the cyclized amide nitrogen) and Example 15 (having an (R)-hydroxymethyl group on the cyclized amide nitrogen), it is believed that Example 13 is (4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-4-(hydroxymethyl)-5-azaspiro[2.3]hexan-5-yl)methanone, while Example 14 is (4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-4-(hydroxymethyl)-5-azaspiro[2.3]hexan-5-yl)methanone.

### Example 15: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-2-(hydroxymethyl)azetidin-1-yl)methanone

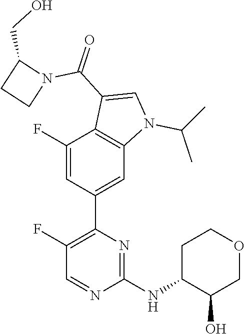 [SMILES: CC(C)n1cc(C(=O)N2CC[C@@H]2CO)c2c(F)cc(-c3nc(N[C@@H]4CCOC[C@H]4O)ncc3F)cc21]

The title compound was prepared according to the procedures described in Example 9, using (R)-azetidin-2-ylmethanol instead of (S)-azetidin-2-ylmethanol. LC-MS calculated for C₂₅H₃₀F₂N₅O₄ (M+H)⁺: m/z=502.2; found 502.3.

### Example 16: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl-d2)-3-methylazetidin-1-yl)methanone

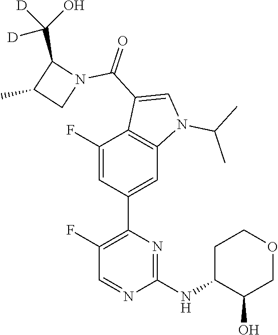 [SMILES: [2H]C([2H])(O)[C@@H]1[C@@H](C)CN1C(=O)c1cn(C(C)C)c2cc(-c3nc(N[C@@H]4CCOC[C@H]4O)ncc3F)cc(F)c12]

A mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 900 mg, 2.08 mmol), HATU (871 mg, 2.29 mmol) and N,N-diisopropylethylamine (1.1 mL, 6.3 mmol) in THF (10.4 mL) was stirred at r.t. for 10 min before ((2S,3S)-3-methylazetidin-2-yl)methan-d₂-ol hydrochloride (Intermediate 14, 436 mg, 3.12 mmol) was added. The reaction mixture was stirred at r.t. for 2 h. The mixture was diluted with sat. aq. NaHCO₃ (15 mL) and extracted with EtOAc (3×15 mL). The combined organic layers were dried over MgSO₄ and concentrated. The residue was purified by flash column chromatography (SiO₂, CH₂Cl₂/MeOH). Fractions containing the desired product were further purified by prep-HPLC (Sunfire C18 column, eluting with a gradient of acetonitrile/water containing 0.1% NH₄OH, at flow rate of 60 mL/min) to afford the desired product. ¹H NMR (500 MHz, DMSO-d₆) δ 8.45-8.40 (m, 1H), 8.08 (s, 1H), 8.02 (s, 1H), 7.58-7.51 (m, 1H), 7.16 (d, J=7.7 Hz, 1H), 4.92 (d, J=5.2 Hz, 1H), 4.88-4.82 (m, 1H), 4.32-4.12 (m, 1H), 4.02-3.98 (m, 1H), 3.86-3.77 (m, 3H), 3.60-3.47 (m, 2H), 3.40-3.32 (m, 1H), 3.11-3.04 (m, 1H), 2.51-2.43 (m, 1H), 2.07-1.96 (m, 1H), 1.55-1.45 (m, 7H), 1.20 (d, J=6.9 Hz, 3H). LC-MS calculated for C₂₆H₃₀D₂FN₅O₄ (M+H)⁺: m/z=518.3; found 518.2.

### Example 17: ((2R,3S)-3-(Difluoromethoxy)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone

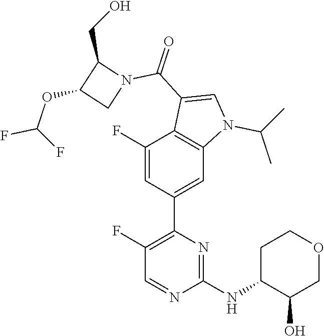 [SMILES: CC(C)n1cc(C(=O)N2C[C@H](OC(F)F)[C@H]2CO)c2c(F)cc(-c3nc(N[C@@H]4CCOC[C@H]4O)ncc3F)cc21]

A mixture of ((2R,3S)-3-(difluoromethoxy)azetidin-2-yl)methanol hydrochloride (Intermediate 16, 29.2 mg, 0.154 mmol), N,N-diisopropylethylamine (134 μL, 0.77 mmol), 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 56.6 mg, 0.131 mmol), and HATU (51.2 mg, 0.135 mmol) in MeCN (0.80 mL) was stirred at r.t. for 30 min. The mixture was diluted with CH₂Cl₂ and water, and the layers were separated. The aqueous layer was extracted with CH₂Cl₂. The combined organic layers were dried over MgSO₄ and concentrated. The residue was purified by prep-HPLC (Sunfire C18 column, eluting with a gradient of acetonitrile/water containing 0.1% NH₄OH, at flow rate of 60 mL/min) to afford the desired product. ¹H NMR (600 MHz, DMSO-d₆) δ 8.46-8.41 (m, 1H), 8.12-8.05 (m, 2H), 7.59-7.52 (m, 1H), 7.18 (d, J=7.7 Hz, 1H), 6.92-6.62 (m, 1H), 5.12-5.04 (m, 1H), 4.93 (d, J=5.0 Hz, 1H), 4.90-4.82 (m, 2H), 4.54-4.39 (m, 2H), 4.00-3.91 (m, 1H), 3.86-3.78 (m, 3H), 3.72-3.48 (m, 3H), 3.39-3.33 (m, 1H), 3.11-3.04 (m, 1H), 2.07-1.96 (m, 1H), 1.57-1.45 (m, 7H). LC-MS calculated for C₂₆H₃₀F₄N₅O₅(M+H)⁺: m/z=568.2; found 568.2.

### Example 18: (6-(5-Fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl-d2)-3-methylazetidin-1-yl)methanone

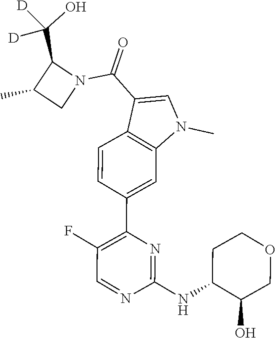 [SMILES: [2H]C([2H])(O)[C@@H]1[C@@H](C)CN1C(=O)c1cn(C)c2cc(-c3nc(N[C@@H]4CCOC[C@H]4O)ncc3F)ccc12]

A mixture of 6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 17, 50 mg, 0.129 mmol), HATU (54 mg, 0.142 mmol) and N,N-diisopropylethylamine (0.226 mL, 1.29 mmol) in DMF (0.624 mL) was stirred at r.t. for 10 min before ((2S,3S)-3-methylazetidin-2-yl)methan-d₂-ol hydrochloride (Intermediate 14, 36 mg, 0.259 mmol) was added. The reaction mixture was stirred at 50° C. for 10 min. After cooling to r.t, the mixture was diluted with CH₂Cl₂, washed with 5% LiCl (aq.) and the layers were separated. The organic layer was concentrated, and the residue was purified with prep-HPLC (Sunfire C18 column, eluting with a gradient of acetonitrile/water containing 0.1% NH₄OH, at flow rate of 60 mL/min) to afford the desired product. ¹H NMR (600 MHz, DMSO-d₆) δ 8.40 (d, J=3.9 Hz, 1H), 8.26 (d, J=8.5 Hz, 1H), 8.13 (s, 1H), 8.00 (s, 1H), 7.85 (d, J=8.7 Hz, 1H), 7.12 (d, J=7.7 Hz, 1H), 5.00 (s, 1H), 4.93 (d, J=4.3 Hz, 1H), 4.51-4.39 (m, 1H), 4.11-4.05 (m, 1H), 3.91 (s, 3H), 3.86-3.74 (m, 4H), 3.56-3.48 (m, 1H), 3.40-3.33 (m, 1H), 3.11-3.03 (m, 1H), 2.58-2.51 (m, 1H), 2.09-1.95 (m, 1H), 1.54-1.44 (m, 1H), 1.22 (d, J=6.9 Hz, 3H). LC-MS calculated for C₂₄H₂₇D2FN₅O₄ (M+H)⁺: m/z=472.2; found 472.2.

### Example 19: (1-Ethyl-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d2)-3-methylazetidin-1-yl)methanone

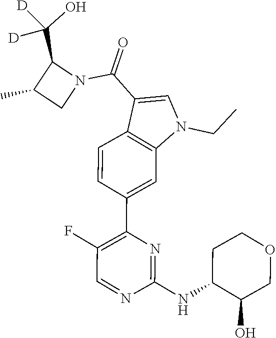 [SMILES: [2H]C([2H])(O)[C@@H]1[C@@H](C)CN1C(=O)c1cn(CC)c2cc(-c3nc(N[C@@H]4CCOC[C@H]4O)ncc3F)ccc12]

A mixture of 1-ethyl-6-(5-fluoro-2-(((3S,4R)-3-hydroxytetrahydro-2H-pyran-4-yl)amino)pyrimidin-4-yl)-1H-indole-3-carboxylic acid (Intermediate 18, 50.0 mg, 0.125 mmol), HATU (59 mg, 0.16 mmol) and N,N-diisopropylethylamine (0.087 mL, 0.5 mmol) in DMF (0.62 mL) was stirred at r.t. for 10 min before ((2S,3S)-3-methylazetidin-2-yl)methan-d₂-ol hydrochloride (Intermediate 14, 26 mg, 0.187 mmol) was added. The reaction mixture was stirred at r.t. for 1 hour. The mixture was diluted with MeCN, filtered, and purified with prep-HPLC (Sunfire C18 column, eluting with a gradient of acetonitrile/water containing 0.1% NH₄OH, at flow rate of 60 mL/min) to afford the desired product. ¹H NMR (600 MHz, DMSO-d₆) δ 8.40 (d, J=3.9 Hz, 1H), 8.27 (d, J=8.5 Hz, 1H), 8.17 (s, 1H), 8.04 (s, 1H), 7.84 (d, J=8.5 Hz, 1H), 7.12 (d, J=7.6 Hz, 1H), 5.21-4.75 (m, 2H), 4.47 (s, 1H), 4.34 (q, J=7.2 Hz, 2H), 4.13-4.05 (m, 1H), 3.90-3.73 (m, 4H), 3.58-3.49 (m, 1H), 3.40-3.33 (m, 1H), 3.11-3.03 (m, 1H), 2.60-2.51 (m, 1H), 2.09-1.98 (m, 1H), 1.56-1.46 (m, 1H), 1.43 (t, J=7.2 Hz, 3H), 1.23 (d, J=6.9 Hz, 3H). LC-MS calculated for C₂₅H₂₉D2FN₅O₄ (M+H)⁺: m/z=486.2; found 486.3.

### Example 20: (4-Fluoro-6-(5-fluoro-2-(((3S,4R)-3-hydroxytetrahydro-2H-pyran-4-yl)amino)pyrimidin-4-yl)-1-methyl-1H-indol-3-yl)(3-(trifluoromethyl)azetidin-1-yl)methanone

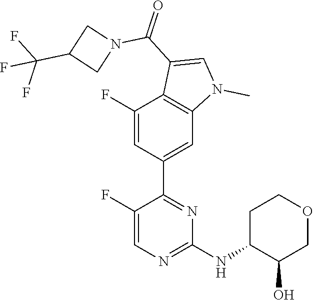 [SMILES: Cn1cc(C(=O)N2CC(C(F)(F)F)C2)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 19, 400 mg, 0.989 mmol), HATU (433 mg, 1.138 mmol) and N,N-diisopropylethylamine (862 μL, 4.95 mmol) in MeCN (10 mL) was stirred at r.t. for 10 min before 3-(trifluoromethyl)azetidine hydrochloride (240 mg, 1.48 mmol) was added. The reaction mixture was stirred at 50° C. for 10 min. After cooling to r.t, the mixture was diluted with CH₂Cl₂, washed with 5% LiCl (aq.) and the layers were separated. The organic layer was concentrated, and the residue was purified with prep-HPLC (Sunfire C18 column, eluting with a gradient of acetonitrile/water containing 0.1% NH₄OH, at flow rate of 60 mL/min) to afford the desired product. LC-MS calculated for C₂₃H₂₃F₅N₅O₃(M+H)⁺: m/z=512.2; found 512.2. ¹H NMR (600 MHz, DMSO-d₆) δ 8.44 (d, J=3.8 Hz, 1H), 8.08 (s, 1H), 8.01 (s, 1H), 7.61 (d, J=12.4 Hz, 1H), 7.20 (d, J=7.7 Hz, 1H), 4.94 (s, 1H), 4.57-4.00 (m, 4H), 3.91 (s, 3H), 3.87-3.77 (m, 3H), 3.74-3.62 (m, 1H), 3.56-3.47 (m, 1H), 3.41-3.34 (m, 1H), 3.14-3.04 (m, 1H), 2.10-1.93 (m, 1H), 1.56-1.45 (m, 1H).

### Example 21: (4-Fluoro-6-(5-fluoro-2-(((3S,4R)-3-hydroxytetrahydro-2H-pyran-4-yl)amino)pyrimidin-4-yl)-1-methyl-1H-indol-3-yl)(3-(2-fluoropropan-2-yl)azetidin-1-yl)methanone

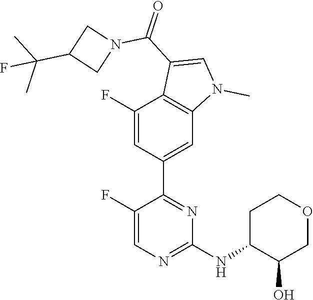 [SMILES: Cn1cc(C(=O)N2CC(C(C)(C)F)C2)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 19, 400 mg, 0.989 mmol), HATU (433 mg, 1.14 mmol) and N,N-diisopropylethylamine (862 μL, 4.95 mmol) in MeCN (10 mL) was stirred at r.t. for 10 min before 3-(2-fluoropropan-2-yl)azetidine hydrochloride (228 mg, 1.48 mmol) was added. The reaction mixture was stirred at 50° C. for 10 min. After cooling to r.t, the mixture was diluted with CH₂Cl₂, washed with 5% LiCl (aq.) and the layers were separated. The organic layer was concentrated, and the residue was purified with prep-HPLC (Sunfire C18 column, eluting with a gradient of acetonitrile/water containing 0.1% TFA, at flow rate of 60 mL/min) to afford the desired product as its TFA salt. LC-MS calculated for C₂₅H₂₉F₃N₅O₃ (M+H)⁺: m/z=504.2; found 504.2. ¹H NMR (600 MHz, DMSO-d₆) δ 8.44 (d, J=3.9 Hz, 1H), 8.04-7.98 (m, 2H), 7.60 (d, J=12.4 Hz, 1H), 7.30-7.08 (m, 1H), 4.39-3.92 (m, 4H), 3.91 (s, 3H), 3.88-3.78 (m, 3H), 3.56-3.47 (m, 1H), 3.42-3.32 (m, 1H), 3.10-3.04 (m, 1H), 2.93-2.79 (m, 1H), 2.10-1.95 (m, 1H), 1.59-1.43 (m, 1H), 1.38-1.23 (m, 6H).

### Example 22: (5,5-Difluoro-2-azaspiro[3.3]heptan-2-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone

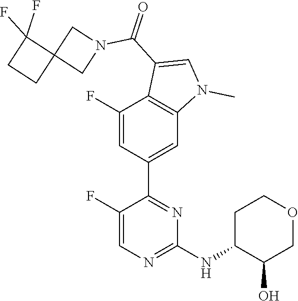 [SMILES: Cn1cc(C(=O)N2CC3(CCC3(F)F)C2)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 19, 200 mg, 0.495 mmol), HATU (216 mg, 0.569 mmol) and N,N-diisopropylethylamine (431 μL, 2.47 mmol) in MeCN (5 mL) was stirred at r.t. for 10 min before 5,5-difluoro-2-azaspiro[3.3]heptane 2,2,2-trifluoroacetate (122 mg, 0.495 mmol) was added. The reaction mixture was stirred at 50° C. for 10 min. After cooling to r.t, the mixture was diluted with CH₂Cl₂, washed with 5% LiCl (aq.) and the layers were separated. The organic layer was concentrated, and the residue was purified with prep-HPLC (Sunfire C18 column, eluting with a gradient of acetonitrile/water containing 0.1% TFA, at flow rate of 60 mL/min) to afford the desired product as its TFA salt. LC-MS calculated for C₂₅H₂₆F₄N₅O₃(M+H)⁺: m/z=520.2; found 520.2. ¹H NMR (600 MHz, DMSO-d₆) δ 8.41 (d, J=4.0 Hz, 1H), 8.02 (s, 1H), 7.97 (s, 1H), 7.60 (d, J=12.5 Hz, 1H), 4.39-4.29 (m, 2H), 4.13-4.03 (m, 2H), 3.92 (s, 3H), 3.89-3.78 (m, 3H), 3.60-3.51 (m, 1H), 3.44-3.36 (m, 1H), 3.12 (t, J=10.2 Hz, 1H), 2.54-2.47 (m, 2H), 2.11-2.04 (m, 3H), 1.61-1.49 (m, 1H).

### Example 23: (4-Fluoro-6-(5-fluoro-2-(((3S,4R)-3-hydroxytetrahydro-2H-pyran-4-yl)amino)pyrimidin-4-yl)-1-methyl-1H-indol-3-yl)((R)-2-methylazetidin-1-yl)methanone

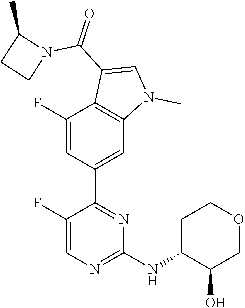 [SMILES: C[C@@H]1CCN1C(=O)c1cn(C)c2cc(-c3nc(N[C@@H]4CCOC[C@H]4O)ncc3F)cc(F)c12]

A mixture of 4-fluoro-6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 19, 400 mg, 0.98 mmol), HATU (433 mg, 1.14 mmol) and N,N-diisopropylethylamine (862 μL, 4.95 mmol) in MeCN (10 mL) was stirred at r.t. for 10 min before (R)-2-methylazetidine hydrochloride (160 mg, 1.48 mmol) was added. The reaction mixture was stirred at 50° C. for 10 min. After cooling to r.t, the mixture was diluted with CH₂Cl₂, washed with 5% LiCl (aq.) and the layers were separated. The organic layer was concentrated, and the residue was purified with prep-HPLC (Sunfire C18 column, eluting with a gradient of acetonitrile/water containing 0.1% TFA, at flow rate of 60 mL/min) to afford the desired product as its TFA salt. LC-MS calculated for C₂₃H₂₆F₂N₅O₃(M+H)⁺: m/z=458.2; found 458.2. ¹H NMR (600 MHz, DMSO-d₆) δ 8.43 (d, J=3.9 Hz, 1H), 7.99 (s, 1H), 7.92 (s, 1H), 7.58 (d, J=12.2 Hz, 1H), 7.28-7.07 (m, 1H), 5.24-4.92 (m, 1H), 4.60-4.46 (m, 1H), 4.25-4.07 (m, 1H), 3.97-3.91 (m, 1H), 3.89 (s, 3H), 3.85-3.78 (m, 3H), 3.55-3.48 (m, 1H), 3.40-3.33 (m, 1H), 3.12-3.03 (m, 1H), 2.46-2.37 (m, 1H), 2.06-1.95 (m, 1H), 1.86-1.77 (m, 1H), 1.56-1.18 (m, 4H).

### Example 24: (1,1-Difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone

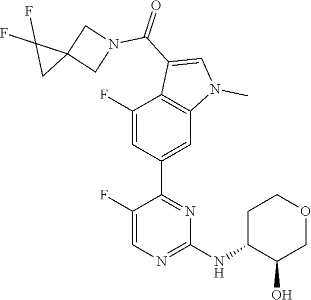 [SMILES: Cn1cc(C(=O)N2CC3(C2)CC3(F)F)c2c(F)cc(-c3nc(N[C@@H]4CCOC[C@H]4O)ncc3F)cc21]

A mixture of 4-fluoro-6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 19, 400 mg, 0.989 mmol), HATU (433 mg, 1.14 mmol) and N,N-diisopropylethylamine (862 μL, 4.95 mmol) in MeCN (10 mL) was stirred at r.t. for 10 min before 1,1-difluoro-5-azaspiro[2.3]hexane hydrochloride (231 mg, 1.48 mmol) was added. The reaction mixture was stirred at 50° C. for 10 min. After cooling to r.t, the mixture was diluted with CH₂Cl₂, washed with 5% LiCl (aq.) and the layers were separated. The organic layer was concentrated, and the residue was purified with prep-HPLC (Sunfire C18 column, eluting with a gradient of acetonitrile/water containing 0.1% TFA, at flow rate of 60 mL/min) to afford the desired product as its TFA salt. LC-MS calculated for C₂₄H₂₄F₄N₅O₃(M+H)⁺: m/z=506.2; found 506.1. ¹H NMR (600 MHz, DMSO-d₆) δ 8.45 (d, J=3.8 Hz, 1H), 8.08 (s, 1H), 8.02 (s, 1H), 7.61 (d, J=12.2 Hz, 1H), 7.33-7.09 (m, 1H), 4.49-4.04 (m, 4H), 3.91 (s, 3H), 3.87-3.79 (m, 3H), 3.57-3.48 (m, 1H), 3.43-3.33 (m, 1H), 3.14-3.04 (m, 1H), 2.06-1.96 (m, 1H), 1.82-1.70 (m, 2H), 1.57-1.42 (m, 1H).

### Example 25. (6-(5-Chloro-2-(((3S,4R)-3-hydroxytetrahydro-2H-pyran-4-yl)amino)pyrimidin-4-yl)-4-fluoro-1-isopropyl-1H-indol-3-yl)((2S,3S)-2-(hydroxymethyl)-3-methylazetidin-1-yl)methanone

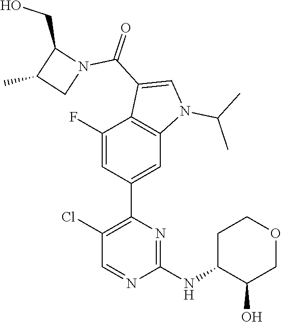 [SMILES: CC(C)n1cc(C(=O)N2C[C@H](C)[C@H]2CO)c2c(F)cc(-c3nc(N[C@@H]4CCOC[C@H]4O)ncc3Cl)cc21]

A mixture of 6-(5-chloro-2-(((3S,4R)-3-hydroxytetrahydro-2H-pyran-4-yl)amino)pyrimidin-4-yl)-4-fluoro-1-isopropyl-1H-indole-3-carboxylic acid (Intermediate 8, 300 mg, 0.67 mmol), N,N-diisopropylethylamine (350 μL, 2.01 mmol), ((2S,3S)-3-methylazetidin-2-yl)methanol hydrochloride (Intermediate 24, 368 mg, 2.67 mmol), and HATU (280 mg, 0.74 mmol) in MeCN (5.57 mL) was stirred at r.t. for 30 min. The mixture was diluted with EtOAc and water, and the layers were separated. The aqueous layer was extracted with EtOAc. The combined organic layers were dried over Na₂SO₄ and concentrated. The residue was purified by flash column chromatography (SiO₂, CH₂Cl₂/MeOH). Fractions containing the desired product were further purified by prep-HPLC (Sunfire C18 column, eluting with a gradient of acetonitrile/water containing 0.1% NH₄OH, at flow rate of 60 mL/min) to afford the desired product. ¹H NMR (500 MHz, DMSO-d₆) δ 8.40 (s, 1H), 7.95 (s, 1H), 7.89 (s, 1H), 7.33 (d, J=11.9 Hz, 1H), 7.28-7.19 (m, 1H), 4.92-4.70 (m, 3H), 4.27-4.16 (m, 1H), 4.08-3.97 (m, 1H), 3.90-3.77 (m, 3H), 3.75-3.46 (m, 4H), 3.41-3.31 (m, 1H), 3.11-3.03 (m, 1H), 2.50-2.43 (m, 1H), 2.09-1.96 (m, 1H), 1.57-1.51 (m, 7H), 1.22 (d, J=6.9 Hz, 3H). LC-MS calculated for C₂₆H₃₂ClFN₅O₄(M+H)⁺: m/z=532.2; found 532.2.

### Example 26. (6-(5-Chloro-2-(((3S,4R)-3-hydroxytetrahydro-2H-pyran-4-yl)amino)pyrimidin-4-yl)-4-fluoro-1-isopropyl-1H-indol-3-yl)((2S,3S)-2-(hydroxymethyl-d2)-3-methylazetidin-1-yl)methanone

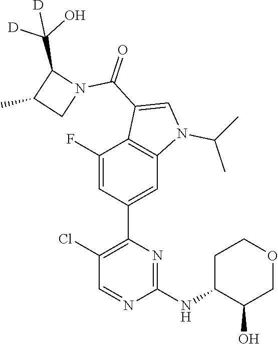 [SMILES: [2H]C([2H])(O)[C@@H]1[C@@H](C)CN1C(=O)c1cn(C(C)C)c2cc(-c3nc(N[C@@H]4CCOC[C@H]4O)ncc3Cl)cc(F)c12]

The title compound was prepared according to the procedures outlined for Example 25, with ((2S,3S)-3-methylazetidin-2-yl)methan-d₂-ol hydrocloride (Intermediate 14) replacing ((2S,3S)-3-methylazetidin-2-yl)methanol hydrochloride. ¹H NMR (500 MHz, DMSO-d₆) δ 8.42 (s, 1H), 8.00 (s, 1H), 7.88 (s, 1H), 7.51-7.39 (m, 1H), 7.31 (d, J=11.7 Hz, 1H), 4.88-4.77 (m, 1H), 4.40-4.08 (m, 1H), 4.01 (d, J=5.1 Hz, 1H), 3.89-3.72 (m, 3H), 3.70-3.44 (m, 2H), 3.38-3.32 (m, 1H), 3.05 (t, J=10.4 Hz, 1H), 2.50-2.41 (m, 1H), 2.08-1.86 (m, 1H), 1.60-1.45 (m, 7H), 1.29-1.15 (m, 3H). LC-MS calculated for C₂₆H₃₀D₂ClFN₅O₄(M+H)⁺: m/z=534.2; found 534.2.

### Example 27. (1-Ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d2)-3-methylazetidin-1-yl)methanone

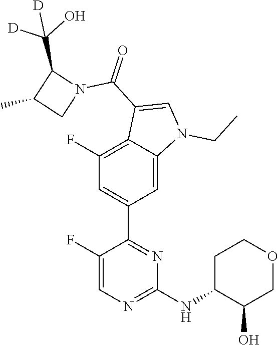 [SMILES: [2H]C([2H])(O)[C@@H]1[C@@H](C)CN1C(=O)c1cn(CC)c2cc(-c3nc(N[C@@H]4CCOC[C@H]4O)ncc3F)cc(F)c12]

A mixture of 1-ethyl-4-fluoro-6-(5-fluoro-2-(((3S,4R)-3-hydroxytetrahydro-2H-pyran-4-yl)amino)pyrimidin-4-yl)-1H-indole-3-carboxylic acid (Intermediate 20, 250 mg, 0.598 mmol), ((2S,3S)-3-methylazetidin-2-yl)methan-d₂-ol hydrochloride (Intermediate 14, 167 mg, 1.20 mmol), N,N-diisopropylethylamine (417 μL, 2.39 mmol), propylphosphonic anhydride solution (˜50% in DMF, 570 mg, 0.896 mmol), methanol (24.2 μL, 0.598 mmol), and MeCN (5.98 mL) was stirred at r.t. for 1 h. The mixture was diluted with EtOAc and water, and the layers were separated. The aqueous layer was extracted with EtOAc. The combined organic layers were dried over Na₂SO₄ and concentrated. The residue was purified by flash column chromatography (SiO₂, CH₂Cl₂/MeOH). Fractions containing the desired product were further purified by prep-HPLC (Sunfire C18 column, eluting with a gradient of acetonitrile/water containing 0.1% NH₄OH, at flow rate of 60 mL/min) to afford the desired product. ¹H NMR (500 MHz, DMSO-d₆) δ 8.44 (d, J=3.8 Hz, 1H), 8.04 (s, 1H), 8.00 (s, 1H), 7.56 (d, J=12.3 Hz, 1H), 7.21-7.14 (m, 1H), 5.03-4.75 (m, 2H), 4.40-4.28 (m, 2H), 4.28-4.11 (m, 1H), 4.04-3.97 (m, 1H), 3.89-3.78 (m, 3H), 3.68-3.47 (m, 2H), 3.41-3.33 (m, 1H), 3.12-3.02 (m, 1H), 2.50-2.45 (m, 1H), 2.09-1.97 (m, 1H), 1.59-1.45 (m, 1H), 1.43 (t, J=7.2 Hz, 3H), 1.28-1.10 (m, 3H). LC-MS calculated for C₂₅H₂₈D2F₂N₅O₄ (M+H)⁺: m/z=504.2; found 504.3.

### Example 28. ((2R,3S)-3-(Difluoromethoxy)-2-(hydroxymethyl)azetidin-1-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone

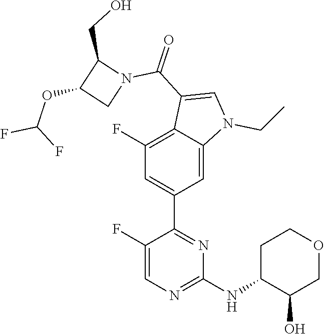 [SMILES: CCn1cc(C(=O)N2C[C@H](OC(F)F)[C@H]2CO)c2c(F)cc(-c3nc(N[C@@H]4CCOC[C@H]4O)ncc3F)cc21]

The title compound was prepared according to the procedures outlined for Example 27, with ((2R,3S)-3-(difluoromethoxy)azetidin-2-yl)methanol hydrochloride (Intermediate 16) replacing ((2S,3S)-3-methylazetidin-2-yl)methan-d₂-ol hydrochloride. LC-MS calculated for C₂₅H₂₈F₄N₅O₅(M+H)⁺: m/z=554.2; found 554.3.

### Example 29. N-((S)-1-Cyclopropyl-2-hydroxyethyl)-1-ethyl-4-fluoro-6-(5-fluoro-2-(((3S,4R)-3-hydroxytetrahydro-2H-pyran-4-yl)amino)pyrimidin-4-yl)-1H-indole-3-carboxamide

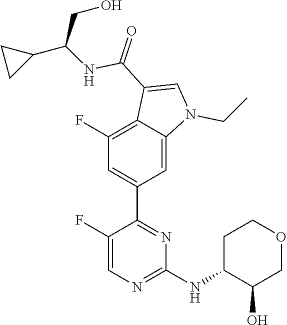 [SMILES: CCn1cc(C(=O)N[C@H](CO)C2CC2)c2c(F)cc(-c3nc(N[C@@H]4CCOC[C@H]4O)ncc3F)cc21]

The title compound was prepared according to the procedures outlined for Example 27, with (S)-2-amino-2-cyclopropylethan-1-ol hydrochloride replacing ((2S,3S)-3-methylazetidin-2-yl)methan-d₂-ol hydrochloride. LC-MS calculated for C₂₅H₃₀F₂N₅O₄(M+H)⁺: m/z=502.2; found 502.3.

### Example 30. (1-Ethyl-4-fluoro-6-(5-fluoro-2-(((3S,4R)-3-hydroxytetrahydro-2H-pyran-4-yl)amino)pyrimidin-4-yl)-1H-indol-3-yl)((S)-2-(trifluoromethyl)azetidin-1-yl)methanone

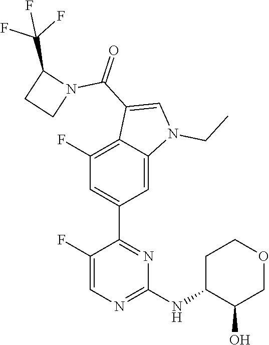 [SMILES: CCn1cc(C(=O)N2CC[C@H]2C(F)(F)F)c2c(F)cc(-c3nc(N[C@@H]4CCOC[C@H]4O)ncc3F)cc21]

The title compound was prepared according to the procedures outlined for Example 27, with (S)-2-(trifluoromethyl)azetidine replacing ((2S,3S)-3-methylazetidin-2-yl)methan-d₂-ol hydrochloride. LC-MS calculated for C₂₄H₂₅F₅N₅O₃(M+H)⁺: m/z=526.2; found 526.3.

### Example 31. (1,1-Difluoro-5-azaspiro[2.3]hexan-5-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone

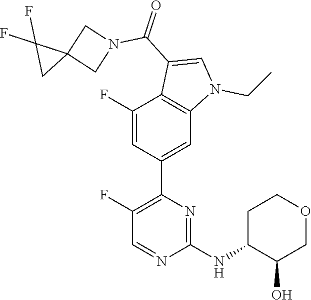 [SMILES: CCn1cc(C(=O)N2CC3(C2)CC3(F)F)c2c(F)cc(-c3nc(N[C@@H]4CCOC[C@H]4O)ncc3F)cc21]

The title compound was prepared according to the procedures outlined for Example 27, with 1,1-difluoro-5-azaspiro[2.3]hexane hydrochloride replacing ((2S,3S)-3-methylazetidin-2-yl)methan-d₂-ol hydrochloride. LC-MS calculated for C₂₅H₂₆F₄N₅O₃(M+H)⁺: m/z=520.2; found 520.3.

### Example 32. (1,1-Difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone

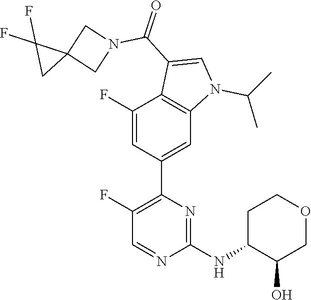 [SMILES: CC(C)n1cc(C(=O)N2CC3(C2)CC3(F)F)c2c(F)cc(-c3nc(N[C@@H]4CCOC[C@H]4O)ncc3F)cc21]

The title compound was prepared according to the procedures outlined for Example 8, with 1,1-difluoro-5-azaspiro[2.3]hexane hydrochloride replacing 3-(trifluoromethyl)azetidine hydrochloride. LC-MS calculated for C₂₆H₂₈F₄N₅O₃(M+H)⁺: m/z=534.2; found 534.3.

### Example 33. (1-Ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl)-3-(trifluoromethyl)azetidin-1-yl)methanone

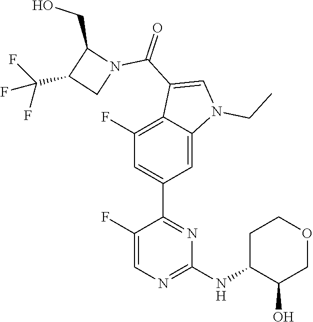 [SMILES: CCn1cc(C(=O)N2C[C@H](C(F)(F)F)[C@H]2CO)c2c(F)cc(-c3nc(N[C@@H]4CCOC[C@H]4O)ncc3F)cc21]

The title compound was prepared according to the procedures outlined for Example 27, with ((2S,3S)-3-(trifluoromethyl)azetidin-2-yl)methanol (Intermediate 21) replacing ((2S,3S)-3-methylazetidin-2-yl)methan-d₂-ol hydrochloride. ¹H NMR (500 MHz, DMSO-d₆) δ 8.44 (d, J=3.8 Hz, 1H), 8.08 (s, 1H), 8.05 (s, 1H), 7.58 (d, J=12.3 Hz, 1H), 7.18 (d, J=7.7 Hz, 1H), 5.15 (s, 1H), 4.98-4.87 (m, 1H), 4.60-4.50 (m, 1H), 4.45-4.27 (m, 3H), 4.09-3.95 (m, 1H), 3.89-3.78 (m, 3H), 3.77-3.42 (m, 4H), 3.41-3.34 (m, 1H), 3.13-3.03 (m, 1H), 2.06-1.95 (m, 1H), 1.58-1.46 (m, 1H), 1.43 (t, J=7.2 Hz, 3H). LC-MS calculated for C₂₅H₂₇F₅N₅O₄(M+H)⁺: m/z=556.2; found 556.2.

### Example 34. (1-Ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d2)-3-(trifluoromethyl)azetidin-1-yl)methanone

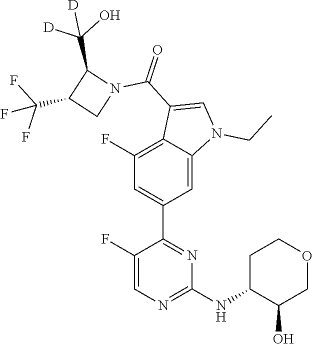 [SMILES: [2H]C([2H])(O)[C@@H]1[C@@H](C(F)(F)F)CN1C(=O)c1cn(CC)c2cc(-c3nc(N[C@@H]4CCOC[C@H]4O)ncc3F)cc(F)c12]

The title compound was prepared according to the procedures outlined for Example 27, with ((2S,3S)-3-(trifluoromethyl)azetidin-2-yl)methan-d₂-ol (Intermediate 22) replacing ((2S,3S)-3-methylazetidin-2-yl)methan-d₂-ol hydrochloride. LC-MS calculated for C₂₅H₂₅D₂F₅N₅O₄ (M+H)⁺: m/z=558.2; found 558.2.

### Example 35. (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-(trifluoromethyl)azetidin-1-yl)methanone

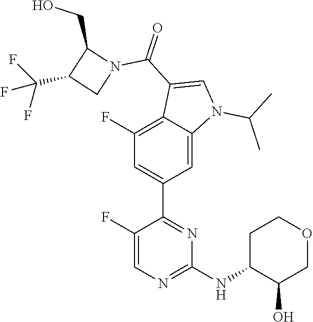 [SMILES: CC(C)n1cc(C(=O)N2C[C@H](C(F)(F)F)[C@H]2CO)c2c(F)cc(-c3nc(N[C@@H]4CCOC[C@H]4O)ncc3F)cc21]

The title compound was prepared according to the procedures outlined for Example 16, with ((2S,3S)-3-(trifluoromethyl)azetidin-2-yl)methanol (Intermediate 21) replacing ((2S,3S)-3-methylazetidin-2-yl)methan-d2-ol hydrochloride. ¹H NMR (600 MHz, DMSO-d6) δ 8.50-8.41 (m, 1H), 8.13-8.06 (m, 2H), 7.63-7.53 (m, 1H), 7.24-7.16 (m, 1H), 5.18-5.11 (m, 1H), 4.95-4.92 (m, 1H), 4.91-4.83 (m, 1H), 4.61-4.48 (m, 1H), 4.42-4.38 (m, 1H), 4.04-3.96 (m, 1H), 3.89-3.78 (m, 3H), 3.74-3.42 (m, 4H), 3.42-3.34 (m, 1H), 3.11-3.04 (m, 1H), 2.12-1.96 (m, 1H), 1.64-1.43 (m, 7H). LC-MS calculated for C₂₆H₂₉F₅N₅O₄ (M+H)⁺: m/z=570.2; found 570.2.

### Example 36. (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl-d2)-3-(trifluoromethyl)azetidin-1-yl)methanone

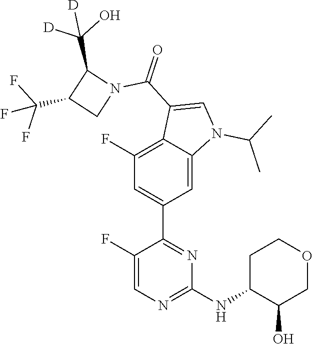 [SMILES: [2H]C([2H])(O)[C@@H]1[C@@H](C(F)(F)F)CN1C(=O)c1cn(C(C)C)c2cc(-c3nc(N[C@@H]4CCOC[C@H]4O)ncc3F)cc(F)c12]

The title compound was prepared according to the procedures outlined for Example 16, with ((2S,3S)-3-(trifluoromethyl)azetidin-2-yl)methan-d₂-ol (Intermediate 22) replacing ((2S,3S)-3-methylazetidin-2-yl)methan-d₂-ol hydrochloride. LC-MS calculated for C₂₆H₂₇D₂F₅N₅O₄ (M+H)⁺: m/z=572.2; found 572.2.

### Example 37. (4-Fluoro-6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-(trifluoromethyl)azetidin-1-yl)methanone

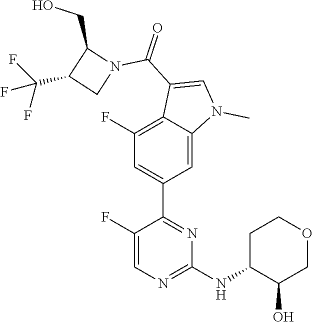 [SMILES: Cn1cc(C(=O)N2C[C@H](C(F)(F)F)[C@H]2CO)c2c(F)cc(-c3nc(N[C@@H]4CCOC[C@H]4O)ncc3F)cc21]

The title compound was prepared according to the procedures outlined for Example 20, with ((2S,3S)-3-(trifluoromethyl)azetidin-2-yl)methanol (Intermediate 21) replacing 3-(trifluoromethyl)azetidine hydrochloride. LC-MS calculated for C₂₄H₂₅F₅N₅O₄(M+H)⁺: m/z=542.2; found 542.2.

### Example 38. (1-Ethyl-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl)-3-(trifluoromethyl)azetidin-1-yl)methanone

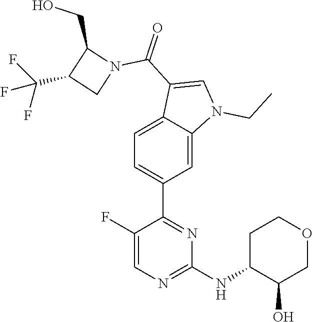 [SMILES: CCn1cc(C(=O)N2C[C@H](C(F)(F)F)[C@H]2CO)c2ccc(-c3nc(N[C@@H]4CCOC[C@H]4O)ncc3F)cc21]

The title compound was prepared according to the procedures outlined for Example 19, with ((2S,3S)-3-(trifluoromethyl)azetidin-2-yl)methanol (Intermediate 21) replacing ((2S,3S)-3-methylazetidin-2-yl)methan-d₂-ol hydrochloride. LC-MS calculated for C₂₅H₂₈F₄N₅O₄ (M+H)⁺: m/z=538.2; found 538.2.

### Example 39. (6-(5-Fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-(trifluoromethyl)azetidin-1-yl)methanone

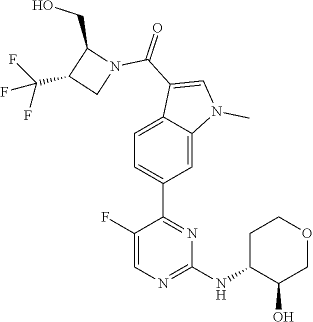 [SMILES: Cn1cc(C(=O)N2C[C@H](C(F)(F)F)[C@H]2CO)c2ccc(-c3nc(N[C@@H]4CCOC[C@H]4O)ncc3F)cc21]

The title compound was prepared according to the procedures outlined for Example 18, with ((2S,3S)-3-(trifluoromethyl)azetidin-2-yl)methanol (Intermediate 21) replacing ((2S,3S)-3-methylazetidin-2-yl)methan-d2-ol hydrochloride. ¹H NMR (500 MHz, DMSO-d₆) δ 8.44-8.37 (m, 1H), 8.29-8.22 (m, 1H), 8.16-8.13 (m, 1H), 8.11-8.08 (m, 1H), 7.92-7.83 (m, 1H), 7.19-7.05 (m, 1H), 5.35-5.10 (m, 1H), 5.00-4.85 (m, 1H), 4.67-4.49 (m, 2H), 4.31-4.10 (m, 1H), 4.05-3.90 (m, 3H), 3.88-3.78 (m, 4H), 3.72-3.62 (m, 1H), 3.62-3.48 (m, 2H), 3.41-3.35 (m, 1H), 3.13-3.01 (m, 1H), 2.11-1.92 (m, 1H), 1.57-1.40 (m, 1H). LC-MS calculated for C₂₄H₂₆F₄N₅O₄(M+H)⁺: m/z=524.2; found 524.2.

### Example 40. (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-methoxy-3-methylazetidin-1-yl)methanone

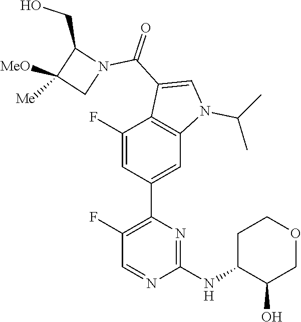 [SMILES: CO[C@]1(C)CN(C(=O)c2cn(C(C)C)c3cc(-c4nc(N[C@@H]5CCOC[C@H]5O)ncc4F)cc(F)c23)[C@@H]1CO]

### Step 1: ((2R,3R)-2-(((tert-Butyldiphenylsilyl)oxy)methyl)-3-methoxy-3-methylazetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone

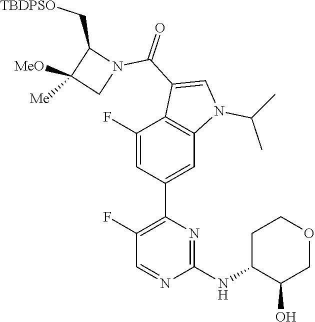

A mixture of 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11, 30 mg, 0.069 mmol), N,N-diisopropylethylamine (18.0 μL, 0.104 mmol), (2R,3R)-2-(((tert-butyldiphenylsilyl)oxy)methyl)-3-methoxy-3-methylazetidine (Intermediate 23, 38.5 mg, 0.104 mmol), and HATU (39.6 mg, 0.104 mmol) in MeCN (0.5 mL) was stirred at r.t. for 30 min. The mixture was diluted with EtOAc and water, and the layers were separated. The aqueous layer was extracted with EtOAc. The combined organic layers were dried over Na₂SO₄ and concentrated. The residue was used for the next step without further purification.

### Step 2: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-methoxy-3-methylazetidin-1-yl)methanone

To a mixture of ((2R,3R)-2-(((tert-butyldiphenylsilyl)oxy)methyl)-3-methoxy-3-methylazetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone (Step 1) in MeOH (0.5 mL) was added acetyl chloride (5.0 μL, 0.07 mmol). The reaction mixture was stirred at r.t. for 30 min. The mixture was diluted with MeCN, filtered, and purified with prep-HPLC (Sunfire C18 column, eluting with a gradient of acetonitrile/water containing 0.1% TFA, at flow rate of 60 mL/min) to afford the desired product as its TFA salt. LC-MS calculated for C₂₇H₃₄F₂N₅O₅ (M+H)⁺: m/z=546.2; found 546.3.

### Example 41. (1-Ethyl-4-fluoro-6-(5-fluoro-2-(((3S,4R)-3-hydroxytetrahydro-2H-pyran-4-yl)amino)pyrimidin-4-yl)-1H-indol-3-yl)((2R,3R)-2-(hydroxymethyl)-3-methoxy-3-methylazetidin-1-yl)methanone

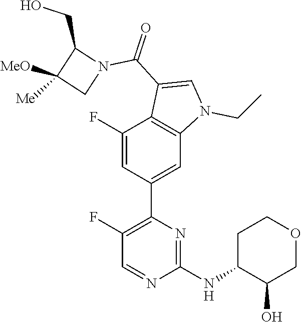 [SMILES: CCn1cc(C(=O)N2C[C@@](C)(OC)[C@H]2CO)c2c(F)cc(-c3nc(N[C@@H]4CCOC[C@H]4O)ncc3F)cc21]

The title compound was prepared according to the procedures outlined for Example 40, with 1-ethyl-4-fluoro-6-(5-fluoro-2-(((3S,4R)-3-hydroxytetrahydro-2H-pyran-4-yl)amino)pyrimidin-4-yl)-1H-indole-3-carboxylic acid (Intermediate 20) replacing 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid in Step 1. After reaction, the residue was purified with prep-HPLC (Sunfire C18 column, eluting with a gradient of acetonitrile/water containing 0.1% NH₄OH, at flow rate of 60 mL/min) to afford the desired product. LC-MS calculated for C₂₆H₃₂F₂N₅O₅ (M+H)⁺: m/z=532.2; found 532.2.

### Example 42. (4-Fluoro-6-(5-fluoro-2-(((3S,4R)-3-hydroxytetrahydro-2H-pyran-4-yl)amino)pyrimidin-4-yl)-1-methyl-1H-indol-3-yl)((2R,3R)-2-(hydroxymethyl)-3-methoxy-3-methylazetidin-1-yl)methanone

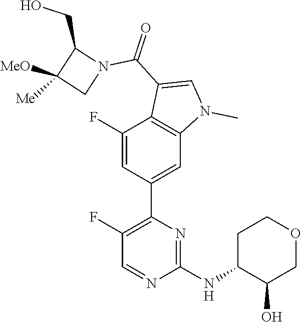 [SMILES: CO[C@]1(C)CN(C(=O)c2cn(C)c3cc(-c4nc(N[C@@H]5CCOC[C@H]5O)ncc4F)cc(F)c23)[C@@H]1CO]

The title compound was prepared according to the procedures outlined for Example 40, with 4-fluoro-6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 19) replacing 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid in Step 1. LC-MS calculated for C₂₅H₃₀F₂N₅O₅(M+H)⁺: m/z=518.2; found 518.1.

### Example 43: (6-(5-Fluoro-2-(((3S,4R)-3-hydroxytetrahydro-2H-pyran-4-yl)amino)pyrimidin-4-yl)-1-methyl-1H-indol-3-yl)((2R,3S)-2-(hydroxymethyl)-3-(trifluoromethoxy)azetidin-1-yl)methanone

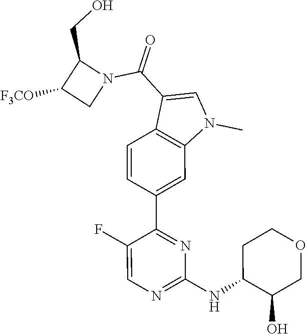 [SMILES: Cn1cc(C(=O)N2C[C@H](OC(F)(F)F)[C@H]2CO)c2ccc(-c3nc(N[C@@H]4CCOC[C@H]4O)ncc3F)cc21]

N,N-diisopropylethylamine (68 μL, 0.39 mmol) and HATU (77 mg, 0.204 mmol) were added sequentially to a mixture of 6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 17, 75 mg, 0.194 mmol) in MeCN (0.97 mL) and DMF (0.97 mL). After 10 minutes, a mixture of ((2R,3S)-3-(trifluoromethoxy)azetidin-2-yl)methanol hydrochloride (Intermediate 31, 52.1 mg, 0.252 mmol) and N,N-diisopropylethylamine (68 μL, 0.39 mmol) in MeCN (0.97 mL) was added to the reaction mixture, which was stirred at r.t. for 10 minutes. The mixture was diluted with EtOAc and water and filtered through celite. The layers were separated, the aqueous layer was extracted with EtOAc, and the combined organic layers were washed with brine, dried over magnesium sulfate, and concentrated in vacuo. The residue was purified by flash column chromatography (24 g SiO₂, MeOH/CH₂Cl₂) and further purified by prep-HPLC (Sunfire C18 column, eluting with a gradient of acetonitrile/water containing 0.1% NH₄OH, at flow rate of 60 mL/min) to afford the title compound as a white solid. LC-MS calculated for C₂₄H₂₆F₄N₅O₅(M+H)⁺: m/z=540.2; found 540.1. ¹H NMR (500 MHz, DMSO-d₆) δ 8.41 (d, J=4.0 Hz, 1H), 8.25 (d, J=8.5 Hz, 1H), 8.15 (s, 1H), 8.08 (s, 1H), 7.88 (d, J=8.5 Hz, 1H), 7.12 (d, J=7.6 Hz, 1H), 5.19 (s, 1H), 5.11 (dt, J=6.9, 3.6 Hz, 1H), 4.93 (s, 1H), 4.73 (t, J=8.4 Hz, 1H), 4.63-4.60 (m, 1H), 4.30-4.25 (m, 1H), 3.92 (s, 3H), 3.87-3.79 (m, 4H), 3.74 (dd, J=11.9, 3.4 Hz, 1H), 3.56-3.49 (m, 1H), 3.42-3.34 (m, 1H), 3.08 (t, J=10.3 Hz, 1H), 2.06-2.00 (m, 1H), 1.56-1.44 (m, 1H). ¹⁹F NMR (471 MHz, DMSO-d₆) δ-58.10 (3F), −154.91 (1F).

### Example 44: (4-Fluoro-6-(5-fluoro-2-(((3S,4R)-3-hydroxytetrahydro-2H-pyran-4-yl)amino)pyrimidin-4-yl)-1-methyl-1H-indol-3-yl)((2R,3S)-2-(hydroxymethyl)-3-(trifluoromethoxy)azetidin-1-yl)methanone

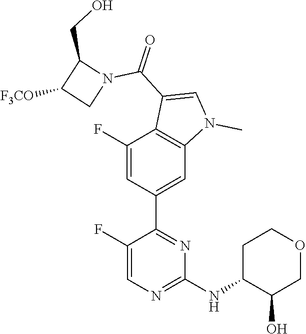 [SMILES: Cn1cc(C(=O)N2C[C@H](OC(F)(F)F)[C@H]2CO)c2c(F)cc(-c3nc(N[C@@H]4CCOC[C@H]4O)ncc3F)cc21]

The title compound was prepared according to the procedure outlined for Example 43, with 4-fluoro-6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 19) replacing 6-(5-fluoro-2-(((3S,4R)-3-hydroxytetrahydro-2H-pyran-4-yl)amino)pyrimidin-4-yl)-1-methyl-1H-indole-3-carboxylic acid. LC-MS calculated for C₂₄H₂₅F₅N₅O₅(M+H)⁺: m/z=558.2; found 558.1. ¹H NMR (600 MHz, DMSO-d₆) δ 8.44 (d, J=3.9 Hz, 1H), 8.05 (s, 1H), 8.01 (d, J=1.2 Hz, 1H), 7.60 (dd, J=12.2, 1.2 Hz, 1H), 7.19 (d, J=7.7 Hz, 1H), 5.15 (t, J=5.4 Hz, 1H), 5.06 (dt, J=6.8, 3.6 Hz, 1H), 4.93 (d, J=5.3 Hz, 1H), 4.60-4.52 (m, 2H), 4.12-4.07 (m, 1H), 3.91 (s, 3H), 3.87-3.79 (m, 3H), 3.73-3.60 (m, 2H), 3.52 (tt, J=9.6, 5.1 Hz, 1H), 3.41-3.34 (m, 1H), 3.08 (t, J=10.3 Hz, 1H), 2.05-1.98 (m, 1H), 1.50 (dtd, J=13.1, 11.4, 4.5 Hz, 1H). ¹⁹F NMR (565 MHz, DMSO-d₆) δ-58.12 (3F), −113.58 (1F), −154.64 (1F).

### Example 45: (4-Fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3S)-2-(hydroxymethyl)-3-(trifluoromethoxy)azetidin-1-yl)methanone

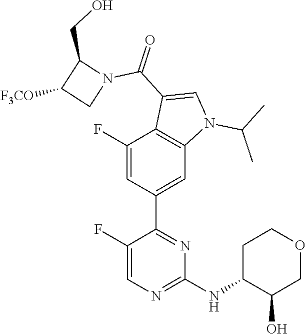 [SMILES: CC(C)n1cc(C(=O)N2C[C@H](OC(F)(F)F)[C@H]2CO)c2c(F)cc(-c3nc(N[C@@H]4CCOC[C@H]4O)ncc3F)cc21]

The title compound was prepared according to the procedure outlined for Example 43, with 4-fluoro-6-(5-fluoro-2-(((3S,4R)-3-hydroxytetrahydro-2H-pyran-4-yl)amino)pyrimidin-4-yl)-1-isopropyl-1H-indole-3-carboxylic acid (Intermediate 11) replacing 6-(5-fluoro-2-(((3S,4R)-3-hydroxytetrahydro-2H-pyran-4-yl)amino)pyrimidin-4-yl)-1-methyl-1H-indole-3-carboxylic acid. LC-MS calculated for C₂₆H₂₉F₅N₅O₅ (M+H)⁺: m/z=586.2; found 586.4. ¹H NMR (500 MHz, DMSO-d₆) δ 8.44 (d, J=4.0 Hz, 1H), 8.12-8.08 (m, 2H), 7.57 (d, J=12.2 Hz, 1H), 7.18 (d, J=7.6 Hz, 1H), 5.05 (dt, J=7.0, 3.6 Hz, 1H), 4.88 (h, J=6.8 Hz, 1H), 4.66-4.49 (m, 2H), 4.12-4.06 (m, 1H), 3.88-3.77 (m, 3H), 3.67-3.63 (m, 2H), 3.53 (td, J=9.4, 4.8 Hz, 1H), 3.37 (td, J=11.7, 2.3 Hz, 1H), 3.08 (t, J=10.3 Hz, 1H), 2.06-2.00 (m, 1H), 1.57-1.45 (m, 8H). ¹⁹F NMR (565 MHz, DMSO-d₆) δ-58.11 (1F), −114.13 (1F), −154.78 (3F).

### Example 46: (6-(5-Chloro-2-(((3S,4R)-3-hydroxytetrahydro-2H-pyran-4-yl)amino)pyrimidin-4-yl)-4-fluoro-1-isopropyl-1H-indol-3-yl)((2R,3S)-2-(hydroxymethyl)-3-(trifluoromethoxy)azetidin-1-yl)methanone

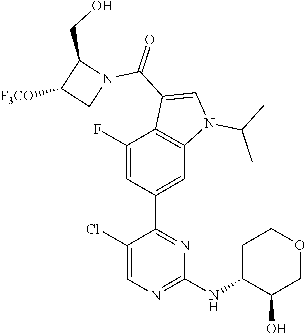 [SMILES: CC(C)n1cc(C(=O)N2C[C@H](OC(F)(F)F)[C@H]2CO)c2c(F)cc(-c3nc(N[C@@H]4CCOC[C@H]4O)ncc3Cl)cc21]

The title compound was prepared according to the procedure outlined for Example 43, with 6-(5-chloro-2-(((3S,4R)-3-hydroxytetrahydro-2H-pyran-4-yl)amino)pyrimidin-4-yl)-4-fluoro-1-isopropyl-1H-indole-3-carboxylic acid (Intermediate 8) replacing 6-(5-fluoro-2-(((3S,4R)-3-hydroxytetrahydro-2H-pyran-4-yl)amino)pyrimidin-4-yl)-1-methyl-1H-indole-3-carboxylic acid. LC-MS calculated for C₂₆H₂₉ClF₄N₅O₅ (M+H)⁺: m/z=602.2; found 602.2.

### Example 47: (1-Ethyl-4-fluoro-6-(5-fluoro-2-(((3S,4R)-3-hydroxytetrahydro-2H-pyran-4-yl)amino)pyrimidin-4-yl)-1H-indol-3-yl)((2R,3S)-2-(hydroxymethyl)-3-(trifluoromethoxy)azetidin-1-yl)methanone

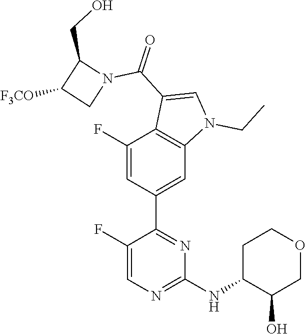 [SMILES: CCn1cc(C(=O)N2C[C@H](OC(F)(F)F)[C@H]2CO)c2c(F)cc(-c3nc(N[C@@H]4CCOC[C@H]4O)ncc3F)cc21]

The title compound was prepared according to the procedure outlined for Example 43, with 1-ethyl-4-fluoro-6-(5-fluoro-2-(((3S,4R)-3-hydroxytetrahydro-2H-pyran-4-yl)amino)pyrimidin-4-yl)-1H-indole-3-carboxylic acid (Intermediate 20) replacing 6-(5-fluoro-2-(((3S,4R)-3-hydroxytetrahydro-2H-pyran-4-yl)amino)pyrimidin-4-yl)-1-methyl-1H-indole-3-carboxylic acid. LC-MS calculated for C₂₅H₂₇F₅N₅O₅(M+H)⁺: m/z=572.2; found 572.1.

### Example 48: (6-(5-chloro-2-(((3S,4R)-3-hydroxytetrahydro-2H-pyran-4-yl)amino)pyrimidin-4-yl)-1-ethyl-4-fluoro-1H-indol-3-yl)((2R,3S)-2-(hydroxymethyl)-3-(trifluoromethoxy)azetidin-1-yl)methanone

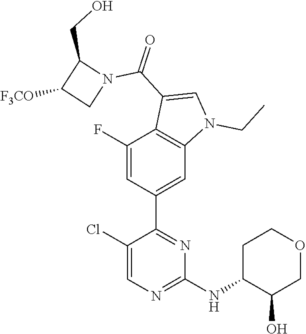 [SMILES: CCn1cc(C(=O)N2C[C@H](OC(F)(F)F)[C@H]2CO)c2c(F)cc(-c3nc(N[C@@H]4CCOC[C@H]4O)ncc3Cl)cc21]

The title compound was prepared according to the procedure outlined for Example 43, with 6-(5-chloro-2-(((3S,4R)-3-hydroxytetrahydro-2H-pyran-4-yl)amino)pyrimidin-4-yl)-1-ethyl-4-fluoro-1H-indole-3-carboxylic acid (Intermediate 32) replacing 6-(5-fluoro-2-(((3S,4R)-3-hydroxytetrahydro-2H-pyran-4-yl)amino)pyrimidin-4-yl)-1-methyl-1H-indole-3-carboxylic acid. LC-MS calculated for C₂₅H₂₇ClF₄N₅O₅ (M+H)⁺: m/z=588.2; found 588.1.

### Example 49. (1-(2,2-Difluoroethyl)-4-fluoro-6-(5-fluoro-2-(((3S,4R)-3-hydroxytetra hydro-2H-pyran-4-yl)amino)pyrimidin-4-yl)-1H-indol-3-yl)(5-azaspiro[2.3]hexan-5-yl) methanone

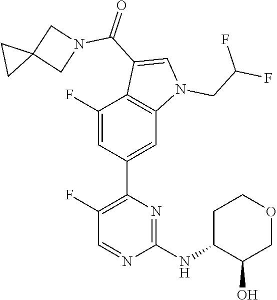 [SMILES: O=C(c1cn(CC(F)F)c2cc(-c3nc(N[C@@H]4CCOC[C@H]4O)ncc3F)cc(F)c12)N1CC2(CC2)C1]

A mixture of 1-(2,2-difluoroethyl)-4-fluoro-6-(5-fluoro-2-(((3S,4R)-3-hydroxytetrahydro-2H-pyran-4-yl)amino)pyrimidin-4-yl)-1H-indole-3-carboxylic acid (Intermediate 33, 15.0 mg, 0.033 mmol), HATU (15 mg, 0.038 mmol) and N,N-diisopropylethylamine (0.150 mL, 0.860 mmol) in DMF (0.30 mL) was stirred at r.t. for 10 min before 5-azaspiro[2.3]hexane hydrochloride (3.95 mg, 0.033 mmol) was added. The reaction mixture was stirred at r.t. for 1 hour. The mixture was diluted with MeCN, filtered, and purified with prep-HPLC (Sunfire C18 column, eluting with a gradient of acetonitrile/water containing 0.1% NH₄OH, at flow rate of 60 mL/min) to afford the desired product. LC-MS calculated for C₂₅H₂₆F₄N₅O₃(M+H)⁺: m/z=520.2; found 520.2.

### Example 50. (1-(2,2-Difluoroethyl)-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d2)-3-methylazetidin-1-yl)methanone

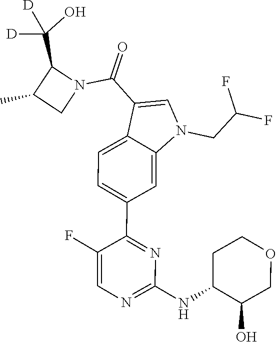 [SMILES: [2H]C([2H])(O)[C@@H]1[C@@H](C)CN1C(=O)c1cn(CC(F)F)c2cc(-c3nc(N[C@@H]4CCOC[C@H]4O)ncc3F)ccc12]

A mixture of 1-(2,2-difluoroethyl)-6-(5-fluoro-2-(((3S,4R)-3-hydroxytetrahydro-2H-pyran-4-yl)amino)pyrimidin-4-yl)-1H-indole-3-carboxylic acid (Intermediate 34, 400.0 mg, 0.917 mmol), HATU (401 mg, 1.05 mmol) and N,N-diisopropylethylamine (0.798 mL, 4.58 mmol) in MeCN (10.0 mL) was stirred at r.t. for 10 min before ((2S,3S)-3-methylazetidin-2-yl)methan-d₂-ol hydrochloride (Intermediate 24, 192 mg, 1.38 mmol) was added. The reaction mixture was stirred at r.t. for 1 hour. The mixture was diluted with MeCN, filtered, and purified with prep-HPLC (Sunfire C18 column, eluting with a gradient of acetonitrile/water containing 0.1% NH₄OH, at flow rate of 60 mL/min) to afford the desired product. LC-MS calculated for C₂₅H₂₇D₂F₃N₅O₄ (M+H)⁺: m/z=522.2; found 522.2.

### Examples 51 and 52: (6-(5-Chloro-2-(((3S,4R)-3-hydroxytetrahydro-2H-pyran-4-yl)amino)pyrimidin-4-yl)-1-ethyl-4-fluoro-1H-indol-3-yl)((S)-4-(hydroxymethyl)-5-azaspiro[2.3]hexan-5-yl)methanone and (6-(5-chloro-2-(((3S,4R)-3-hydroxytetrahydro-2H-pyran-4-yl)amino)pyrimidin-4-yl)-1-ethyl-4-fluoro-1H-indol-3-yl)((R)-4-(hydroxymethyl)-5-azaspiro[2.3]hexan-5-yl)methanone

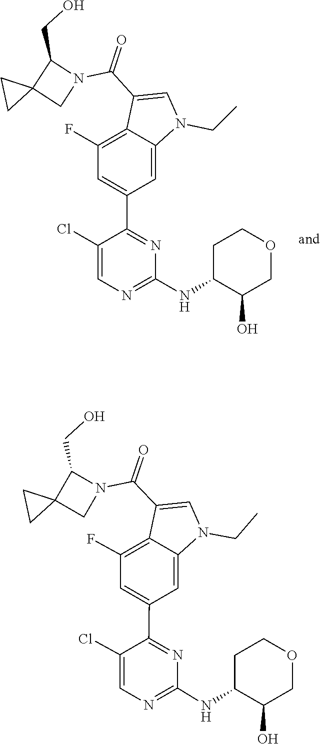 [SMILES: CCn1cc(C(=O)N2CC3(CC3)[C@H]2CO)c2c(F)cc(-c3nc(N[C@@H]4CCOC[C@H]4O)ncc3Cl)cc21 ; CCn1cc(C(=O)N2CC3(CC3)[C@@H]2CO)c2c(F)cc(-c3nc(N[C@@H]4CCOC[C@H]4O)ncc3Cl)cc21]

A mixture of 6-(5-chloro-2-(((3S,4R)-3-hydroxytetrahydro-2H-pyran-4-yl)amino)pyrimidin-4-yl)-1-ethyl-4-fluoro-1H-indole-3-carboxylic acid (Intermediate 32, 35 mg, 0.080 mmol), (±)-(5-azaspiro[2.3]hexan-4-yl)methanol hydrochloride (Intermediate 13, 24.1 mg, 0.161 mmol), and N,N-diisopropylethylamine (56 μL, 0.32 mmol) in acetonitrile (2 mL) was added propylphosphonic anhydride solution (50 wt % in DMF, 70.6 μL, 0.121 mmol, Aldrich 81801). The mixture was stirred at r.t. for 15 min. The reaction mixture was diluted with MeCN, filtered, and purified with prep-HPLC (Sunfire C18 column, eluting with a gradient of acetonitrile/water containing 0.1% NH₄OH, at flow rate of 60 mL/min) to afford the desired product as a mixture of diastereomers. The diastereomeric mixture was further purified by chiral SFC (CHIRALPAK 1H 5 μm, 21×250 mm, eluting with an isocratic mobile phase of 25% MeOH in CO₂, at flow rate of 65 mL/min) to afford the desired product.

### Example 51

Retention time on chiral SFC tᵣ=14.0 min, LC-MS calculated for C₂₆H₃₀ClFN₅O₄(M+H)⁺: m/z=530.2; found 530.2

### Example 52

Retention time on chiral SFC tᵣ=18.4 min, LC-MS calculated for C₂₆H₃₀ClFN₅O₄(M+H)⁺: m/z=530.2; found 530.2.

Based on the difference in activity between Example 9 (having an (S)-hydroxymethyl group on the cyclized amide nitrogen) and Example 15 (having an (R)-hydroxymethyl group on the cyclized amide nitrogen), it is believed that Example 51 is (6-(5-chloro-2-(((3S,4R)-3-hydroxytetrahydro-2H-pyran-4-yl)amino)pyrimidin-4-yl)-1-ethyl-4-fluoro-1H-indol-3-yl)((S)-4-(hydroxymethyl)-5-azaspiro[2.3]hexan-5-yl)methanone, while Example 52 is (6-(5-chloro-2-(((3S,4R)-3-hydroxytetrahydro-2H-pyran-4-yl)amino)pyrimidin-4-yl)-1-ethyl-4-fluoro-1H-indol-3-yl)((R)-4-(hydroxymethyl)-5-azaspiro[2.3]hexan-5-yl)methanone.

### Example 53: (3-(tert-Butyl)-3-hydroxyazetidin-1-yl)(6-(5-chloro-2-(((3S,4R)-3-hydroxytetrahydro-2H-pyran-4-yl)amino)pyrimidin-4-yl)-1-ethyl-4-fluoro-1H-indol-3-yl)methanone

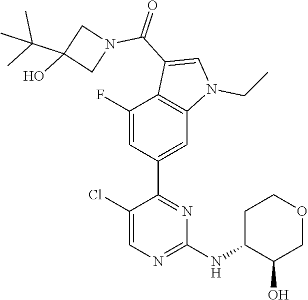 [SMILES: CCn1cc(C(=O)N2CC(O)(C(C)(C)C)C2)c2c(F)cc(-c3nc(N[C@@H]4CCOC[C@H]4O)ncc3Cl)cc21]

A mixture of 6-(5-chloro-2-(((3S,4R)-3-hydroxytetrahydro-2H-pyran-4-yl)amino)pyrimidin-4-yl)-1-ethyl-4-fluoro-1H-indole-3-carboxylic acid (Intermediate 32, 10 mg, 0.023 mmol), 3-(tert-butyl)azetidin-3-ol (5.9 mg, 0.046 mmol), and N,N-diisopropylethylamine (12 μL, 0.069 mmol) in acetonitrile (0.4 mL) was added propylphosphonic anhydride solution (50 wt % in DMF, 20 μL, 0.034 mmol, Aldrich 81801). The mixture was stirred at r.t. for 1 h. The reaction mixture was diluted with MeCN, filtered, and purified with prep-HPLC (Sunfire C18 column, eluting with a gradient of acetonitrile/water containing 0.1% NH₄OH, at flow rate of 60 mL/min) to afford the desired product. LC-MS calculated for C₂₇H₃₄ClFN₅O₄(M+H)⁺: m/z=546.2; found 546.4.

### Example 54: (6-(5-Chloro-2-(((3S,4R)-3-hydroxytetrahydro-2H-pyran-4-yl)amino)pyrimidin-4-yl)-1-ethyl-4-fluoro-1H-indol-3-yl)(3-((difluoromethoxy)methyl)azetidin-1-yl)methanone

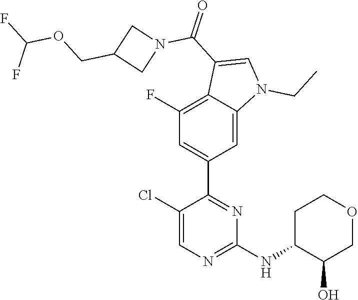 [SMILES: CCn1cc(C(=O)N2CC(COC(F)F)C2)c2c(F)cc(-c3nc(N[C@@H]4CCOC[C@H]4O)ncc3Cl)cc21]

The title compound was prepared according to the procedure outlined for Example 53, with 3-((difluoromethoxy)methyl)azetidine replacing 3-(tert-butyl)azetidin-3-ol. LC-MS calculated for C₂₅H₂₈ClF₃N₅O₄ (M+H)⁺: m/z=554.2; found 554.1.

### Example 55: (6-(5-Chloro-2-(((3S,4R)-3-hydroxytetrahydro-2H-pyran-4-yl)amino)pyrimidin-4-yl)-1-ethyl-4-fluoro-1H-indol-3-yl)((2S,3S)-2-(hydroxymethyl-d2)-3-methylazetidin-1-yl)methanone

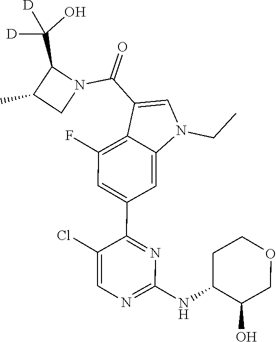 [SMILES: [2H]C([2H])(O)[C@@H]1[C@@H](C)CN1C(=O)c1cn(CC)c2cc(-c3nc(N[C@@H]4CCOC[C@H]4O)ncc3Cl)cc(F)c12]

The title compound was prepared according to the procedure outlined for Example 53, with ((2S,3S)-3-methylazetidin-2-yl)methan-d₂-ol hydrochloride (Intermediate 14) replacing 3-(tert-butyl)azetidin-3-ol. LC-MS calculated for C₂₅H₂₈D2ClFN₅O₄(M+H)⁺: m/z=520.2; found 520.2.

### Example 56: (6-(5-Chloro-2-(((3S,4R)-3-hydroxytetrahydro-2H-pyran-4-yl)amino)pyrimidin-4-yl)-1-ethyl-4-fluoro-1H-indol-3-yl)(3-(difluoromethyl)azetidin-1-yl)methanone

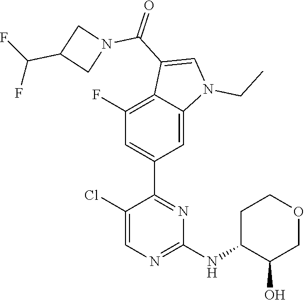 [SMILES: CCn1cc(C(=O)N2CC(C(F)F)C2)c2c(F)cc(-c3nc(N[C@@H]4CCOC[C@H]4O)ncc3Cl)cc21]

The title compound was prepared according to the procedure outlined for Example 53, with 3-(difluoromethyl)azetidine replacing 3-(tert-butyl)azetidin-3-ol. LC-MS calculated for C₂₄H₂₆ClF₃N₅O₃ (M+H)⁺: m/z=524.2; found 524.1.

### Example 57: (6-(5-Chloro-2-(((3S,4R)-3-hydroxytetrahydro-2H-pyran-4-yl)amino)pyrimidin-4-yl)-1-ethyl-4-fluoro-1H-indol-3-yl)((2R,3S)-3-(difluoromethoxy)-2-(hydroxymethyl)azetidin-1-yl)methanone

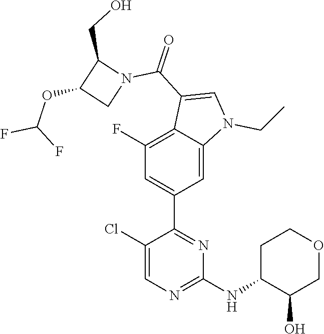 [SMILES: CCn1cc(C(=O)N2C[C@H](OC(F)F)[C@H]2CO)c2c(F)cc(-c3nc(N[C@@H]4CCOC[C@H]4O)ncc3Cl)cc21]

The title compound was prepared according to the procedure outlined for Example 53, with ((2R,3S)-3-(difluoromethoxy)azetidin-2-yl)methanol hydrochloride (Intermediate 16) replacing 3-(tert-butyl)azetidin-3-ol. LC-MS calculated for C₂₅H₂₈ClF₃N₅O₅ (M+H)⁺: m/z=570.2; found 570.1.

### Examples 58 and 59: (1-Ethyl-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone and (1-ethyl-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone

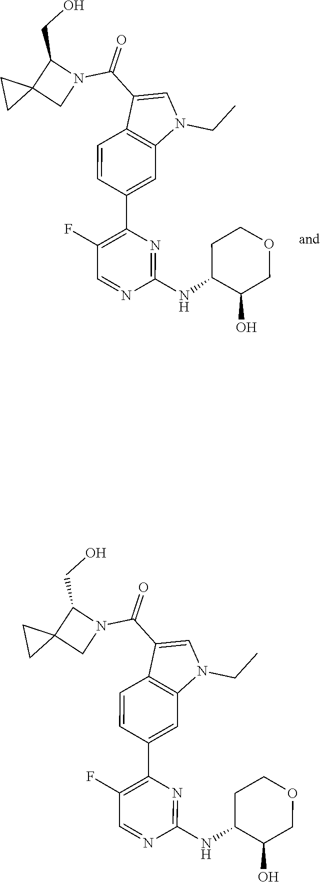 [SMILES: CCn1cc(C(=O)N2CC3(CC3)[C@H]2CO)c2ccc(-c3nc(N[C@@H]4CCOC[C@H]4O)ncc3F)cc21 ; CCn1cc(C(=O)N2CC3(CC3)[C@@H]2CO)c2ccc(-c3nc(N[C@@H]4CCOC[C@H]4O)ncc3F)cc21]

To a mixture of 1-ethyl-6-(5-fluoro-2-(((3S,4R)-3-hydroxytetrahydro-2H-pyran-4-yl)amino)pyrimidin-4-yl)-1H-indole-3-carboxylic acid (Intermediate 18, 750 mg, 1.87 mmol), (±)-(5-azaspiro[2.3]hexan-4-yl)methanol hydrochloride (Intermediate 13, 336 mg, 2.25 mmol) and N,N-diisopropylethylamine (1.31 mL, 7.49 mmol) in N,N-dimethylformamide (1.7 mL) and acetonitrile (17 mL) was added HATU (0.89 g, 2.34 mmol). The mixture was stirred at r.t. for 1 h. The reaction mixture was diluted with EtOAc (50 mL) and H₂O (50 mL). The layers were separated and the aqueous layer was extracted with EtOAc (3×50 mL). The combined organic fractions were dried over MgSO₄ and concentrated. The crude residue was purified by flash column chromatography (SiO₂, 0-20% CH₂Cl₂/MeOH) to afford the desired product (882 mg, 95% yield) as a mixture of diastereomers. The diastereomeric mixture was further purified by chiral SFC (CHIRALPAK 1H 5 μm, 20×250 mm, eluting with an isocratic mobile phase of 30% MeOH in CO₂, at flow rate of 60 mL/min) to afford each diastereomer.

Example 58: Retention time on chiral SFC tᵣ=7.5 min, LC-MS calculated for C₂₆H₃₁FN₅O₄(M+H)⁺: m/z=496.2; found 496.4. ¹H NMR (600 MHz, DMSO-d₆) δ 8.40 (d, J=3.8 Hz, 1H), 8.28 (d, J=8.5 Hz, 1H), 8.18 (s, 1H), 8.04 (s, 1H), 7.85 (d, J=8.5 Hz, 1H), 7.12 (d, J=7.6 Hz, 1H), 5.00-4.88 (m, 2H), 4.57-4.50 (m, 1H), 4.47-4.43 (m, 1H), 4.37-4.27 (m, 3H), 3.87-3.78 (m, 3H), 3.75-3.69 (m, 1H), 3.68-3.61 (m, 1H), 3.58-3.50 (m, 1H), 3.37 (td, J=11.6, 2.3 Hz, 1H), 3.11-3.05 (m, 1H), 2.08-1.98 (m, 1H), 1.56-1.46 (m, 1H), 1.43 (t, J=7.2 Hz, 3H), 0.95-0.89 (m, 1H), 0.70-0.61 (m, 2H), 0.57-0.50 (m, 1H).

### Example 59

Retention time on chiral SFC tᵣ=10 min, LC-MS calculated for C₂₆H₃₁FN₅O₄(M+H)⁺: m/z=496.2; found 496.4.

Based on the difference in activity between Example 9 (having an (S)-hydroxymethyl group on the cyclized amide nitrogen) and Example 15 (having an (R)-hydroxymethyl group on the cyclized amide nitrogen), it is believed that Example 58 is (1-ethyl-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone, while Example 59 is (1-ethyl-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone.

### Examples 60 and 61: (6-(5-Fluoro-2-(((3S,4R)-3-hydroxytetrahydro-2H-pyran-4-yl)amino)pyrimidin-4-yl)-1-methyl-1H-indol-3-yl)((S)-4-(hydroxymethyl)-5-azaspiro[2.3]hexan-5-yl)methanone and (6-(5-fluoro-2-(((3S,4R)-3-hydroxytetrahydro-2H-pyran-4-yl)amino)pyrimidin-4-yl)-1-methyl-1H-indol-3-yl)((R)-4-(hydroxymethyl)-5-azaspiro[2.3]hexan-5-yl)methanone

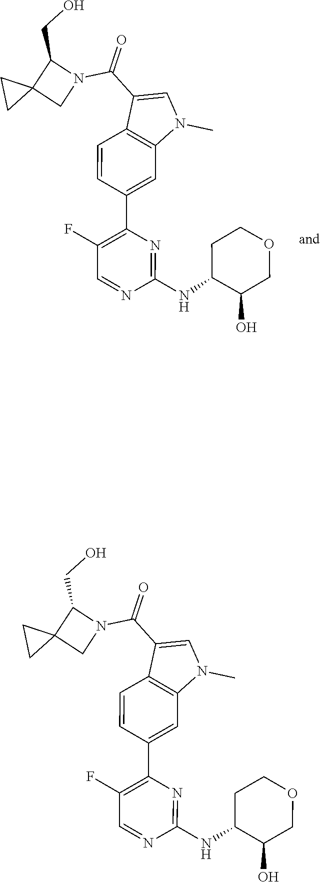 [SMILES: Cn1cc(C(=O)N2CC3(CC3)[C@H]2CO)c2ccc(-c3nc(N[C@@H]4CCOC[C@H]4O)ncc3F)cc21 ; Cn1cc(C(=O)N2CC3(CC3)[C@@H]2CO)c2ccc(-c3nc(N[C@@H]4CCOC[C@H]4O)ncc3F)cc21]

To a mixture of 6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 17, 200 mg, 0.518 mmol), (±)-(5-azaspiro[2.3]hexan-4-yl)methanol hydrochloride (Intermediate 13, 155 mg, 1.04 mmol) and N,N-diisopropylethylamine (0.904 mL, 5.18 mmol) in N,N-dimethylformamide (3.4 mL) was added HATU (0.22 g, 0.569 mmol). The mixture was stirred at 50° C. for 10 min. The reaction mixture was cooled to r.t. and diluted with CH₂Cl₂ (50 mL) and washed sequentially with 5% LiCl (aq) (2×50 mL) and brine (100 mL). The layers were separated and the organic fraction was dried over Na₂SO₄ and concentrated. The crude residue was purified by flash column chromatography (SiO₂, 0-10% CH₂Cl₂/MeOH) to afford the desired product (178 mg, 71% yield) as a mixture of diastereomers. The diastereomeric mixture was further purified by chiral SFC (CHIRALPAK 1H 5 μm, 20×250 mm, eluting with an isocratic mobile phase of 50% MeOH in CO₂, at flow rate of 60 mL/min) to afford each diastereomer.

Example 60

Retention time on chiral SFC tᵣ=4.5 min, LC-MS calculated for C₂₅H₂₉FN₅O₄ (M+H)⁺: m/z=482.2; found 482.4. ¹H NMR (500 MHz, DMSO-d₆) δ 8.38 (d, J=3.9 Hz, 1H), 8.25 (d, J=8.5 Hz, 1H), 8.12 (d, J=1.5 Hz, 1H), 7.98 (s, 1H), 7.85 (d, J=8.5 Hz, 1H), 7.10 (d, J=7.6 Hz, 1H), 5.00-4.88 (m, 2H), 4.55-4.48 (m, 1H), 4.41 (s, 1H), 4.29 (s, 1H), 3.89 (s, 3H), 3.85-3.76 (m, 3H), 3.73-3.67 (m, 1H), 3.66-3.59 (m, 1H), 3.56-3.45 (m, 1H), 3.35 (t, J=11.6, 1H), 3.10-3.00 (m, 1H), 2.05-1.95 (m, 1H), 1.54-1.41 (m, 1H), 0.95-0.85 (m, 1H), 0.71-0.58 (m, 2H), 0.57-0.48 (m, 1H).

### Example 61

Retention time on chiral SFC tᵣ=5.5 min, LC-MS calculated for C₂₅H₂₉FN₅O₄ (M+H)⁺: m/z=482.2; found 482.4.

Based on the difference in activity between Example 9 (having an (S)-hydroxymethyl group on the cyclized amide nitrogen) and Example 15 (having an (R)-hydroxymethyl group on the cyclized amide nitrogen), it is believed that Example 60 is (6-(5-fluoro-2-(((3S,4R)-3-hydroxytetrahydro-2H-pyran-4-yl)amino)pyrimidin-4-yl)-1-methyl-1H-indol-3-yl)((S)-4-(hydroxymethyl)-5-azaspiro[2.3]hexan-5-yl)methanone, while Example 61 is (6-(5-fluoro-2-(((3S,4R)-3-hydroxytetrahydro-2H-pyran-4-yl)amino)pyrimidin-4-yl)-1-methyl-1H-indol-3-yl)((R)-4-(hydroxymethyl)-5-azaspiro[2.3]hexan-5-yl)methanone.

### Examples 62 and 63: ((4S)-1,1-Difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone and ((4R)-1,1-Difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone

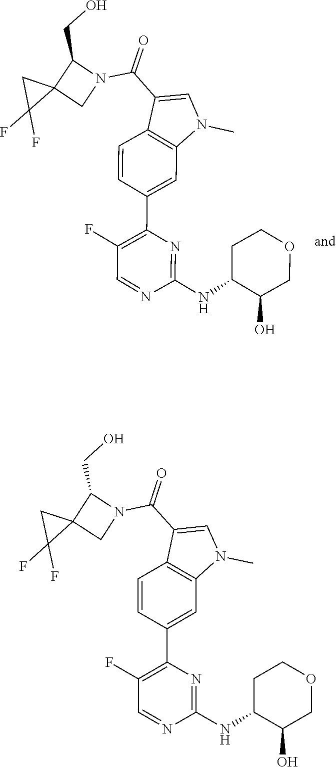 [SMILES: Cn1cc(C(=O)N2CC3(CC3(F)F)[C@H]2CO)c2ccc(-c3nc(N[C@@H]4CCOC[C@H]4O)ncc3F)cc21 ; Cn1cc(C(=O)N2CC3(CC3(F)F)[C@@H]2CO)c2ccc(-c3nc(N[C@@H]4CCOC[C@H]4O)ncc3F)cc21]

To a mixture of 6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 17, 20 mg, 0.052 mmol), (1,1-difluoro-5-azaspiro[2.3]hexan-4-yl)methanol hydrochloride (19 mg, 0.104 mmol) and N,N-diisopropylethylamine (0.090 mL, 0.518 mmol) in N,N-dimethylformamide (0.350 mL) was added HATU (22 mg, 0.057 mmol). The mixture was stirred at r.t for 18 h. The reaction mixture diluted with CH₂Cl₂ (5 mL) and washed with 5% LiCl (aq) (5 mL). The layers were separated and the organic fraction was dried over Na₂SO₄ and concentrated. The crude residue was purified by flash column chromatography (SiO₂, 0-10% CH₂Cl₂/MeOH) to afford the desired product (13 mg, 48% yield) as a mixture of diastereomers. The diastereomeric mixture was further purified by chiral SFC (Phenomenex Cellulose 5 μm, 20×250 mm, eluting with an isocratic mobile phase of 40% MeOH in C₀₂, at flow rate of 70 mL/min) to afford each diastereomer.

### Example 62

Retention time on chiral SFC tᵣ=4.3 min, LC-MS calculated for C₂₅H₂₇F₃N₅O₄(M+H)⁺: m/z=518.2; found 518.2.

### Example 63

Retention time on chiral SFC tᵣ=5.3 min, LC-MS calculated for C₂₅H₂₇F₃N₅O₄(M+H)⁺: m/z=518.2; found 518.2.

### Examples 64 and 65: (6-(5-Fluoro-2-(((3S,4R)-3-hydroxytetrahydro-2H-pyran-4-yl)amino)pyrimidin-4-yl)-1-methyl-11H-indol-3-yl)((S)-1-(hydroxymethyl)-2-azaspiro[3.3]heptan-2-yl)methanone and (6-(5-Fluoro-2-(((3S,4R)-3-hydroxytetrahydro-2H-pyran-4-yl)amino)pyrimidin-4-yl)-1-methyl-1H-indol-3-yl)((R)-1-(hydroxymethyl)-2-azaspiro[3.3]heptan-2-yl)methanone

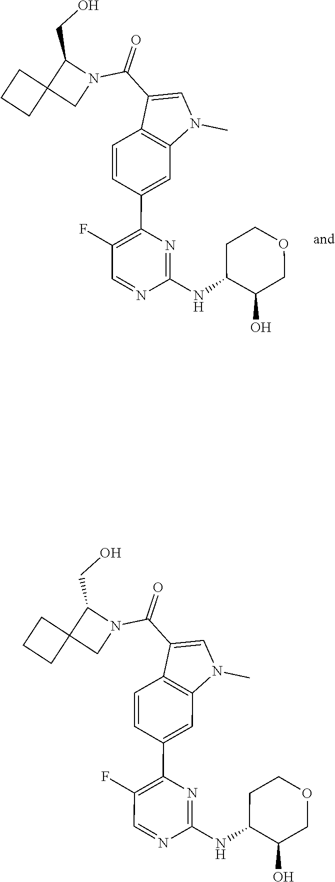 [SMILES: Cn1cc(C(=O)N2CC3(CCC3)[C@H]2CO)c2ccc(-c3nc(N[C@@H]4CCOC[C@H]4O)ncc3F)cc21 ; Cn1cc(C(=O)N2CC3(CCC3)[C@@H]2CO)c2ccc(-c3nc(N[C@@H]4CCOC[C@H]4O)ncc3F)cc21]

To a mixture of 6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 17, 40 mg, 0.10 mmol), (2-azaspiro[3.3]heptan-1-yl)methanol, 26 mg, 0.207 mmol) and N,N-diisopropylethylamine (0.18 mL, 1.0 mmol) in N,N-dimethylformamide (0.69 mL) was added HATU (43 mg, 0.11 mmol). The mixture was stirred at 50° C. for 10 min. After cooling to rt, the reaction mixture diluted with CH₂Cl₂ (5 mL) and washed with 5% LiCl (aq) (5 mL). The layers were separated, and the organic fraction was dried over Na₂SO₄ and concentrated. The crude residue was purified by flash column chromatography (SiO₂, 0-10% CH₂Cl₂/MeOH) to afford the desired product (35 mg, 68% yield) as a mixture of diastereomers. The diastereomeric mixture was further purified by chiral SFC (CHIRALPAK 1H 5 μm, 20×250 mm, eluting with an isocratic mobile phase of 50% MeOH in CO₂, at flow rate of 60 mL/min) to afford each diastereomer.

### Example 64

Retention time on chiral SFC tᵣ=4.2 min, LC-MS calculated for C₂₆H₃₁FN₅O₄(M+H)⁺: m/z=496.2; found 496.2. ¹H NMR (600 MHz, DMSO-d₆) δ 8.39 (d, J=3.8 Hz, 1H), 8.24 (d, J=8.5 Hz, 1H), 8.13 (s, 1H), 8.00 (s, 1H), 7.85 (d, J=8.5 Hz, 1H), 7.10 (d, J=7.7 Hz, 1H), 4.39 (s, 1H), 4.32-4.19 (m, 2H), 3.91 (s, 3H), 3.85-3.70 (m, 5H), 3.52 (s, 1H), 3.12-3.02 (m, 1H), 2.42-2.35 (m, 1H), 2.22-2.10 (m, 3H), 2.06-1.93 (m, 3H), 1.86-1.76 (m, 3H), 1.54-1.44 (m, 1H).

### Example 65

Retention time on chiral SFC tᵣ=5.1 min, LC-MS calculated for C₂₆H₃₁FN₅O₄(M+H)⁺: m/z=496.2; found 496.2.

Based on the difference in activity between Example 9 (having an (S)-hydroxymethyl group on the cyclized amide nitrogen) and Example 15 (having an (R)-hydroxymethyl group on the cyclized amide nitrogen), it is believed that Example 64 is (6-(5-fluoro-2-(((3S,4R)-3-hydroxytetrahydro-2H-pyran-4-yl)amino)pyrimidin-4-yl)-1-methyl-1H-indol-3-yl)((S)-1-(hydroxymethyl)-2-azaspiro[3.3]heptan-2-yl)methanone, while Example 65 is (6-(5-fluoro-2-(((3S,4R)-3-hydroxytetrahydro-2H-pyran-4-yl)amino)pyrimidin-4-yl)-1-methyl-1H-indol-3-yl)((R)-1-(hydroxymethyl)-2-azaspiro[3.3]heptan-2-yl)methanone.

### Example 66: (6-(5-Fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-methylazetidin-1-yl)methanone

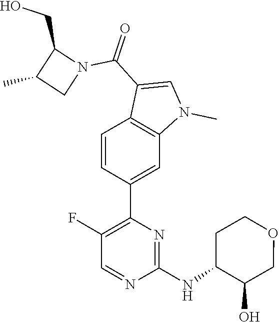 [SMILES: C[C@H]1CN(C(=O)c2cn(C)c3cc(-c4nc(N[C@@H]5CCOC[C@H]5O)ncc4F)ccc23)[C@@H]1CO]

To a mixture of 6-(5-fluoro-2-(((3S,4R)-3-hydroxytetrahydro-2H-pyran-4-yl)amino)pyrimidin-4-yl)-1-methyl-1H-indole-3-carboxylic acid (Intermediate 17, 20 mg, 0.052 mmol), ((2S,3S)-3-methylazetidin-2-yl) methanol hydrochloride (Intermediate 24, 14 mg, 0.104 mmol) and N,N-diisopropylethylamine (0.090 mL, 0.518 mmol) in N,N-dimethylformamide (0.345 mL) was added HATU (22 mg, 0.057 mmol). The mixture was stirred at r.t for 18 h. The mixture was diluted with MeCN and purified by prep-HPLC (Sunfire C18 column, eluting with a gradient of acetonitrile/water containing 0.1% NH₄OH, at flow rate of 60 mL/min) to afford the desired product. LC-MS calculated for C₂₄H₂₉FN₅O₄ (M+H)⁺: m/z=470.2; found 470.1.

### Example 67: 2—((S)-1-(6-(5-Fluoro-2-(((3S,4R)-3-hydroxytetrahydro-2H-pyran-4-yl)amino)pyrimidin-4-yl)-1-methyl-1H-indole-3-carbonyl)azetidin-2-yl)acetonitrile

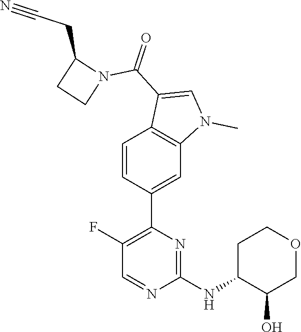 [SMILES: Cn1cc(C(=O)N2CC[C@H]2CC#N)c2ccc(-c3nc(N[C@@H]4CCOC[C@H]4O)ncc3F)cc21]

The title compound was prepared according to the procedure outlined for Example 66, with (S)-2-(azetidine-2-yl)acetonitrile 2,2,2-trifluoroacetate replacing ((2S,3S)-3-methylazetidin-2-yl) methanol hydrochloride. LC-MS calculated for C₂₄H₂₆FN₆O₃(M+H)⁺: m/z=465.2; found 465.2.

### Example 68: ((2R,3S)-3-(Difluoromethoxy)-2-(hydroxymethyl)azetidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone

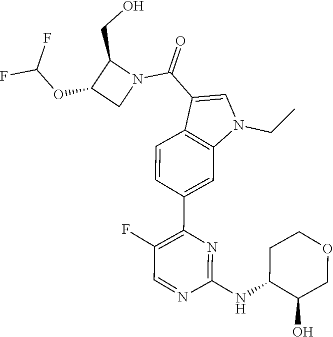 [SMILES: CCn1cc(C(=O)N2C[C@H](OC(F)F)[C@H]2CO)c2ccc(-c3nc(N[C@@H]4CCOC[C@H]4O)ncc3F)cc21]

The title compound was prepared according to the procedure outlined for Example 66, with Intermediate 18 replacing Intermediate 17, and Intermediate 16 replacing Intermediate 24. LC-MS calculated for C₂₅H₂₉F₃N₅O₅(M+H)⁺: m/z=536.2; found 536.2.

### Example 69: (6-(5-Chloro-2-(((3S,4R)-3-hydroxytetrahydro-2H-pyran-4-yl)amino)pyrimidin-4-yl)-1-ethyl-1H-indol-3-yl)((2S,3S)-2-(hydroxymethyl-d2)-3-methylazetidin-1-yl)methanone

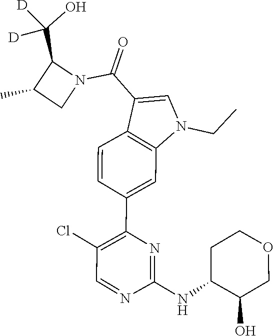 [SMILES: [2H]C([2H])(O)[C@@H]1[C@@H](C)CN1C(=O)c1cn(CC)c2cc(-c3nc(N[C@@H]4CCOC[C@H]4O)ncc3Cl)ccc12]

To a mixture of 6-(5-chloro-2-(((3S,4R)-3-hydroxytetrahydro-2H-pyran-4-yl)amino)pyrimidin-4-yl)-1-ethyl-1H-indole-3-carboxylic acid (Intermediate 35, 150 mg, 0.360 mmol), ((2S,3S)-3-methylazetidin-2-yl)methan-d₂-ol hydrochloride (Intermediate 14, 100 mg, 0.720 mmol) and N,N-diisopropylethylamine (0.628 mL, 3.60 mmol) in N,N-dimethylformamide (3.6 mL) was added HATU (151 mg, 0.396 mmol). The mixture was stirred at r.t for 10 min. The mixture was diluted with CH₂Cl₂ and washed with 5% LiCl (aqueous). The layers were separated and the organic layer concentrated under reduced pressure. The crude residue was purified by prep-HPLC (Sunfire C18 column, eluting with a gradient of acetonitrile/water containing 0.1% NH₄OH, at flow rate of 60 mL/min) to afford the desired product. LC-MS calculated for C₂₅H₂₉D₂ClN₅O₄ (M+H)⁺: m/z=502.1; found 502.2. ¹H NMR (600 MHz, DMSO-d₆) δ 8.40 (s, 1H), 8.24 (d, J=8.4 Hz, 1H), 8.01 (s, 1H), 7.95 (s, 1H), 7.57 (d, J=8.4 Hz, 1H), 7.40 (s, 1H), 5.08-4.85 (m, 2H), 4.46 (s, 1H), 4.33 (q, J=7.2 Hz, 2H), 4.09 (s, 1H), 3.88-3.75. (m, 4H), 3.51 (s, 1H), 3.38-3.32 (m, 1H), 3.09-3.00 (m, 1H), 2.60-2.53 (m, 1H), 1.98 (s, 1H), 1.57-1.46 (m, 1H), 1.42 (t, J=7.2 Hz, 3H), 1.23 (d, J=6.9 Hz, 3H).

### Example 70: (6-(5-Chloro-2-(((3S,4R)-3-hydroxytetrahydro-2H-pyran-4-yl)amino)pyrimidin-4-yl)-1-ethyl-1H-indol-3-yl)((2R,3S)-3-(difluoromethoxy)-2-(hydroxymethyl)azetidin-1-yl)methanone

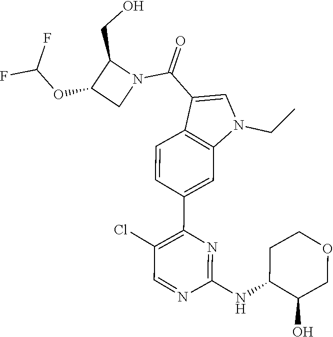 [SMILES: CCn1cc(C(=O)N2C[C@H](OC(F)F)[C@H]2CO)c2ccc(-c3nc(N[C@@H]4CCOC[C@H]4O)ncc3Cl)cc21]

The title compound was prepared according to the procedures outlined for Example 66, with Intermediate 35 replacing Intermediate 17 and Intermediate 16 replacing Intermediate 24. LC-MS calculated for C₂₅H₂₉ClF₂N₅O₅ (M+H)⁺: m/z=552.2; found 552.3.

TABLE 1
The compounds in Table 1 were prepared in accordance with the synthetic protocols set
forth in Example 18 using the appropriate starting materials.

| | | | Analytical |
|---|---|---|---|
| Ex. | Name | Structure | data |
| 71 | (6-(5-Fluoro-2- (((3S,4R)-3- hydroxytetrahydro-2H- pyran-4- yl)amino)pyrimidin-4- yl)-1-methyl-1H-indol- 3-yl)(5- azaspiro[2.3]hexan-5- yl)methanone | 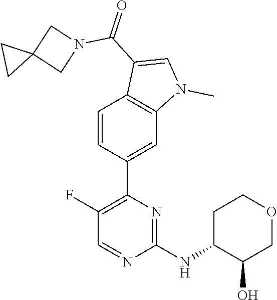 [SMILES: Cn1cc(C(=O)N2CC3(CC3)C2)c2ccc(-c3nc(N[C@@H]4CCOC[C@H]4O)ncc3F)cc21] | LCMS found 452.2 |
| 72 | (6-(5-Fluoro-2- (((3S,4R)-3- hydroxytetrahydro-2H- pyran-4- yl)amino)pyrimidin-4- yl)-1-methyl-1H-indol- 3-yl)(3- (trifluoromethyl)azetidin- 1-yl)methanone | 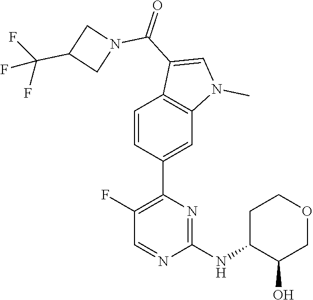 [SMILES: Cn1cc(C(=O)N2CC(C(F)(F)F)C2)c2ccc(-c3nc(N[C@@H]4CCOC[C@H]4O)ncc3F)cc21] | LCMS found 494.3 |
| 73 | (6-(5-Fluoro-2- (((3S,4R)-3- hydroxytetrahydro-2H- pyran-4- yl)amino)pyrimidin-4- yl)-1-methyl-1H-indol- 3-yl)((R)-2- methylazetidin-1- yl)methanone | 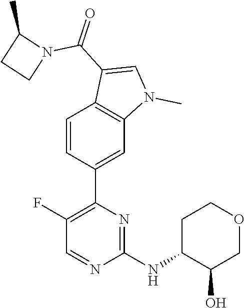 [SMILES: C[C@@H]1CCN1C(=O)c1cn(C)c2cc(-c3nc(N[C@@H]4CCOC[C@H]4O)ncc3F)ccc12] | LCMS found 440.3 |
| 74 | (6-(5-Fluoro-2- (((3S,4R)-3- hydroxytetrahydro-2H- pyran-4- yl)amino)pyrimidin-4- yl)-1-methyl-1H-indol- 3-yl)((S)-2- (hydroxymethyl)azetidin- 1-yl)methanone | 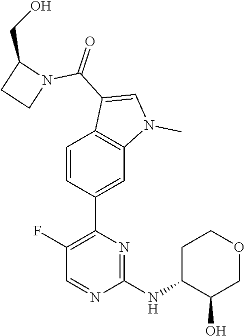 [SMILES: Cn1cc(C(=O)N2CC[C@H]2CO)c2ccc(-c3nc(N[C@@H]4CCOC[C@H]4O)ncc3F)cc21] | LCMS found 456.2 |
| 75 | (6-(5-Fluoro-2- (((3S,4R)-3- hydroxytetrahydro-2H- pyran-4- yl)amino)pyrimidin-4- yl)-1-methyl-1H-indol- 3-yl)((S)-2- ((methylsulfonyl)methyl) azetidin-1- yl)methanone | 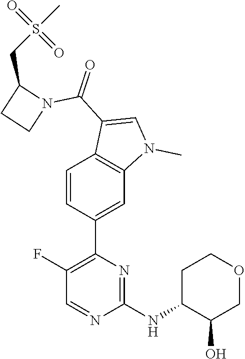 [SMILES: Cn1cc(C(=O)N2CC[C@H]2CS(C)(=O)=O)c2ccc(-c3nc(N[C@@H]4CCOC[C@H]4O)ncc3F)cc21] | LCMS found 518.2 |
| 76 | (5,5-Difluoro-2- azaspiro[3.3]heptan-2- yl)(6-(5-fluoro-2- (((3S,4R)-3- hydroxytetrahydro-2H- pyran-4- yl)amino)pyrimidin-4- yl)-1-methyl-1H-indol- 3-yl)methanone | 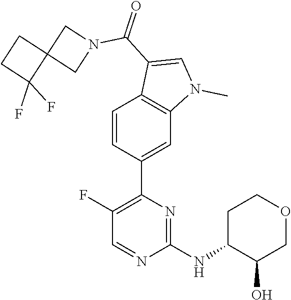 [SMILES: Cn1cc(C(=O)N2CC3(CCC3(F)F)C2)c2ccc(-c3nc(N[C@@H]4CCOC[C@H]4O)ncc3F)cc21] | LCMS found 502.3 |
| 77 | N-((S)-1-Cyclopropyl- 2-hydroxyethyl)-6-(5- fluoro-2-(((3S,4R)-3- hydroxytetrahydro-2H- pyran-4- yl)amino)pyrimidin-4- yl)-1-methyl-1H- indole-3-carboxamide | 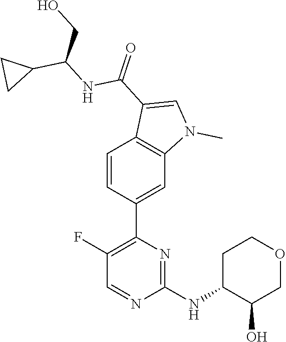 [SMILES: Cn1cc(C(=O)N[C@H](CO)C2CC2)c2ccc(-c3nc(N[C@@H]4CCOC[C@H]4O)ncc3F)cc21] | LCMS found 470.2 |
| 78 | (6-(5-Fluoro-2- (((3S,4R)-3- hydroxytetrahydro-2H- pyran-4- yl)amino)pyrimidin-4- yl)-1-methyl-1H-indol- 3-yl)(3-hydroxy-3- (trifluoromethyl)azetidin- 1-yl)methanone | 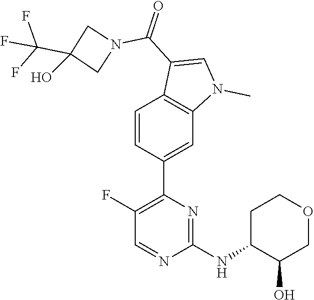 [SMILES: Cn1cc(C(=O)N2CC(O)(C(F)(F)F)C2)c2ccc(-c3nc(N[C@@H]4CCOC[C@H]4O)ncc3F)cc21] | LCMS found 510.2 |
| 79 | (6-(5-Fluoro-2- (((3S,4R)-3- hydroxytetrahydro-2H- pyran-4- yl)amino)pyrimidin-4- yl)-1-methyl-1H-indol- 3-yl)((2R,3R)-3- hydroxy-2- methylazetidin-1- yl)methanone | 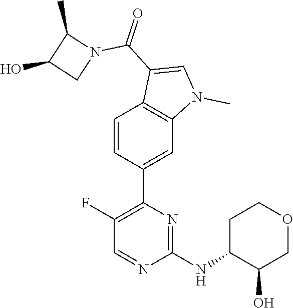 [SMILES: C[C@@H]1[C@H](O)CN1C(=O)c1cn(C)c2cc(-c3nc(N[C@@H]4CCOC[C@H]4O)ncc3F)ccc12] | LCMS found 456.3 |
| 80 | (6-(5-Fluoro-2- (((3S,4R)-3- hydroxytetrahydro-2H- pyran-4- yl)amino)pyrimidin-4- yl)-1-methyl-1H-indol- 3-yl)((2R,3S)-3- hydroxy-2- methylazetidin-1- yl)methanone | 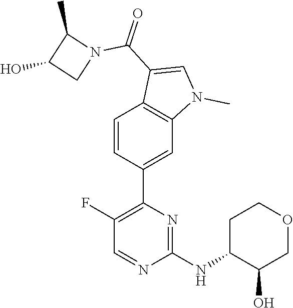 [SMILES: C[C@@H]1[C@@H](O)CN1C(=O)c1cn(C)c2cc(-c3nc(N[C@@H]4CCOC[C@H]4O)ncc3F)ccc12] | LCMS found 456.3 |
| 81 | ((2S,3S)-2- ((Cyclopropylsulfonyl) methyl)-3- methylazetidin-1-yl)(6- (5-fluoro-2-(((3S,4R)- 3-hydroxytetrahydro- 2H-pyran-4- yl)amino)pyrimidin-4- yl)-1-methyl-1H-indol- 3-yl)methanone | 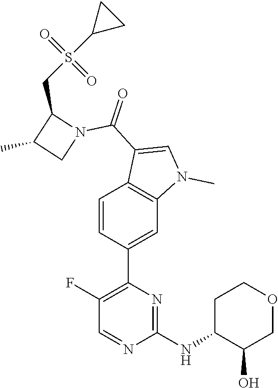 [SMILES: C[C@H]1CN(C(=O)c2cn(C)c3cc(-c4nc(N[C@@H]5CCOC[C@H]5O)ncc4F)ccc23)[C@@H]1CS(=O)(=O)C1CC1] | LCMS found 558.3 |
| 82 | ((2R,3S)-3- (Difluoromethoxy)-2- (hydroxymethyl)azetidin- 1-yl)(6-(5-fluoro-2- (((3S,4R)-3- hydroxytetrahydro-2H- pyran-4- yl)amino)pyrimidin-4- yl)-1-methyl-1H-indol- 3-yl)methanone | 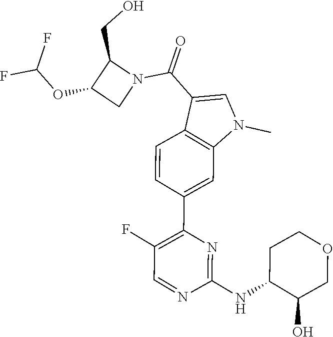 [SMILES: Cn1cc(C(=O)N2C[C@H](OC(F)F)[C@H]2CO)c2ccc(-c3nc(N[C@@H]4CCOC[C@H]4O)ncc3F)cc21] | LCMS found 522.2 |
| 83 | ((2S,4R)-4- (Difluoromethoxy)-2- (hydroxymethyl)pyrrolidin- 1-yl)(6-(5-fluoro-2- (((3S,4R)-3- hydroxytetrahydro-2H- pyran-4- yl)amino)pyrimidin-4- yl)-1-methyl-1H-indol- 3-yl)methanone | 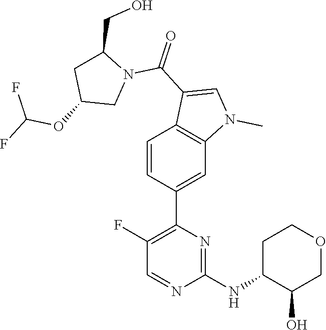 [SMILES: Cn1cc(C(=O)N2C[C@H](OC(F)F)C[C@H]2CO)c2ccc(-c3nc(N[C@@H]4CCOC[C@H]4O)ncc3F)cc21] | LCMS found 536.3 |
| 84 | (6-(5-Fluoro-2- (((3S,4R)-3- hydroxytetrahydro-2H- pyran-4- yl)amino)pyrimidin-4- yl)-1-methyl-1H-indol- 3-yl)((2S,4R)-2- (hydroxymethyl)-4- (trifluoromethoxy) pyrrolidin-1-yl)methanone | 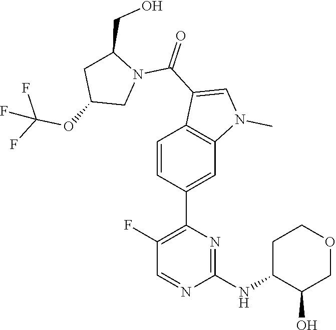 [SMILES: Cn1cc(C(=O)N2C[C@H](OC(F)(F)F)C[C@H]2CO)c2ccc(-c3nc(N[C@@H]4CCOC[C@H]4O)ncc3F)cc21] | LCMS found 554.2 |
| 85 | (6-(5-Fluoro-2- (((3S,4R)-3- hydroxytetrahydro-2H- pyran-4- yl)amino)pyrimidin-4- yl)-1-methyl-1H-indol- 3-yl)((2S,4R)-2- (hydroxymethyl)-4- (trifluoromethyl)pyrrolidin- 1-yl)methanone | 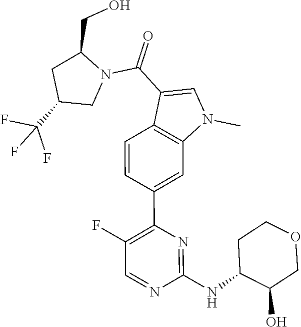 [SMILES: Cn1cc(C(=O)N2C[C@H](C(F)(F)F)C[C@H]2CO)c2ccc(-c3nc(N[C@@H]4CCOC[C@H]4O)ncc3F)cc21] | LCMS found 538.2 |
| 86 | ((S)-4,4-Difluoro-2- (hydroxymethyl)pyrrolidin- 1-yl)(6-(5-fluoro-2- (((3S,4R)-3- hydroxytetrahydro-2H- pyran-4- yl)amino)pyrimidin-4- yl)-1-methyl-1H-indol- 3-yl)methanone | 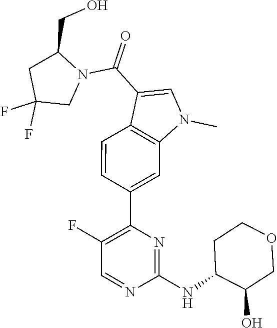 [SMILES: Cn1cc(C(=O)N2CC(F)(F)C[C@H]2CO)c2ccc(-c3nc(N[C@@H]4CCOC[C@H]4O)ncc3F)cc21] | LCMS found 506.2 |
| 87 | (6-(5-Fluoro-2- (((3S,4R)-3- hydroxytetrahydro-2H- pyran-4- yl)amino)pyrimidin-4- yl)-1-methyl-1H-indol- 3-yl)((S)-2- (hydroxymethyl)-4,4- dimethylpyrrolidin-1- yl)methanone | 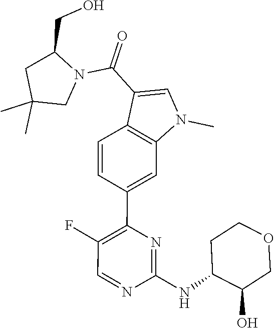 [SMILES: Cn1cc(C(=O)N2CC(C)(C)C[C@H]2CO)c2ccc(-c3nc(N[C@@H]4CCOC[C@H]4O)ncc3F)cc21] | LCMS found 498.3 |

TABLE 2
The compounds in Table 2 were prepared in accordance with the synthetic protocols set
forth in Example 19 using the appropriate starting materials.

| | | | Analytical |
|---|---|---|---|
| Ex. | Name | Structure | data |
| 88 | (1-Ethyl-6-(5-fluoro-2- (((3S,4R)-3- hydroxytetrahydro-2H- pyran-4- yl)amino)pyrimidin-4- yl)-1H-indol-3-yl)(3- (trifluoromethyl)azetidin- 1-yl)methanone | 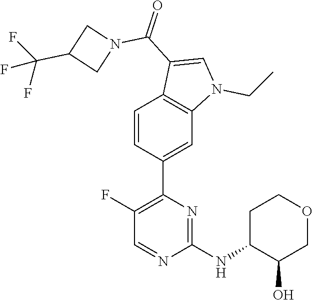 [SMILES: CCn1cc(C(=O)N2CC(C(F)(F)F)C2)c2ccc(-c3nc(N[C@@H]4CCOC[C@H]4O)ncc3F)cc21] | LCMS found 508.2 |
| 89 | ((2S,4R)-4- (Difluoromethoxy)-2- (hydroxymethyl) pyrrolidin-1-yl) (1-ethyl-6-(5- fluoro-2-(((3S,4R)-3- hydroxytetrahydro-2H- pyran-4- yl)amino)pyrimidin-4- yl)-1H-indol-3- yl)methanone | 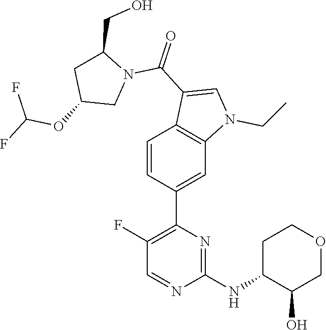 [SMILES: CCn1cc(C(=O)N2C[C@H](OC(F)F)C[C@H]2CO)c2ccc(-c3nc(N[C@@H]4CCOC[C@H]4O)ncc3F)cc21] | LCMS found 550.2 |
| 90 | (1-Ethyl-6-(5-fluoro-2- (((3S,4R)-3- hydroxytetrahydro-2H- pyran-4- yl)amino)pyrimidin-4- yl)-1H-indol-3- yl)((2S,4R)-2- (hydroxymethyl)-4- (trifluoromethoxy) pyrrolidin-1-yl)methanone | 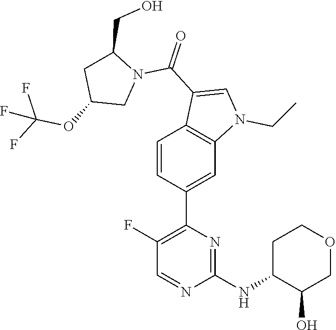 [SMILES: CCn1cc(C(=O)N2C[C@H](OC(F)(F)F)C[C@H]2CO)c2ccc(-c3nc(N[C@@H]4CCOC[C@H]4O)ncc3F)cc21] | LCMS found 568.2 |
| 91 | (1-Ethyl-6-(5-fluoro-2- (((3S,4R)-3- hydroxytetrahydro-2H- pyran-4- yl)amino)pyrimidin-4- yl)-1H-indol-3- yl)((2S,4R)-2- (hydroxymethyl)-4- (trifluoromethyl)pyrrolidin- 1-yl)methanone | 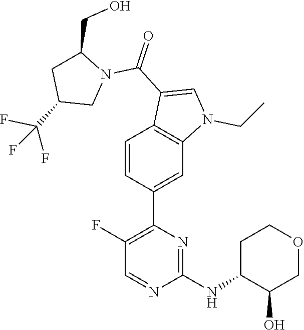 [SMILES: CCn1cc(C(=O)N2C[C@H](C(F)(F)F)C[C@H]2CO)c2ccc(-c3nc(N[C@@H]4CCOC[C@H]4O)ncc3F)cc21] | LCMS found 552.2 |
| 92 | (1-Ethyl-6-(5-fluoro-2- (((3S,4R)-3- hydroxytetrahydro-2H- pyran-4- yl)amino)pyrimidin-4- yl)-1H-indol-3- yl)((2S,4R)-4-fluoro-2- (hydroxymethyl) pyrrolidin- 1-yl)methanone | 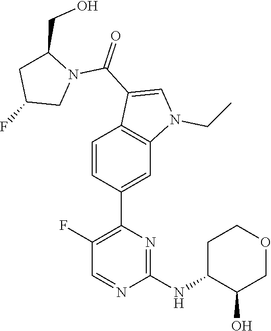 [SMILES: CCn1cc(C(=O)N2C[C@H](F)C[C@H]2CO)c2ccc(-c3nc(N[C@@H]4CCOC[C@H]4O)ncc3F)cc21] | LCMS found 502.2 |
| 93 | (1-Ethyl-6-(5-fluoro-2- (((3S,4R)-3- hydroxytetrahydro-2H- pyran-4- yl)amino)pyrimidin-4- yl)-1H-indol-3-yl)((S)- 2-(hydroxymethyl)-4,4- dimethylpyrrolidin-1- yl)methanone | 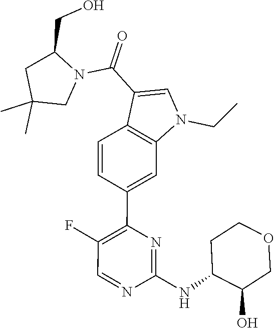 [SMILES: CCn1cc(C(=O)N2CC(C)(C)C[C@H]2CO)c2ccc(-c3nc(N[C@@H]4CCOC[C@H]4O)ncc3F)cc21] | LCMS found 512.3 |
| 94 | (1-Ethyl-6-(5-fluoro-2- (((3S,4R)-3- hydroxytetrahydro-2H- pyran-4- yl)amino)pyrimidin-4- yl)-1H-indol-3-yl)((S)- 6-(hydroxymethyl)-5- azaspiro[2.4] heptan-5- yl)methanone | 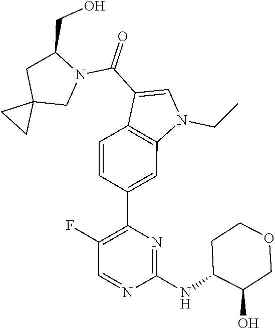 [SMILES: CCn1cc(C(=O)N2CC3(CC3)C[C@H]2CO)c2ccc(-c3nc(N[C@@H]4CCOC[C@H]4O)ncc3F)cc21] | LCMS found 510.2 |
| 95 | ((S)-4,4-Difluoro-2- (hydroxymethyl)pyrrolidin- 1-yl)(1-ethyl-6-(5- fluoro-2-(((3S,4R)-3- hydroxytetrahydro-2H- pyran-4- yl)amino)pyrimidin-4- yl)-1H-indol-3- yl)methanone | 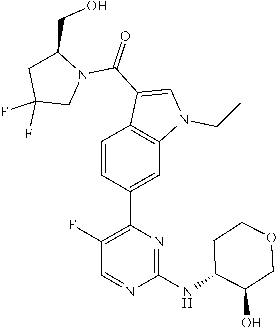 [SMILES: CCn1cc(C(=O)N2CC(F)(F)C[C@H]2CO)c2ccc(-c3nc(N[C@@H]4CCOC[C@H]4O)ncc3F)cc21] | LCMS found 520.2 |
| 96 | ((3S,6S)-1,1-Difluoro- 6-(hydroxymethyl)-5- azaspiro[2.4]heptan-5- yl)(1-ethyl-6-(5-fluoro- 2-(((3S,4R)-3- hydroxytetrahydro-2H- pyran-4- yl)amino)pyrimidin-4- yl)-1H-indol-3- yl)methanone | 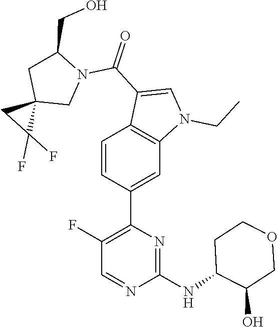 [SMILES: CCn1cc(C(=O)N2C[C@]3(C[C@H]2CO)CC3(F)F)c2ccc(-c3nc(N[C@@H]4CCOC[C@H]4O)ncc3F)cc21] | LCMS found 546.2 |
| 97 | 2-((S)-1-(1-Ethyl-6-(5- fluoro-2-(((3S,4R)-3- hydroxytetrahydro-2H- pyran-4- yl)amino)pyrimidin-4- yl)-1H-indole-3- carbonyl)azetidin-2- yl)acetonitrile | 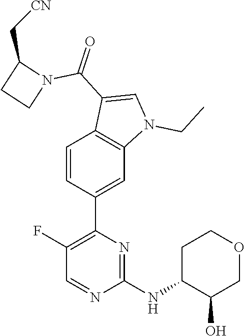 [SMILES: CCn1cc(C(=O)N2CC[C@H]2CC#N)c2ccc(-c3nc(N[C@@H]4CCOC[C@H]4O)ncc3F)cc21] | LCMS found 479.2 |
| 98 | (1-Ethyl-6-(5-fluoro-2- (((3S,4R)-3- hydroxytetrahydro-2H- pyran-4- yl)amino)pyrimidin-4- yl)-1H-indol-3-yl)((S)- 2- ((methylsulfonyl) methyl)azetidin-1- yl)methanone | 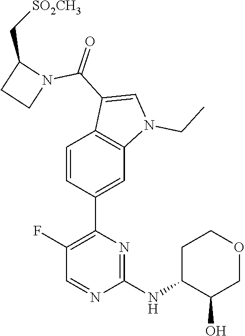 [SMILES: CCn1cc(C(=O)N2CC[C@H]2CS(C)(=O)=O)c2ccc(-c3nc(N[C@@H]4CCOC[C@H]4O)ncc3F)cc21] | LCMS found 532.2 |
| 99 | (1,1-Difluoro-5- azaspiro[2.3]hexan-5- yl)(1-ethyl-6-(5-fluoro- 2-(((3S,4R)-3- hydroxytetrahydro-2H- pyran-4- yl)amino)pyrimidin-4- yl)-1H-indol-3- yl)methanone | 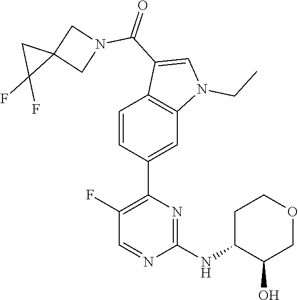 [SMILES: CCn1cc(C(=O)N2CC3(C2)CC3(F)F)c2ccc(-c3nc(N[C@@H]4CCOC[C@H]4O)ncc3F)cc21] | LCMS found 502.2 |
| 100 | (5,5-Difluoro-2- azaspiro[3.3]heptan-2- yl)(1-ethyl-6-(5-fluoro- 2-(((3S,4R)-3- hydroxytetrahydro-2H- pyran-4- yl)amino)pyrimidin-4- yl)-1H-indol-3- yl)methanone | 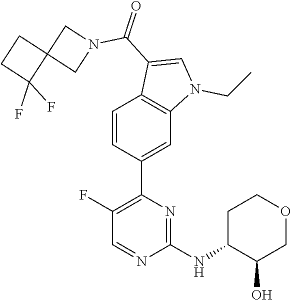 [SMILES: CCn1cc(C(=O)N2CC3(CCC3(F)F)C2)c2ccc(-c3nc(N[C@@H]4CCOC[C@H]4O)ncc3F)cc21] | LCMS found 516.2 |
| 101 | (1-Ethyl-6-(5-fluoro-2- (((3S,4R)-3- hydroxytetrahydro-2H- pyran-4- yl)amino)pyrimidin-4- yl)-1H-indol-3-yl)(3- (2-fluoropropan-2- yl)azetidin-1- yl)methanone | 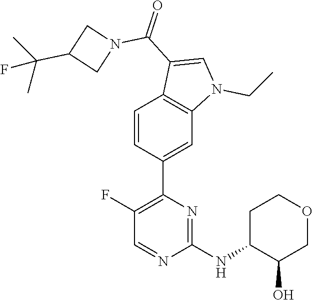 [SMILES: CCn1cc(C(=O)N2CC(C(C)(C)F)C2)c2ccc(-c3nc(N[C@@H]4CCOC[C@H]4O)ncc3F)cc21] | LCMS found 500.2 |
| 102 | (1-Ethyl-6-(5-fluoro-2- (((3S,4R)-3- hydroxytetrahydro-2H- pyran-4- yl)amino)pyrimidin-4- yl)-1H-indol-3-yl)((R)- 3- (trifluoromethoxy) pyrrolidin-1-yl)methanone | 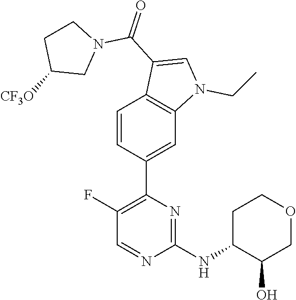 | LCMS found 538.2 |
| 103 | (1-Ethyl-6-(5-fluoro-2- (((3S,4R)-3- hydroxytetrahydro-2H- pyran-4- yl)amino)pyrimidin-4- yl)-1H-indol-3-yl)(5- azaspiro[2.3]hexan-5- yl)methanone | 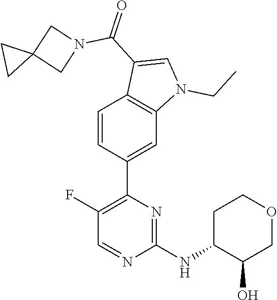 [SMILES: CCn1cc(C(=O)N2CC3(CC3)C2)c2ccc(-c3nc(N[C@@H]4CCOC[C@H]4O)ncc3F)cc21] | LCMS found 466.2 |
| 104 | N-((S)-1-Cyclopropyl- 2-hydroxyethyl)-1- ethyl-6-(5-fluoro-2- (((3S,4R)-3- hydroxytetrahydro-2H- pyran-4- yl)amino)pyrimidin-4- yl)-1H-indole-3- carboxamide | 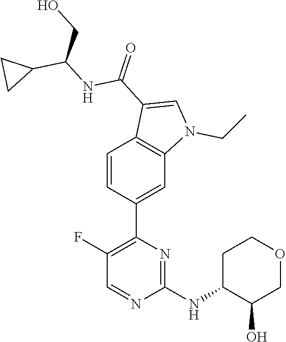 [SMILES: CCn1cc(C(=O)N[C@H](CO)C2CC2)c2ccc(-c3nc(N[C@@H]4CCOC[C@H]4O)ncc3F)cc21] | LCMS found 484.2 |
| 105 | (1-Ethyl-6-(5-fluoro-2- (((3S,4R)-3- hydroxytetrahydro-2H- pyran-4- yl)amino)pyrimidin-4- yl)-1H-indol-3-yl)(5- azaspiro[2.4]heptan-5- yl)methanone | 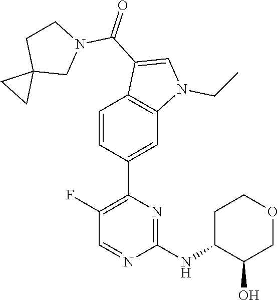 [SMILES: CCn1cc(C(=O)N2CCC3(CC3)C2)c2ccc(-c3nc(N[C@@H]4CCOC[C@H]4O)ncc3F)cc21] | LCMS found 480.2 |
| 106 | (3-(Difluoromethyl)-3- hydroxyazetidin-1- yl)(1-ethyl-6-(5-fluoro- 2-(((3S,4R)-3- hydroxytetrahydro-2H- pyran-4- yl)amino)pyrimidin-4- yl)-1H-indol-3- yl)methanone | 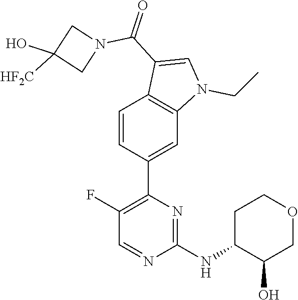 | LCMS found 506.2 |
| 107 | (1-Ethyl-6-(5-fluoro-2- (((3S,4R)-3- hydroxytetrahydro-2H- pyran-4- yl)amino)pyrimidin-4- yl)-1H-indol-3-yl)(3- hydroxy-3- (trifluoromethyl)azetidin- 1-yl)methanone | 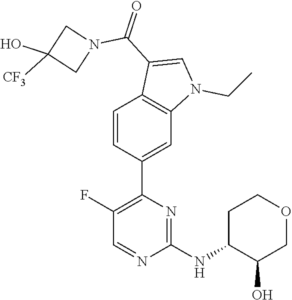 [SMILES: CCn1cc(C(=O)N2CC(O)(C(F)(F)F)C2)c2ccc(-c3nc(N[C@@H]4CCOC[C@H]4O)ncc3F)cc21] | LCMS found 524.2 |

TABLE 3
The compounds in Table 3 were prepared in accordance with the synthetic protocols set
forth in Example 20 using the appropriate starting materials.

| | | | Analytical |
|---|---|---|---|
| Ex. | Name | Structure | data |
| 108 | ((4S)-1,1-Difluoro-4- (hydroxymethyl)-5- azaspiro[2.3]hexan-5- yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 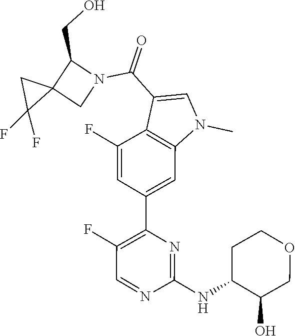 [SMILES: Cn1cc(C(=O)N2CC3(CC3(F)F)[C@H]2CO)c2c(F)cc(-c3nc(N[C@@H]4CCOC[C@H]4O)ncc3F)cc21] | LCMS found 536.2 |
| 109 | ((4R)-1,1-Difluoro-4- (hydroxymethyl)-5- azaspiro[2.3]hexan-5- yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 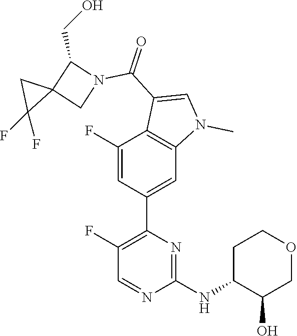 [SMILES: Cn1cc(C(=O)N2CC3(CC3(F)F)[C@@H]2CO)c2c(F)cc(-c3nc(N[C@@H]4CCOC[C@H]4O)ncc3F)cc21] | LCMS found 536.2 |
| 110 | ((2S,3S)-2- ((Cyclopropylsulfonyl) methyl)-3- methylazetidin-1-yl)(4- fluoro-6-(5-fluoro-2- (((3S,4R)-3- hydroxytetrahydro-2H- pyran-4- yl)amino)pyrimidin-4- yl)-1-methyl-1H-indol- 3-yl)methanone | 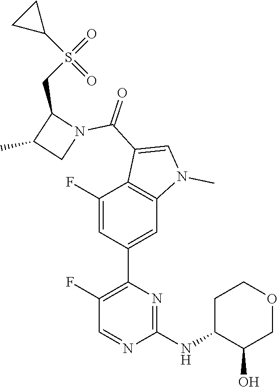 [SMILES: C[C@H]1CN(C(=O)c2cn(C)c3cc(-c4nc(N[C@@H]5CCOC[C@H]5O)ncc4F)cc(F)c23)[C@@H]1CS(=O)(=O)C1CC1] | LCMS found 576.3 |
| 111 | 2-((S)-1-(4-Fluoro-6- (5-fluoro-2-(((3S,4R)- 3-hydroxytetrahydro- 2H-pyran-4- yl)amino)pyrimidin-4- yl)-1-methyl-1H- indole-3- carbonyl)azetidin-2- yl)acetonitrile | 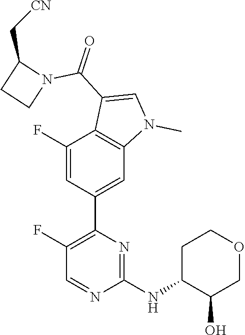 [SMILES: Cn1cc(C(=O)N2CC[C@H]2CC#N)c2c(F)cc(-c3nc(N[C@@H]4CCOC[C@H]4O)ncc3F)cc21] | LCMS found 483.2 |
| 112 | ((2S,4R)-4- (Difluoromethoxy)-2- (hydroxymethyl)pyrroli- din-1-yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 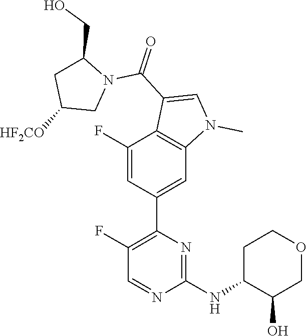 | LCMS found 554.2 |
| 113 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((2S,4R)-2- (hydroxymethyl)-4- (trifluoromethoxy)pyrro- lidin-1-yl)methanone | 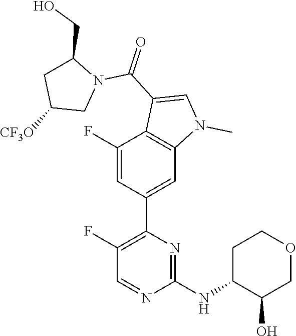 | LCMS found 572.2 |
| 114 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((2S,4R)-2- (hydroxymethyl)-4- (trifluoromethyl)pyrroli- din-1-yl)methanone | 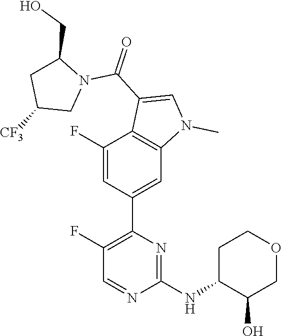 [SMILES: Cn1cc(C(=O)N2C[C@H](C(F)(F)F)C[C@H]2CO)c2c(F)cc(-c3nc(N[C@@H]4CCOC[C@H]4O)ncc3F)cc21] | LCMS found 556.2 |
| 115 | ((3S,6S)-1,1-Difluoro- 6-(hydroxymethyl)-5- azaspiro[2.4]heptan-5- yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 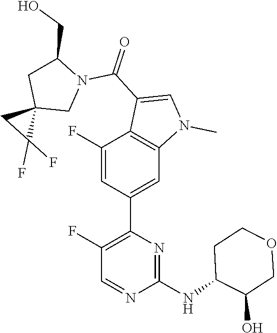 [SMILES: Cn1cc(C(=O)N2C[C@]3(C[C@H]2CO)CC3(F)F)c2c(F)cc(-c3nc(N[C@@H]4CCOC[C@H]4O)ncc3F)cc21] | LCMS found 550.2 |
| 116 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((2S,4S)-2- (hydroxymethyl)-4- methoxy-4- (trifluoromethyl)pyrroli- din-1-yl)methanone | 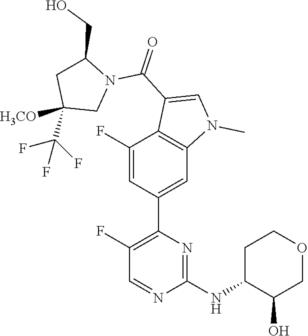 [SMILES: CO[C@@]1(C(F)(F)F)C[C@@H](CO)N(C(=O)c2cn(C)c3cc(-c4nc(N[C@@H]5CCOC[C@H]5O)ncc4F)cc(F)c23)C1] | LCMS found 586.2 |
| 117 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((2S,4S)-4- hydroxy-2- (hydroxymethyl)-4- (trifluoromethyl)pyrroli- din-1-yl)methanone | 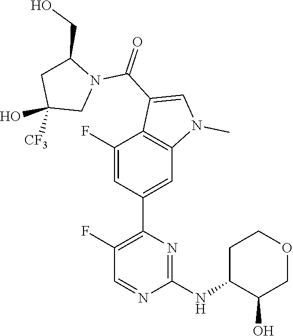 [SMILES: Cn1cc(C(=O)N2C[C@](O)(C(F)(F)F)C[C@H]2CO)c2c(F)cc(-c3nc(N[C@@H]4CCOC[C@H]4O)ncc3F)cc21] | LCMS found 572.2 |
| 118 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((R)-3- (trifluoromethoxy)pyrro- lidin-1-yl)methanone | 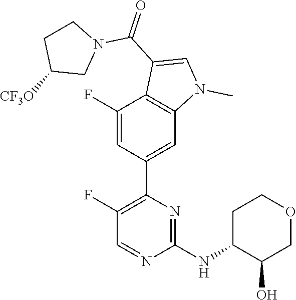 | LCMS found 542.2 |
| 119 | ((R)-3- (Difluoromethoxy)pyrro- lidin-1-yl)(4-fluoro-6- (5-fluoro-2-(((3S,4R)- 3-hydroxytetrahydro- 2H-pyran-4- yl)amino)pyrimidin-4- yl)-1-methyl-1H-indol- 3-yl)methanone | 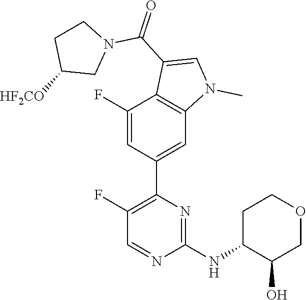 | LCMS found 524.2 |
| 120 | (4-fluoro-6-(5-fluoro-2- (((3S,4R)-3- hydroxytetrahydro-2H- pyran-4- yl)amino)pyrimidin-4- yl)-1-methyl-1H-indol- 3-yl)(5- azaspiro[2.3]hexan-5- yl)methanone | 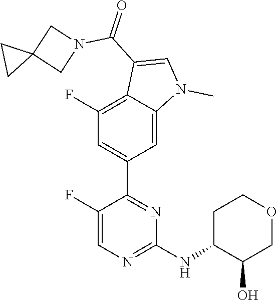 [SMILES: Cn1cc(C(=O)N2CC3(CC3)C2)c2c(F)cc(-c3nc(N[C@@H]4CCOC[C@H]4O)ncc3F)cc21] | LCMS found 470.3 |
| 121 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((2S,3S)-2- (hydroxymethyl-d2)-3- methylazetidin-1- yl)methanone | 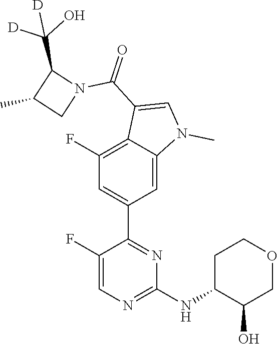 [SMILES: [2H]C([2H])(O)[C@@H]1[C@@H](C)CN1C(=O)c1cn(C)c2cc(-c3nc(N[C@@H]4CCOC[C@H]4O)ncc3F)cc(F)c12] | LCMS found 490.3 |
| 122 | ((2R,3S)-3- (Difluoromethoxy)-2- (hydroxymethyl)azetidin- 1-yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 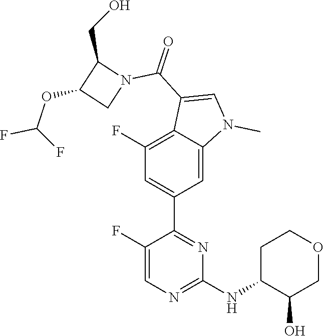 [SMILES: Cn1cc(C(=O)N2C[C@H](OC(F)F)[C@H]2CO)c2c(F)cc(-c3nc(N[C@@H]4CCOC[C@H]4O)ncc3F)cc21] | LCMS found 540.3 |
| 123 | ((S)-2- (Difluoromethyl)azetidin- 1-yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 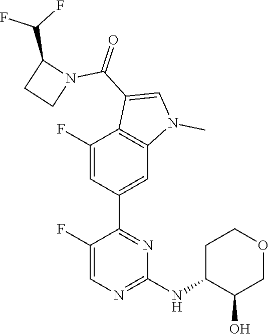 [SMILES: Cn1cc(C(=O)N2CC[C@H]2C(F)F)c2c(F)cc(-c3nc(N[C@@H]4CCOC[C@H]4O)ncc3F)cc21] | LCMS found 494.4 |
| 124 | (S)-1-(4-Fluoro-6-(5- fluoro-2-(((3S,4R)-3- hydroxytetrahydro-2H- pyran-4- yl)amino)pyrimidin-4- yl)-1-methyl-1H- indole-3- carbonyl)azetidine-2- carbonitrile | 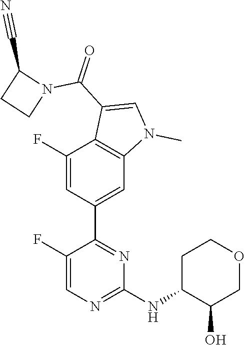 [SMILES: Cn1cc(C(=O)N2CC[C@H]2C#N)c2c(F)cc(-c3nc(N[C@@H]4CCOC[C@H]4O)ncc3F)cc21] | LCMS found 469.2 |
| 125 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)(3- hydroxyazetidin-1- yl)methanone | 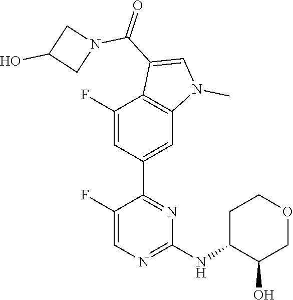 [SMILES: Cn1cc(C(=O)N2CC(O)C2)c2c(F)cc(-c3nc(N[C@@H]4CCOC[C@H]4O)ncc3F)cc21] | LCMS found 460.4 |
| 126 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)(3-methylazetidin- 1-yl)methanone | 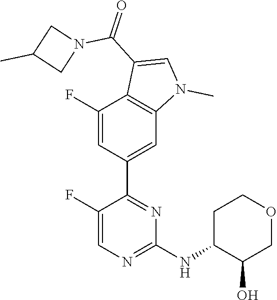 [SMILES: CC1CN(C(=O)c2cn(C)c3cc(-c4nc(N[C@@H]5CCOC[C@H]5O)ncc4F)cc(F)c23)C1] | LCMS found 458.5 |
| 127 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)(3- methoxyazetidin-1- yl)methanone | 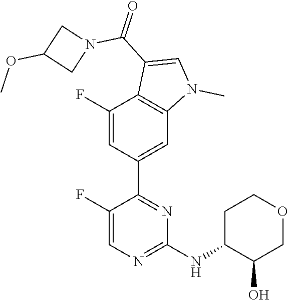 [SMILES: COC1CN(C(=O)c2cn(C)c3cc(-c4nc(N[C@@H]5CCOC[C@H]5O)ncc4F)cc(F)c23)C1] | LCMS found 474.4 |
| 128 | (3-(1,1-Difluoroethyl)- 3-methylazetidin-1- yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 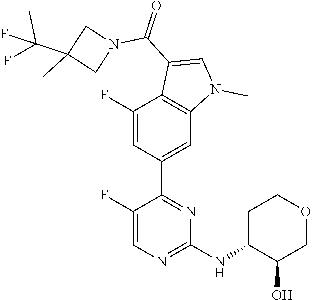 [SMILES: Cn1cc(C(=O)N2CC(C)(C(C)(F)F)C2)c2c(F)cc(-c3nc(N[C@@H]4CCOC[C@H]4O)ncc3F)cc21] | LCMS found 522.2 |
| 129 | (1,1-Difluoro-2,2- dimethyl-5- azaspiro[2.3]hexan-5- yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 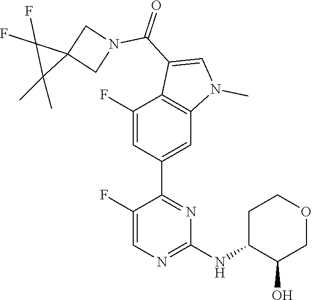 [SMILES: Cn1cc(C(=O)N2CC3(C2)C(C)(C)C3(F)F)c2c(F)cc(-c3nc(N[C@@H]4CCOC[C@H]4O)ncc3F)cc21] | LCMS found 534.2 |

TABLE 4
The compounds in Table 4 were prepared in accordance with the synthetic protocols set
forth in Example 69 using the appropriate starting materials.

| | | | Analytical |
|---|---|---|---|
| Ex. | Name | Structure | data |
| 130 | (6-(5-Chloro-2- (((3S,4R)-3- hydroxytetrahydro-2H- pyran-4- yl)amino)pyrimidin-4- yl)-1-ethyl-1H-indol-3- yl)(3- (trifluoromethyl)azetidin- 1-yl)methanone | 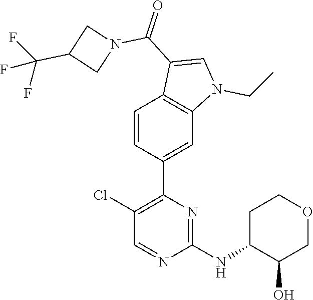 [SMILES: CCn1cc(C(=O)N2CC(C(F)(F)F)C2)c2ccc(-c3nc(N[C@@H]4CCOC[C@H]4O)ncc3Cl)cc21] | LCMS found 524.2 |
| 131 | (6-(5-Chloro-2- (((3S,4R)-3- hydroxytetrahydro-2H- pyran-4- yl)amino)pyrimidin-4- yl)-1-ethyl-1H-indol-3- yl)((R)-2- methylazetidin-1- yl)methanone | 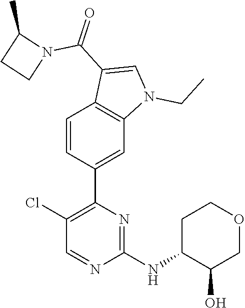 [SMILES: CCn1cc(C(=O)N2CC[C@H]2C)c2ccc(-c3nc(N[C@@H]4CCOC[C@H]4O)ncc3Cl)cc21] | LCMS found 470.3 |
| 132 | (6-(5-Chloro-2- (((3S,4R)-3- hydroxytetrahydro-2H- pyran-4- yl)amino)pyrimidin-4- yl)-1-ethyl-1H-indol-3- yl)((2S,4R)-4- (difluoromethoxy)-2- (hydroxymethyl)pyrroli- din-1-yl)methanone | 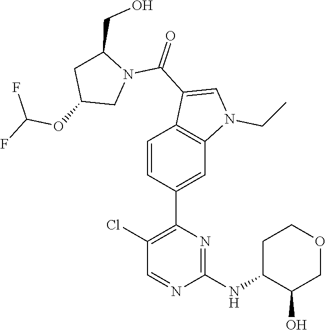 [SMILES: CCn1cc(C(=O)N2C[C@H](OC(F)F)C[C@H]2CO)c2ccc(-c3nc(N[C@@H]4CCOC[C@H]4O)ncc3Cl)cc21] | LCMS found 566.2 |
| 133 | (6-(5-Chloro-2- (((3S,4R)-3- hydroxytetrahydro-2H- pyran-4- yl)amino)pyrimidin-4- yl)-1-ethyl-1H-indol-3- yl)((2S,4R)-4-fluoro-2- (hydroxymethyl)pyrroli- din-1-yl)methanone | 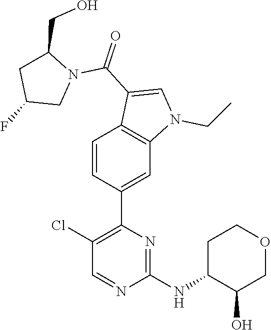 [SMILES: CCn1cc(C(=O)N2C[C@H](F)C[C@H]2CO)c2ccc(-c3nc(N[C@@H]4CCOC[C@H]4O)ncc3Cl)cc21] | LCMS found 518.2 |

TABLE 5
The compounds in Table 5 were prepared in accordance with the synthetic protocols set
forth in Example 49 using the appropriate starting materials.

| | | | Analytical |
|---|---|---|---|
| Ex. | Name | Structure | data |
| 134 | (5,5-Difluoro-2- azaspiro[3.3]heptan-2- yl)(1-(2,2- difluoroethyl)-4-fluoro- 6-(5-fluoro-2- (((3S,4R)-3- hydroxytetrahydro-2H- pyran-4- yl)amino)pyrimidin-4- yl)-1H-indol-3- yl)methanone | 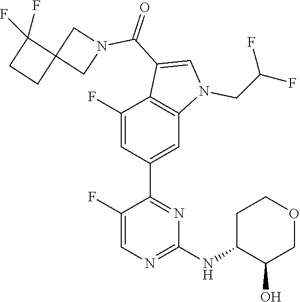 [SMILES: O=C(c1cn(CC(F)F)c2cc(-c3nc(N[C@@H]4CCOC[C@H]4O)ncc3F)cc(F)c12)N1CC2(CCC2(F)F)C1] | LCMS found 570.4 |
| 135 | (1-(2,2-Difluoroethyl)- 4-fluoro-6-(5-fluoro-2- (((3S,4R)-3- hydroxytetrahydro-2H- pyran-4- yl)amino)pyrimidin-4- yl)-1H-indol-3- yl)((2S,3S)-2- (hydroxymethyl-d2)-3- methylazetidin-1- yl)methanone | 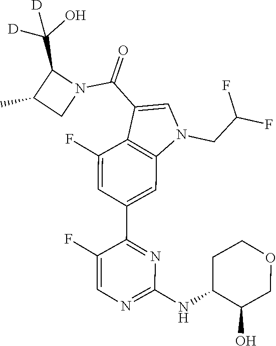 [SMILES: [2H]C([2H])(O)[C@@H]1[C@@H](C)CN1C(=O)c1cn(CC(F)F)c2cc(-c3nc(N[C@@H]4CCOC[C@H]4O)ncc3F)cc(F)c12] | LCMS found 540.4 |
| 136 | (1-(2,2-Difluoroethyl)- 4-fluoro-6-(5-fluoro-2- (((3S,4R)-3- hydroxytetrahydro-2H- pyran-4- yl)amino)pyrimidin-4- yl)-1H-indol-3-yl)(3- (2-fluoropropan-2- yl)azetidin-1- yl)methanone | 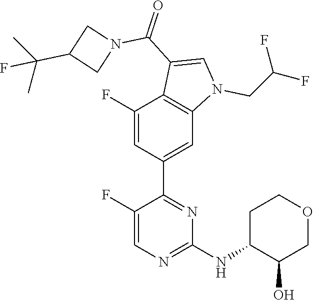 [SMILES: CC(C)(F)C1CN(C(=O)c2cn(CC(F)F)c3cc(-c4nc(N[C@@H]5CCOC[C@H]5O)ncc4F)cc(F)c23)C1] | LCMS found 554.4 |
| 137 | (1,1-Difluoro-5- azaspiro[2.3]hexan-5- yl)(1-(2,2- difluoroethyl)-4-fluoro- 6-(5-fluoro-2- (((3S,4R)-3- hydroxytetrahydro-2H- pyran-4- yl)amino)pyrimidin-4- yl)-1H-indol-3- yl)methanone | 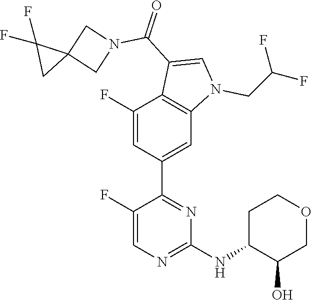 [SMILES: O=C(c1cn(CC(F)F)c2cc(-c3nc(N[C@@H]4CCOC[C@H]4O)ncc3F)cc(F)c12)N1CC2(C1)CC2(F)F] | LCMS found 556.4 |

TABLE 6
The compounds in Table 6 were prepared in accordance with the synthetic protocols set
forth in Example 21 using the appropriate starting materials.

| | | | Analytical |
|---|---|---|---|
| Ex. | Name | Structure | data |
| 138 | (6-(5-Chloro-2- (((3S,4R)-3- hydroxytetrahydro-2H- pyran-4- yl)amino)pyrimidin-4- yl)-4-fluoro-1-methyl- 1H-indol-3-yl)(3-(2- fluoropropan-2- yl)azetidin-1- yl)methanone | 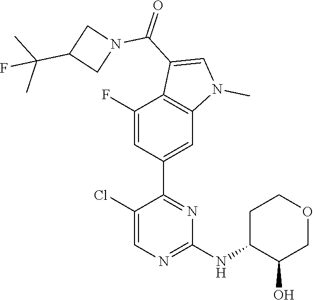 [SMILES: Cn1cc(C(=O)N2CC(C(C)(C)F)C2)c2c(F)cc(-c3nc(N[C@@H]4CCOC[C@H]4O)ncc3Cl)cc21] | LCMS found 520.4 |
| 139 | (6-(5-Chloro-2- (((3S,4R)-3- hydroxytetrahydro-2H- pyran-4- yl)amino)pyrimidin-4- yl)-4-fluoro-1-methyl- 1H-indol-3-yl)(1,1- difluoro-2,2-dimethyl- 5-azaspiro[2.3]hexan- 5-yl)methanone | 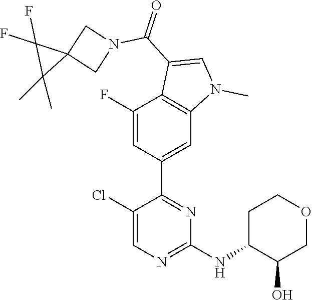 [SMILES: Cn1cc(C(=O)N2CC3(C2)C(C)(C)C3(F)F)c2c(F)cc(-c3nc(N[C@@H]4CCOC[C@H]4O)ncc3Cl)cc21] | LCMS found 550.4 |

TABLE 7
The compounds in Table 7 were prepared in accordance with the synthetic protocols set
forth in Examples 58 and 59 using the appropriate starting materials.

| | | | Analytical |
|---|---|---|---|
| Ex. | Name | Structure | data |
| 140 | (1-(2,2-Difluoroethyl)- 6-(5-fluoro-2- (((3S,4R)-3- hydroxytetrahydro-2H- pyran-4- yl)amino)pyrimidin-4- yl)-1H-indol-3-yl)((S)- 4-(hydroxymethyl)-5- azaspiro[2.3]hexan-5- yl)methanone | 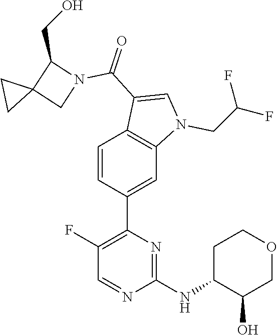 [SMILES: O=C(c1cn(CC(F)F)c2cc(-c3nc(N[C@@H]4CCOC[C@H]4O)ncc3F)ccc12)N1CC2(CC2)[C@H]1CO] | LCMS found 532.2 Retention time on chiral SFC tᵣ = 4.5 min |
| 141 | (1-(2,2-Difluoroethyl)- 6-(5-fluoro-2- (((3S,4R)-3- hydroxytetrahydro-2H- pyran-4- yl)amino)pyrimidin-4- yl)-1H-indol-3-yl)((R)- 4-(hydroxymethyl)-5- azaspiro[2.3]hexan-5- yl)methanone | 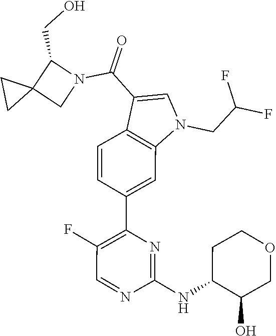 [SMILES: O=C(c1cn(CC(F)F)c2cc(-c3nc(N[C@@H]4CCOC[C@H]4O)ncc3F)ccc12)N1CC2(CC2)[C@@H]1CO] | LCMS found 532.2 Retention time on chiral SFC tᵣ = 4.0 min |
| 142 | ((4S)-1,1-Difluoro-4- (hydroxymethyl)-5- azaspiro[2.3]hexan-5- yl)(1-(2,2- difluoroethyl)-6-(5- fluoro-2-(((3S,4R)-3- hydroxytetrahydro-2H- pyran-4- yl)amino)pyrimidin-4- yl)-1H-indol-3- yl)methanone | 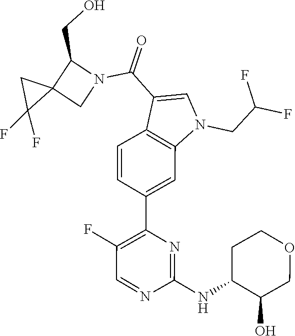 [SMILES: O=C(c1cn(CC(F)F)c2cc(-c3nc(N[C@@H]4CCOC[C@H]4O)ncc3F)ccc12)N1CC2(CC2(F)F)[C@H]1CO] | LCMS found 568.2 Retention time on chiral SFC tᵣ = 4.4 min |
| 143 | ((4R)-1,1-Difluoro-4- (hydroxymethyl)-5- azaspiro[2.3]hexan-5- yl)(1-(2,2- difluoroethyl)-6-(5- fluoro-2-(((3S,4R)-3- hydroxytetrahydro-2H- pyran-4- yl)amino)pyrimidin-4- yl)-1H-indol-3- yl)methanone | 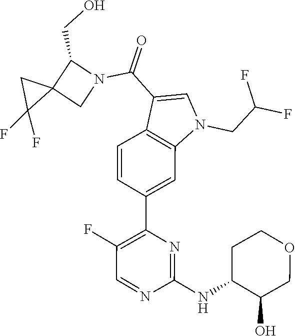 [SMILES: O=C(c1cn(CC(F)F)c2cc(-c3nc(N[C@@H]4CCOC[C@H]4O)ncc3F)ccc12)N1CC2(CC2(F)F)[C@@H]1CO] | LCMS found 568.2 Retention time on chiral SFC tᵣ = 3.8 min |
| 144 | (1-(2,2-Difluoroethyl)- 4-fluoro-6-(5-fluoro-2- (((3S,4R)-3- hydroxytetrahydro-2H- pyran-4- yl)amino)pyrimidin-4- yl)-1H-indol-3-yl)((S)- 4-(hydroxymethyl)-5- azaspiro[2.3]hexan-5- yl)methanone | 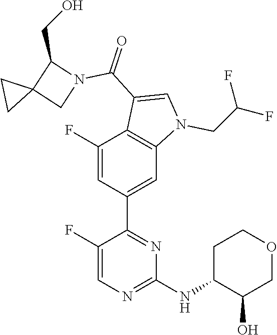 [SMILES: O=C(c1cn(CC(F)F)c2cc(-c3nc(N[C@@H]4CCOC[C@H]4O)ncc3F)cc(F)c12)N1CC2(CC2)[C@H]1CO] | LCMS found 550.4 Retention time on chiral SFC tᵣ = 8.0 min |
| 145 | (1-(2,2-Difluoroethyl)- 4-fluoro-6-(5-fluoro-2- (((3S,4R)-3- hydroxytetrahydro-2H- pyran-4- yl)amino)pyrimidin-4- yl)-1H-indol-s3- yl)((R)-4- (hydroxymethyl)-5- azaspiro[2.3]hexan-5- yl)methanone | 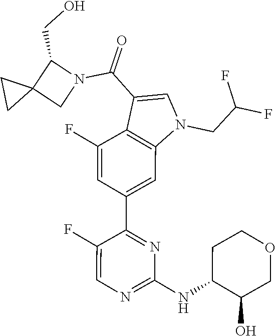 [SMILES: O=C(c1cn(CC(F)F)c2cc(-c3nc(N[C@@H]4CCOC[C@H]4O)ncc3F)cc(F)c12)N1CC2(CC2)[C@@H]1CO] | LCMS found 550.4 Retention time on chiral SFC tᵣ = 6.4 min |
| 146 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((S)-1- (hydroxymethyl)-2- azaspiro[3.3]heptan-2- yl)methanone | 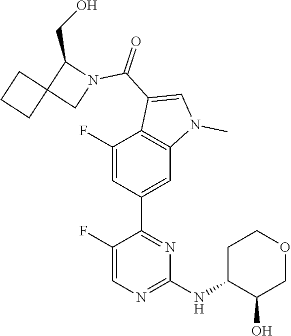 [SMILES: Cn1cc(C(=O)N2CC3(CCC3)[C@H]2CO)c2c(F)cc(-c3nc(N[C@@H]4CCOC[C@H]4O)ncc3F)cc21] | LCMS found 514.4 Retention time on chiral SFC tᵣ = 7.0 min |
| 147 | (4-Fluoro-6-(5-fluoro- 2-(((3S,4R)-3- hydroxytetrahydro-2H- pyran-4- yl)amino)pyrimidin-4- yl)-1-methyl-1H-indol- 3-yl)((R)-1- (hydroxymethyl)-2- azaspiro[3.3]heptan-2- yl)methanone | 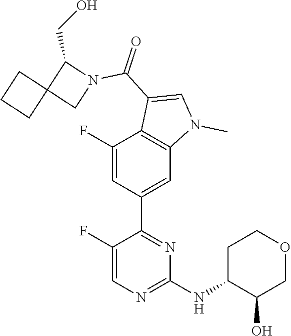 [SMILES: Cn1cc(C(=O)N2CC3(CCC3)[C@@H]2CO)c2c(F)cc(-c3nc(N[C@@H]4CCOC[C@H]4O)ncc3F)cc21] | LCMS found 514.4 Retention time on chiral SFC tᵣ = 7.0 min |
| 148 | ((4S)-1,1-difluoro-4- (hydroxymethyl)-5- azaspiro[2.3]hexan-5- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 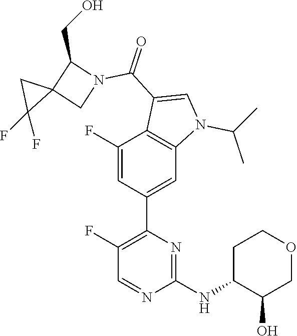 [SMILES: CC(C)n1cc(C(=O)N2CC3(CC3(F)F)[C@H]2CO)c2c(F)cc(-c3nc(N[C@@H]4CCOC[C@H]4O)ncc3F)cc21] | LCMS found 564.3 Retention time on chiral SFC tᵣ = 7.1 min |
| 149 | ((4R)-1,1-difluoro-4- (hydroxymethyl)-5- azaspiro[2.3]hexan-5- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 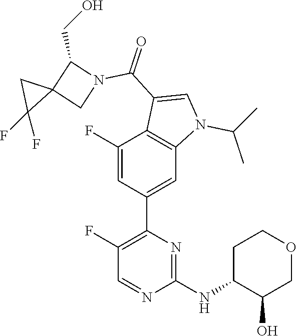 [SMILES: CC(C)n1cc(C(=O)N2CC3(CC3(F)F)[C@@H]2CO)c2c(F)cc(-c3nc(N[C@@H]4CCOC[C@H]4O)ncc3F)cc21] | LCMS found 564.2 Retention time on chiral SFC tᵣ = 7.2 min |
| 150 | ((2S,3S)-3- (difluoromethyl)-2- (hydroxymethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 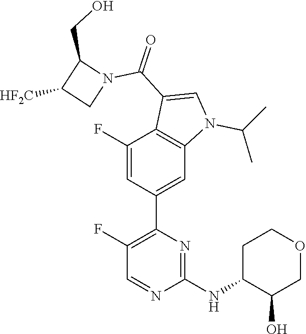 | LCMS found 552.2 Retention time on chiral SFC tᵣ = 7.5 min |
| 151 | ((2R,3R)-3- (difluoromethyl)-2- (hydroxymethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-y1)methanone | 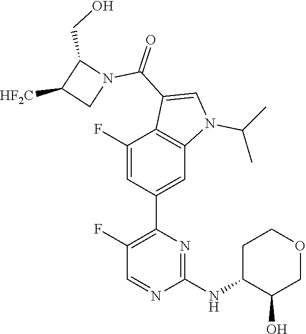 | LCMS found 552.2 Retention time on chiral SFC tᵣ = 8.0 min |
| 152 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((1R,4S,5R)- 5-(hydroxymethyl)-2- azabicyclo[2.1.0]pentan- 2-yl)methanone | 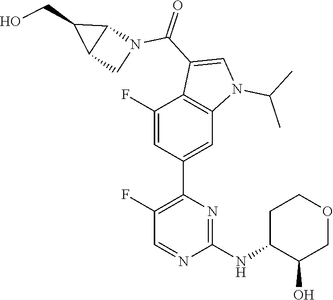 [SMILES: CC(C)n1cc(C(=O)N2C[C@@H]3[C@@H](CO)[C@@H]32)c2c(F)cc(-c3nc(N[C@@H]4CCOC[C@H]4O)ncc3F)cc21] | LCMS found 514.3 Retention time on chiral SFC tᵣ = 3.2 min |
| 153 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((1S,4R,5S)- 5-(hydroxymethyl)-2- azabicyclo[2.1.0]pentan- 2-yl)methanone | 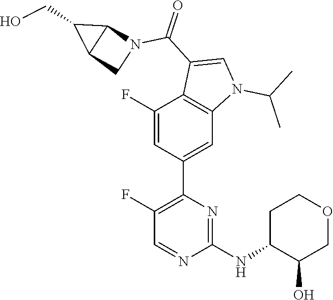 [SMILES: CC(C)n1cc(C(=O)N2C[C@H]3[C@H](CO)[C@H]32)c2c(F)cc(-c3nc(N[C@@H]4CCOC[C@H]4O)ncc3F)cc21] | LCMS found 514.3 Retention time on chiral SFC tᵣ = 4.2 min |
| 154 | (2S,3S)-1-(4-fluoro-6- (5-fluoro-2-(((3S,4R)- 3-hydroxytetrahydro- 2H-pyran-4- yl)amino)pyrimidin-4- yl)-1-isopropyl-1H- indole-3-carbonyl)-2- (hydroxymethyl)azetidine- 3-carbonitrile | 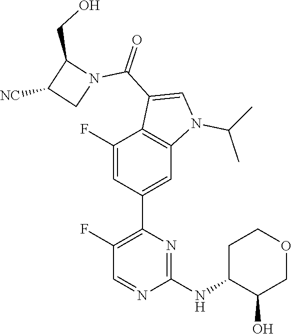 [SMILES: CC(C)n1cc(C(=O)N2C[C@H](C#N)[C@H]2CO)c2c(F)cc(-c3nc(N[C@@H]4CCOC[C@H]4O)ncc3F)cc21] | LCMS found 527.3 Retention time on chiral SFC tᵣ = 4.6 min |
| 155 | (2R,3R)-1-(4-fluoro-6- (5-fluoro-2-(((3S,4R)- 3-hydroxytetrahydro- 2H-pyran-4- yl)amino)pyrimidin-4- yl)-1-isopropyl-1H- indole-3-carbonyl)-2- (hydroxymethyl)azetidine- 3-carbonitrile | 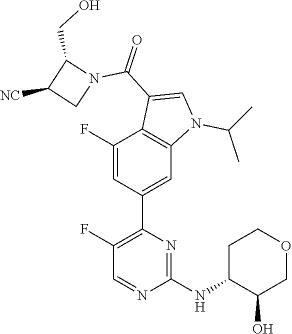 [SMILES: CC(C)n1cc(C(=O)N2C[C@@H](C#N)[C@@H]2CO)c2c(F)cc(-c3nc(N[C@@H]4CCOC[C@H]4O)ncc3F)cc21] | LCMS found 527.3 Retention time on chiral SFC tᵣ = 4.9 min |
| 156 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((2R,3R)-2- (hydroxymethyl)-3- (methylsulfonyl)azetidin- 1-yl)methanone | 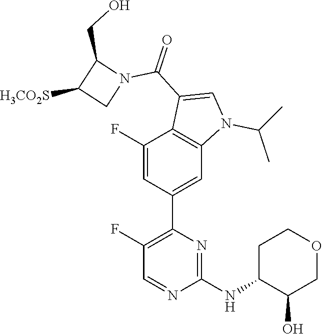 | LCMS found 580.4 Retention time on chiral SFC tᵣ = 6.8 min |
| 157 | (4-fluoro-6-(5-fluoro-2- (((3S,4R)-3- hydroxytetrahydro-2H- pyran-4- yl)amino)pyrimidin-4- yl)-1-isopropyl-1H- indol-3-y1)((2S,3S)-2- (hydroxymethyl)-3- (methylsulfonyl)azetidin- 1-yl)methanone | 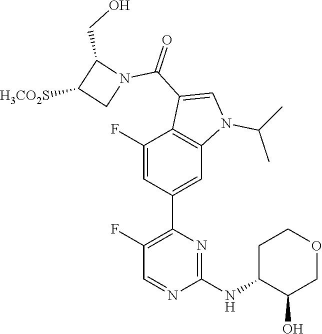 | LCMS found 580.4 Retention time on chiral SFC tᵣ = 7.5 min |
| 158 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((2R,3S)-2- (hydroxymethyl)-3- (methylsulfonyl)azetidin- 1-yl)methanone | 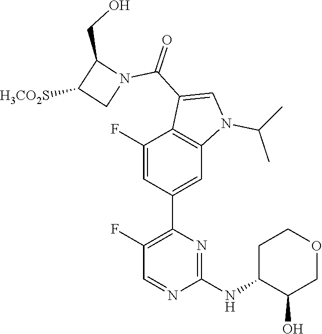 | LCMS found 580.4 Retention time on chiral SFC tᵣ = 4.2 min |
| 159 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((2S,3R)-2- (hydroxymethyl)-3- (methylsulfonyl)azetidin- 1-yl)methanone | 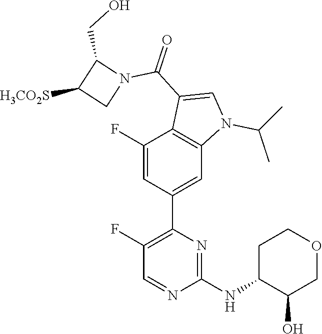 | LCMS found 580.4 Retention time on chiral SFC tᵣ = 6.2 min |
| 160 | ((4R)-1,1-difluoro-4- (hydroxymethyl)-5- azaspiro[2.3 ]hexan-5- yl)(1-ethyl-6-(5-fluoro- 2-(((3S,4R)-3- hydroxytetrahydro-2H- pyran-4- yl)amino)pyrimidin-4- yl)-1H-indol-3- yl)methanone | 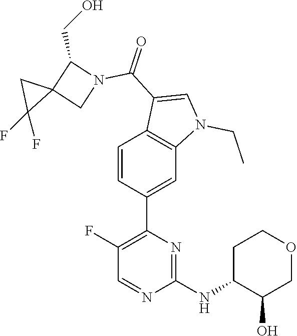 [SMILES: CCn1cc(C(=O)N2CC3(CC3(F)F)[C@@H]2CO)c2ccc(-c3nc(N[C@@H]4CCOC[C@H]4O)ncc3F)cc21] | LCMS found 532.4 Retention time on chiral SFC tᵣ = 4.3 min |
| 161 | ((4S)-1,1-difluoro-4- (hydroxymethyl)-5- azaspiro[2.3]hexan-5- yl)(1-ethyl-6-(5-fluoro- 2-(((3S,4R)-3- hydroxytetrahydro-2H- pyran-4- yl)amino)pyrimidin-4- yl)-1H-indol-3- yl)methanone | 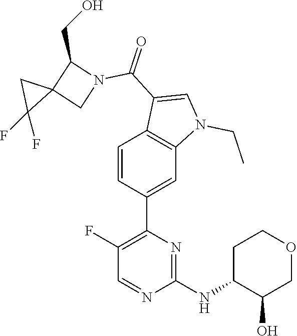 [SMILES: CCn1cc(C(=O)N2CC3(CC3(F)F)[C@H]2CO)c2ccc(-c3nc(N[C@@H]4CCOC[C@H]4O)ncc3F)cc21] | LCMS found 532.5 Retention time on chiral SFC tᵣ = 5.3 min |
| 162 | ((4R)-1,1-difluoro-4- (hydroxymethyl)-5- azaspiro[2.3]hexan-5- yl)(6-(5-fluoro-2- (((3S,4R)-3- hydroxytetrahydro-2H- pyran-4- yl)amino)pyrimidin-4- yl)-1-isopropyl-1H- indol-3-yl)methanone | 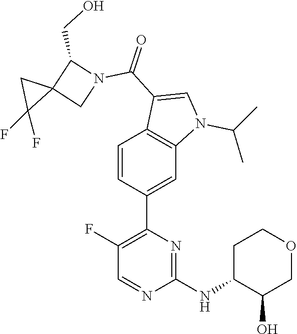 [SMILES: CC(C)n1cc(C(=O)N2CC3(CC3(F)F)[C@@H]2CO)c2ccc(-c3nc(N[C@@H]4CCOC[C@H]4O)ncc3F)cc21] | LCMS found 546.4 Retention time on chiral SFC tᵣ = 4.1 min |
| 163 | ((4S)-1,1-difluoro-4- (hydroxymethyl)-5- azaspiro[2.3]hexan-5- yl)(6-(5-fluoro-2- (((3S,4R)-3- hydroxytetrahydro-2H- pyran-4- yl)amino)pyrimidin-4- yl)-1-isopropyl-1H- indol-3-yl)methanone | 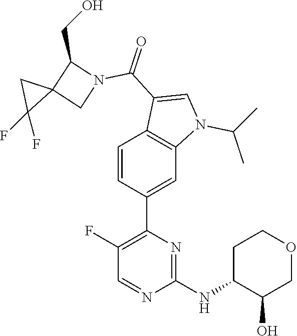 [SMILES: CC(C)n1cc(C(=O)N2CC3(CC3(F)F)[C@H]2CO)c2ccc(-c3nc(N[C@@H]4CCOC[C@H]4O)ncc3F)cc21] | LCMS found 546.4 Retention time on chiral SFC tᵣ = 4.8 min |
| 164 | (6-(5-fluoro-2- (((3S,4R)-3- hydroxytetrahydro-2H- pyran-4- yl)amino)pyrimidin-4- yl)-1-isopropyl-1H- indol-3-yl)((S)-4- (hydroxymethyl)-5- azaspiro[2.3]hexan-5- yl)mehthanone | 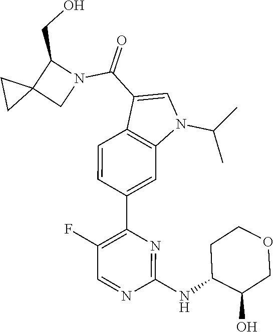 [SMILES: CC(C)n1cc(C(=O)N2CC3(CC3)[C@H]2CO)c2ccc(-c3nc(N[C@@H]4CCOC[C@H]4O)ncc3F)cc21] | LCMS found 510.5 Retention time on chiral SFC tᵣ = 2.1 min |
| 165 | (6-(5-fluoro-2- (((3S,4R)-3- hydroxytetrahydro-2H- pyran-4- yl)amino)pyrimidin-4- yl)-1-isopropyl-1H- indol-3-yl)((R)-4- (hydroxymethyl)-5- azaspiro[2.3]hexan-5- yl)methanone | 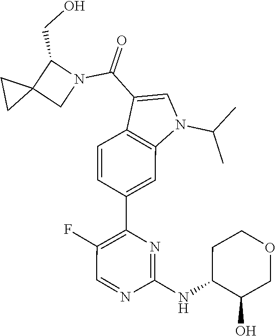 [SMILES: CC(C)n1cc(C(=O)N2CC3(CC3)[C@@H]2CO)c2ccc(-c3nc(N[C@@H]4CCOC[C@H]4O)ncc3F)cc21] | LCMS found 510.5 Retention time on chiral SFC tᵣ = 2.8 min |
| 166 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((2S,3S)-2- (hydroxymethyl)-3- ((trifluoromethoxy)meth- yl)azetidin-1- yl)methanone | 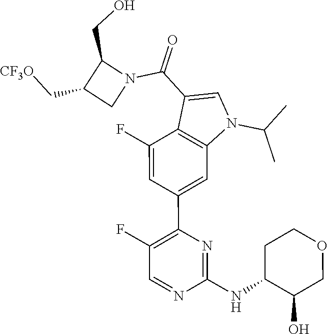 | LCMS found 600.5 Retention time on chiral SFC tᵣ = 4.7 min |
| 167 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((2R,3R)-2- (hydroxymethyl)-3- ((trifluoromethoxy)meth- yl)azetidin-1- yl)methanone | 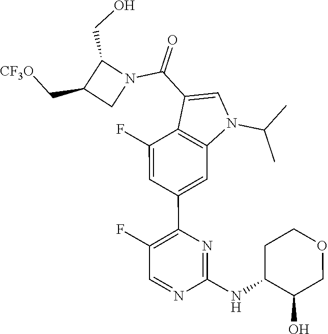 | LCMS found 600.5 Retention time on chiral SFC tᵣ = 5.6 min |
| 168 | ((2S,3S)-3- ((difluoromethoxy)meth- yl)-2- (hydroxymethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 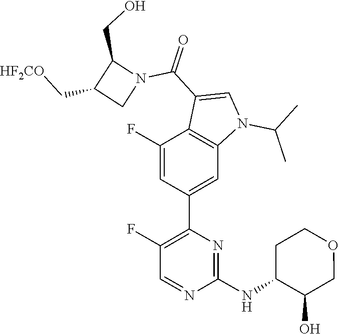 | LCMS found 582.5 Retention time on chiral SFC tᵣ = 6.0 min |
| 169 | ((2R,3R)-3- ((difluoromethoxy)meth- yl)-2- (hydroxymethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 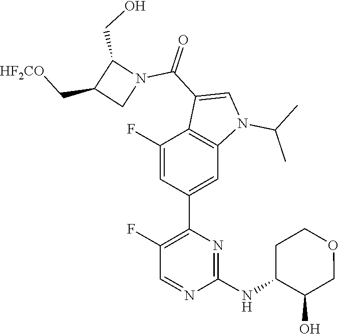 | LCMS found 582.5 Retention time on chiral SFC tᵣ = 7.5 min |
| 170 | ((4S)-1,1-difluoro-4- (hydroxymethyl)-5- azaspiro[2.3]hexan-5- yl)(1-ethy1-4-fluoro-6- (5-fluoro-2-(((3S,4R)- 3-hydroxytetrahydro- 2H-pyran-4- yl)amino)pyrimidin-4- yl)-1H-indol-3- yl)methanone | 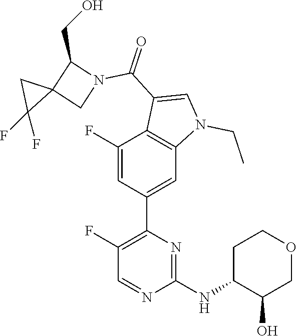 [SMILES: CCn1cc(C(=O)N2CC3(CC3(F)F)[C@H]2CO)c2c(F)cc(-c3nc(N[C@@H]4CCOC[C@H]4O)ncc3F)cc21] | LCMS found 550.4 Retention time on chiral SFC tᵣ = 4.3 min |
| 171 | ((4R)-1,1-difluoro-4- (hydroxymethyl)-5- azaspiro[2.3]hexan-5- yl)(1-ethyl-4-fluoro-6- (5-fluoro-2-(((35,4R)- 3-hydroxytetrahydro- 2H-pyran-4- yl)amino)pyrimidin-4- yl)-1H-indol-3- yl)methanone | 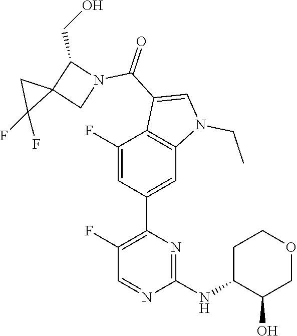 [SMILES: CCn1cc(C(=O)N2CC3(CC3(F)F)[C@@H]2CO)c2c(F)cc(-c3nc(N[C@@H]4CCOC[C@H]4O)ncc3F)cc21] | LCMS found 550.4 Retention time on chiral SFC tᵣ = 5.3 min |
| 172 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((R)-1- (hydroxymethyl)-2- azaspiro[3.3]heptan-2- yl)methanon | 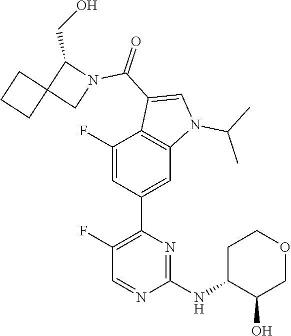 [SMILES: CC(C)n1cc(C(=O)N2CC3(CCC3)[C@@H]2CO)c2c(F)cc(-c3nc(N[C@@H]4CCOC[C@H]4O)ncc3F)cc21] | LCMS found 542.3 Retention time on chiral SFC tᵣ = 5.6 min |
| 173 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((S)-1- (hydroxymethyl)-2- azaspiro[3.3]heptan-2- yl)methanon | 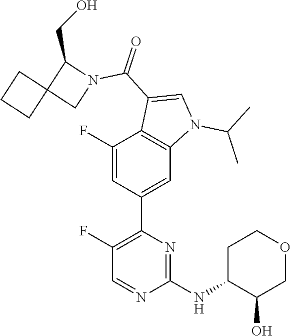 [SMILES: CC(C)n1cc(C(=O)N2CC3(CCC3)[C@H]2CO)c2c(F)cc(-c3nc(N[C@@H]4CCOC[C@H]4O)ncc3F)cc21] | LCMS found 542.3 Retention time on chiral SFC tᵣ = 6.5 min |
| 174 | ((R)-3,3-Difluoro-2- (hydroxymethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 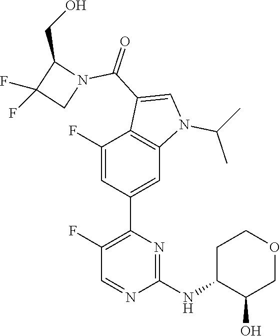 [SMILES: CC(C)n1cc(C(=O)N2CC(F)(F)[C@H]2CO)c2c(F)cc(-c3nc(N[C@@H]4CCOC[C@H]4O)ncc3F)cc21] | LCMS found 538.3 Retention time on chiral SFC tᵣ = 8.4 min |
| 175 | ((S)-3,3-Difluoro-2- (hydroxymethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 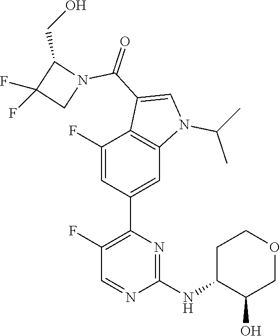 [SMILES: CC(C)n1cc(C(=O)N2CC(F)(F)[C@@H]2CO)c2c(F)cc(-c3nc(N[C@@H]4CCOC[C@H]4O)ncc3F)cc21] | LCMS found 538.3 Retention time on chiral SFC tᵣ = 10 min |

### Example 176. 4-fluoro-6-(5-fluoro-2-(((3R,4R)-3-hydroxy-1-(methylsulfonyl)piperidin-4-yl)amino)pyrimidin-4-yl)-1-isopropyl-N,N-dimethyl-1H-indole-3-carboxamide

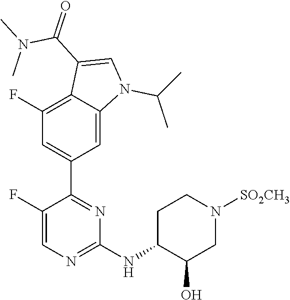 [SMILES: CC(C)n1cc(C(=O)N(C)C)c2c(F)cc(-c3nc(N[C@@H]4CCN(S(C)(=O)=O)C[C@H]4O)ncc3F)cc21]

The title compound was prepared according to the procedures outlined in Example 3, with dimethylamine hydrochloride replacing 2-(pyrimidin-2-yl)ethan-1-amine dihydrochloride. LC-MS calculated for C₂₄H₃₁F₂N₆O₄S (M+H)⁺: m/z=537.2; found 537.3.

TABLE 8
The compounds in Table 8 were prepared in accordance with the synthetic protocols set
forth in Example 8 using the appropriate starting materials.

| | | | Analytical |
|---|---|---|---|
| Ex. | Name | Structure | data |
| 177 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((S)-2- (methoxymethyl)azetidin- 1-yl)methanone | 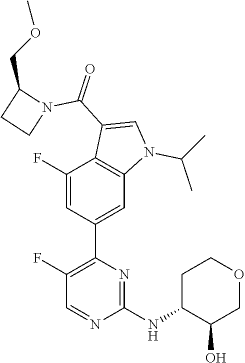 [SMILES: COC[C@@H]1CCN1C(=O)c1cn(C(C)C)c2cc(-c3nc(N[C@@H]4CCOC[C@H]4O)ncc3F)cc(F)c12] | LCMS found 516.3 |
| 178 | 2-((S)-1-(4-fluoro-6-(5- fluoro-2-(((3S,4R)-3- hydroxytetrahydro-2H- pyran-4- yl)amino)pyrimidin-4- yl)-1-isopropyl-1H- indole-3- carbonyl)azetidin-2- yl)acetonitrile | 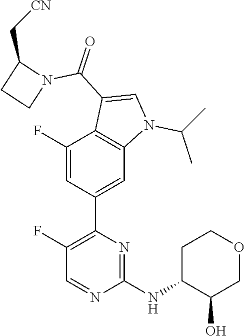 [SMILES: CC(C)n1cc(C(=O)N2CC[C@H]2CC#N)c2c(F)cc(-c3nc(N[C@@H]4CCOC[C@H]4O)ncc3F)cc21] | LCMS found 511.3 |
| 179 | ((S)-2- ((dimethylamino)methyl) azetidin-1-yl)(4- fluoro-6-(5-fluoro-2- (((3S,4R)-3- hydroxytetrahydro-2H- pyran-4- yl)amino)pyrimidin-4- yl)-1-isopropyl-1H- indol-3-yl)methanone | 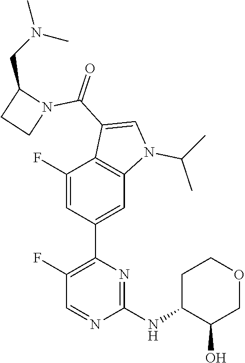 [SMILES: CC(C)n1cc(C(=O)N2CC[C@H]2CN(C)C)c2c(F)cc(-c3nc(N[C@@H]4CCOC[C@H]4O)ncc3F)cc21] | LCMS found 529.3 |
| 180 | ((S)-2-(2,2- difluoroethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 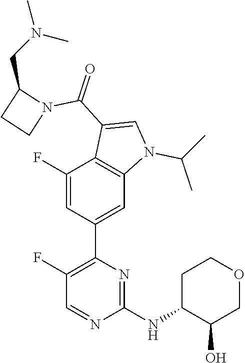 | LCMS found 536.4 |
| 181 | (1,1-difluoro-5- azaspiro[2.4]heptan-5- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 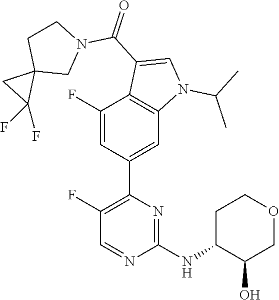 [SMILES: CC(C)n1cc(C(=O)N2CCC3(C2)CC3(F)F)c2c(F)cc(-c3nc(N[C@@H]4CCOC[C@H]4O)ncc3F)cc21] | LCMS found 548.5 |
| 182 | (5,5-difluoro-2- azaspiro[3.3 ]heptan-2- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 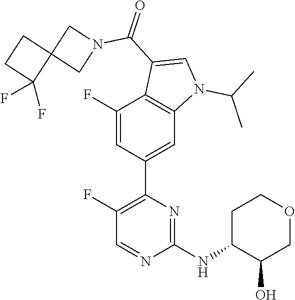 [SMILES: CC(C)n1cc(C(=O)N2CC3(CCC3(F)F)C2)c2c(F)cc(-c3nc(N[C@@H]4CCOC[C@H]4O)ncc3F)cc21] | LCMS found 548.5 |
| 183 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((S)-2- (hydroxymethyl)-2- methylazetidin-1- yl)methanone | 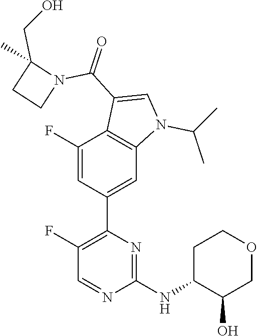 [SMILES: CC(C)n1cc(C(=O)N2CC[C@@]2(C)CO)c2c(F)cc(-c3nc(N[C@@H]4CCOC[C@H]4O)ncc3F)cc21] | LCMS found 516.3 |
| 184 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((2S,3R)-2- (hydroxymethyl)-3- methylazetidin-1- yl)methanone | 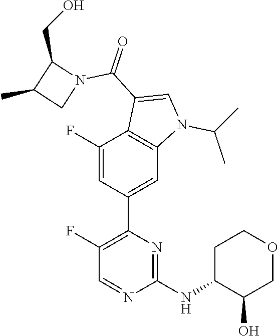 [SMILES: CC(C)n1cc(C(=O)N2C[C@@H](C)[C@H]2CO)c2c(F)cc(-c3nc(N[C@@H]4CCOC[C@H]4O)ncc3F)cc21] | LCMS found 516.3 |
| 185 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((S)-2-(2- hydroxyethyl)azetidin- 1-yl)methanone | 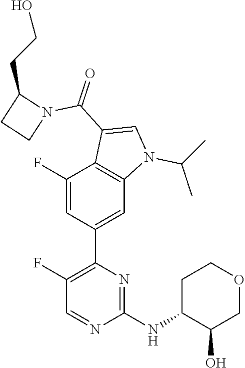 [SMILES: CC(C)n1cc(C(=O)N2CC[C@H]2CCO)c2c(F)cc(-c3nc(N[C@@H]4CCOC[C@H]4O)ncc3F)cc21] | LCMS found 516.3 |
| 186 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((S)-2-(2- hydroxypropan-2- yl)azetidin-1- yl)methanone | 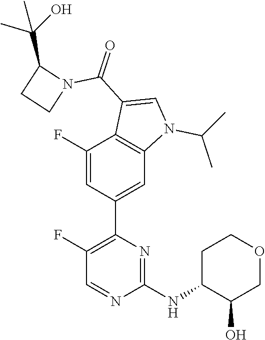 [SMILES: CC(C)n1cc(C(=O)N2CC[C@H]2C(C)(C)O)c2c(F)cc(-c3nc(N[C@@H]4CCOC[C@H]4O)ncc3F)cc21] | LCMS found 530.2 |
| 187 | (4-fluoro-6-(5-fluoro-2- (((3S,4R)-3- hydroxytetrahydro-2H- pyran-4- yl)amino)pyrimidin-4- yl)-1-isopropyl-1H- indol-3-yl)((2S,3S)-3- methyl-2- ((methylsulfonyl)methyl) azetidin-1- yl)methanone | 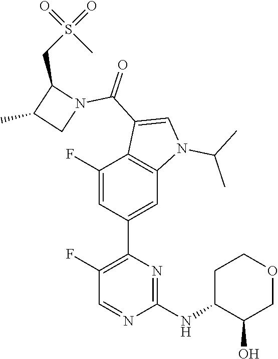 [SMILES: CC(C)n1cc(C(=O)N2C[C@H](C)[C@H]2CS(C)(=O)=O)c2c(F)cc(-c3nc(N[C@@H]4CCOC[C@H]4O)ncc3F)cc21] | LCMS found 578.2 |
| 188 | ((S)-2- ((cyclopropylsulfonyl) methyl)azetidin-1- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 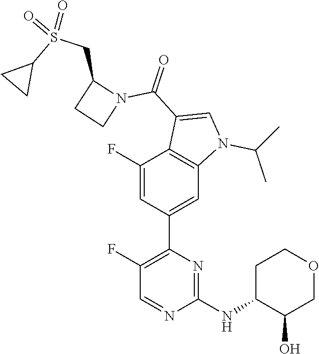 [SMILES: CC(C)n1cc(C(=O)N2CC[C@H]2CS(=O)(=O)C2CC2)c2c(F)cc(-c3nc(N[C@@H]4CCOC[C@H]4O)ncc3F)cc21] | LCMS found 590.3 |
| 189 | (1,1-difluoro-5- azaspiro[2.3 ]hexan-5- yl)(6-(5-fluoro-2- (((3S,4R)-3- hydroxytetrahydro-2H- pyran-4- yl)amino)pyrimidin-4- yl)-1-methyl-1H-indol- 3-yl)methanone | 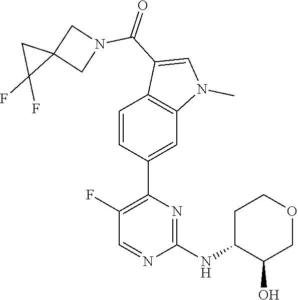 [SMILES: Cn1cc(C(=O)N2CC3(C2)CC3(F)F)c2ccc(-c3nc(N[C@@H]4CCOC[C@H]4O)ncc3F)cc21] | LCMS found 488.2 |
| 190 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3- (pyrrolidin-1- yl)azetidin-1- yl)methanone | 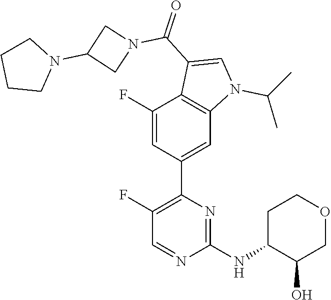 [SMILES: CC(C)n1cc(C(=O)N2CC(N3CCCC3)C2)c2c(F)cc(-c3nc(N[C@@H]4CCOC[C@H]4O)ncc3F)cc21] | LCMS found 541.2 |
| 191 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(6-methyl- 2,6- diazaspiro[3.3]heptan- 2-yl)methanone | 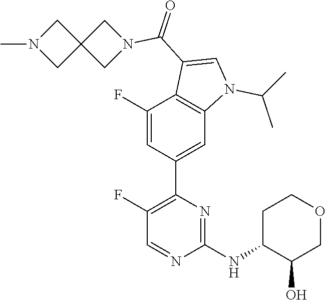 [SMILES: CC(C)n1cc(C(=O)N2CC3(CN(C)C3)C2)c2c(F)cc(-c3nc(N[C@@H]4CCOC[C@H]4O)ncc3F)cc21] | LCMS found 527.3 |
| 192 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(6-oxa-2- azaspiro[3.4]octan-2- yl)methanone | 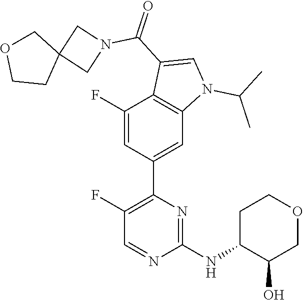 [SMILES: CC(C)n1cc(C(=O)N2CC3(CCOC3)C2)c2c(F)cc(-c3nc(N[C@@H]4CCOC[C@H]4O)ncc3F)cc21] | LCMS found 528.3 |
| 193 | (3- (Dimethylamino)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 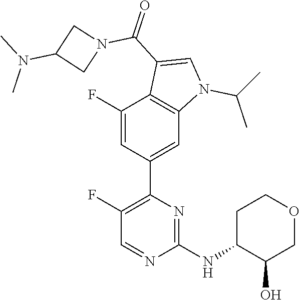 [SMILES: CC(C)n1cc(C(=O)N2CC(N(C)C)C2)c2c(F)cc(-c3nc(N[C@@H]4CCOC[C@H]4O)ncc3F)cc21] | LCMS found 515.3 |
| 194 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((S)-2- (fluoromethyl)azetidin- 1-yl)methanon | 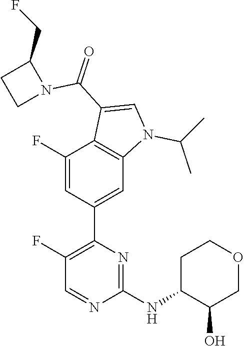 [SMILES: CC(C)n1cc(C(=O)N2CC[C@H]2CF)c2c(F)cc(-c3nc(N[C@@H]4CCOC[C@H]4O)ncc3F)cc21] | LCMS found 504.3 |
| 195 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3- hydroxyazetidin-1- yl)methanon | 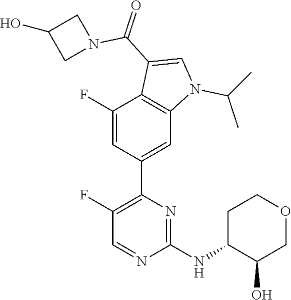 [SMILES: CC(C)n1cc(C(=O)N2CC(O)C2)c2c(F)cc(-c3nc(N[C@@H]4CCOC[C@H]4O)ncc3F)cc21] | LCMS found 488.3 |
| 196 | (3- (Difluoromethoxy)azeti- din-1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 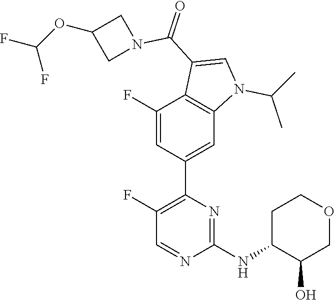 [SMILES: CC(C)n1cc(C(=O)N2CC(OC(F)F)C2)c2c(F)cc(-c3nc(N[C@@H]4CCOC[C@H]4O)ncc3F)cc21] | LCMS found 538.2 |
| 197 | (4-Fluoro-6-(5-fluoro- 2-(((3R,4R)-3- hydroxytetrahydro-2H- pyran-4- yl)amino)pyrimidin-4- yl)-1-isopropyl-1H- indol-3-yl)(3- (pyrrolidin-1- yl)azetidin-1- yl)methanone | 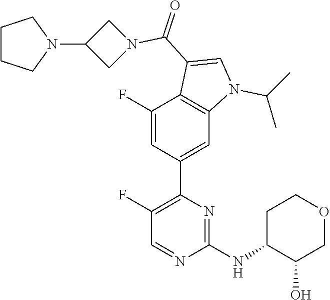 [SMILES: CC(C)n1cc(C(=O)N2CC(N3CCCC3)C2)c2c(F)cc(-c3nc(N[C@@H]4CCOC[C@@H]4O)ncc3F)cc21] | LCMS found 541.3 |
| 198 | Azetidin-1-yl(4-fluoro- 6-(5-fluoro-2- (((3S,4R)-3- hydroxytetrahydro-2H- pyran-4- yl)amino)pyrimidin-4- yl)-1-isopropyl-1H- indol-3-yl)methanone | 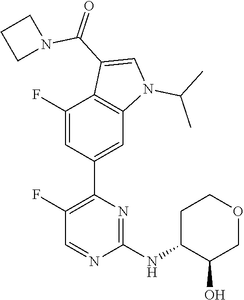 [SMILES: CC(C)n1cc(C(=O)N2CCC2)c2c(F)cc(-c3nc(N[C@@H]4CCOC[C@H]4O)ncc3F)cc21] | LCMS found 472.2 |
| 199 | (3-Aminoazetidin-1- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 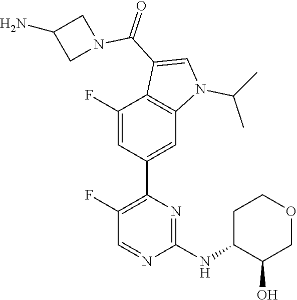 [SMILES: CC(C)n1cc(C(=O)N2CC(N)C2)c2c(F)cc(-c3nc(N[C@@H]4CCOC[C@H]4O)ncc3F)cc21] | LCMS found 489.2 |
| 200 | (3- (Difluoromethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 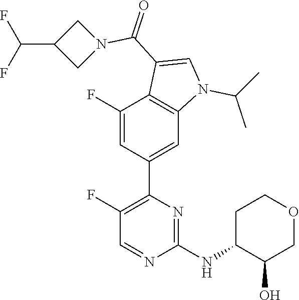 [SMILES: CC(C)n1cc(C(=O)N2CC(C(F)F)C2)c2c(F)cc(-c3nc(N[C@@H]4CCOC[C@H]4O)ncc3F)cc21] | LCMS found 522.2 |
| 201 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3- fluoroazetidin-1- yl)methanone | 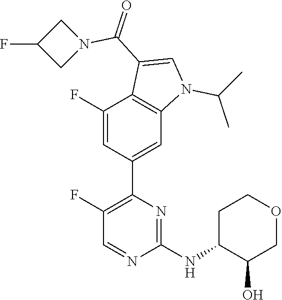 [SMILES: CC(C)n1cc(C(=O)N2CC(F)C2)c2c(F)cc(-c3nc(N[C@@H]4CCOC[C@H]4O)ncc3F)cc21] | LCMS found 490.2 |
| 202 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3- methylazetidin-1- yl)methanone | 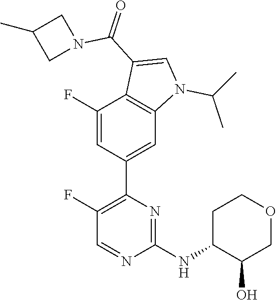 [SMILES: CC1CN(C(=O)c2cn(C(C)C)c3cc(-c4nc(N[C@@H]5CCOC[C@H]5O)ncc4F)cc(F)c23)C1] | LCMS found 486.2 |
| 203 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((R)-2- methylazetidin-1- yl)methanone | 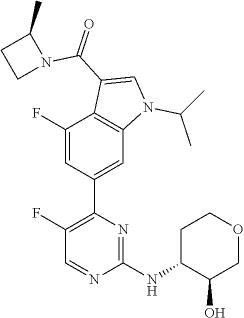 [SMILES: CC(C)n1cc(C(=O)N2CC[C@H]2C)c2c(F)cc(-c3nc(N[C@@H]4CCOC[C@H]4O)ncc3F)cc21] | LCMS found 486.2 |
| 204 | ((2R,4R)-2,4- Dimethylazetidin-1- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 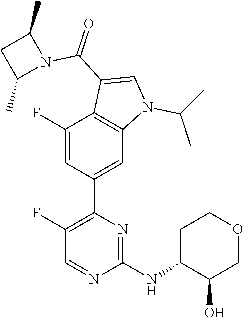 [SMILES: CC(C)n1cc(C(=O)N2[C@H](C)C[C@H]2C)c2c(F)cc(-c3nc(N[C@@H]4CCOC[C@H]4O)ncc3F)cc21] | LCMS found 500.2 |
| 205 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(5- azaspiro[2.3]hexan-5- yl)methanone | 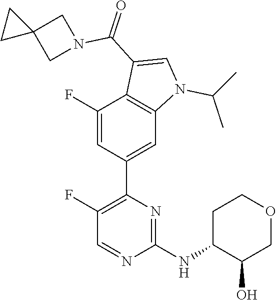 [SMILES: CC(C)n1cc(C(=O)N2CC3(CC3)C2)c2c(F)cc(-c3nc(N[C@@H]4CCOC[C@H]4O)ncc3F)cc21] | LCMS found 498.2 |
| 206 | 1-(4-Fluoro-6-(5- fluoro-2-(((3S,4R)-3- hydroxytetrahydro-2H- pyran-4- yl)amino)pyrimidin-4- yl)-1-isopropyl-1H- indole-3- carbonyl)azetidine-3- carbonitrile | 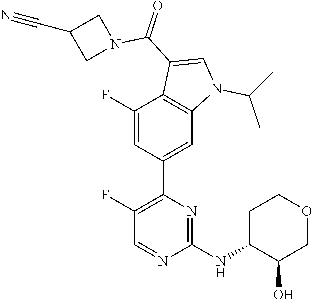 [SMILES: CC(C)n1cc(C(=O)N2CC(C#N)C2)c2c(F)cc(-c3nc(N[C@@H]4CCOC[C@H]4O)ncc3F)cc21] | LCMS found 497.2 |
| 207 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(pyrrolidin- 1-yl)methanone | 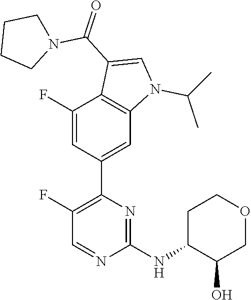 [SMILES: CC(C)n1cc(C(=O)N2CCCC2)c2c(F)cc(-c3nc(N[C@@H]4CCOC[C@H]4O)ncc3F)cc21] | LCMS found 486.2 |
| 208 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((R)-3- fluoropyrrolidin-1- yl)methanone | 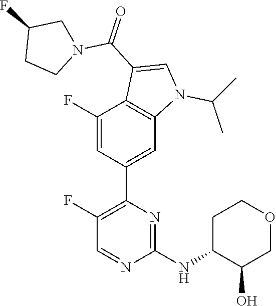 [SMILES: CC(C)n1cc(C(=O)N2CC[C@@H](F)C2)c2c(F)cc(-c3nc(N[C@@H]4CCOC[C@H]4O)ncc3F)cc21] | LCMS found 504.2 |
| 209 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((R)-3- hydroxypyrrolidin-1- yl)methanone | 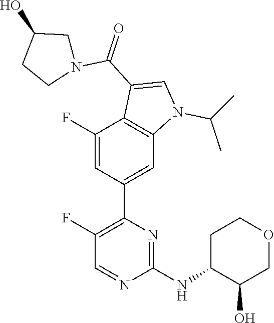 [SMILES: CC(C)n1cc(C(=O)N2CC[C@@H](O)C2)c2c(F)cc(-c3nc(N[C@@H]4CCOC[C@H]4O)ncc3F)cc21] | LCMS found 502.2 |
| 210 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((S)-3- hydroxypyrrolidin-1- yl)methanon | 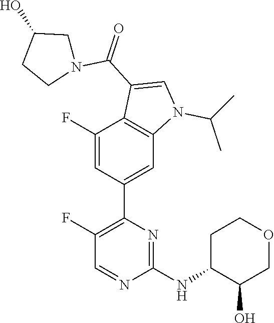 [SMILES: CC(C)n1cc(C(=O)N2CC[C@H](O)C2)c2c(F)cc(-c3nc(N[C@@H]4CCOC[C@H]4O)ncc3F)cc21] | LCMS found 502.2 |
| 211 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3-hydroxy- 3- (trifluoromethyl)azetidin- 1-yl)methanone | 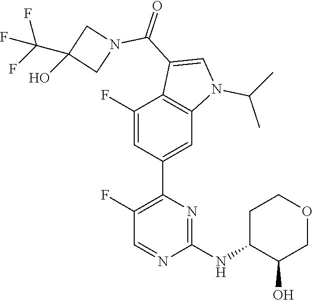 [SMILES: CC(C)n1cc(C(=O)N2CC(O)(C(F)(F)F)C2)c2c(F)cc(-c3nc(N[C@@H]4CCOC[C@H]4O)ncc3F)cc21] | LCMS found 556.2 |
| 212 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((S)-2- (hydroxymethyl)pyrroli- din-1-yl)methanone | 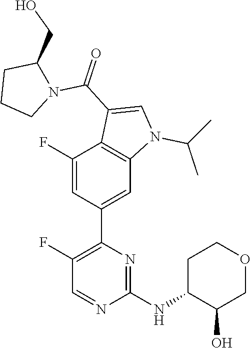 [SMILES: CC(C)n1cc(C(=O)N2CCC[C@H]2CO)c2c(F)cc(-c3nc(N[C@@H]4CCOC[C@H]4O)ncc3F)cc21] | LCMS found 516.2 |
| 213 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((R)-2- (hydroxymethyl)pyrroli- din-1-yl)methanone | 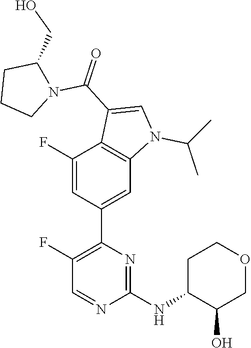 [SMILES: CC(C)n1cc(C(=O)N2CCC[C@@H]2CO)c2c(F)cc(-c3nc(N[C@@H]4CCOC[C@H]4O)ncc3F)cc21] | LCMS found 516.2 |
| 214 | (3,3- Difluoropyrrolidin-1- yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanon | 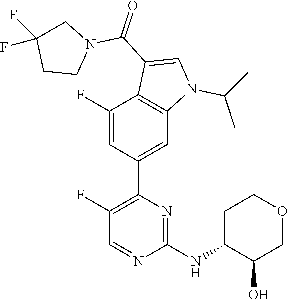 [SMILES: CC(C)n1cc(C(=O)N2CCC(F)(F)C2)c2c(F)cc(-c3nc(N[C@@H]4CCOC[C@H]4O)ncc3F)cc21] | LCMS found 522.2 |
| 215 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)((1R,2S,5S)- 2-(hydroxymethyl)-3- azabicyclo[3.1.0]hexan- 3-yl)methanone | 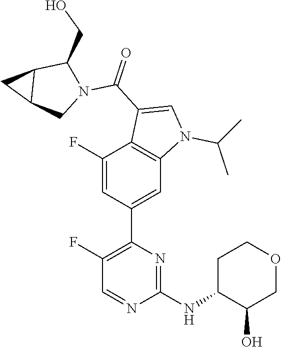 [SMILES: CC(C)n1cc(C(=O)N2C[C@H]3C[C@H]3[C@H]2CO)c2c(F)cc(-c3nc(N[C@@H]4CCOC[C@H]4O)ncc3F)cc21] | LCMS found 528.2 |
| 216 | (S)-1-(4-Fluoro-6-(5- fluoro-2-(((3S,4R)-3- hydroxytetrahydro-2H- pyran-4- yl)amino)pyrimidin-4- yl)-1-isopropyl-1H- indole-3- carbonyl)pyrrolidine-3- carbonitrile | 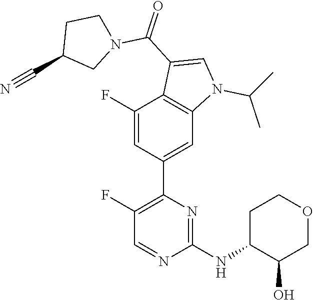 [SMILES: CC(C)n1cc(C(=O)N2CC[C@H](C#N)C2)c2c(F)cc(-c3nc(N[C@@H]4CCOC[C@H]4O)ncc3F)cc21] | LCMS found 511.2 |
| 217 | (R)-1-(4-Fluoro-6-(5- fluoro-2-(((3S,4R)-3- hydroxytetrahydro-2H- pyran-4- yl)amino)pyrimidin-4- yl)-1-isopropyl-1H- indole-3- carbonyl)pyrrolidine-3- carbonitrile | 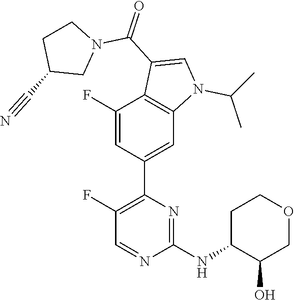 [SMILES: CC(C)n1cc(C(=O)N2CC[C@@H](C#N)C2)c2c(F)cc(-c3nc(N[C@@H]4CCOC[C@H]4O)ncc3F)cc21] | LCMS found 511.2 |
| 218 | (3-Fluoro-3- (trifluoromethyl)azetidin- 1-yl)(4-fluoro-6-(5- fluoro-2-(((3S,4R)-3- hydroxytetrahydro-2H- pyran-4- yl)amino)pyrimidin-4- yl)-1-isopropyl-1H- indol-3-yl)methanone | 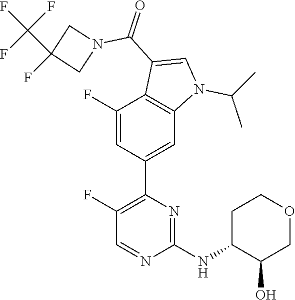 [SMILES: CC(C)n1cc(C(=O)N2CC(F)(C(F)(F)F)C2)c2c(F)cc(-c3nc(N[C@@H]4CCOC[C@H]4O)ncc3F)cc21] | LCMS found 558.2 |
| 219 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(5- azaspiro[2.4]heptan-5- yl)methanone | 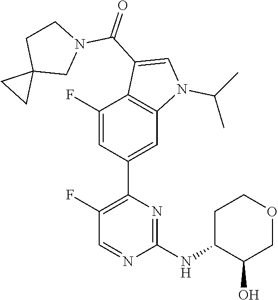 [SMILES: CC(C)n1cc(C(=O)N2CCC3(CC3)C2)c2c(F)cc(-c3nc(N[C@@H]4CCOC[C@H]4O)ncc3F)cc21] | LCMS found 512.3 |
| 220 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3-methyl-3- (trifluoromethyl)azetidin- 1-yl)methanone | 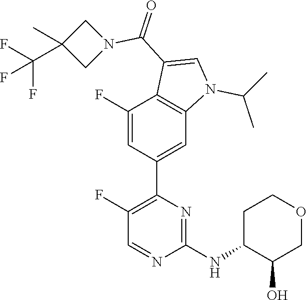 [SMILES: CC(C)n1cc(C(=O)N2CC(C)(C(F)(F)F)C2)c2c(F)cc(-c3nc(N[C@@H]4CCOC[C@H]4O)ncc3F)cc21] | LCMS found 554.2 |
| 221 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3- isopropoxyazetidin-1- yl)methanone | 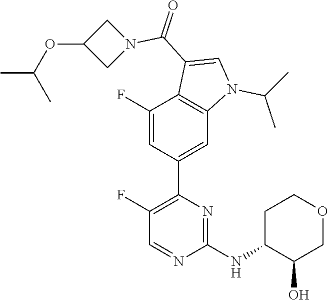 [SMILES: CC(C)OC1CN(C(=O)c2cn(C(C)C)c3cc(-c4nc(N[C@@H]5CCOC[C@H]5O)ncc4F)cc(F)c23)C1] | LCMS found 530.3 |
| 222 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3- (hydroxymethyl)azetidin- 1-yl)methanone | 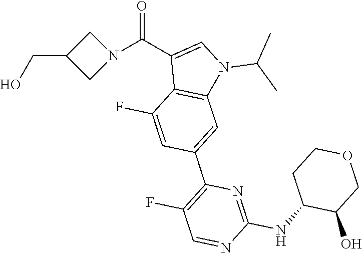 [SMILES: CC(C)n1cc(C(=O)N2CC(CO)C2)c2c(F)cc(-c3nc(N[C@@H]4CCOC[C@H]4O)ncc3F)cc21] | LCMS found 502.2 |
| 223 | (3- ((Difluoromethoxy)meth- yl)azetidin-1-yl)(4- fluoro-6-(5-fluoro-2- (((3S,4R)-3- hydroxytetrahydro-2H- pyran-4- yl)amino)pyrimidin-4- yl)-1-isopropyl-1H- indol-3-yl)methanone | 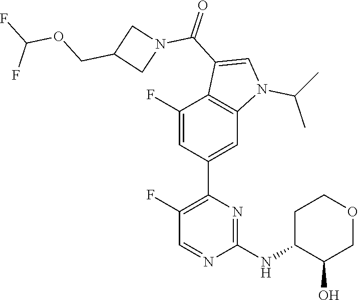 [SMILES: CC(C)n1cc(C(=O)N2CC(COC(F)F)C2)c2c(F)cc(-c3nc(N[C@@H]4CCOC[C@H]4O)ncc3F)cc21] | LCMS found 552.2 |
| 224 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3-(2- (trifluoromethoxy)ethyl) azetidin-1- yl)methanone | 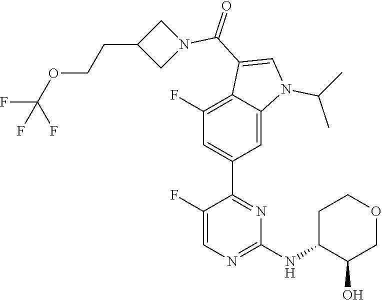 [SMILES: CC(C)n1cc(C(=O)N2CC(CCOC(F)(F)F)C2)c2c(F)cc(-c3nc(N[C@@H]4CCOC[C@H]4O)ncc3F)cc21] | LCMS found 584.2 |
| 225 | (1-Ethyl-4-fluoro-6-(5- fluoro-2-(((3S,4R)-3- hydroxytetrahydro-2H- pyran-4- yl)amino)pyrimidin-4- yl)-1H-indol-3-yl)((S)- 2- (hydroxymethyl)azetidin- 1-yl)methanone | 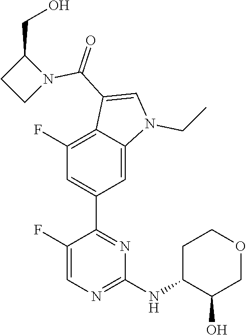 [SMILES: CCn1cc(C(=O)N2CC[C@H]2CO)c2c(F)cc(-c3nc(N[C@@H]4CCOC[C@H]4O)ncc3F)cc21] | LCMS found 488.2 |
| 226 | (4-Fluoro-6-(5-fluoro- 2-(((35,4R)-3- hydroxytetrahydro-2H- pyran-4- yl)amino)pyrimidin-4- yl)-1-isopropyl-1H- indol-3-yl)(3- (trifluoromethoxy)azeti- din-1-yl)methanone | 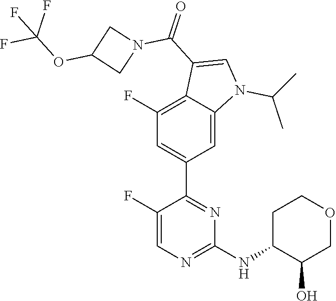 [SMILES: CC(C)n1cc(C(=O)N2CC(OC(F)(F)F)C2)c2c(F)cc(-c3nc(N[C@@H]4CCOC[C@H]4O)ncc3F)cc21] | LCMS found 556.2 |
| 227 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-yl)(3- ((trifluoromethoxy)meth- yl)azetidin-1- yl)methanone | 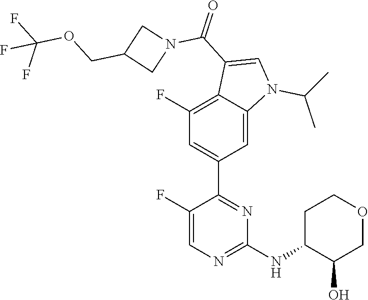 [SMILES: CC(C)n1cc(C(=O)N2CC(COC(F)(F)F)C2)c2c(F)cc(-c3nc(N[C@@H]4CCOC[C@H]4O)ncc3F)cc21] | LCMS found 570.2 |
| 228 | (3- (Difluoromethoxy)azeti- din-1-yl)(4-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 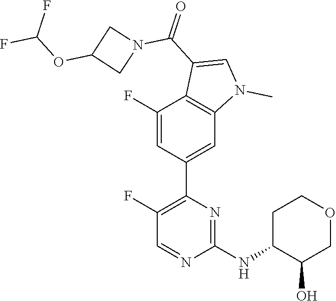 [SMILES: Cn1cc(C(=O)N2CC(OC(F)F)C2)c2c(F)cc(-c3nc(N[C@@H]4CCOC[C@H]4O)ncc3F)cc21] | LCMS found 510.2 |
| 229 | (4-Fluoro-6-(5-fluoro- 2-(((3S,4R)-3- hydroxytetrahydro-2H- pyran-4- yl)amino)pyrimidin-4- yl)-1-isopropyl-1H- indol-3-y1)((S)-2-(1- hydroxycyclopropyl)aze- tidin-1-yl)methanone | 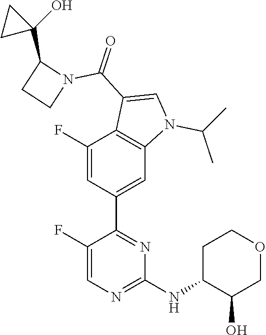 [SMILES: CC(C)n1cc(C(=O)N2CC[C@H]2C2(O)CC2)c2c(F)cc(-c3nc(N[C@@H]4CCOC[C@H]4O)ncc3F)cc21] | LCMS found 528.1 |

TABLE 9
The compounds in Table 9 were prepared in accordance with the synthetic protocols set
forth in Examples 7 using the appropriate starting materials.

| | | | Analytical |
|---|---|---|---|
| Ex. | Name | Structure | data |
| 230 | (4,7-Difluoro-6-(5- fluoro-2-(((3S,4R)-3- hydroxytetrahydro-2H- pyran-4- yl)amino)pyrimidin-4- yl)-1-methyl-1H-indol- 3-yl)(1,1-difluoro-5- azaspiro[2.3]hexan-5- yl)methanone | 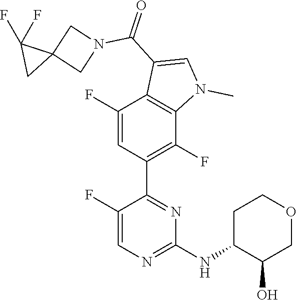 [SMILES: Cn1cc(C(=O)N2CC3(C2)CC3(F)F)c2c(F)cc(-c3nc(N[C@@H]4CCOC[C@H]4O)ncc3F)c(F)c21] | LCMS found 524.2 |
| 231 | (1,1-Difluoro-5- azaspiro[2.3]hexan-5- yl)(7-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 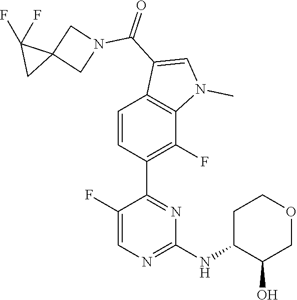 [SMILES: Cn1cc(C(=O)N2CC3(C2)CC3(F)F)c2ccc(-c3nc(N[C@@H]4CCOC[C@H]4O)ncc3F)c(F)c21] | LCMS found 506.1 |
| 232 | (7-Fluoro-6-(5-fluoro- 2-(((3S,4R)-3- hydroxytetrahydro-2H- pyran-4- yl)amino)pyrimidin-4- yl)-1-methyl-1H-indol- 3-yl)((2S,3S)-2- (hydroxymethyl-d₂)-3- methylazetidin-1- yl)methanone | 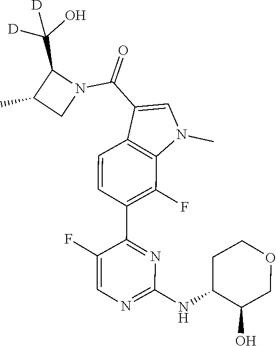 [SMILES: [2H]C([2H])(O)[C@@H]1[C@@H](C)CN1C(=O)c1cn(C)c2c(F)c(-c3nc(N[C@@H]4CCOC[C@H]4O)ncc3F)ccc12] | LCMS found 490.2 |
| 233 | (4-Fluoro-6-(5-fluoro- 2-(((3S,4R)-3- hydroxytetrahydro-2H- pyran-4- yl)amino)pyrimidin-4- yl)-1,7-dimethyl-1H- indol-3-yl)((2S,3S)-2- (hydroxymethyl-d₂)-3- methylazetidin-1- yl)methanone | 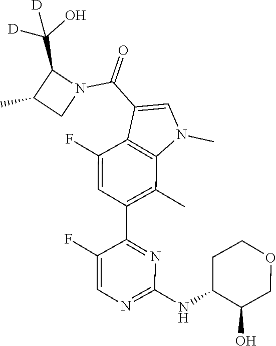 [SMILES: [2H]C([2H])(O)[C@@H]1[C@@H](C)CN1C(=O)c1cn(C)c2c(C)c(-c3nc(N[C@@H]4CCOC[C@H]4O)ncc3F)cc(F)c12] | LCMS found 504.2 |
| 234 | (1,1-Difluoro-5- azaspiro[2.3]hexan-5- yl)(6-(5-fluoro-2- (((3S,4R)-3- hydroxytetrahydro-2H- pyran-4- yl)amino)pyrimidin-4- yl)-1,7-dimethyl-1H- indol-3-yl)methanone | 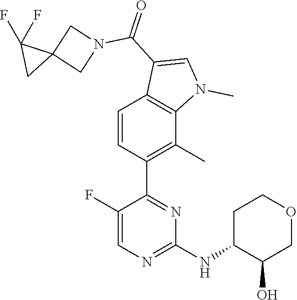 [SMILES: Cc1c(-c2nc(N[C@@H]3CCOC[C@H]3O)ncc2F)ccc2c(C(=O)N3CC4(C3)CC4(F)F)cn(C)c12] | LCMS found 502.5 |
| 235 | (4,5-Difluoro-6-(5- fluoro-2-(((3S,4R)-3- hydroxytetrahydro-2H- pyran-4- yl)amino)pyrimidin-4- yl)-1-methyl-1H-indol- 3-yl)(1,1-difluoro-5- azaspiro[2.3]hexan-5- yl)methanone | 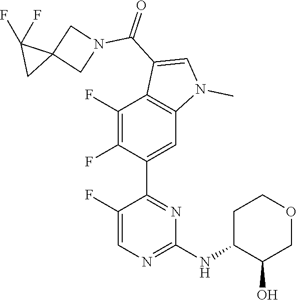 [SMILES: Cn1cc(C(=O)N2CC3(C2)CC3(F)F)c2c(F)c(F)c(-c3nc(N[C@@H]4CCOC[C@H]4O)ncc3F)cc21] | LCMS found 524.2 |
| 236 | (1,1-Difluoro-5- azaspiro[2.3]hexan-5- yl)(5-fluoro-6-(5- fluoro-2-(((3S,4R)-3- hydroxytetrahydro-2H- pyran-4- yl)amino)pyrimidin-4- yl)-1-methyl-1H-indol- 3-yl)methanone | 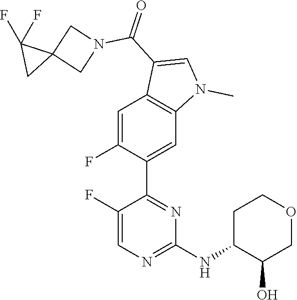 [SMILES: Cn1cc(C(=O)N2CC3(C2)CC3(F)F)c2cc(F)c(-c3nc(N[C@@H]4CCOC[C@H]4O)ncc3F)cc21] | LCMS found 506.1 |
| 237 | (5-Fluoro-6-(5-fluoro- 2-(((3S,4R)-3- hydroxytetrahydro-2H- pyran-4- yl)amino)pyrimidin-4- yl)-1-methyl-1H-indol- 3-yl)((2S,3S)-2- (hydroxymethyl-d₂)-3- methylazetidin-1- yl)methanone | 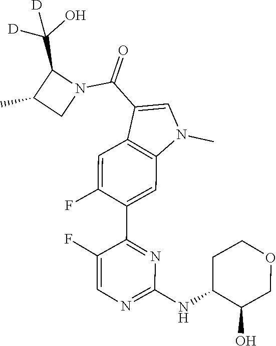 [SMILES: [2H]C([2H])(O)[C@@H]1[C@@H](C)CN1C(=O)c1cn(C)c2cc(-c3nc(N[C@@H]4CCOC[C@H]4O)ncc3F)c(F)cc12] | LCMS found 490.1 |
| 238 | (1,1-Difluoro-5- azaspiro[2.3]hexan-5- yl)(4-fluoro-6-(5- fluoro-2-(((3S,4R)-3- hydroxytetrahydro-2H- pyran-4- yl)amino)pyrimidin-4- yl)-5-methoxy-1- methyl-1H-indol-3- yl)methanone | 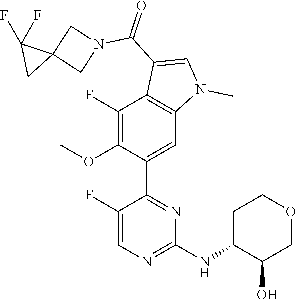 [SMILES: COc1c(-c2nc(N[C@@H]3CCOC[C@H]3O)ncc2F)cc2c(c(C(=O)N3CC4(C3)CC4(F)F)cn2C)c1F] | LCMS found 536.1 |

### Example A. CDK/Cyclin HTRF Enzyme Activity Assay

CDK/Cyclin enzyme activity assays utilize N-terminally tagged (GST, HIS, or Flag) full-length human CDK and Cyclin proteins co-expressed in a baculovirus expression system (CDK4/Cyclin D1, ProQinase 0142-0143-1; CDK6/Cyclin D3, ProQinase 0051-0373-1; CDK2/Cyclin E1, Carna Biosciences 04-165-500; CDK1/Cycin B1, Carna Biosciences 04-102-100; CDK9/Cyclin T1, Carna Biosciences 04-110-100; CDK7/Cyclin H/MAT1, Millipore-Sigma 14-476).

Assays were conducted in white 384-well polystyrene plates in a final reaction volume of 8 μL. CDK/Cyclin was pre-incubated with the compounds of the Examples (10 or 40 nL serially diluted in DMSO) in 4 μL assay buffer (containing 50 mM HEPES pH 7.5, I mM EGTA, 10 mM MgCl, 2 mM DTT, 0.05 mg/mL BSA, and 0.01% Tween 20) for 30 minutes at room temperature, after which reactions were started by the addition of 4 μL ATP and U-light peptide ((eIF4E-Binding Protein, Revvity 0128, or Myelin Basic Protein, Revvity 0109), for a final concentration of 1 mM and 50 nM, respectively. The reactions were stopped after 60 minutes by the addition of EDTA and Europium-labeled anti-phospho-peptide antibody, for a final concentration of 15 mM and 1 nM, respectively. TR-FRET signals were read after 15 minutes at room temperature on a PheraSTAR FSX plate reader (BMG Labtech). Data was analyzed with Genedata Screener software using a three or four parameter dose response curve to determine IC₅₀ for each compound.

Compounds of the present disclosure, as exemplified in Examples 1-17, showed IC₅₀ values in the following ranges: +=IC₅₀≤10 nM; ++=10 nM<IC₅₀ 100 nM; +++=100 nM<IC₅₀≤1000 nM; ++++=IC₅₀>1000 nM;

Data obtained for the Example compounds using the CDK4/Cyclin D1 homogenous time-resolved fluorescence (HTRF) enzyme activity assay described in Example A is provided in Table A.

TABLE A

| Example | CDK4/Cyclin D1 IC₅₀ (nM) |
|---|---|
| 1 | + |
| 2 | + |
| 3 | + |
| 4 | + |
| 5 | + |
| 6 | ++ |
| 7 | + |
| 8 | + |
| 9 | + |
| 10 | + |
| 11 | + |
| 12 | + |
| 13 | + |
| 14 | ++ |
| 15 | ++ |
| 16 | + |
| 17 | + |
| 18 | + |
| 19 | + |
| 20 | + |
| 21 | + |
| 22 | + |
| 23 | + |
| 24 | + |
| 25 | + |
| 26 | + |
| 27 | + |
| 28 | + |
| 29 | + |
| 30 | + |
| 31 | + |
| 32 | + |
| 33 | + |
| 34 | + |
| 35 | + |
| 36 | + |
| 37 | + |
| 38 | + |
| 39 | + |
| 40 | + |
| 41 | + |
| 42 | + |
| 43 | + |
| 44 | + |
| 45 | + |
| 46 | + |
| 47 | + |
| 48 | + |
| 49 | + |
| 50 | + |
| 51 | + |
| 52 | ++ |
| 53 | + |
| 54 | + |
| 55 | + |
| 56 | + |
| 57 | + |
| 58 | + |
| 59 | ++ |
| 60 | + |
| 61 | ++ |
| 62 | ++ |
| 63 | + |
| 64 | + |
| 65 | +++ |
| 66 | + |
| 67 | + |
| 68 | + |
| 69 | + |
| 70 | + |
| 71 | + |
| 72 | + |
| 73 | + |
| 74 | + |
| 75 | + |
| 76 | + |
| 77 | + |
| 78 | + |
| 79 | + |
| 80 | + |
| 81 | + |
| 82 | + |
| 83 | + |
| 84 | + |
| 85 | + |
| 86 | + |
| 87 | + |
| 88 | + |
| 89 | + |
| 90 | + |
| 91 | + |
| 92 | + |
| 93 | + |
| 94 | + |
| 95 | + |
| 96 | + |
| 97 | + |
| 98 | ++ |
| 99 | + |
| 100 | + |
| 101 | ++ |
| 102 | + |
| 103 | ++ |
| 104 | + |
| 105 | ++ |
| 106 | + |
| 107 | + |
| 108 | + |
| 109 | + |
| 110 | + |
| 111 | + |
| 112 | + |
| 113 | + |
| 114 | + |
| 115 | + |
| 116 | + |
| 117 | + |
| 118 | + |
| 119 | + |
| 120 | + |
| 121 | + |
| 122 | + |
| 123 | + |
| 124 | + |
| 125 | + |
| 126 | + |
| 127 | ++ |
| 128 | + |
| 129 | + |
| 130 | + |
| 131 | + |
| 132 | + |
| 133 | + |
| 134 | + |
| 135 | + |
| 136 | + |
| 137 | + |
| 138 | + |
| 139 | + |
| 140 | + |
| 141 | + |
| 142 | + |
| 143 | + |
| 144 | + |
| 145 | ++ |
| 146 | + |
| 147 | ++ |
| 148 | + |
| 149 | + |
| 150 | + |
| 151 | ++ |
| 152 | ++ |
| 153 | + |
| 154 | + |
| 155 | ++ |
| 156 | ++ |
| 157 | +++ |
| 158 | + |
| 159 | +++ |
| 160 | + |
| 161 | + |
| 162 | ++ |
| 163 | + |
| 164 | + |
| 165 | ++ |
| 166 | + |
| 167 | ++ |
| 168 | + |
| 169 | ++ |
| 170 | + |
| 171 | + |
| 172 | ++ |
| 173 | + |
| 174 | + |
| 175 | ++ |
| 176 | + |
| 177 | ++ |
| 178 | + |
| 179 | +++ |
| 180 | + |
| 181 | + |
| 182 | + |
| 183 | ++ |
| 184 | + |
| 185 | ++ |
| 186 | ++ |
| 187 | + |
| 188 | + |
| 189 | + |
| 190 | + |
| 191 | +++ |
| 192 | ++ |
| 193 | + |
| 194 | + |
| 195 | + |
| 196 | ++ |
| 197 | ++ |
| 198 | + |
| 199 | + |
| 200 | + |
| 201 | + |
| 202 | + |
| 203 | + |
| 204 | + |
| 205 | + |
| 206 | ++ |
| 207 | + |
| 208 | + |
| 209 | ++ |
| 210 | ++ |
| 211 | + |
| 212 | + |
| 213 | +++ |
| 214 | ++ |
| 215 | ++ |
| 216 | + |
| 217 | + |
| 218 | ++ |
| 219 | + |
| 220 | + |
| 221 | ++ |
| 222 | ++ |
| 223 | + |
| 224 | ++ |
| 225 | + |
| 226 | ++ |
| 227 | + |
| 228 | + |
| 229 | ++ |
| 230 | + |
| 231 | + |
| 232 | + |
| 233 | ++ |
| 234 | ++ |
| 235 | + |
| 236 | + |
| 237 | + |
| 238 | ++ |

### Example B. CDK2 pRb S780 HTRF Assay in COV318 Cells

COV318 cells (Sigma-Aldrich, cat #07071903) are cultured in 5% FBS DMEM and plated in a 96-well flat-bottom plate (Corning-Costar, cat #3596) at 25,000 cells per well in 100 μL volume. At a starting concentration of 5 mM (final assay concentration 20 μM), compounds are 3-fold serially diluted in DMSO. 249 μL of 5% FBS DMEM media is added to 1 μL of compound, media is aspirated from cells, and 100 μL of diluted compound are added to the cell plate. The plates are then incubated overnight for 22 hours at 37° C. and 5% CO₂. The next day, the plates are washed to remove supernatant and 50 μL of 4× Revvity lysis buffer #2 from the phospho-Rb (Ser780) cellular assay kit (Revvity Rb phosphor-S780 kit, cat #64RBS780PEH) is diluted to 1× in distilled water and supplemented with blocking buffer (Revvity Rb phosphor-S780 kit, cat #64RBS780PEH) and protease inhibitor cocktail set III, EDTA-free (Calbiochem, cat #539134) and added to each well. The plate is then gently shaken at room temperature for 45 minutes to 1 hour until cell lysis was completed. Then, 4 μL of a 1:1 mixture of diluted donor fluorophore solution and diluted acceptor fluorophore solution (Revvity Rb phosphor-S780 kit, cat #64RBS780PEH) is added into a 384-well white plate (Greiner, cat #784075). 16 μL of cell lysate is transferred into each well for a final volume of 20 μL per well. After a 1 hour incubation at room temperature, the HTRF signal is measured by the PHERAstar microplate reader. Data analysis is performed in GeneData.

### Example C. CDK1 pNPM T199 In-Cell Western Assay

OVCAR3 cells (ATCC, cat #HTB-161) are seeded at 3×10⁵ cells/mL, or 30,000 cell per well, in 100 μL of 5% FBS RPMI medium overnight in 96-well plates (Corning, cat #07-201-96) at 37° C. with 5% CO₂. The next day, the medium is replaced with 100 μL per well of 5% FBS RPMI containing 20 ng/mL of nocodazole (Cell Signaling, cat #2190S) and incubated for 20 to 21 hours at 37° C. with 5% CO₂ to synchronize the cells in G2/M. After synchronization, the medium is removed. Then, 80 μL of the 3-fold serially diluted compound are added starting at 20 μM (final assay concentration 20 μM) and incubated at 37° C. with 5% CO₂ for another 2 hours. The medium is removed, and the cells are immediately fixed with freshly diluted 4% paraformaldehyde/PBS for 20 minutes at room temperature. The fixing solution is removed, and the cells are washed 3× with PBS containing 0.05% Tween-20 for 5 to 10 minutes per wash with gentle shaking. Next, Odyssey blocking buffer (Licor P/N: 927-60001) with 0.1% Triton X-100 is added at 40 μL per well with 0.1% Triton X-100, and the plates are rocked gently at room temperature for 1 hour. The blocking buffer is removed, and 40 μL per well of the primary antibody phosphor-NPM T199 (Cell Signaling, cat #3541S) is added at 1:400 dilution in Odyssey blocking buffer with 0.1% Triton X-100. The plate is then incubated overnight with gentle shaking at 4° C. The next day, the primary antibody is removed, and cells are washed 3× with 140 μL of PBS containing 0.05% Tween-20 for 5 minutes per wash with gentle shaking. The secondary antibody (IRDye® 800CW Goat anti-Rabbit) is then added at a 1:2000 dilution and CellTag 700 stain (Licor CellTaq 700 Stain ICW kit II, cat #P/N 926-41092) at a 1:600 dilution in Odyssey blocking buffer with 0.1% Triton X-100. Plates are rocked gently for 2 hours at room temperature in dark and washed 3× with 140 μL of PBS containing 0.05% Tween-20 for 5 minutes per wash with gentle shaking. During all wash steps, the plate is protected from light. After the final wash, the plate is scanned in both 700 and 800 nm channels on the Odyssey CLx. Data was analyzed with Genedata Screener software using a three or four parameter dose response curve to determine IC₅₀ for each compound.

### Example D. CDK4 pRb S780 HTRF Assay in JeKo-1 Cells

JeKo-1 cells (ATCC, cat #CRL-3006) cultured in 10% FBS RPMI are plated in a 384-well plate (Greiner, cat #784080) at 12,000 cells per well in 8 μL volume. Next, the compound is added at a starting final assay concentration of 10 μM and 3-fold serially diluted down to 0.17 nM in 4 μL for a final volume of 12 μL in each well. The plate is incubated for 24 hours at 37° C. with 5% CO₂. Next, 4 μL of 4× Revvity Lysis #2 (Revvity Rb phospho-S780 kit, cat #64RBS780PEH) supplemented with 4× blocking buffer (Revvity Rb phospho-S780 kit, cat #64RBS780PEH) is added to each well. The plate is gently rocked at room temperature for 30 to 45 minutes until cell lysis is completed. To each well of cell lysate, 4 μL of a 1:1 mixture of diluted donor fluorophore solution and diluted acceptor fluorophore solution (Revvity Rb phospho-S780 kit, cat #64RBS780PEH) is added to a final volume of 20 μL per well. The plate is incubated overnight at room temperature in the dark. The HTRF signal is measured by the PHERAstar microplate reader. Data analysis is performed in Genedata.

### Example E. CDK6 pRb S780 HTRF Assay in MV-4-11 Cells

MV-4-11 cells (ATCC, cat #CRL-9591) cultured in 10% FBS RPMI are plated in a 384-well plate (Greiner, cat #784080) at 12,000 cells per well in 8 μL volume. Next, the compound is added at a starting final assay concentration of 10 M and 3-fold serially diluted down to 0.17 nM in 4 μL for a final volume of 12 μL in each well. The plate is then incubated for 24 hours at 37° C. with 5% CO₂. Next, 4 μL of 4× Revvity Lysis #2 (Revvity Rb phospho-S780 kit, cat #64RBS780PEH) supplemented with 4× blocking buffer (Revvity Rb phospho-S780 kit, cat #64RBS780PEH) is added to each well. The plate is gently rocked at room temperature for 30 to 45 minutes until cell lysis was completed. To each well of cell lysate, 4 μL of a 1:1 mixture of diluted donor fluorophore solution and diluted acceptor fluorophore solution (Revvity Rb phospho-S780 kit, cat #64RBS780PEH) are added to a final volume of 20 μL. The plate was incubated overnight at room temperature in the dark. The HTRF signal is measured by the PHERAstar microplate reader. Data analysis is performed in Genedata.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula (I): 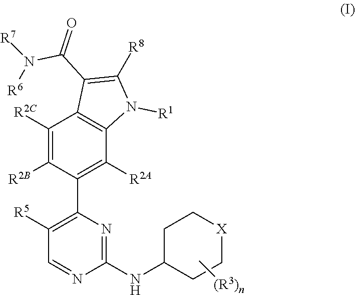
or a pharmaceutically acc b s hereof, wherein
n is 0, 1, 2, 3, 4, 5, or 6;
X is O, S, S(O)₂, S(O)NR^{c}, or N(-L-R⁴);
L is S(O)₂, S(O)(═NR^{b}), or S(O)₂NR^{a};
R^{a}, R^{b}, and R^{c} are each independently selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
R¹ is selected from C₁₋₆ alkyl, C₁₋₆ haloalkyl, C₃₋₁₀ cycloalkyl, 6-14 membered aryl, 4-15 membered heterocycloalkyl, 5-14 membered heteroaryl, C₃₋₁₀ cycloalkyl-C₁₋₄ alkyl, 6-14 membered aryl-C₁₋₄ alkyl, 4-15 membered heterocycloalkyl-C₁₋₄ alkyl, and 5-14 membered heteroaryl-C₁₋₄ alkyl, which are each optionally substituted with 1, 2, 3, or 4 independently selected R^{1A} substituents;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{a11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹) and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹¹ substituents;
each R^{a11}, R^{c11}, and R^{d11}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{C11} and R^{d11}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or, any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{e12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{e12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12} NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{e12} and R^{d12}, attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12}, is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or, any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{2A}, R^{2B}, and R^{2C} are each independently selected from H, D, halo, CN, C₁₋₆ alkyl, C₁₋₆ haloalkyl, C₃₋₇ cycloalkyl, phenyl, 4-8 membered heterocycloalkyl, 5-6 membered heteroaryl, C₃-7 cycloalkyl-C₁₋₄ alkyl, phenyl-C₁₋₄ alkyl, 4-8 membered heterocycloalkyl-C₁₋₄ alkyl, 5-6 membered heteroaryl-C₁₋₄ alkyl, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2} NR^{c2}NR^{c2}R^{d2}, NR^{c2}C(O)R^{b2}, NR^{c2}C(O)OR^{a2} NR^{c2}C(O)NR^{c2}R^{d2}, C(═NR^{e2})R^{b2}, C(═NR^{e2})R^{c2}R^{d2} NR^{c2}C(═NR^{e2})NR^{c2}R^{d2} NR^{c2}C(═NR^{e2})R^{b2} NR^{c2}S(O)NR^{c2}R^{d2} NR^{e2}S(O)R^{b2} NR^{c2}S(O)₂R^{b2}, NR^{c2}S(O)(═NR^{e2})R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a2}, R^{c2}, and R^{d2} is independently selected from H, C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c2} and R^{d2} attached to the same N atom, together with the N atom to which they are attached, form a 4-8 membered heterocycloalkyl group, which is optionally substituted with 1, 2, 3, or 4 independently selected R^{G} substituents;
each R^{b2} is independently selected from C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e2} is independently selected from H, OH, CN, C₁₋₆ alkyl, C₁₋₆ alkoxy, C₁₋₆ haloalkyl, and C₁₋₆ haloalkoxy;
each R³ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3} NR^{c3}NR^{c3}R^{d3} NR^{c3}C(O)R^{b3} NR^{c3}C(O)OR^{a3} NR^{c3}C(O)NR^{c3}R^{d3}, C(═NR^{e3})R^{b3}, C(═NR^{e3})NR^{c3}R^{d3} NR^{c3}C(═NR^{e3})NR^{c3}R^{d3} NR^{c3}C(═NR^{e3})R^{b3} NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, NHOR^{a41}, C(O)R^{b41}, C(O)NR^{c41}R^{d41}, C(O)NR^{c41}(OR^{a41}) C(O)OR^{a41}, OC(O)R^{b41}, OC(O)NR^{c41}R^{d41}, NR^{c41}R^{d41} NR^{c41}NR^{c41}R^{d41} NR^{c41}C(O)R^{b41} NR^{c41}C(O)OR^{a41} NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41} NR^{c41}C(═NR^{e41})R^{b41}, NR^{c41}S(O)NR^{c41}R^{d41}, NR^{c41}S(O)R^{b41} NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, and S(O)(═NR^{e41})R^{b41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{4B} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{e42}(OR^{a42}) C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)NR^{c42}R^{d42}, NR^{c42}S(O)R^{b42}, NR^{c42}S(O)₂R^{b42}, NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, 0S(O)(═NR^{e42})R^{b42}, OS(O)₂R^{b42}, and S(O)(═NR^{e42})R^{b42}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b42} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
R⁵ is selected from halo, CN, C₁₋₄ alkyl, C₁₋₄ haloalkyl, C₂₋₄ alkenyl, C₂₋₄ alkynyl, C₁₋₃ alkoxy, C₁₋₃ haloalkoxy, amino, C₁₋₃ alkylamino, di(C₁₋₃ alkyl)amino, cyano-C₁₋₄ alkyl, HO—C₁₋₄ alkyl, C₁₋₃ alkoxy-C₁₋₄ alkyl, and C₃₋₄ cycloalkyl;
R⁶ is selected from C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, 4, 5, or 6 independently selected R^{6A} substituents;
R⁷ is selected from H, C₁₋₆ alkyl, C₂₋₆ alkenyl, C₂₋₆ alkynyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, R⁶ and R⁷, together with the nitrogen atom to which they are attached, form a 4-15 membered heterocycloalkyl moiety or a 5-10 membered heteroaryl moiety, each of which is optionally substituted by 1, 2, 3, 4, 5, or 6 independently selected R^{6A} substituents;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61} NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R^{d61}, NR^{c61}R^{d61}, NR^{c61}NR^{c61}R^{d61} NR^{c61}C(O)R^{b61}, NR^{c61}C(O)OR^{a61}, NR^{c61}C(O)NR^{e61}R^{d61}, C(═NR^{e61})R^{b61}, C(═NR^{e61})NR^{c61}R^{d61}, NR^{c61}C(═NR^{e61})NR^{e61}R^{d61}, NR^{e61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, OS(O)(═NR^{e61})R^{b61}, OS(O)₂R^{b61}, S(O)(═NR^{e61})R^{b61}, SF₅, P(O)R^{f61}R^{g61} OP(O)(OR^{h61})(ORⁱ⁶¹), P(O)(OR^{h61})(ORⁱ⁶¹), and BR^{j61}R^{k61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, which is optionally substituted with 1, 2, 3, or 4 independently selected R⁶¹ substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f61} and R^{g61} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j61} and R^{k61} is independently selected from OH, C₁₋₆ alkoxy, and C₁₋₆ haloalkoxy;
or, any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a62}, SR^{a62}, NHOR^{a62}, C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{e62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62})NR^{c62}R^{d62}, NR^{e62}C(═NR^{e62})R^{b62}, NR^{c62}S(O)NR^{c62}R^{d62}, NR^{e62}S(O)R^{b62}, NR^{c62}S(O)₂R^{b62}, NR^{c62}S(O)(═NR^{e62})R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62} OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6C} substituents;
or, any R^{c62} and R^{d62}, attached to the same N atom, together with the N atom to which they are attached, form a 4-7 membered heterocycloalkyl group, which is optionally substituted with 1, 2, 3, or 4 independently selected R^{6C} substituents;
each R^{b62}, is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6C} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f62} and R^{g62} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j62} and R^{k62} is independently selected from OH, C₁₋₆ alkoxy, and C₁₋₆ haloalkoxy;
or, any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6C} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a63}, SR^{a63}, NHOR^{a63}, C(O)R^{b63}, C(O)NR^{c63}R^{d63}, C(O)NR^{c63}(OR^{a63}), C(O)OR^{a63}, OC(O)R^{b63}, OC(O)NR^{c63}R^{d63}, NR^{c63}R^{d63}, NR⁶³NR^{c63}R^{d63}, NR^{c63}C(O)R^{b63}, NR^{c63}C(O)OR^{a63}, NR^{c63}C(O)NR^{c63}R^{d63}, C(═NR^{e63})R^{b63}, C(═NR^{e63})NR^{c63}R^{d63}, NR^{c63}C(═NR^{e63})NR^{c63}R^{d63}, NR^{c63}C(═NR^{e63})R^{b63}, NR^{c63}S(O)NR^{c63}R^{d63}, NR^{c63}S(O)R^{b63}, NR^{c63}S(O)₂R^{b63}, NR^{c63}S(O)(═NR^{e63})R^{b63}, NR^{c63}S(O)₂NR^{c63}R^{d63}, S(O)R^{b63}, S(O)NR^{c63}R^{d63}, S(O)₂R^{b63}, S(O)₂NR^{c63}R^{d63}, OS(O)(═NR^{e63})R^{b63}, OS(O)₂R^{b63}, S(O)(═NR^{e63})R^{b63}, SF₅, P(O)R^{f63}R^{g63}, OP(O)(OR^{h63})(ORⁱ⁶³), P(O)(OR^{h63})(ORⁱ⁶³) and BR^{j63}R^{k63}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a63}, R^{c63}, and R^{d63}, is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c63} and R^{d63}, attached to the same N atom, together with the N atom to which they are attached, form a 4-7 membered heterocycloalkyl group, which is optionally substituted with 1, 2, 3, or 4 independently selected R^{G} substituents;
each R^{b63} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e63} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f63} and R^{g63} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h63} and Rⁱ⁶³ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j63} and R^{k63} is independently selected from OH, C₁₋₆ alkoxy, and C₁₋₆ haloalkoxy;
or, any R^{j63} and R^{k63} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁸ is selected from H, D, halo, CN, C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8} NR^{c8}C(O)R^{b8} NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{e8}S(O)R^{b8} NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a8}, R^{c8}, and R^{d8} is independently selected from H, C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c8} and R^{d8} attached to the same N atom, together with the N atom to which they are attached, form a 4-8 membered heterocycloalkyl group, which is optionally substituted with 1, 2, 3, or 4 independently selected R^{G} substituents;
each R^{b8} is independently selected from C₁₋₆ alkyl, C₁₋₆ haloalkyl, C₃₋₇ cycloalkyl, phenyl, 4-8 membered heterocycloalkyl, 5-6 membered heteroaryl, C₃₋₇ cycloalkyl-C₁₋₄ alkyl, phenyl-C₁₋₄ alkyl, 4-8 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, and C₁₋₆ haloalkoxy; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein n is 0, 1, or 2.

3. The compound of claim 1, or a pharmaceutically acceptable salt thereof, wherein n is 1.

4. The compound of claim 1, or a pharmaceutically acceptable salt thereof, wherein X is O, S(O)₂, or N(-L-R⁴).

5. The compound of claim 1, or a pharmaceutically acceptable salt thereof, wherein X is O.

6. The compound of claim 1, or a pharmaceutically acceptable salt thereof, wherein X is N(-L-R⁴).

7. The compound of claim 1, or a pharmaceutically acceptable salt thereof, wherein L is S(O)₂.

8. The compound of claim 1, or a pharmaceutically acceptable salt thereof, wherein R¹ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents.

9. The compound of claim 1, or a pharmaceutically acceptable salt thereof, wherein R¹ is selected from C₁₋₆ alkyl, C₁₋₆ haloalkyl, C₃₋₇ cycloalkyl, and 4-7 membered heterocycloalkyl, which are each optionally substituted with 1, 2, 3, or 4 independently selected R^{1A} substituents.

10. The compound of claim 1, or a pharmaceutically acceptable salt thereof, wherein R¹ is selected from C₁₋₆ alkyl, C₁₋₆ haloalkyl, and C₃₋₇ cycloalkyl, which are each optionally substituted with 1, 2, 3, or 4 independently selected R^{1A} substituents.

11. The compound of claim 1, or a pharmaceutically acceptable salt thereof, wherein R¹ is selected from C₁₋₆ alkyl, C₁₋₆ haloalkyl, and C₃₋₇ cycloalkyl.

12. The compound of claim 1, or a pharmaceutically acceptable salt thereof, wherein:
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}CHC(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{e12}R^{d12}, C(O)OR^{a12}, OC(O)R^{b12} OC(O)NR^{c12}R^{d12}, NR^{c12}R^{d12}, NR^{c12}C(O)R^{b12} NR^{c12}C(O)OR^{a12} NR^{e12}C(O)NR^{e12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl; and
each R^{b12}, is independently selected from C₁₋₆ alkyl and C₁₋₆ haloalkyl.

13. The compound of claim 1, or a pharmaceutically acceptable salt thereof, wherein:
each R^{1A} is independently selected from H, D, halo, CN, C₁₋₆ alkyl, C₁₋₆ haloalkyl, OR^{a11}, and NR^{c11}R_{d11}; and
each R^{a11}, R^{c11}, and R^{d11}, is independently selected from H, C₁₋₆ alkyl, and C₁₋₆ haloalkyl.

14. The compound of claim 1, or a pharmaceutically acceptable salt thereof, wherein R^{2A}R^{2B}, and R^{2C} are each independently selected from H, D, halo, CN, C₁₋₆ alkyl, C₁₋₆ haloalkyl, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl and C₁₋₆ haloalkyl are each optionally substituted with 1 or 2 independently selected R^{G} substituents.

15. The compound of claim 1, or a pharmaceutically acceptable salt thereof, wherein R^{2A}R^{2B}, and R^{2C} are each independently selected from H, halo, and OR^{a2}.

16. The compound of claim 1, or a pharmaceutically acceptable salt thereof, wherein:
each R^{a2}, R^{c2}, and R^{d2} is independently selected from H, C₁₋₆ alkyl, and C₁₋₆ haloalkyl; and
each R^{b2} is independently selected from C₁₋₆ alkyl and C₁₋₆ haloalkyl.

17. The compound of claim 1, or a pharmaceutically acceptable salt thereof, wherein each R³ is independently selected from —C₁₋₄ alkylene-OR^{a3}, —C₁₋₄ alkylene-NR^{c3}R^{d3}, OR^{a3}, and NR^{c3}R^{d3}.

18. The compound of claim 1, or a pharmaceutically acceptable salt thereof, wherein each R³ is OH.

19. The compound of claim 1, or a pharmaceutically acceptable salt thereof, wherein R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃-7 cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents.

20. The compound of claim 1, or a pharmaceutically acceptable salt thereof, wherein R⁴ is selected from C₁₋₆ alkyl and C₁₋₆ haloalkyl.

21. The compound of claim 1, or a pharmaceutically acceptable salt thereof, wherein:
each R^{4A} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41} OC(O)NR^{c41}R^{d41}, NR^{c41}R^{d41} NR^{c41}C(O)R^{b41}, NR^{c41}C(O)OR^{a41} NR^{c41}C(O)NR^{c41}R^{d41} NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from D, halo, CN, C₁₋₆ alkyl, C₁₋₆ haloalkyl, OR^{a42} SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR^{c42}S(O)₂NR^{c42}R^{d42}, S(O)₂R^{b42}, and S(O)₂NR^{c42}R^{d42};
each R^{a42}, R^{c42}, and R^{d42}, is independently selected from H, C₁₋₆ alkyl, and C₁₋₆ haloalkyl; and
each R^{b42} is independently selected from C₁₋₆ alkyl and C₁₋₆ haloalkyl.

22. The compound of claim 1, or a pharmaceutically acceptable salt thereof, wherein:
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R⁴¹, and S(O)₂NR^{c41}R^{d41},
each R^{a41}, R^{c41}, and R^{d41} is independently selected from H, C₁₋₆ alkyl, and C₁₋₆ haloalkyl; and
each R^{b41} is independently selected from C₁₋₆ alkyl and C₁₋₆ haloalkyl.

23. The compound of claim 1, or a pharmaceutically acceptable salt thereof, wherein R⁵ is selected from halo, CN, C₁₋₄ alkyl, C₁₋₄ haloalkyl, cyano-C₁₋₄ alkyl, HO—C₁₋₄ alkyl, C₁₋₃ alkoxy-C₁₋₄ alkyl, and C₃₋₄ cycloalkyl.

24. The compound of claim 1, or a pharmaceutically acceptable salt thereof, wherein R⁵ is selected from halo and CN.

25. The compound of claim 1, or a pharmaceutically acceptable salt thereof, wherein:
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents.

26. The compound of claim 1, or a pharmaceutically acceptable salt thereof, wherein R⁶ and R⁷, together with the nitrogen atom to which they are attached, form a 4-6 membered heterocycloalkyl moiety or a 5-6 membered heteroaryl moiety, each of which is optionally substituted by 1, 2, 3, or 4 independently selected R^{6A} substituents.

27. The compound of claim 1, or a pharmaceutically acceptable salt thereof, wherein each R^{6A} is independently selected from halo, CN, C₁₋₆ alkyl, C₁₋₆ haloalkyl, C₃₋₁₀ cycloalkyl, 6-10 membered aryl, 4-10 membered heterocycloalkyl, 5-10 membered heteroaryl, OR^{a61}, NR^{c61}R^{d61}, and S(O)₂R^{b61}, wherein said C₁₋₆ alkyl, C₁₋₆ haloalkyl, C₃₋₁₀ cycloalkyl, 6-10 membered aryl, 4-10 membered heterocycloalkyl, and 5-10 membered heteroaryl are each optionally substituted with 1, 2, 3, or 4 independently selected R^{6B} substituents.

28. The compound of claim 1, or a pharmaceutically acceptable salt thereof, wherein each R^{6A} is independently selected from halogen, C₁₋₆ alkyl, and C₁₋₆ haloalkyl, wherein said C₁₋₆ alkyl and C₁₋₆ haloalkyl are each optionally substituted with 1, 2, 3, or 4 substituents selected from D, halo, CN, NO₂, and OH.

29. The compound of claim 1, or a pharmaceutically acceptable salt thereof, wherein:
each R^{6A} is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61} NR^{c61}C(O)R^{b61}, NR^{c61}C(O)OR^{a61}, NR^{c61}C(O)NR^{c61}R^{d61}, NR^{e61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{e62}R^{d62}, NR^{c62}R^{d62}, NR^{c62}C(O)R^{b62}, NR^{c62}C(O)OR^{a62} NR^{c62}C(O)NR^{e62}R^{d62}, NR^{e62}S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, OR^{a63} SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63}, NR^{c63}C(O)NR^{e63}R^{d63}, NR^{c63}S(O)₂R^{b63}, NR^{c63}S(O)₂NR^{c63}R^{d63}, S(O)₂R^{b63}, and S(O)₂NR^{c63}R^{d63};
each R^{a63}, R^{c63}, and R^{d63}, is independently selected from H, C₁₋₆ alkyl, and C₁₋₆ haloalkyl; and
each R^{b63} is independently selected from C₁₋₆ alkyl and C₁₋₆ haloalkyl.

30. The compound of claim 1, or a pharmaceutically acceptable salt thereof, wherein:
each R^{6A} is independently selected from halo, CN, C₁₋₆ alkyl, C₁₋₆ haloalkyl, C₃₋₁₀ cycloalkyl, 4-10 membered heterocycloalkyl, 5-10 membered heteroaryl, OR^{a61}, NR^{c61}R^{d61}, and S(O)₂R^{b61}, wherein said C₁₋₆ alkyl, C₃₋₁₀ cycloalkyl, and 5-10 membered heteroaryl are each optionally substituted with 1, 2, or 3 independently selected R^{6B} substituents;
each R^{a61}, R^{c61}, and R^{d61} is independently selected from H, C₁₋₆ alkyl, and C₁₋₆ haloalkyl;
each R^{b61} is independently selected from C₁₋₆ alkyl and C₃₋₁₀ cycloalkyl;
each R^{6B} is independently selected from D, halo, CN, C₁₋₆ alkyl, OR^{a62}, and NR^{c62}R^{d62}; and
each R^{a62}, R^{c62}, and R^{d62}, is independently selected from H, C₁₋₆ alkyl, and C₁₋₆ haloalkyl.

31. The compound of claim 1, or a pharmaceutically acceptable salt thereof, wherein R⁸ is selected from H, D, halo, CN, C₁₋₆ alkyl, and C₁₋₆ haloalkyl, wherein said C₁₋₆ alkyl and C₁₋₆ haloalkyl are each optionally substituted with 1 or 2 independently selected R^{G} substituents.

32. The compound of claim 1, or a pharmaceutically acceptable salt thereof, wherein R⁸ is selected from H, D, halo, CN, C₁₋₃ alkyl, and C₁₋₃ haloalkyl.

33. The compound of claim 1, or a pharmaceutically acceptable salt thereof, wherein:
each R^{a8}, R^{c8}, and R^{d8} is independently selected from H, C₁₋₆ alkyl, and C₁₋₆ haloalkyl; and
each R^{b8} is independently selected from C₁₋₆ alkyl and C₁₋₆ haloalkyl.

34. The compound of claim 1, or a pharmaceutically acceptable salt thereof, wherein:
n is 0, 1, or 2;
X is O, S(O)₂, or N(-L-R⁴);
L is S(O)₂ or S(O)₂NR^{a};
R^{a} is selected from H, C₁₋₆ alkyl and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
R¹ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}CHC(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, C(O)R^{b12}, C(O)NR^{e12}R^{d12}, C(O)OR^{a12}, OC(O)R^{b12} OC(O)NR^{c12}R^{d12}, NR^{c12}R^{d12}, NR^{c12}C(O)R^{b12}, NR^{e12}C(O)OR^{a12}, NR^{e12}C(O)NR^{e12}R^{d12}, NR^{c12}S(O)₂R^{b12}, NR^{e12}S(O)₂NR^{c12}R^{d12}, S(O)₂R^{b12}, and S(O)₂NR^{c12}R^{d12};
each R^{a12}, R^{c12}, and R^{d12}, is independently selected from H, C₁₋₆ alkyl, and C₁₋₆ haloalkyl;
each R^{b12}, is independently selected from C₁₋₆ alkyl and C₁₋₆ haloalkyl;
R^{2A}, R^{2B}, and R^{2C} are each independently selected from H, D, halo, CN, C₁₋₆ alkyl, C₁₋₆ haloalkyl, OR^{a2}, C(O)R^{b2}, C(O)NR^{c2}R¹², C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl and C₁₋₆ haloalkyl are each optionally substituted with 1 or 2 independently selected R^{G} substituents;
each R^{a2}, R^{c2}, and R^{d2} is independently selected from H, C₁₋₆ alkyl, and C₁₋₆ haloalkyl;
each R^{b2} is independently selected from C₁₋₆ alkyl and C₁₋₆ haloalkyl;
each R³ is independently selected from D, halo, CN, C₁₋₆ alkyl, C₁₋₆ haloalkyl, OR^{a3}, and NR^{c3}R^{d3} wherein said C₁₋₆ alkyl and C₁₋₆ haloalkyl are each optionally substituted with 1 or 2 independently selected R^{G} substituents;
each R^{a3}, R^{c3}, and R^{d3} is independently selected from H, C₁₋₆ alkyl, and C₁₋₆ haloalkyl;
each R^{b3} is independently selected from C₁₋₆ alkyl and C₁₋₆ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41} OC(O)NR^{c41}R^{d41}, NR^{c41}R^{d41} NR^{c41}C(O)R^{b41}, NR^{c41}C(O)OR^{a41} NR^{c41}C(O)NR^{c41}R^{d41} NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from D, halo, CN, C₁₋₆ alkyl, C₁₋₆ haloalkyl, OR^{a42} SR^{a42}, C(O)R^{b42}, C(O)NR^{c42}R^{c42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR^{c42}S(O)₂NR^{c42}R^{d42}, S(O)₂R^{b42}, and S(O)₂NR^{c42}R^{d42};
each R^{a42}, R^{c42}, and R^{d42}, is independently selected from H, C₁₋₆ alkyl, and C₁₋₆ haloalkyl;
each R^{b42} is independently selected from C₁₋₆ alkyl and C₁₋₆ haloalkyl;
R⁵ is selected from halo, CN, C₁₋₄ alkyl, and C₁₋₄ haloalkyl;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61} NR^{c61}C(O)R^{b61}, NR^{c61}C(O)OR^{a61}, NR^{c61}C(O)NR^{c61}R^{d61}, NR^{e61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, which is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a63}, SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63} NR^{c63}C(O)NR^{c63}R^{d63}, NR^{c63}S(O)₂R^{b63} NR^{c63}S(O)₂NR^{c63}R^{d63}, S(O)₂R^{b63}, and S(O)₂NR^{c63}R^{d63}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a63}, R^{c63}, and R^{d63} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; and
each R^{b63} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
R⁸ is selected from H, D, halo, CN, C₁₋₆ alkyl, and C₁₋₆ haloalkyl, wherein said C₁₋₆ alkyl and C₁₋₆ haloalkyl are each optionally substituted with 1 or 2 independently selected R^{G} substituents; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

35. The compound of claim 1, or a pharmaceutically acceptable salt thereof, wherein:
n is 0, 1, or 2;
X is O, S(O)₂, or N(-L-R⁴);
L is S(O)₂ or S(O)₂NR^{a};
R^{a} is selected from H, C₁₋₆ alkyl and C₁₋₆ haloalkyl;
R¹ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11}, is independently selected from H, C₁₋₆ alkyl, and C₁₋₆ haloalkyl, wherein said C₁₋₆ alkyl and C₁₋₆ haloalkyl are each optionally substituted with 1 or 2 independently selected R^{1B} substituents;
each R^{b11} is independently selected from C₁₋₆ alkyl and C₁₋₆ haloalkyl, which are each optionally substituted with 1 or 2 independently selected R^{1B} substituents;
each R^{1B} is independently selected from H, D, halo, CN, C₁₋₆ alkyl, C₁₋₆ haloalkyl, OR^{a12} SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12} NR¹²C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12} is independently selected from H, C₁₋₆ alkyl, and C₁₋₆ haloalkyl;
each R^{b12}, is independently selected from C₁₋₆ alkyl and C₁₋₆ haloalkyl;
R^{2A}, R^{2B}, and R^{2C} are each independently selected from H, D, halo, CN, C₁₋₆ alkyl, C₁₋₆ haloalkyl, and OR^{a2};
each R^{a2} is independently selected from H and C₁₋₆ alkyl;
each R³ is independently selected from halo, CN, C₁₋₃ alkyl, C₁₋₃ haloalkyl, OR^{a3}, and NR^{c3}R^{d3} wherein said C₁₋₃ alkyl and C₁₋₃ haloalkyl are each optionally substituted with 1 or 2 independently selected R^{G} substituents.
each R^{a3}, R^{c3}, and R^{d3} is independently selected from H, C₁₋₆ alkyl, and C₁₋₆ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41},
each R^{a41}, R^{c41}, and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from halo and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a6}1 NR^{c61}C(O)NR^{e61}R^{d61}, NR^{c61}S(O)₂R^{b61}, NR^{c61}S(O)₂NR^{c61}R^{d61}, S(O)₂R^{b61}, and S(O)₂NR^{e61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{a61}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{e62}R^{d62}, NR^{c62}R^{d62}, NR^{c62}C(O)R^{b62}, NR^{c62}C(O)OR^{a62} NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{a62}, R^{c62}, and R^{d62}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6C} substituents;
each R^{b62}, is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6C} substituents;
each R^{6C} is independently selected from D, halo, CN, C₁₋₆ alkyl, C₁₋₆ haloalkyl, OR^{a63}, SR^{a63}, C(O)R^{b63}, C(O)NR^{c63}R^{d63}, C(O)OR^{a63}, OC(O)R^{b63}, OC(O)NR^{c63}R^{d63}, NR^{c63}R^{d63}, NR^{c63}C(O)R^{b63}, NR^{c63}C(O)OR^{a63} NR^{c63}C(O)NR^{c63}R^{d63}, NR^{c63}S(O)₂R^{b63}, NR^{c63}S(O)₂NR^{c63}R^{d63}, S(O)₂R^{b63}, and S(O)₂NR^{c63}R^{d63};
each R^{a63}, R^{c63}, and R^{d63}, is independently selected from H, C₁₋₆ alkyl, and C₁₋₆ haloalkyl;
each R^{b63} is independently selected from C₁₋₆ alkyl and C₁₋₆ haloalkyl;
R⁸ is selected from H, D, halo, CN, C₁₋₆ alkyl, and C₁₋₆ haloalkyl; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

36. The compound of claim 1, or a pharmaceutically acceptable salt thereof, wherein:
n is 0 or 1;
X is O, S(O)₂, or N(-L-R⁴);
L is S(O)₂;
R¹ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₅ cycloalkyl, 4-5 membered heterocycloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11} NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11}, is independently selected from H, C₁₋₆ alkyl, and C₁₋₆ haloalkyl;
each R^{b11} is independently selected from C₁₋₆ alkyl and C₁₋₆ haloalkyl;
R^{2A}, R^{2B}, and R^{2C} are each independently selected from H, halo, CN, and OR^{a2};
each R^{a2} is independently selected from H and C₁₋₆ alkyl;
each R³ is independently selected from halo, CN, C₁₋₃ alkyl, C₁₋₃ haloalkyl, OR^{a3}, and NR^{c3}R^{d3} wherein said C₁₋₃ alkyl and C₁₋₃ haloalkyl are each optionally substituted a substituent selected from OH, C₁₋₃ alkoxy, amino, C₁₋₃ alkylamino, and di(C₁₋₃ alkyl)amino;
each R^{a3}, R^{c3}, and R^{d3} is independently selected from H, C₁₋₃ alkyl, and C₁₋₃ haloalkyl;
R⁴ is selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41},
each R^{a41}, R^{c41}, and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from F, Cl, and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a6}1 NR^{c61}C(O)NR^{e61}R^{d61}, NR^{c61}S(O)₂R^{b61}, NR^{c61}S(O)₂NR^{c61}R⁶¹, S(O)₂R^{b61}, and S(O)₂NR^{c61}R^{d61}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61}, is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{b61} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{c62}, C(O)OR^{a62}, OC(O)R^{b62}, OC(O)NR^{c62}R^{d62}, NR^{c62}R^{d62}, NR^{c62}C(O)R^{b62}, N₁R^{c62}C(O)OR^{a62} NR^{c62}C(O)NR^{c62}R^{d62}, NR^{e62}S(O)₂R^{b62}, NR^{c62}S(O)₂NR^{c62}R^{d62}, S(O)₂R^{b62}, and S(O)₂NR^{c62}R^{d62};
each R^{a62}, R^{c62}, and R^{d62}, is independently selected from H, C₁₋₆ alkyl, and C₁₋₆ haloalkyl;
each R^{b62}, is independently selected from C₁₋₆ alkyl and C₁₋₆ haloalkyl; and
R⁸ is selected from H, D, halo, CN, C₁₋₃ alkyl, and C₁₋₃ haloalkyl.

37. The compound of claim 1, or a pharmaceutically acceptable salt thereof, wherein:
n is 0 or 1;
X is O, S(O)₂, or N(-L-R⁴);
L is S(O)₂;
R¹ is selected from C₁₋₆ alkyl, C₁₋₆ haloalkyl, C₃₋₁₀ cycloalkyl, and 4-10 membered heterocycloalkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R¹¹, and S(O)₂NR^{c11}R^{d11};
each R^{a11}, R^{c11}, and R^{d11}, is independently selected from H, C₁₋₆ alkyl, and C₁₋₆ haloalkyl;
each R^{b11} is independently selected from C₁₋₆ alkyl and C₁₋₆ haloalkyl;
R^{2A}, R^{2B}, and R^{2C} are each independently selected from H, halo, and OR^{a2};
each R^{a2} is C₁₋₆ alkyl;
each R³ is independently selected from —C₁₋₄ alkylene-OR^{a3}, —C₁₋₄ alkylene-NR^{c3}R^{d3} OR^{a3}, and NR^{c3}R^{d3};
each R^{a3} is H;
each R^{c3} and R^{d3} is independently selected from H and C₁₋₃ alkyl;
R⁴ is selected from C₁₋₆ alkyl and C₁₋₆ haloalkyl, wherein said C₁₋₆ alkyl and C₁₋₆ haloalkyl are each optionally substituted by 1, 2, 3, or 4 independently selected R^{4A} substituents;
each R^{4A} is independently selected from D, halo, CN, NO₂, C₁₋₆ alkyl, C₁₋₆ haloalkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41},
each R^{a41}, R^{c41}, and R^{d41} is independently selected from H, C₁₋₆ alkyl, and C₁₋₆ haloalkyl;
each R^{b41} is independently selected from C₁₋₆ alkyl and C₁₋₆ haloalkyl;
R⁵ is selected from F, Cl, and CN;
R⁶ is selected from C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted by 1, 2, 3, or 4 independently selected R^{6A} substituents;
R⁷ is selected from H, C₁₋₆ alkyl, and C₁₋₆ haloalkyl;
or, R⁶ and R⁷, together with the nitrogen atom to which they are attached, form a 4-10 membered heterocycloalkyl moiety or a 5-6 membered heteroaryl moiety, each of which is optionally substituted by 1, 2, 3, 4, 5, or 6 independently selected R^{6A} substituents;
each R^{6A} is independently selected from D, halo, CN, C₁₋₆ alkyl, C₁₋₆ haloalkyl, C₃₋₅ cycloalkyl, 4-5 membered heterocycloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{d61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₁₋₆ haloalkyl, C₃₋₅ cycloalkyl, and 4-5 membered heterocycloalkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₃₋₅ cycloalkyl, and 4-5 membered heterocycloalkyl, wherein said C₁₋₆ alkyl, C₁₋₆ haloalkyl, C₁₋₆ alkyl, C₁₋₆ haloalkyl, C₃₋₅ cycloalkyl, and 4-5 membered heterocycloalkyl are each optionally substituted with 1 or 2 independently selected R^{6B} substituents;
each R^{b61} is independently selected from C₁₋₆ alkyl, C₁₋₆ haloalkyl, C₁₋₆ alkyl, C₁₋₆ haloalkyl, C₃₋₅ cycloalkyl, and 4-5 membered heterocycloalkyl, which are each optionally substituted with 1 or 2 independently selected R^{6B} substituents;
each R^{6B} is independently selected from D, halo, CN, C₁₋₆ alkyl, C₁₋₆ haloalkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62} NR^{c62}C(O)NR^{e62}R^{d62}, NR^{e62}S(O)₂R^{b62}, NR^{c62}S(O)₂NR^{e62}R^{d62}, S(O)₂R^{b62}, and S(O)₂NR^{c62}R^{d62};
each R^{a62}, R^{c62}, and R^{d62}, is independently selected from H, C₁₋₆ alkyl, and C₁₋₆ haloalkyl;
each R^{b62}, is independently selected from C₁₋₆ alkyl and C₁₋₆ haloalkyl; and
R⁸ is selected from H, C₁₋₃ alkyl, and C₁₋₃ haloalkyl.

38. The compound of claim 1, or a pharmaceutically acceptable salt thereof, wherein:
n is 0 or 1;
X is O or N(-L-R⁴);
L is S(O)₂;
R¹ is selected from C₁₋₆ alkyl, C₁₋₆ haloalkyl, and C₃₋₁₀ cycloalkyl;
R^{2A}, R^{2B}, and R^{2C} are each independently selected from H, halo, and OR^{a2};
each R^{a2} is C₁₋₆ alkyl;
each R³ is OR^{a3};
each R^{a3} is H;
R⁴ is C₁₋₆ alkyl;
R⁵ is selected from halo and CN;
R⁶ is selected from C₁₋₆ alkyl and C₃₋₁₀ cycloalkyl-C₁₋₄ alkyl; which are each optionally substituted by 1 or 2 independently selected R^{6A} substituents;
R⁷ is selected from H and C₁₋₆ alkyl;
or, R⁶ and R⁷, together with the nitrogen atom to which they are attached, form a 4-15 membered heterocycloalkyl moiety, which is optionally substituted by 1, 2, 3, 4, or 5 independently selected R^{6A} substituents;
each R^{6A} is independently selected from halo, CN, C₁₋₆ alkyl, C₁₋₆ haloalkyl, C₃₋₁₀ cycloalkyl, 4-10 membered heterocycloalkyl, 5-10 membered heteroaryl, OR^{a61}, NR^{c61}R^{d61}, and S(O)₂R^{b61}, wherein said C₁₋₆ alkyl, C₃₋₁₀ cycloalkyl, and 5-10 membered heteroaryl are each optionally substituted with 1, 2, 3, or 4 independently selected R^{6B} substituents;
each R^{a61}, R^{c61}, and R^{d61} is independently selected from H, C₁₋₆ alkyl, and C₁₋₆ haloalkyl;
each R^{b61} is independently selected from C₁₋₆ alkyl and C₃₋₁₀ cycloalkyl;
each R^{6B} is independently selected from D, halo, CN, C₁₋₆ alkyl, OR^{a62}, and NR^{c62}R^{d62};
each R^{a62}, R^{c62}, and R^{d62}, is independently selected from H, C₁₋₆ alkyl, and C₁₋₆ haloalkyl; and
R⁸ is selected from H and C₁₋₆ alkyl.

39. The compound of claim 1, wherein the compound is a compound of Formula (Ia): 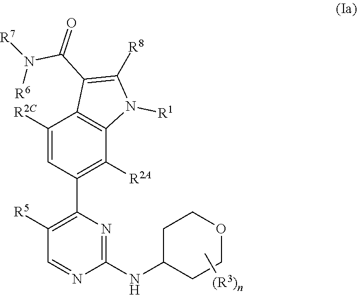
or a pharmaceutically acceptable salt thereof.

40. The compound of claim 1, wherein the compound is a compound of Formula (Ib): 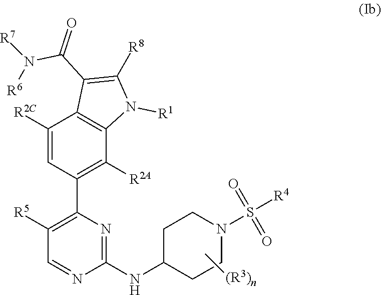
or a pharmaceutically acceptable salt thereof.

41. The compound of claim 1, wherein the compound comprises from 1 to 6 deuterium atoms.

42. The compound of claim 1, selected from:
4-fluoro-6-(5-fluoro-2-(((3R,4R)-3-hydroxy-1-(methylsulfonyl)piperidin-4-yl)amino)pyrimidin-4-yl)-1-isopropyl-N-methyl-1H-indole-3-carboxamide;
4-fluoro-6-(5-fluoro-2-(((3S,4R)-3-hydroxytetrahydro-2H-pyran-4-yl)amino)pyrimidin-4-yl)-1-isopropyl-N-methyl-1H-indole-3-carboxamide;
4-fluoro-6-(5-fluoro-2-(((3R,4R)-3-hydroxy-1-(methylsulfonyl)piperidin-4-yl)amino)pyrimidin-4-yl)-1-isopropyl-N-(2-(pyrimidin-2-yl)ethyl)-1H-indole-3-carboxamide;
6-(5-chloro-2-(((3S,4R)-3-hydroxytetrahydro-2H-pyran-4-yl)amino)pyrimidin-4-yl)-4-fluoro-1-isopropyl-N-((1-methyl-1H-indazol-6-yl)methyl)-1H-indole-3-carboxamide;
6-(5-cyano-2-(((3S,4R)-3-hydroxytetrahydro-2H-pyran-4-yl)amino)pyrimidin-4-yl)-4-fluoro-1-isopropyl-N-((1-methyl-1H-indazol-6-yl)methyl)-1H-indole-3-carboxamide;
6-(5-chloro-2-(((3S,4R)-3-hydroxytetrahydro-2H-pyran-4-yl)amino)pyrimidin-4-yl)-4-fluoro-1-isopropyl-N,2-dimethyl-1H-indole-3-carboxamide;
1-cyclopentyl-4,7-difluoro-6-(5-fluoro-2-(((3S,4R)-3-hydroxytetrahydro-2H-pyran-4-yl)amino)pyrimidin-4-yl)-N-methyl-1H-indole-3-carboxamide;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(hydroxymethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-methylazetidin-1-yl)methanone;
((2S,4R)-4-fluoro-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone; and
((S)-4,4-difluoro-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
or a pharmaceutically acceptable salt thereof.

43. The compound of claim 1, selected from:
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-4-(hydroxymethyl)-5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-4-(hydroxymethyl)-5-azaspiro[2.3]hexan-5-yl)methanone; and
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-2-(hydroxymethyl)azetidin-1-yl)methanone;
or a pharmaceutically acceptable salt thereof.

44. The compound of claim 1, selected from:
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl-d₂)-3-methylazetidin-1-yl)methanone;
((2R,3S)-3-(difluoromethoxy)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
or a pharmaceutically acceptable salt thereof.

45. The compound of claim 1, selected from:
(6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl-d₂)-3-methylazetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-(2-fluoropropan-2-yl)azetidin-1-yl)methanone;
(5,5-difluoro-2-azaspiro[3.3]heptan-2-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((R)-2-methylazetidin-1-yl)methanone;
(1,1-difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-chloro-2-(((3S,4R)-3-hydroxytetrahydro-2H-pyran-4-yl)amino)pyrimidin-4-yl)-4-fluoro-1-isopropyl-1H-indol-3-yl)((2S,3S)-2-(hydroxymethyl)-3-methylazetidin-1-yl)methanone;
(6-(5-chloro-2-(((3S,4R)-3-hydroxytetrahydro-2H-pyran-4-yl)amino)pyrimidin-4-yl)-4-fluoro-1-isopropyl-1H-indol-3-yl)((2S,3S)-2-(hydroxymethyl-d₂)-3-methylazetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-methylazetidin-1-yl)methanone;
((2R,3S)-3-(difluoromethoxy)-2-(hydroxymethyl)azetidin-1-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
N-((S)-1-cyclopropyl-2-hydroxyethyl)-1-ethyl-4-fluoro-6-(5-fluoro-2-(((3S,4R)-3-hydroxytetrahydro-2H-pyran-4-yl)amino)pyrimidin-4-yl)-1H-indole-3-carboxamide;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((S)-2-(trifluoromethyl)azetidin-1-yl)methanone;
(1,1-difluoro-5-azaspiro[2.3]hexan-5-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
(1,1-difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl)-3-(trifluoromethyl)azetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3S)-2-(hydroxymethyl-d₂)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-(trifluoromethyl)azetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl)-3-(trifluoromethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-methoxy-3-methylazetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2R,3R)-2-(hydroxymethyl)-3-methoxy-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2R,3R)-2-(hydroxymethyl)-3-methoxy-3-methylazetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2R,3S)-2-(hydroxymethyl)-3-(trifluoromethoxy)azetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2R,3S)-2-(hydroxymethyl)-3-(trifluoromethoxy)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3S)-2-(hydroxymethyl)-3-(trifluoromethoxy)azetidin-1-yl)methanone;
(6-(5-chloro-2-(((3S,4R)-3-hydroxytetrahydro-2H-pyran-4-yl)amino)pyrimidin-4-yl)-4-fluoro-1-isopropyl-1H-indol-3-yl)((2R,3S)-2-(hydroxymethyl)-3-(trifluoromethoxy)azetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((2R,3S)-2-(hydroxymethyl)-3-(trifluoromethoxy)azetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((2R,3S)-2-(hydroxymethyl)-3-(trifluoromethoxy)azetidin-1-yl)methanone;
(1-(2,2-difluoroethyl)-4-fluoro-6-(5-fluoro-2-(((3S,4R)-3-hydroxytetra hydro-2H-pyran-4-yl)amino)pyrimidin-4-yl)-1H-indol-3-yl)(5-azaspiro[2.3]hexan-5-yl)methanone;
(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)((2S,3S)-2-(hydroxymethyl-d₂)-3-methylazetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((S)-4-(hydroxymethyl)-5-azaspiro[2.3]hexan-5-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((R)-4-(hydroxymethyl)-5-azaspiro[2.3]hexan-5-yl)methanone;
(3-(tert-butyl)-3-hydroxyazetidin-1-yl)(6-(5-chloro-2-(((3S,4R)-3-hydroxytetrahydro-2H-pyran-4-yl)amino)pyrimidin-4-yl)-1-ethyl-4-fluoro-1H-indol-3-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)(3-((difluoromethoxy)methyl)azetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((2S,3S)-2-(hydroxymethyl-d₂)-3-methylazetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)(3-(difluoromethyl)azetidin-1-yl)methanone;
(6-(5-chloro-2-(((3S,4R)-3-hydroxytetrahydro-2H-pyran-4-yl)amino)pyrimidin-4-yl)-1-ethyl-4-fluoro-1H-indol-3-yl)((2R,3S)-3-(difluoromethoxy)-2-(hydroxymethyl)azetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-4-(hydroxymethyl)-5-azaspiro[2.3]hexan-5-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((R)-4-(hydroxymethyl)-5-azaspiro[2.3]hexan-5-yl)methanone;
((4S)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone;
((4R)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-1-(hydroxymethyl)-2-azaspiro[3.3]heptan-2-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((R)-1-(hydroxymethyl)-2-azaspiro[3.3]heptan-2-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,3S)-2-(hydroxymethyl)-3-methylazetidin-1-yl)methanone;
2-((S)-1-(6-(5-fluoro-2-(((3S,4R)-3-hydroxytetrahydro-2H-pyran-4-yl)amino)pyrimidin-4-yl)-1-methyl-1H-indole-3-carbonyl)azetidin-2-yl)acetonitrile;
((2R,3S)-3-(difluoromethoxy)-2-(hydroxymethyl)azetidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((2S,3S)-2-(hydroxymethyl-d₂)-3-methylazetidin-1-yl)methanone; and
(6-(5-chloro-2-(((3S,4R)-3-hydroxytetrahydro-2H-pyran-4-yl)amino)pyrimidin-4-yl)-1-ethyl-1H-indol-3-yl)((2R,3S)-3-(difluoromethoxy)-2-(hydroxymethyl)azetidin-1-yl)methanone;
or a pharmaceutically acceptable salt thereof.

46. The compound of claim 1, selected from:
(6-(5-fluoro-2-(((3S,4R)-3-hydroxytetrahydro-2H-pyran-4-yl)amino)pyrimidin-4-yl)-1-methyl-1H-indol-3-yl)(5-azaspiro[2.3]hexan-5-yl)methanone;
(6-(5-fluoro-2-(((3S,4R)-3-hydroxytetrahydro-2H-pyran-4-yl)amino)pyrimidin-4-yl)-1-methyl-1H-indol-3-yl)(3-(trifluoromethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((R)-2-methylazetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-2-(hydroxymethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-2-((methylsulfonyl)methyl)azetidin-1-yl)methanone;
(5,5-difluoro-2-azaspiro[3.3]heptan-2-yl)(6-(5-fluoro-2-(((3S,4R)-3-hydroxytetrahydro-2H-pyran-4-yl)amino)pyrimidin-4-yl)-1-methyl-1H-indol-3-yl)methanone;
N-((S)-1-cyclopropyl-2-hydroxyethyl)-6-(5-fluoro-2-(((3S,4R)-3-hydroxytetrahydro-2H-pyran-4-yl)amino)pyrimidin-4-yl)-1-methyl-1H-indole-3-carboxamide;
(6-(5-fluoro-2-(((3S,4R)-3-hydroxytetrahydro-2H-pyran-4-yl)amino)pyrimidin-4-yl)-1-methyl-1H-indol-3-yl)(3-hydroxy-3-(trifluoromethyl)azetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2R,3R)-3-hydroxy-2-methylazetidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2R,3 S)-3-hydroxy-2-methylazetidin-1-yl)methanone;
((2S,3S)-2-((cyclopropylsulfonyl)methyl)-3-methylazetidin-1-yl)(6-(5-fluoro-2-(((3S,4R)-3-hydroxytetrahydro-2H-pyran-4-yl)amino)pyrimidin-4-yl)-1-methyl-1H-indol-3-yl)methanone;
((2R,3 S)-3-(difluoromethoxy)-2-(hydroxymethyl)azetidin-1-yl)(6-(5-fluoro-2-(((3 S,4R)-3-hydroxytetrahydro-2H-pyran-4-yl)amino)pyrimidin-4-yl)-1-methyl-1H-indol-3-yl)methanone;
((2S,4R)-4-(difluoromethoxy)-2-(hydroxymethyl)pyrrolidin-1-yl)(6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethoxy)pyrrolidin-1-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethyl)pyrrolidin-1-yl)methanone;
((S)-4,4-difluoro-2-(hydroxymethyl)pyrrolidin-1-yl)(6-(5-fluoro-2-(((3 S,4R)-3-hydroxytetrahydro-2H-pyran-4-yl)amino)pyrimidin-4-yl)-1-methyl-1H-indol-3-yl)methanone;
(6-(5-fluoro-2-(((3S,4R)-3-hydroxytetrahydro-2H-pyran-4-yl)amino)pyrimidin-4-yl)-1-methyl-1H-indol-3-yl)((S)-2-(hydroxymethyl)-4,4-dimethylpyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(3-(trifluoromethyl)azetidin-1-yl)methanone;
((2S,4R)-4-(difluoromethoxy)-2-(hydroxymethyl)pyrrolidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,4R)-2-(hydroxymethyl)-4-(trifluoromethoxy)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,4R)-2-(hydroxymethyl)-4-(trifluoromethyl)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((2S,4R)-4-fluoro-2-(hydroxymethyl)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((S)-2-(hydroxymethyl)-4,4-dimethylpyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((S)-6-(hydroxymethyl)-5-azaspiro[2.4]heptan-5-yl)methanone;
((S)-4,4-difluoro-2-(hydroxymethyl)pyrrolidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
((3S,6S)-1,1-difluoro-6-(hydroxymethyl)-5-azaspiro[2.4]heptan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
2-((S)-1-(1-ethyl-6-(5-fluoro-2-(((3S,4R)-3-hydroxytetrahydro-2H-pyran-4-yl)amino)pyrimidin-4-yl)-1H-indole-3-carbonyl)azetidin-2-yl)acetonitrile;
(1-ethyl-6-(5-fluoro-2-(((3S,4R)-3-hydroxytetrahydro-2H-pyran-4-yl)amino)pyrimidin-4-yl)-1H-indol-3-yl)((S)-2-((methylsulfonyl)methyl)azetidin-1-yl)methanone;
(1,1-difluoro-5-azaspiro[2.3]hexan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(5,5-difluoro-2-azaspiro[3.3]heptan-2-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(3-(2-fluoropropan-2-yl)azetidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)((R)-3-(trifluoromethoxy)pyrrolidin-1-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(5-azaspiro[2.3]hexan-5-yl)methanone;
N-((S)-1-cyclopropyl-2-hydroxyethyl)-1-ethyl-6-(5-fluoro-2-(((3S,4R)-3-hydroxytetrahydro-2H-pyran-4-yl)amino)pyrimidin-4-yl)-1H-indole-3-carboxamide;
(1-ethyl-6-(5-fluoro-2-(((3S,4R)-3-hydroxytetrahydro-2H-pyran-4-yl)amino)pyrimidin-4-yl)-1H-indol-3-yl)(5-azaspiro[2.4]heptan-5-yl)methanone;
(3-(difluoromethyl)-3-hydroxyazetidin-1-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
(1-ethyl-6-(5-fluoro-2-(((3S,4R)-3-hydroxytetrahydro-2H-pyran-4-yl)amino)pyrimidin-4-yl)-1H-indol-3-yl)(3-hydroxy-3-(trifluoromethyl)azetidin-1-yl)methanone;
((4S)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
((4R)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
((2S,3S)-2-((cyclopropylsulfonyl)methyl)-3-methylazetidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
2-((S)-1-(4-fluoro-6-(5-fluoro-2-(((3 S,4R)-3-hydroxytetrahydro-2H-pyran-4-yl)amino)pyrimidin-4-yl)-1-methyl-1H-indole-3-carbonyl)azetidin-2-yl)acetonitrile;
((2S,4R)-4-(difluoromethoxy)-2-(hydroxymethyl)pyrrolidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethoxy)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4R)-2-(hydroxymethyl)-4-(trifluoromethyl)pyrrolidin-1-yl)methanone;
((3S,6S)-1,1-difluoro-6-(hydroxymethyl)-5-azaspiro[2.4]heptan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4S)-2-(hydroxymethyl)-4-methoxy-4-(trifluoromethyl)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,4S)-4-hydroxy-2-(hydroxymethyl)-4-(trifluoromethyl)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((R)-3-(trifluoromethoxy)pyrrolidin-1-yl)methanone;
((R)-3-(difluoromethoxy)pyrrolidin-1-yl)(4-fluoro-6-(5-fluoro-2-(((3 S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3 S)-2-(hydroxymethyl-d2)-3-methylazetidin-1-yl)methanone;
((2R,3 S)-3-(difluoromethoxy)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
((S)-2-(difluoromethyl)azetidin-1-yl)(4-fluoro-6-(5-fluoro-2-(((3 S,4R)-3-hydroxytetrahydro-2H-pyran-4-yl)amino)pyrimidin-4-yl)-1-methyl-1H-indol-3-yl)methanone;
(S)-1-(4-fluoro-6-(5-fluoro-2-(((3S,4R)-3-hydroxytetrahydro-2H-pyran-4-yl)amino)pyrimidin-4-yl)-1-methyl-1H-indole-3-carbonyl)azetidine-2-carbonitrile;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-hydroxyazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)(3-methoxyazetidin-1-yl)methanone;
(3-(1,1-difluoroethyl)-3-methylazetidin-1-yl)(4-fluoro-6-(5-fluoro-2-(((3 S,4R)-3-hydroxytetrahydro-2H-pyran-4-yl)amino)pyrimidin-4-yl)-1-methyl-1H-indol-3-yl)methanone;
(1,1-difluoro-2,2-dimethyl-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(6-(5-chloro-2-(((3S,4R)-3-hydroxytetrahydro-2H-pyran-4-yl)amino)pyrimidin-4-yl)-1-ethyl-1H-indol-3-yl)(3-(trifluoromethyl)azetidin-1-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((R)-2-methylazetidin-1-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((2S,4R)-4-(difluoromethoxy)-2-(hydroxymethyl)pyrrolidin-1-yl)methanone;
(6-(5-chloro-2-(((3S,4R)-3-hydroxytetrahydro-2H-pyran-4-yl)amino)pyrimidin-4-yl)-1-ethyl-1H-indol-3-yl)((2S,4R)-4-fluoro-2-(hydroxymethyl)pyrrolidin-1-yl)methanone;
(5,5-difluoro-2-azaspiro[3.3]heptan-2-yl)(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)((2S,3 S)-2-(hydroxymethyl-d2)-3-methylazetidin-1-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)(3-(2-fluoropropan-2-yl)azetidin-1-yl)methanone;
(1,1-difluoro-5-azaspiro[2.3]hexan-5-yl)(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)methanone;
(6-(5-chloro-2-(((3S,4R)-3-hydroxytetrahydro-2H-pyran-4-yl)amino)pyrimidin-4-yl)-4-fluoro-1-methyl-1H-indol-3-yl)(3-(2-fluoropropan-2-yl)azetidin-1-yl)methanone;
(6-(5-chloro-2-(((3S,4R)-3-hydroxytetrahydro-2H-pyran-4-yl)amino)pyrimidin-4-yl)-4-fluoro-1-methyl-1H-indol-3-yl)(1,1-difluoro-2,2-dimethyl-5-azaspiro[2.3]hexan-5-yl)methanone;
(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone;
(1-(2,2-difluoroethyl)-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone;
((4S)-1,1-difluoro-4-(hydroxymethyl)-5-azaspiro[2.3]hexan-5-yl)(1-(2,2-difluoroethyl)-6-(5-fluoro-2-(((3 S,4R)-3-hydroxytetrahydro-2H-pyran-4-yl)amino)pyrimidin-4-yl)-1H-indol-3-yl)methanone;
((4R)-1,1-difluoro-4-(hydroxymethyl)-5-azaspiro[2.3]hexan-5-yl)(1-(2,2-difluoroethyl)-6-(5-fluoro-2-(((3 S,4R)-3-hydroxytetrahydro-2H-pyran-4-yl)amino)pyrimidin-4-yl)-1H-indol-3-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)((S)-4-(hydroxymethyl)-5-azaspiro[2.3]hexan-5-yl)methanone;
(1-(2,2-difluoroethyl)-4-fluoro-6-(5-fluoro-2-(((3S,4R)-3-hydroxytetrahydro-2H-pyran-4-yl)amino)pyrimidin-4-yl)-1H-indol-3-yl)((R)-4-(hydroxymethyl)-5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((S)-1-(hydroxymethyl)-2-azaspiro[3.3]heptan-2-yl)methanone;
(4-fluoro-6-(5-fluoro-2-(((3S,4R)-3-hydroxytetrahydro-2H-pyran-4-yl)amino)pyrimidin-4-yl)-1-methyl-1H-indol-3-yl)((R)-1-(hydroxymethyl)-2-azaspiro[3.3]heptan-2-yl)methanone;
((4S)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((4R)-1,1-difluoro-4-(hydroxymethyl)-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((2S,3S)-3-(difluoromethyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((2R,3R)-3-(difluoromethyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((1R,4S,5R)-5-(hydroxymethyl)-2-azabicyclo[2.1.0]pentan-2-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((1S,4R,5S)-5-(hydroxymethyl)-2-azabicyclo[2.1.0]pentan-2-yl)methanone;
(2S,3S)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)-2-(hydroxymethyl)azetidine-3-carbonitrile;
(2R,3R)-1-(4-fluoro-6-(5-fluoro-2-(((3 S,4R)-3-hydroxytetrahydro-2H-pyran-4-yl)amino)pyrimidin-4-yl)-1-isopropyl-1H-indole-3-carbonyl)-2-(hydroxymethyl)azetidine-3-carbonitrile;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-(methylsulfonyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3 S)-2-(hydroxymethyl)-3-(methylsulfonyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3 S)-2-(hydroxymethyl)-3-(methylsulfonyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3R)-2-(hydroxymethyl)-3-(methylsulfonyl)azetidin-1-yl)methanone;
((4R)-1,1-difluoro-4-(hydroxymethyl)-5-azaspiro[2.3]hexan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
((4S)-1,1-difluoro-4-(hydroxymethyl)-5-azaspiro[2.3]hexan-5-yl)(1-ethyl-6-(5-fluoro-2-(((3S,4R)-3-hydroxytetrahydro-2H-pyran-4-yl)amino)pyrimidin-4-yl)-1H-indol-3-yl)methanone;
((4R)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-isopropyl-1H-indol-3-yl)methanone;
((4S)-1,1-difluoro-4-(hydroxymethyl)-5-azaspiro[2.3]hexan-5-yl)(6-(5-fluoro-2-(((3S,4R)-3-hydroxytetrahydro-2H-pyran-4-yl)amino)pyrimidin-4-yl)-1-isopropyl-1H-indol-3-yl)methanone;
(6-(5-fluoro-2-(((3S,4R)-3-hydroxytetrahydro-2H-pyran-4-yl)amino)pyrimidin-4-yl)-1-isopropyl-1H-indol-3-yl)((S)-4-(hydroxymethyl)-5-azaspiro[2.3]hexan-5-yl)mehthanone;
(6-(5-fluoro-2-(((3S,4R)-3-hydroxytetrahydro-2H-pyran-4-yl)amino)pyrimidin-4-yl)-1-isopropyl-1H-indol-3-yl)((R)-4-(hydroxymethyl)-5-azaspiro[2.3]hexan-5-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3 S)-2-(hydroxymethyl)-3-((trifluoromethoxy)methyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2R,3R)-2-(hydroxymethyl)-3-((trifluoromethoxy)methyl)azetidin-1-yl)methanone;
((2S,3S)-3-((difluoromethoxy)methyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((2R,3R)-3-((difluoromethoxy)methyl)-2-(hydroxymethyl)azetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
((4S)-1,1-difluoro-4-(hydroxymethyl)-5-azaspiro[2.3]hexan-5-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
((4R)-1,1-difluoro-4-(hydroxymethyl)-5-azaspiro[2.3]hexan-5-yl)(1-ethyl-4-fluoro-6-(5-fluoro-2-(((3S,4R)-3-hydroxytetrahydro-2H-pyran-4-yl)amino)pyrimidin-4-yl)-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-1-(hydroxymethyl)-2-azaspiro[3.3]heptan-2-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-1-(hydroxymethyl)-2-azaspiro[3.3]heptan-2-yl)methanone;
((R)-3,3-difluoro-2-(hydroxymethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
((S)-3,3-difluoro-2-(hydroxymethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
4-fluoro-6-(5-fluoro-2-(((3R,4R)-3-hydroxy-1-(methylsulfonyl)piperidin-4-yl)amino)pyrimidin-4-yl)-1-isopropyl-N,N-dimethyl-1H-indole-3-carboxamide;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(methoxymethyl)azetidin-1-yl)methanone;
2-((S)-1-(4-fluoro-6-(5-fluoro-2-(((3 S,4R)-3-hydroxytetrahydro-2H-pyran-4-yl)amino)pyrimidin-4-yl)-1-isopropyl-1H-indole-3-carbonyl)azetidin-2-yl)acetonitrile;
((S)-2-((dimethylamino)methyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
((S)-2-(2,2-difluoroethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(1,1-difluoro-5-azaspiro[2.4]heptan-5-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(5,5-difluoro-2-azaspiro[3.3]heptan-2-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(hydroxymethyl)-2-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3R)-2-(hydroxymethyl)-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(2-hydroxyethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(2-hydroxypropan-2-yl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((2S,3 S)-3-methyl-2-((methylsulfonyl)methyl)azetidin-1-yl)methanone;
((S)-2-((cyclopropylsulfonyl)methyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(1,1-difluoro-5-azaspiro[2.3]hexan-5-yl)(6-(5-fluoro-2-(((3S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(pyrrolidin-1-yl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(6-methyl-2,6-diazaspiro[3.3]heptan-2-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(6-oxa-2-azaspiro[3.4]octan-2-yl)methanone;
(3-(dimethylamino)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(fluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-hydroxyazetidin-1-yl)methanone;
(3-(difluoromethoxy)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3R,4R)-3-hydroxytetrahydro-2H-pyran-4-yl)amino)pyrimidin-4-yl)-1-isopropyl-1H-indol-3-yl)(3-(pyrrolidin-1-yl)azetidin-1-yl)methanone;
azetidin-1-yl(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(3-aminoazetidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(3-(difluoromethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-fluoroazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-2-methylazetidin-1-yl)methanone;
((2R,4R)-2,4-dimethylazetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(5-azaspiro[2.3]hexan-5-yl)methanone;
1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)azetidine-3-carbonitrile;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-3-fluoropyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-3-hydroxypyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-3-hydroxypyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-hydroxy-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(hydroxymethyl)pyrrolidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((R)-2-(hydroxymethyl)pyrrolidin-1-yl)methanone;
(3,3-difluoropyrrolidin-1-yl)(4-fluoro-6-(5-fluoro-2-(((3S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((1R,2S,5S)-2-(hydroxymethyl)-3-azabicyclo[3.1.0]hexan-3-yl)methanone;
(S)-1-(4-fluoro-6-(5-fluoro-2-(((3S,4R)-3-hydroxytetrahydro-2H-pyran-4-yl)amino)pyrimidin-4-yl)-1-isopropyl-1H-indole-3-carbonyl)pyrrolidine-3-carbonitrile;
(R)-1-(4-fluoro-6-(5-fluoro-2-(((3 S,4R)-3-hydroxytetrahydro-2H-pyran-4-yl)amino)pyrimidin-4-yl)-1-isopropyl-1H-indole-3-carbonyl)pyrrolidine-3-carbonitrile;
(3-fluoro-3-(trifluoromethyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(5-azaspiro[2.4]heptan-5-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-methyl-3-(trifluoromethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-isopropoxyazetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(hydroxymethyl)azetidin-1-yl)methanone;
(3-((difluoromethoxy)methyl)azetidin-1-yl)(4-fluoro-6-(5-fluoro-2-(((3 S,4R)-3-hydroxytetrahydro-2H-pyran-4-yl)amino)pyrimidin-4-yl)-1-isopropyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(2-(trifluoromethoxy)ethyl)azetidin-1-yl)methanone;
(1-ethyl-4-fluoro-6-(5-fluoro-2-(((3S,4R)-3-hydroxytetrahydro-2H-pyran-4-yl)amino)pyrimidin-4-yl)-1H-indol-3-yl)((S)-2-(hydroxymethyl)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-(trifluoromethoxy)azetidin-1-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)(3-((trifluoromethoxy)methyl)azetidin-1-yl)methanone;
(3-(difluoromethoxy)azetidin-1-yl)(4-fluoro-6-(5-fluoro-2-(((3 S,4R)-3-hydroxytetrahydro-2H-pyran-4-yl)amino)pyrimidin-4-yl)-1-methyl-1H-indol-3-yl)methanone;
(4-fluoro-6-(5-fluoro-2-(((3S,4R)-3-hydroxytetrahydro-2H-pyran-4-yl)amino)pyrimidin-4-yl)-1-isopropyl-1H-indol-3-yl)((S)-2-(1-hydroxycyclopropyl)azetidin-1-yl)methanone;
(4,7-difluoro-6-(5-fluoro-2-(((3S,4R)-3-hydroxytetrahydro-2H-pyran-4-yl)amino)pyrimidin-4-yl)-1-methyl-1H-indol-3-yl)(1,1-difluoro-5-azaspiro[2.3]hexan-5-yl)methanone;
(1,1-difluoro-5-azaspiro[2.3]hexan-5-yl)(7-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(7-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3 S)-2-(hydroxymethyl-d2)-3-methylazetidin-1-yl)methanone;
(4-fluoro-6-(5-fluoro-2-(((3S,4R)-3-hydroxytetrahydro-2H-pyran-4-yl)amino)pyrimidin-4-yl)-1,7-dimethyl-1H-indol-3-yl)((2S,3 S)-2-(hydroxymethyl-d2)-3-methylazetidin-1-yl)methanone;
(1,1-difluoro-5-azaspiro[2.3]hexan-5-yl)(6-(5-fluoro-2-(((3S,4R)-3-hydroxytetrahydro-2H-pyran-4-yl)amino)pyrimidin-4-yl)-1,7-dimethyl-1H-indol-3-yl)methanone;
(4,5-difluoro-6-(5-fluoro-2-(((3S,4R)-3-hydroxytetrahydro-2H-pyran-4-yl)amino)pyrimidin-4-yl)-1-methyl-1H-indol-3-yl)(1,1-difluoro-5-azaspiro[2.3]hexan-5-yl)methanone;
(1,1-difluoro-5-azaspiro[2.3]hexan-5-yl)(5-fluoro-6-(5-fluoro-2-(((3S,4R)-3-hydroxytetrahydro-2H-pyran-4-yl)amino)pyrimidin-4-yl)-1-methyl-1H-indol-3-yl)methanone;
(5-fluoro-6-(5-fluoro-2-(((3S,4R)-3-hydroxytetrahydro-2H-pyran-4-yl)amino)pyrimidin-4-yl)-1-methyl-1H-indol-3-yl)((2S,3 S)-2-(hydroxymethyl-d2)-3-methylazetidin-1-yl)methanone; and
(1,1-difluoro-5-azaspiro[2.3]hexan-5-yl)(4-fluoro-6-(5-fluoro-2-(((3S,4R)-3-hydroxytetrahydro-2H-pyran-4-yl)amino)pyrimidin-4-yl)-5-methoxy-1-methyl-1H-indol-3-yl)methanone;
or a pharmaceutically acceptable salt thereof.

47. A pharmaceutical composition comprising the compound of claim 1, or a pharmaceutically acceptable salt thereof, and a pharmaceutically acceptable carrier.

48. A method of inhibiting CDK4, comprising contacting the CDK4 with the compound of claim 1, or a pharmaceutically acceptable salt thereof.

49. A method of inhibiting CDK4 in a patient, comprising administering to the patient a compound of claim 1, or a pharmaceutically acceptable salt thereof.

50. A method of treating a disease or disorder associated with CDK4 in a patient, comprising administering to the patient a therapeutically effective amount of the compound of claim 1, or pharmaceutically acceptable salt thereof.

51. The method of claim 50, wherein the disease or disorder is cancer.

52. The method of claim 51, wherein the cancer is selected from breast cancer, ovarian cancer, a gynecological cancer, a gastrointestinal cancer, prostate cancer, melanoma, colorectal cancer, endometrial cancer, esophageal cancer, gastric cancer, liposarcoma, Ewing sarcoma, or melanoma.

53. The method of claim 50, wherein the administering further comprises administering one or more additional pharmaceutical agents.

54. The method of claim 53, wherein the one or more additional pharmaceutical agents is an aromatase inhibitor, luteinizing hormone-releasing hormone (LHRH) agonist, an anti-androgen, therapy targeting the PI3K/AKT/mTOR (PAM) pathway, a selective estrogen receptor degrader (SERD), a CDK2 inhibitor, a CDK4/6 inhibitor, a CDK4 inhibitor, or VEGF-A inhibitor, or a combination thereof.
